(19) United States
(12) Patent Application Publication
(10) Pub. No.: US 2024/0052349 A1
(43) Pub. Date: Feb. 15, 2024

(54) TARGETED CONJUGATES COMPRISING MODIFIED SIRNA

(71) Applicant: ARBUTUS BIOPHARMA CORPORATION, Warminster, PA (US)

(72) Inventors: Owen M. DALY, Burnaby (CA); Amy C. H. LEE, Burnaby (CA); Michael J. SOFIA, Doylestown, PA (US); Emily P. THI, Horsham, PA (US)

(73) Assignee: ARBUTUS BIOPHARMA CORPORATION, Warminster, PA (US)

(21) Appl. No.: 18/035,695

(22) PCT Filed: Nov. 5, 2021
(86) PCT No.: PCT/US2021/058232

§ 371 Date: May 5, 2023

Related U.S. Application Data
(60) Provisional application No. 63/110,837, filed on Nov. 6, 2020.

(51) Int. Cl. C12N 15/113; A61K 47/54; A61K 45/06; A61P 31/20

(52) U.S. Cl. CPC C12N 15/1131; A61K 47/549; A61K 45/06; A61P 31/20

(57) ABSTRACT

The invention provides certain nucleic acids (e.g., double stranded siRNA molecules), as well as conjugates that comprise a targeting moiety, an siRNA, and optional linking groups. The conjugates are useful to target siRNA.

### CROSS-REFERENCE TO RELATED APPLICATION(S)

This patent application claims the benefit of priority of U.S. application Ser. No. 63/110,837, filed Nov. 6, 2020, which application is herein incorporated by reference.

### BACKGROUND

Nucleic acids, including siRNA, are useful as therapeutic agents. Currently there is a need for compositions and methods that can be used to deliver (e.g., target) siRNA, in living subjects.

### BRIEF SUMMARY

The invention provides conjugate of Formula (I):

R¹-L-R² (I)

or a salt thereof, wherein:
R¹ is a targeting ligand that comprises one or more saccharide groups;
L is an optional linker; and
R² is an siRNA molecule that comprises at least one unlocked nucleic acid (UNA) of the following formula:

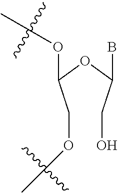

wherein B is a nucleobase.

The invention also provides synthetic intermediates and methods disclosed herein that are useful to prepare compounds of formula I.

Other objects, features, and advantages of the present invention will be apparent to one of skill in the art from the following detailed description and figures.

### BRIEF DESCRIPTION OF THE FIGURES

FIG. 1: shows data described in Example 2.

FIG. 2: shows data described in Example 3.

FIG. 3: shows data described in Example 4.

FIG. 4: shows data described in Example 5.

FIG. 5: shows data described in Example 6.

### DETAILED DESCRIPTION

As used herein, the following terms have the meanings ascribed to them unless specified otherwise.

The term “conjugate” as used herein includes compounds of formula (I) that comprise an siRNA molecule that comprises at least one unlocked nucleic acid (UNA) linked to a targeting ligand. Thus, the terms compound and conjugate may be used herein interchangeably.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and is preferably about 20-24, 21-22, or 21-23 (duplex) nucleotides in length. siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand. The siRNA used herein include at least one UNA.

In certain embodiments, the 5′ and/or 3′ overhang on one or both strands of the siRNA comprises 1-4 (e.g., 1, 2, 3, or 4) modified and/or unmodified deoxythymidine (t or dT) nucleotides. 1-4 (e.g., 1, 2, 3, or 4) modified (e.g., 2′OMe) and/or unmodified uridine (U) ribonucleotides, and/or 1-4 (e.g., 1, 2, 3, or 4) modified (e.g., 2′OMe) and/or unmodified ribonucleotides or deoxyribonucleotides having complementarity to the target sequence (e.g., 3′overhang in the antisense strand) or the complementary strand thereof (e.g., 3′ overhang in the sense strand).

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA. 99:9942-9947 (2002); Calegari et al., Proc. Natl. Acad. Sci. USA. 99:14236 (2002); Byrom et al., Ambion TechNotes, 10(1):4-6 (2003); Kawasaki et al., Nucleic Acids Res., 31:981-987 (2003); Knight et al., Science. 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA of the invention to silence, reduce, or inhibit expression of a target gene. To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “synthetic activating group” refers to a group that can be attached to an atom to activate that atom to allow it to form a covalent bond with another reactive group. It is understood that the nature of the synthetic activating group may depend on the atom that it is activating. For example, when the synthetic activating group is attached to an oxygen atom, the synthetic activating group is a group that will activate that oxygen atom to form a bond (e.g. an ester, carbamate, or ether bond) with another reactive group. Such synthetic activating groups are known. Examples of synthetic activating groups that can be attached to an oxygen atom include, but are not limited to, acetate, succinate, triflate, and mesylate. When the synthetic activating group is attached to an oxygen atom of a carboxylic acid, the synthetic activating group can be a group that is derivable from a known coupling reagent (e.g. a known amide coupling reagent). Such coupling reagents are known. Examples of such coupling reagents include, but are not limited to, N,N′-Dicyclohexylcarbodimide (DCC), hydroxybenzotriazole (HOBt), N-(3-Dimethylaminopropyl)-N′-ethylcarbonate (EDC), (Benzotriazol-1-yloxy)tris(dimethylamino)phosphonium hexafluorophosphate (BOP), benzotriazol-1-yl-oxytripyrrolidinophosphonium hexafluorophosphate (PyBOP) or O-benzotriazol-1-yl-N,N,N′,N′-tetramethyluronium hexafluorophosphate (HBTU).

An “effective amount” or “therapeutically effective amount” of a therapeutic nucleic acid such as siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization. ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Accordingly, in the context of this invention, the terms “polynucleotide” and “oligonucleotide” refer to a polymer or oligomer of nucleotide or nucleoside monomers consisting of naturally occurring bases, sugars and intersugar (backbone) linkages. The terms “polynucleotide” and “oligonucleotide” also include polymers or oligomers comprising non-naturally occurring monomers, or portions thereof; which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91 98 (1994)).

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

As used herein, the term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkenyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane (including straight and branched alkanes), as exemplified by —CH₂CH₂CH₂CH₂— and —CH(CH₃)CH₂CH₂—.

The term “cycloalkyl,” “carbocyclic,” or “carbocycle” refers to hydrocarbon ringsystem having 3 to 20 overall number of ring atoms (e.g., 3-20 membered cycloalkyl is a cycloalkyl with 3 to 20 ring atoms, or C₃₋₂₀ cycloalkyl is a cycloalkyl with 3-20 carbon ring atoms) and for a 3-5 membered cycloalkyl being fully saturated or having no more than one double bond between ring vertices and for a 6 membered cycloalkyl or larger being fully saturated or having no more than two double bonds between ring vertices. As used herein, “cycloalkyl,” “carbocyclic,” or “carbocycle” is also meant to refer to bicyclic, polycyclic and spirocyclic hydrocarbon ring system, such as, for example, bicyclo[2.2.1]heptane, pinane, bicyclo[2.2.2]octane, adamantane, norborene, spirocyclic C₅₋₁₂ alkane, etc. As used herein, the terms, “alkenyl,” “alkynyl,” “cycloalkyl,”, “carbocycle,” and “carbocyclic,” are meant to include mono and polyhalogenated variants thereof.

The term “heterocycloalkyl,” “heterocyclic,” or “heterocycle” refers to a saturated or partially unsaturated ring system radical having the overall having from 3-20 ring atoms (e.g., 3-20 membered heterocycloalkyl is a heterocycloalkyl radical with 3-20 ring atoms, a C₂₋₁₉ heterocycloalkyl is a heterocycloalkyl having 3-10 ring atoms with between 2-19 ring atoms being carbon) that contain from one to ten heteroatoms selected from N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and the like A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The terms “alkoxy,” and “alkylthio”, are used in their conventional sense, and refer to those alkyl groups attached to the remainder of the molecule via an oxygen atom (“oxy”) or thio group, and further include mono- and poly-halogenated variants thereof.

The terms “halo” or “halogen,” by themselves or as part of another substituent, mean, unless otherwise stated, a fluorine, chlorine, bromine, or iodine atom. The term “(halo)alkyl” is meant to include both a “alkyl” and “haloalkyl” substituent. Additionally, the term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, difluoromethyl, and the like.

The term “aryl” means a carbocyclic aromatic group having 6-14 carbon atoms, whether or not fused to one or more groups. Examples of aryl groups include phenyl, naphthyl, biphenyl and the like unless otherwise stated.

The term “heteroaryl” refers to aryl ring(s) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furl, thienyl and the like.

The term saccharide includes monosaccharides, disaccharides and trisaccharides. The term includes glucose, sucrose fructose, galactose and ribose, as well as deoxy sugars such as deoxyribose and amino sugar such as galactosamine. Saccharide derivatives can conveniently be prepared as described in International Patent Applications Publication Numbers WO 96/34005 and 97/03995. A saccharide can conveniently be linked to the remainder of a compound of formula I through an ether bond, a thioether bond (e.g. an S-glycoside), an amine nitrogen (e.g., an N-glycoside), or a carbon-carbon bond (e.g. a C-glycoside). In one embodiment the saccharide can conveniently be linked to the remainder of a compound of formula I through an ether bond. In one embodiment the term saccharide includes a group of the formula:

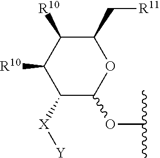

wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)allyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy. In another embodiment the saccharide can be selected from the group consisting of:

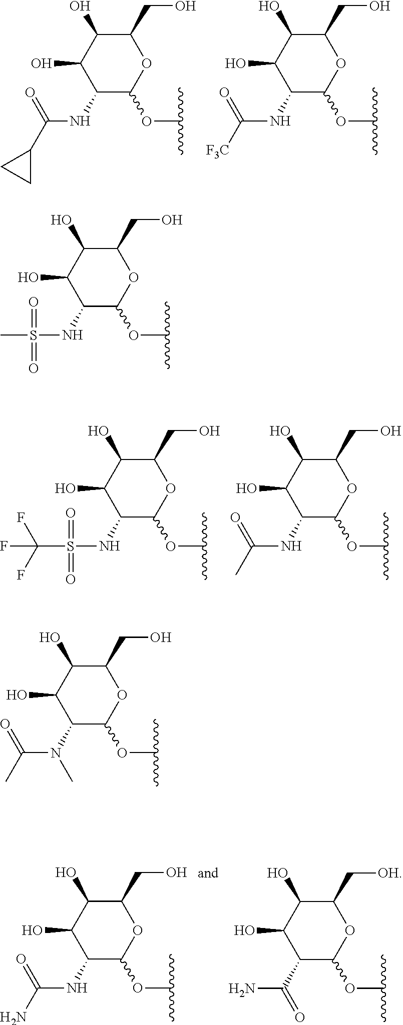

In another embodiment the saccharide can be:

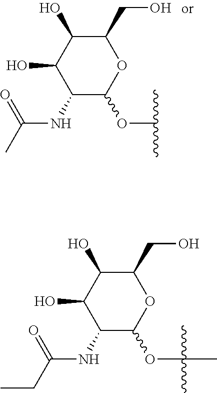 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

The term “animal” includes mammalian species, such as a human, mouse, rat, dog, cat, hamster, guinea pig, rabbit, livestock, and the like.

In one embodiment, the unlocked nucleic acid (UNA) has the following formula:

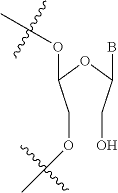

wherein B is a nucleobase. In one embodiment, B is an unnatural nucleobase. In one embodiment, B is a natural nucleobase. In one embodiment, B is a nucleobase that comprises a purine or a pyrimidine. In one embodiment, B is a nucleobase selected from:

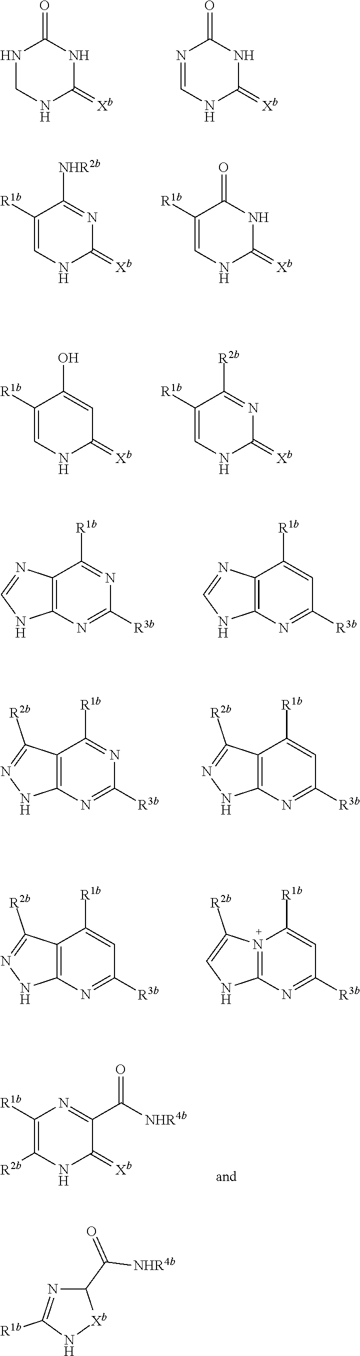

wherein:
R^{1b} is selected from the group consisting of H. Me, F, Cl, Br, I, OH, NH₂, SH, OMe, NO₂, NHOH, NHOMe, NHNH₂, C═ONH₂, C₁-C₈ alkyl, and 5- or 6-membered heteroaryl;
R^{2b} is selected from the group consisting of H, OH, OMe, NH₂, NHMe, C═ONH₂, C₁-C₈ alkyl, and 5- or 6-membered heteroaryl;
R^{3b} is selected from the group consisting of H, F, Cl, Br, I, OH, S, NH₂, SH, OMe, NO₂, NHOH, NHOMe, NHNH₂, C═ONH₂, C₁-C₈ alkyl, and 5- or 6-membered heteroaryl;
R^{4b} is selected from the group consisting of H, NH₂ and C₁-C₈ alkyl; and
X^{b} is NR^{2b}, O or S.

In one embodiment, B is selected from adenine (A), cytosine (C), guanine (G) and uracil (U).

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfate, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

The term “fusogenic” refers to the ability of a lipid particle, such as a SNALP, to fuse with the membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

“Distal site,” as used herein, refers to a physically separated site, which is not limited to an adjacent capillary bed, but includes sites broadly distributed throughout an organism.

“Serum-stable” in relation to nucleic acid-lipid particles such as SNALP means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of lipid particles that leads to a broad biodistribution of an active agent such as an siRNA within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent such as an siRNA directly to a target site within an organism. For example, an agent can be locally delivered by direct injection into a disease site, other target site, or a target organ such as the liver, heart, pancreas, kidney, and the like.

When used herein to describe the ratio of lipid:siRNA, the term “lipid” refers to the total lipid in the particle.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. Unless otherwise specifically noted, when a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Unless stated otherwise herein, the term “about”, when used in connection with a value or range of values, means plus or minus 5% of the stated value or range of values.

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein. siRNA, including siRNA with at least one UNA, and conjugates thereof, can be prepared, e.g., using methods described in International Publication Numbers WO 2017/177326 and WO 2018/191278.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983): Sambrook et al., supra: Ausubel et al., supra), as are PCR methods (see, U.S. Pat. Nos. 4,683,195 and 4,681,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning. A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677 2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, CA). However, a larger or smaller scale of synthesis is also within the scope of this invention. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Embodiments of the Invention

One aspect of the invention is a compound of formula I, as set forth about in the Summary of the Invention, or a salt thereof.

In one embodiment R¹ is —C(H)₍₃₋ₚ₎(L³-saccharide)ₚ, wherein each L³ is independently a linking group; p is 1, 2, or 3; and saccharide is a monosaccharide or disaccharide.

In one embodiment the saccharide is:

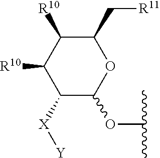

wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴. R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;
or a salt thereof.

In one embodiment the saccharide is selected from the group consisting of:

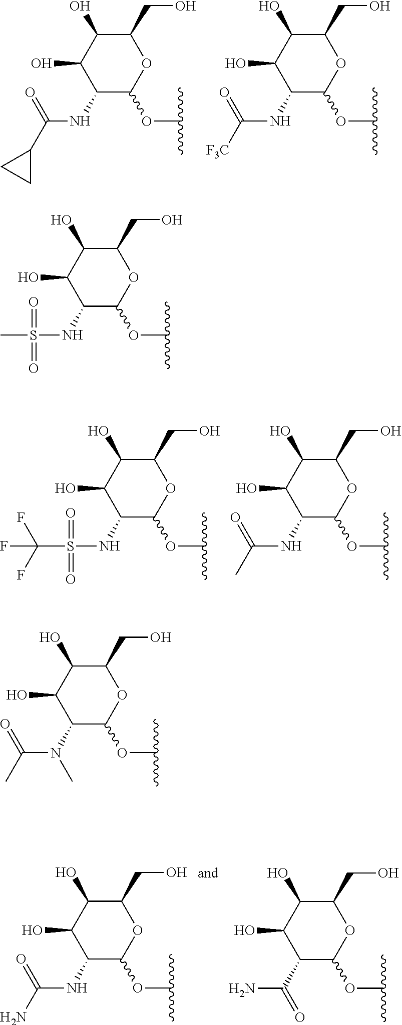

and salts thereof.

In one embodiment the saccharide is:

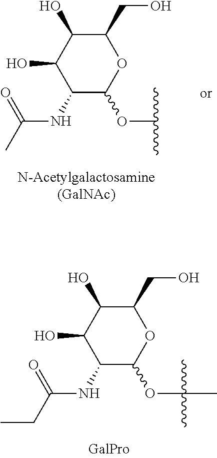 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₈)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)allyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L³ is:

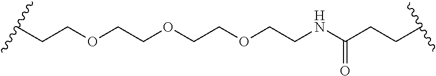 [SMILES: *CCOCCOCCOCCNC(=O)CC*]

or a salt thereof.

In one embodiment R¹ is:

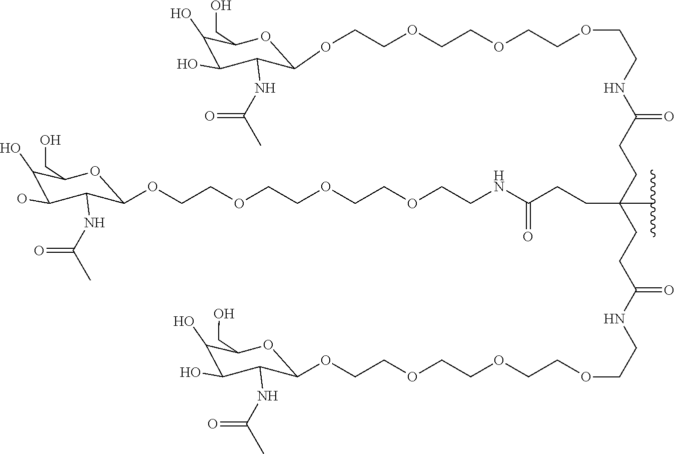 [SMILES: *C(CCC(=O)NCCOCCOCCOCCOC1OC(CO)C(O)C(O)CC1NC(C)=O)(CCC(=O)NCCOCCOCCOCCOC1OC(CO)C(O)C(O)CC1NC(C)=O)CCC(=O)NCCOCCOCCOCCOC1OC(CO)C(O)C(O)CC1NC(C)=O]

or a salt thereof.

In one embodiment R¹ is:

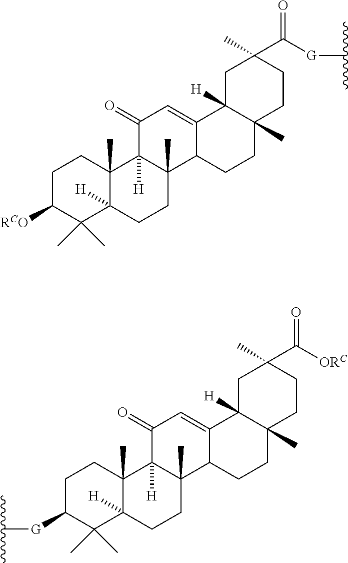

wherein G is —NH— or —O—;
R^{C} is hydrogen, (C₁-C₈)alkyl, (C₁-C₈)haloalkyl, (C₁-C₈)alkoxy, (C₁-C₆)alkanoyl, (C₃-C₂₀)cycloalkyl, (C₃-C₂₀)heterocycle, aryl, heteroaryl, monosaccharide, disaccharide or trisaccharide; and wherein the cycloalkyl, heterocyle, ary, heteroaryl and saccharide are optionally substituted with one or more groups independently selected from the group consisting of halo, carboxyl, hydroxyl, amino, (C₁-C₄)alkyl, (C₁-C₄)haloalkyl, (C₁-C₄)alkoxy and (C₁-C₄)haloalkoxy;
or a salt thereof.

In one embodiment R^{C} is:

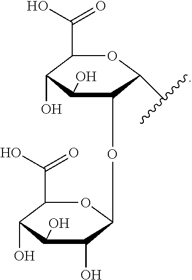

In one embodiment R¹ is:

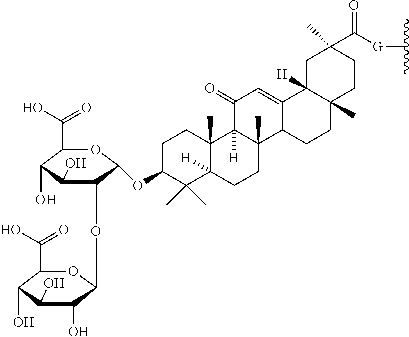

In one embodiment R^{C} is:

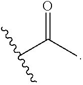 [SMILES: *C(C)=O]

In one embodiment G is —NH—.

In one embodiment R¹ is:

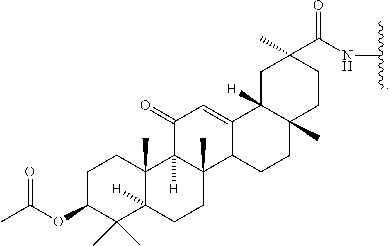

In one embodiment R¹ is:

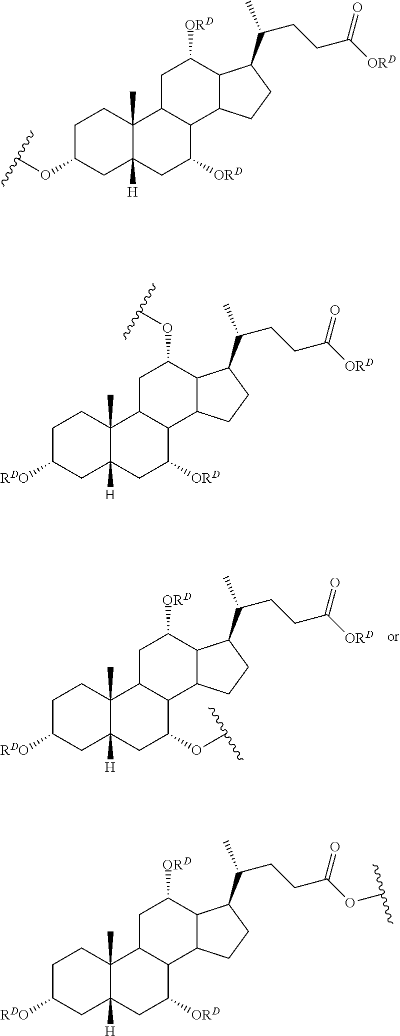

wherein each R^{D} is independently selected from the group consisting of hydrogen, (C₁-C₆)alkyl, (C₉-C₂₀)alkylsilyl, (R^{W})₃Si—, (C₂-C₆)alkenyl, tetrahydropyranyl, (C₁-C₆)alkanoyl, benzoyl, aryl(C₁-C₃)alkyl, TMTr (Trimethoxytrityl), DMTr (Dimethoxytrityl), MMTr (Monomethoxytrityl), and Tr (Trityl); and
each R^{W} is independently selected from the group consisting of (C₁-C₄)alkyl and aryl.

In one embodiment linking groups L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)allylthio, azido, cyano, nitro, halo, hydroxy, oxo (═O), carboxy, aryl, aryloxy, heteroaryl, and heteroaryloxy.

In one embodiment L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyan, nitro, halo, hydroxy, oxo (═O), carboxy, aryl, aryloxy, heteroaryl, and heteroaryloxy.

In one embodiment L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

In one embodiment L² is connected to R² through —O—.

In one embodiment L¹ is selected from the group consisting of:

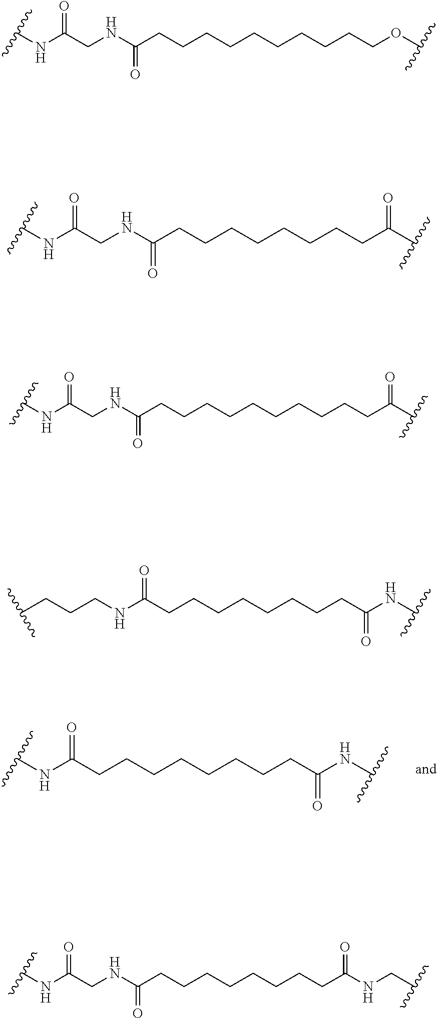 [SMILES: *NC(=O)CNC(=O)CCCCCCCCCCO* ; *NC(=O)CNC(=O)CCCCCCCCC(*)=O ; *NC(=O)CNC(=O)CCCCCCCCCCC(*)=O ; *CCCNC(=O)CCCCCCCCC(=O)N* ; *NC(=O)CCCCCCCCC(=O)N* ; *CNC(=O)CCCCCCCCC(=O)NCC(=O)N*]

In one embodiment L¹ is selected from the group consisting of:

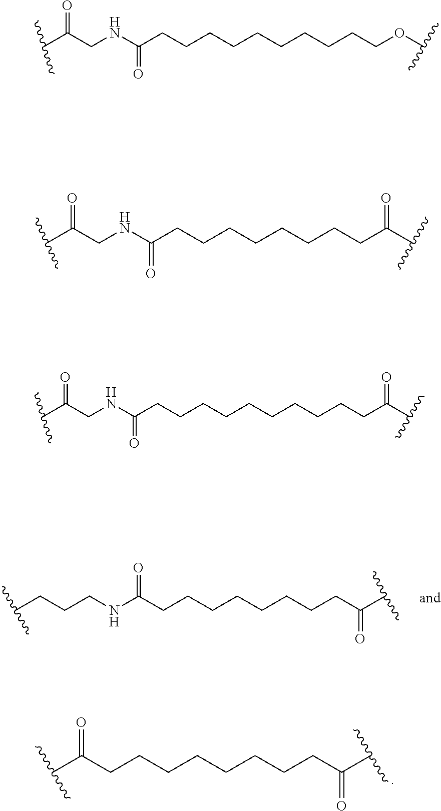 [SMILES: *OCCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(*)=O ; *C(=O)CCCCCCCCC(*)=O]

and salts thereof.

In one embodiment L² is —CH₂—O— or —CH₂—CH₂—O—.

In one embodiment a compound of formula II has the following formula (IIa):

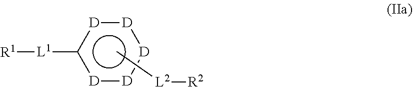

wherein:
each D is independently selected from the group consisting of

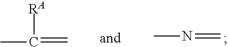

or a salt thereof.

In one embodiment a compound of formula (IIa) is selected from the group consisting of:

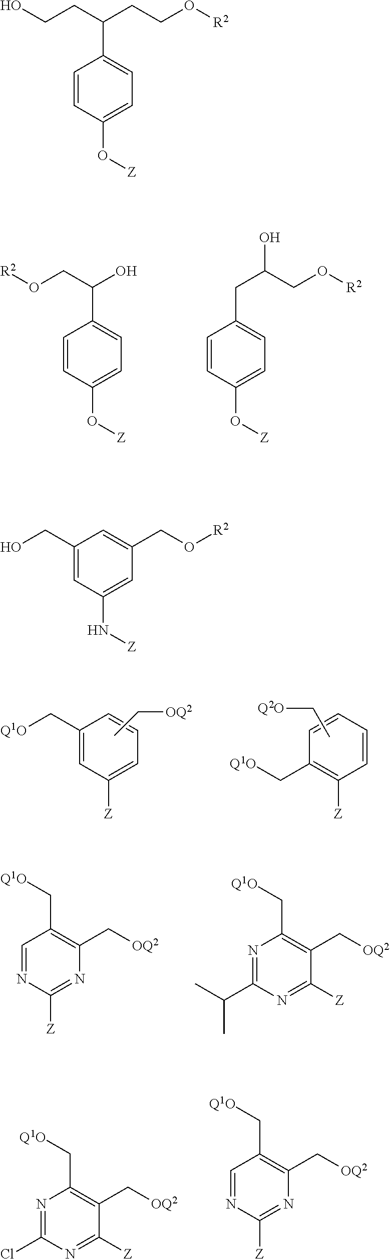

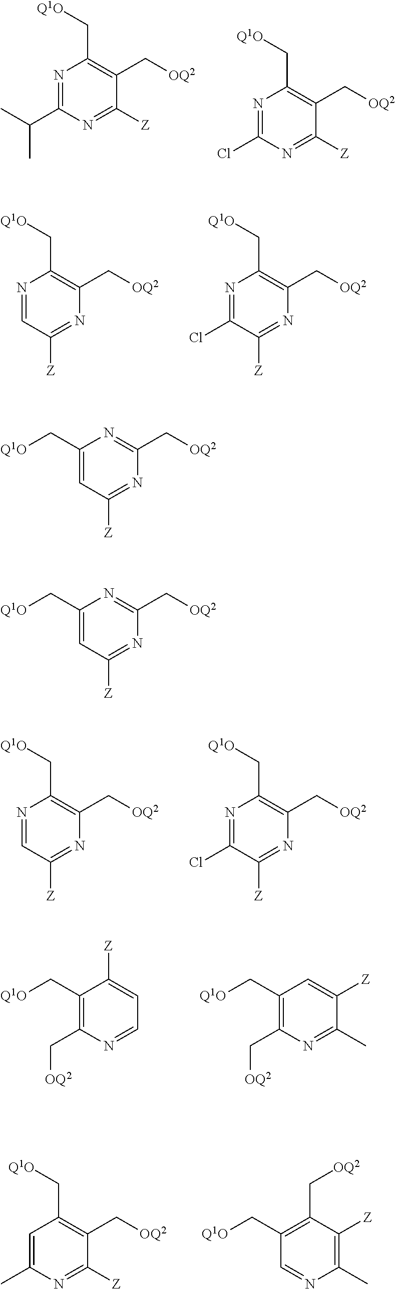

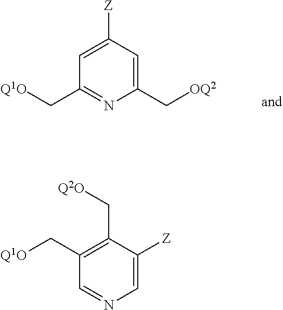

wherein:
Q¹ is hydrogen and Q² is R²; or Q¹ is R² and Q² is hydrogen;
Z is -L¹-R¹;
and salts thereof.

In one embodiment a compound of formula I has the following formula (IIIb):

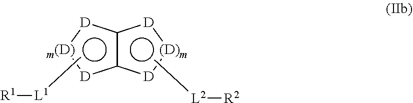

wherein:
each D is independently selected from the group consisting of

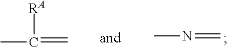

each m is independently 1 or 2; or a salt thereof.

In one embodiment a compound of formula Ib is selected from the group consisting of:

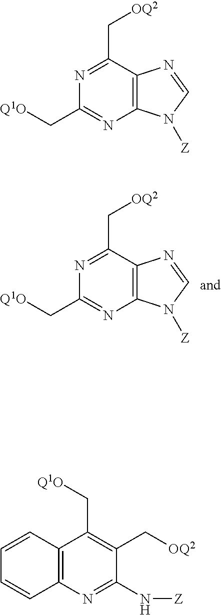

wherein:
Q¹ is hydrogen and Q² is R², or Q¹ is R² and Q² is hydrogen;
Z is -L¹-R¹;
and salts thereof.

In one embodiment a compound of formula I has the following formula (IIc):

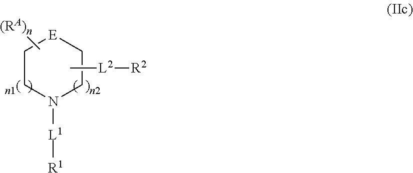

wherein E is —O— or —CH₂—;
n is selected from the group consisting of 0, 1, 2, 3, and 4; and
n1 and n2 are each independently selected from the group consisting of 0, 1, 2, and 3;
or a salt thereof.

In certain embodiments a compound of formula (IIc) is selected from the group consisting of:

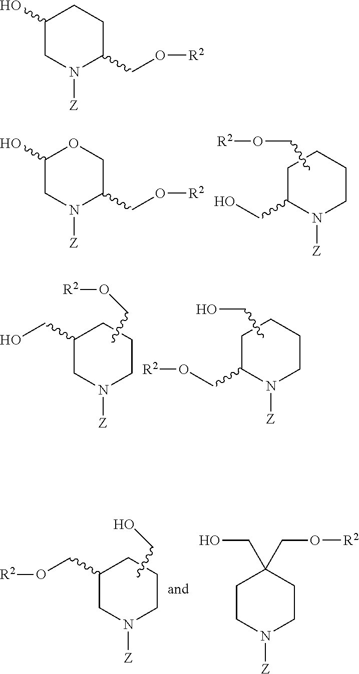

wherein Z is -L¹-R¹;
and salts thereof.

In one embodiment the -A-L²-R² moiety is:

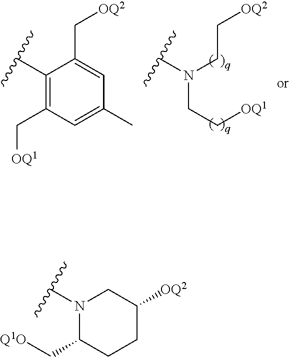

wherein:
Q¹ is hydrogen and Q² is R²; or Q¹ is R² and Q² is hydrogen; and
each q is independently 0, 1, 2, 3, 4 or 5;
or a salt thereof.

In one embodiment a compound of formula (I) is selected from the group consisting of:

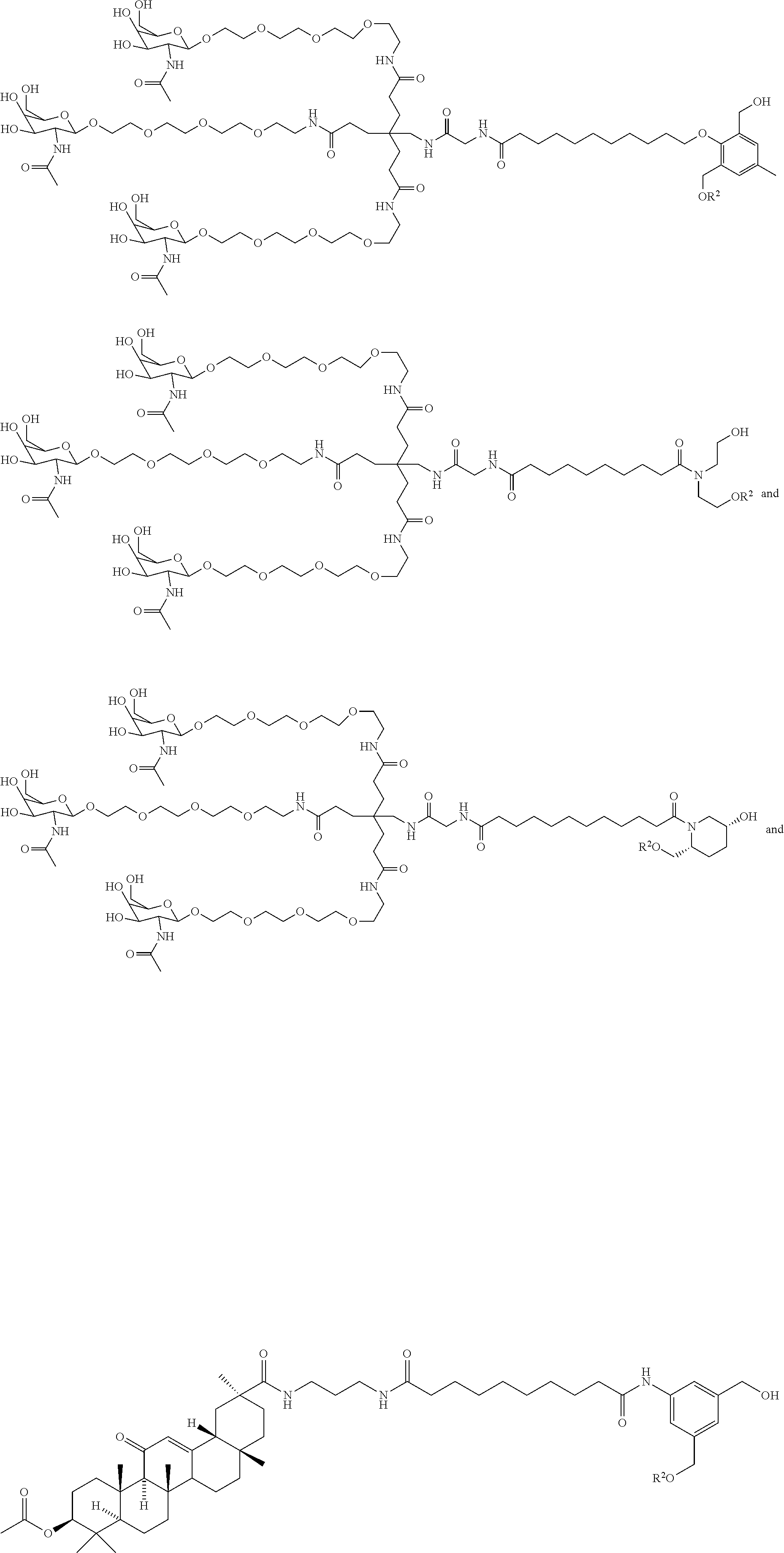

and salts thereof.

In one embodiment R¹ is selected from the group consisting of:

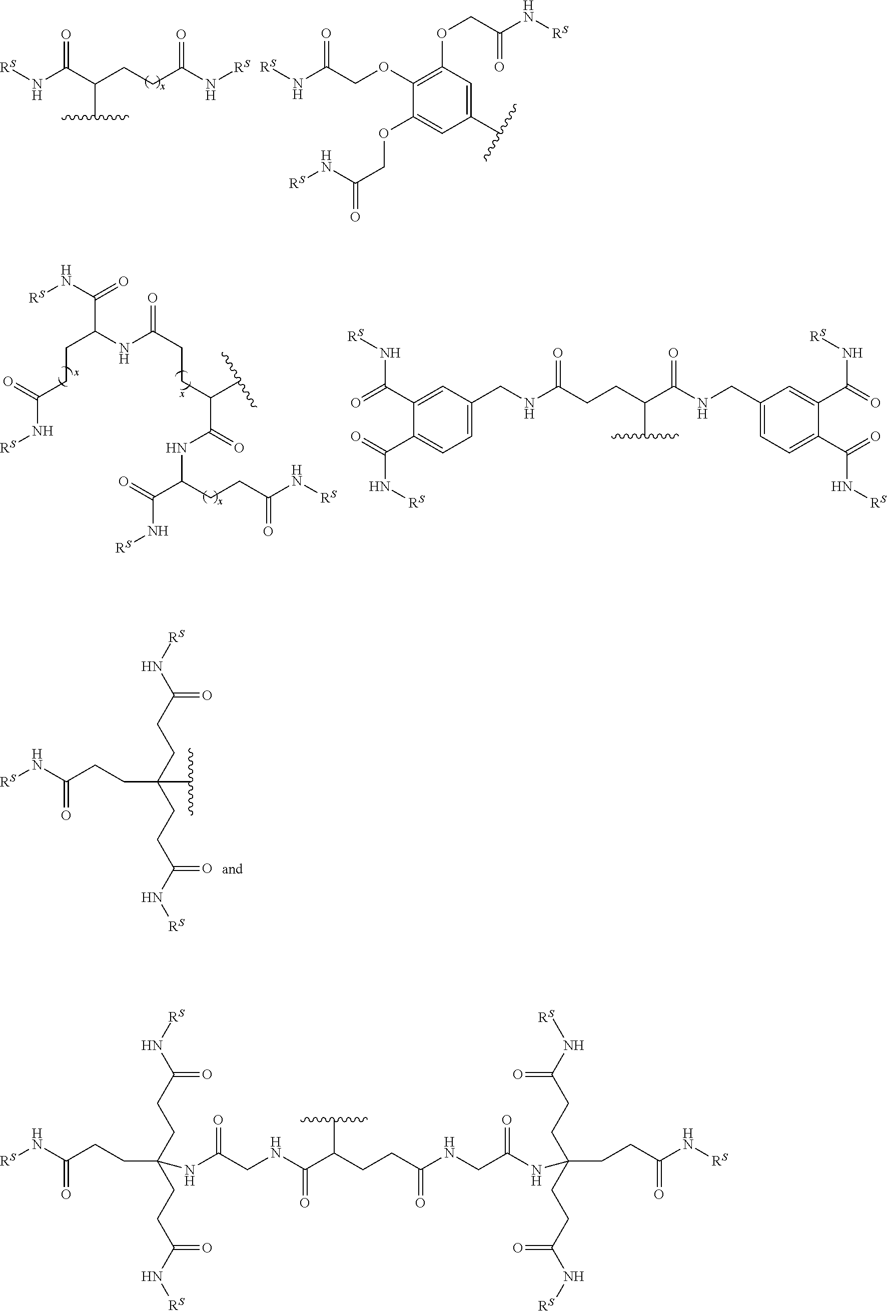

wherein R^{S} is

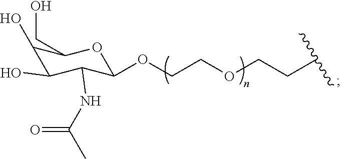

n is 2, 3, or 4;
x is 1 or 2.

In one embodiment L¹ is selected from the group consisting of:

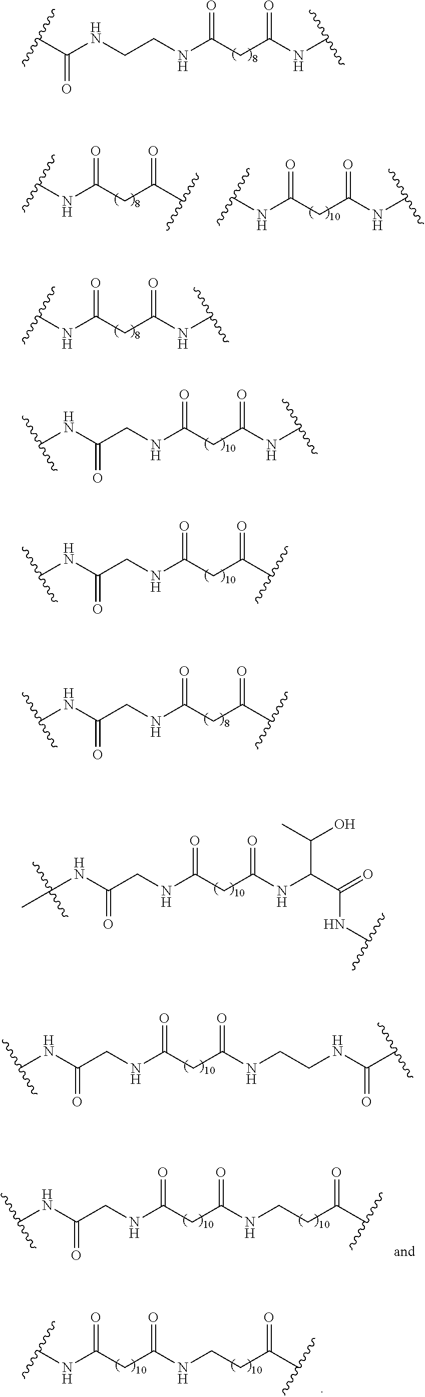

In one embodiment L¹ is absent, phenyl, pyrrolidinyl, or cyclopentyl.

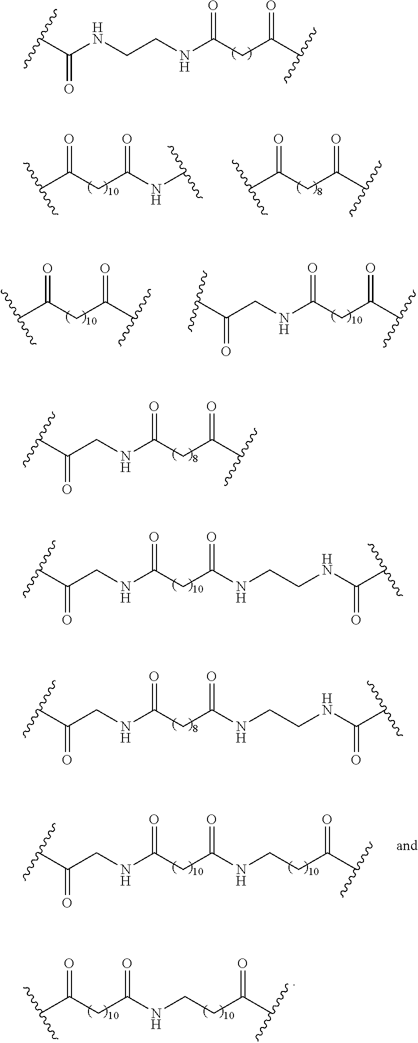

In one embodiment L² is C₁₋₄ alkylene-O— that is optionally substituted with hydroxy.

In one embodiment L² is —CH₂O—, —CH₂CH₂O—, or —CH(OH)CH₂O—.

In one embodiment each R^{A} is independently hydroxy or C₁₋₈ alkyl that is optionally substituted with hydroxyl.

In one embodiment each R^{A} is independently selected from the group consisting of hydroxy, methyl and —CH₂OH.

In one embodiment a compound of formula I has the following formula (IIg):

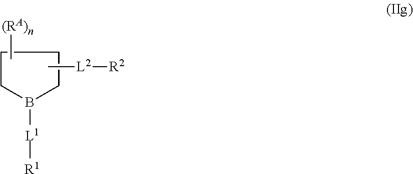

wherein B is —N— or —CH—;
L¹ is absent or —NH—;
L² is C₁₋₄ alkylene-O— that is optionally substituted with hydroxyl or halo;
n is 0, 1, or 2;
or a salt thereof.

In one embodiment a compound of formula I has the following formula (IIg):

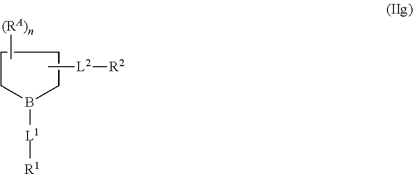

wherein B is —N— or —CH—;
L¹ is absent or —NH—;
L² is C₁₋₄ alkylene-O— that is optionally substituted with hydroxyl or halo;
n is 0, 1, 2, 3, 4, 5, 6, or 7;
or a salt thereof.

In one embodiment a compound of formula I has the following formula (IIg):

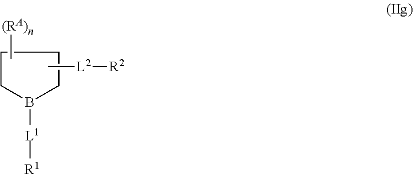

wherein B is —N— or —CH—;
L¹ is absent or —NH—,
L² is C₁₋₄ alkylene-O— that is optionally substituted with hydroxyl or halo;
n is 0, 1, 2, 3, or 4;
or a salt thereof.

In one embodiment a compound of formula (IIg) is selected from the group consisting of:

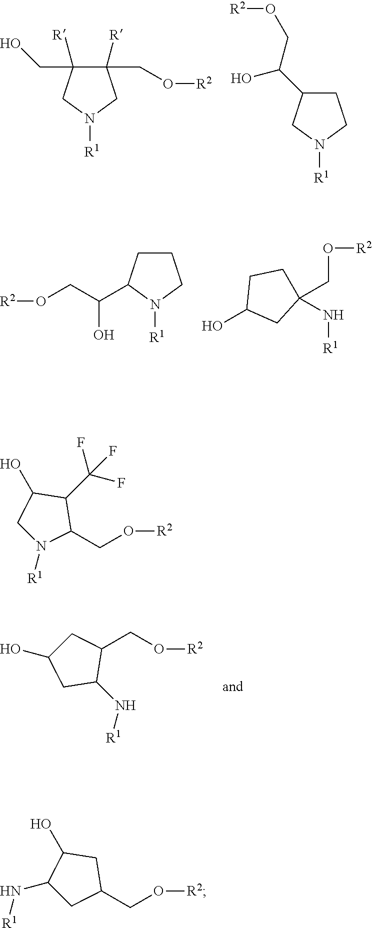

wherein R′ is C₁₋₉ alkyl, C₂₋₉ alkenyl or C₂₋₉ alkenyl; wherein the C₁₋₉ alkyl, C₂₋₉ alkenyl or C₂₋₉ alkenyl are optionally substituted with halo or hydroxyl;
and salts thereof.

In one embodiment a compound of formula is selected from the group consisting of:

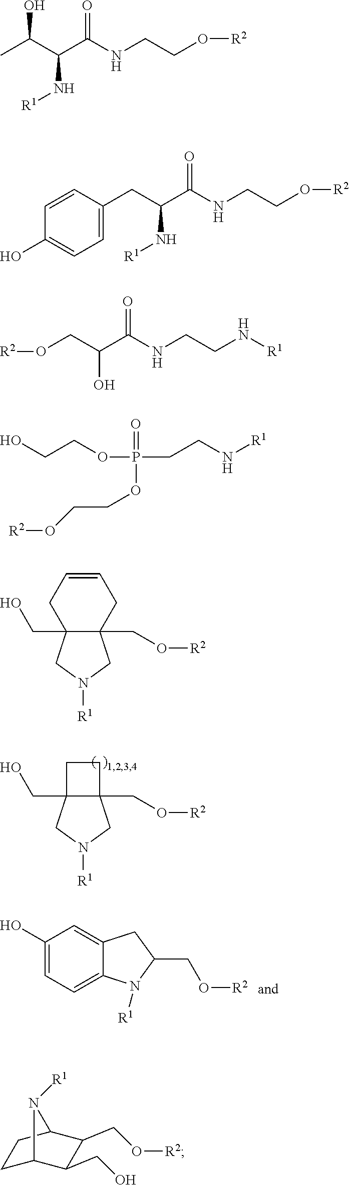

and salts thereof.

In one embodiment the compound of formula I or the salt thereof is selected from the group consisting of:

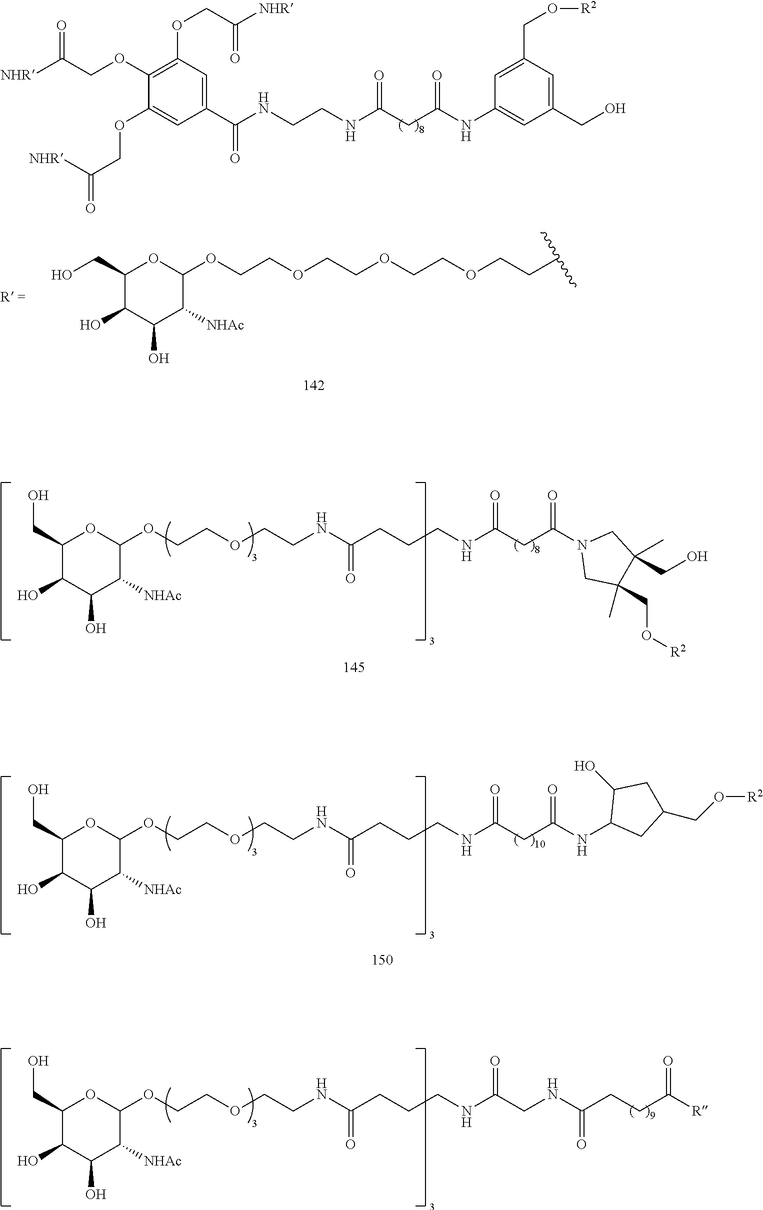

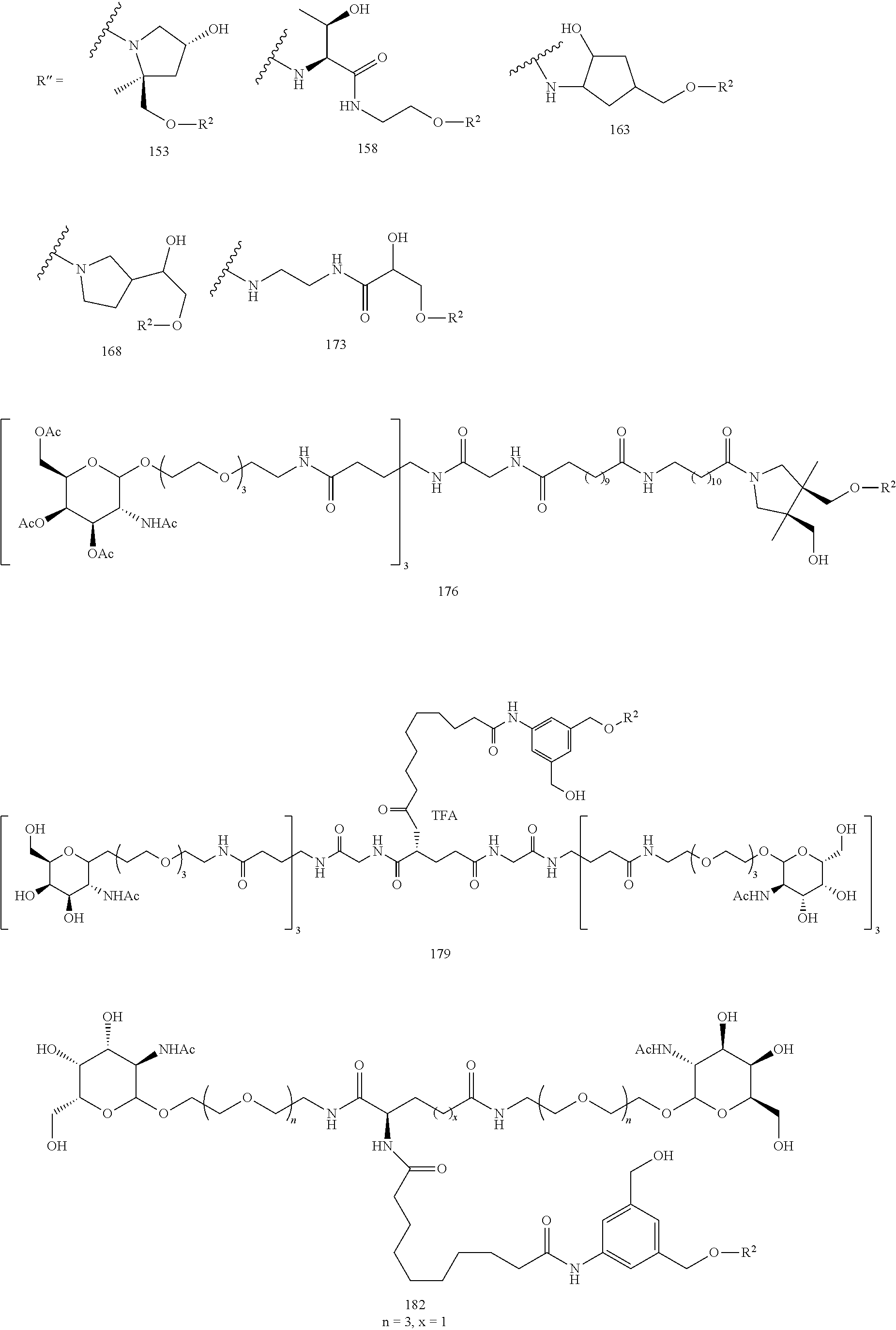

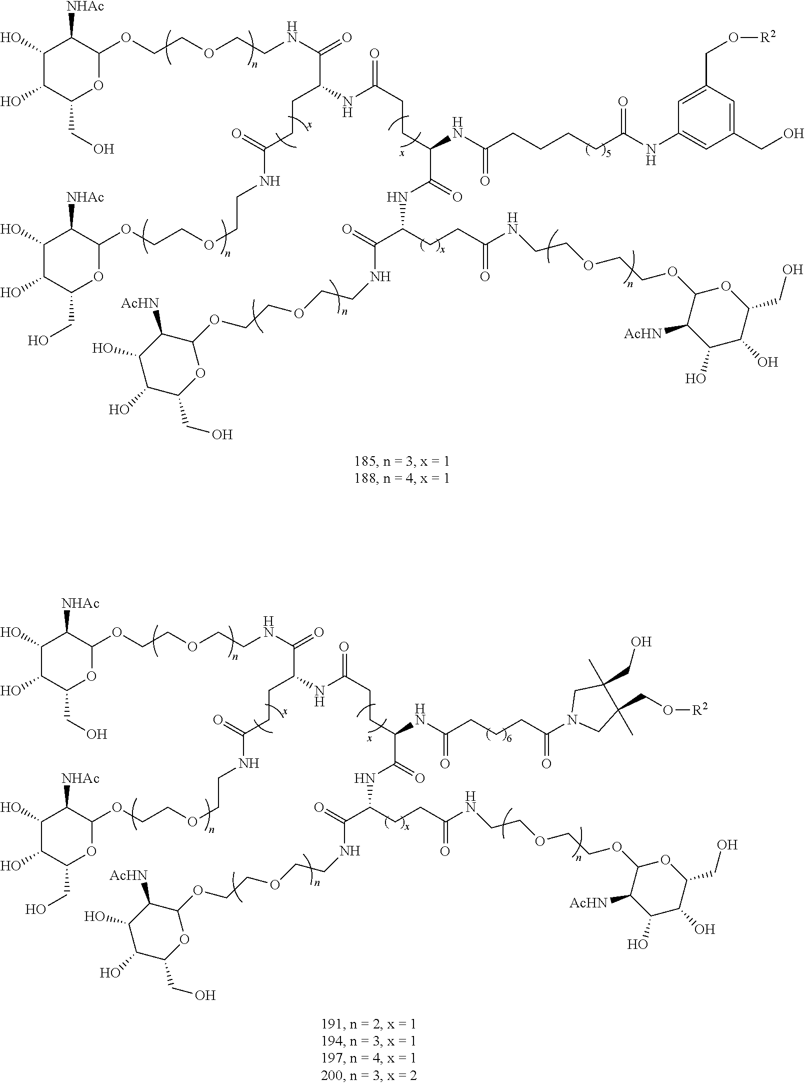

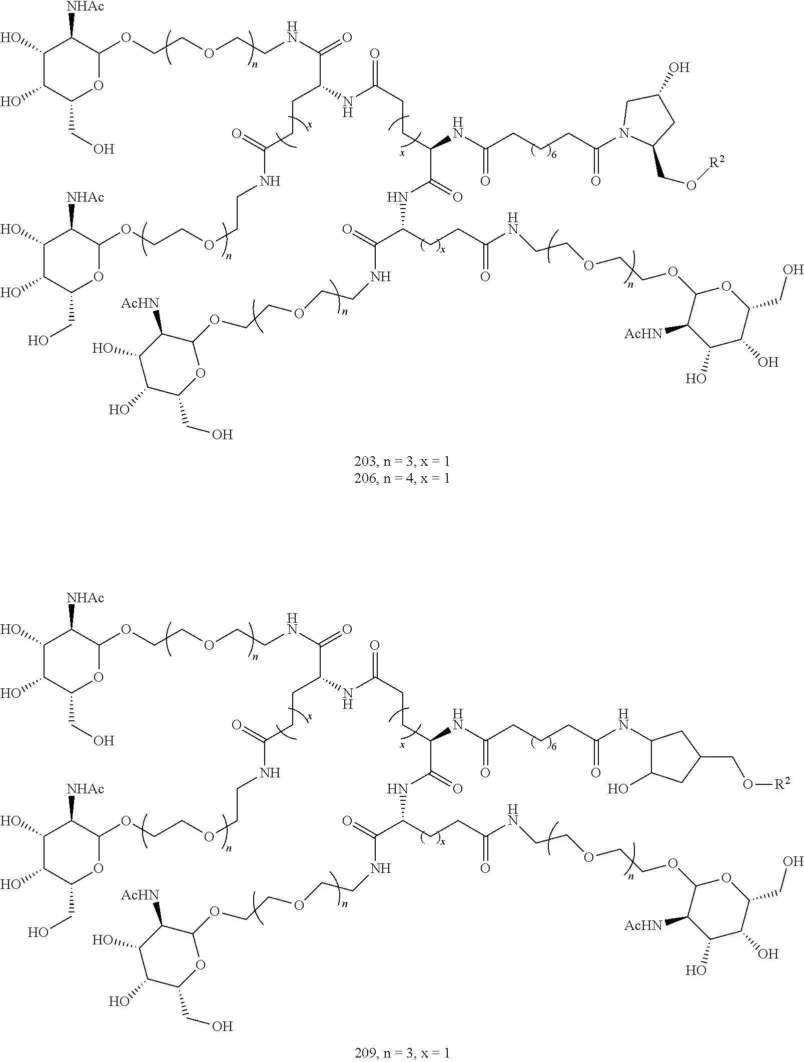

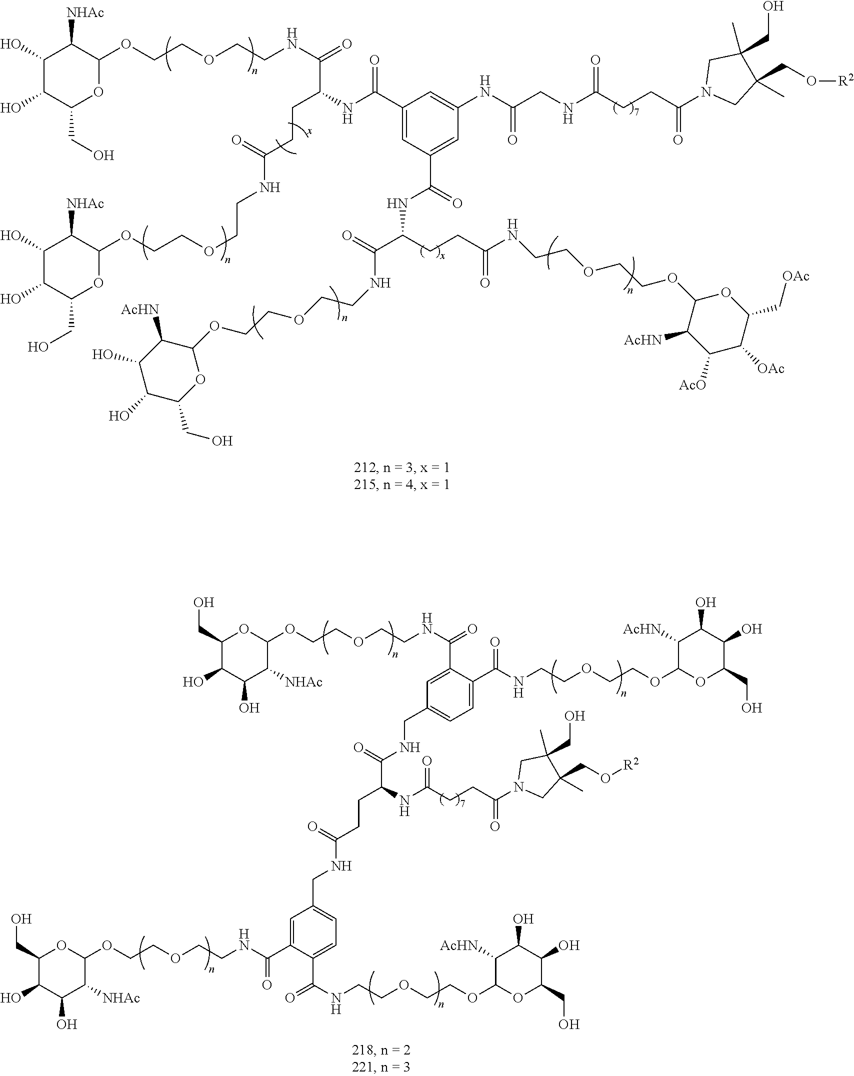

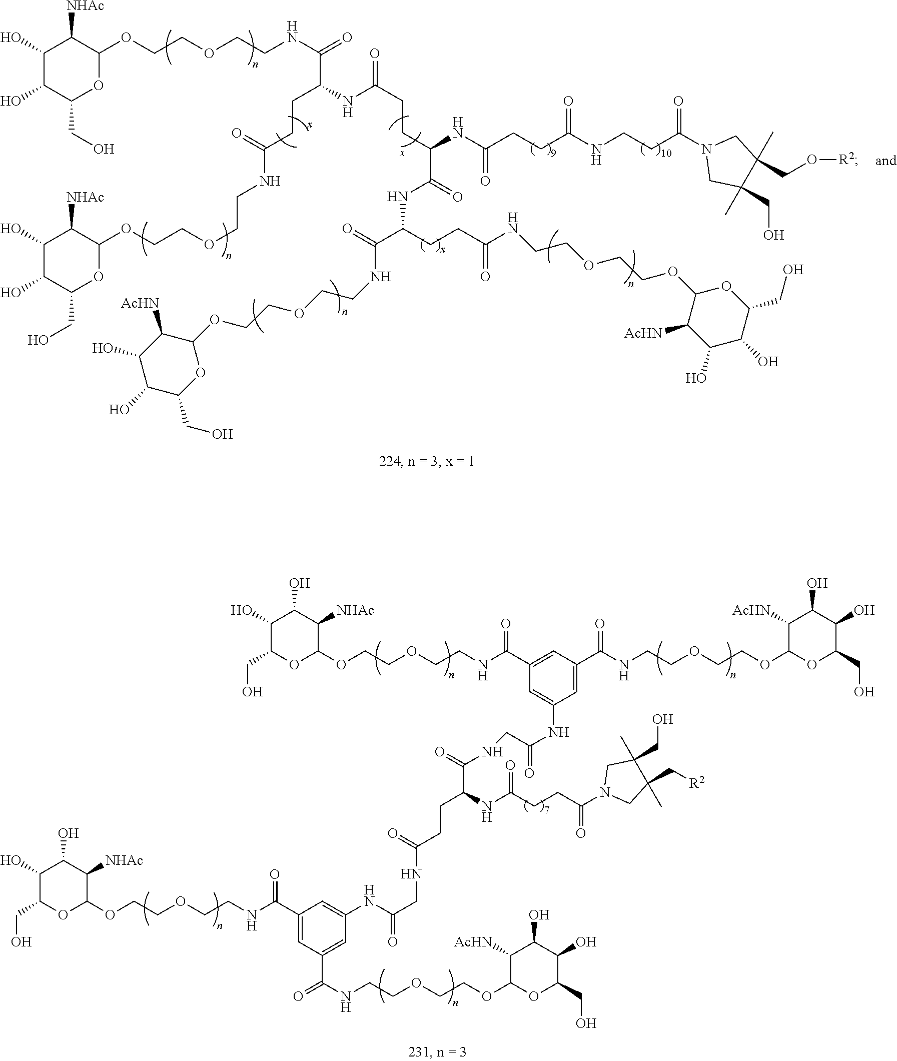

In one embodiment the compound of formula I or the salt thereof is selected from the group consisting of:

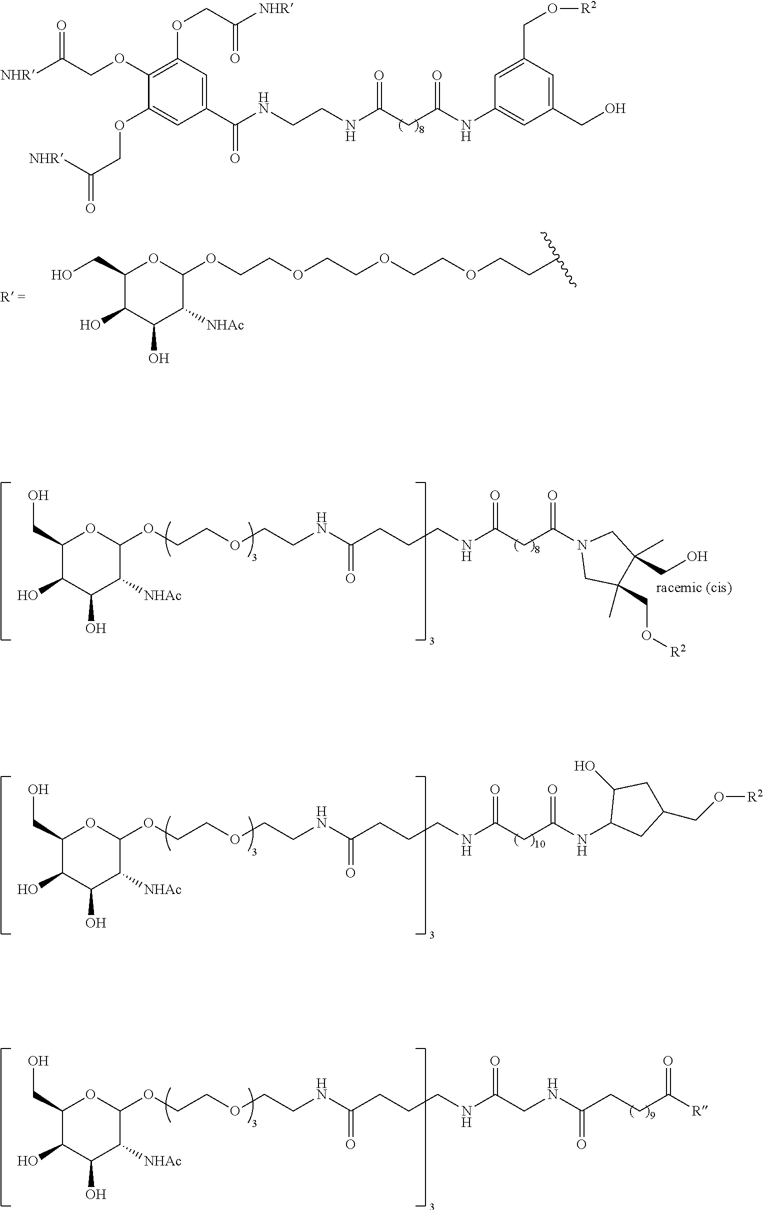

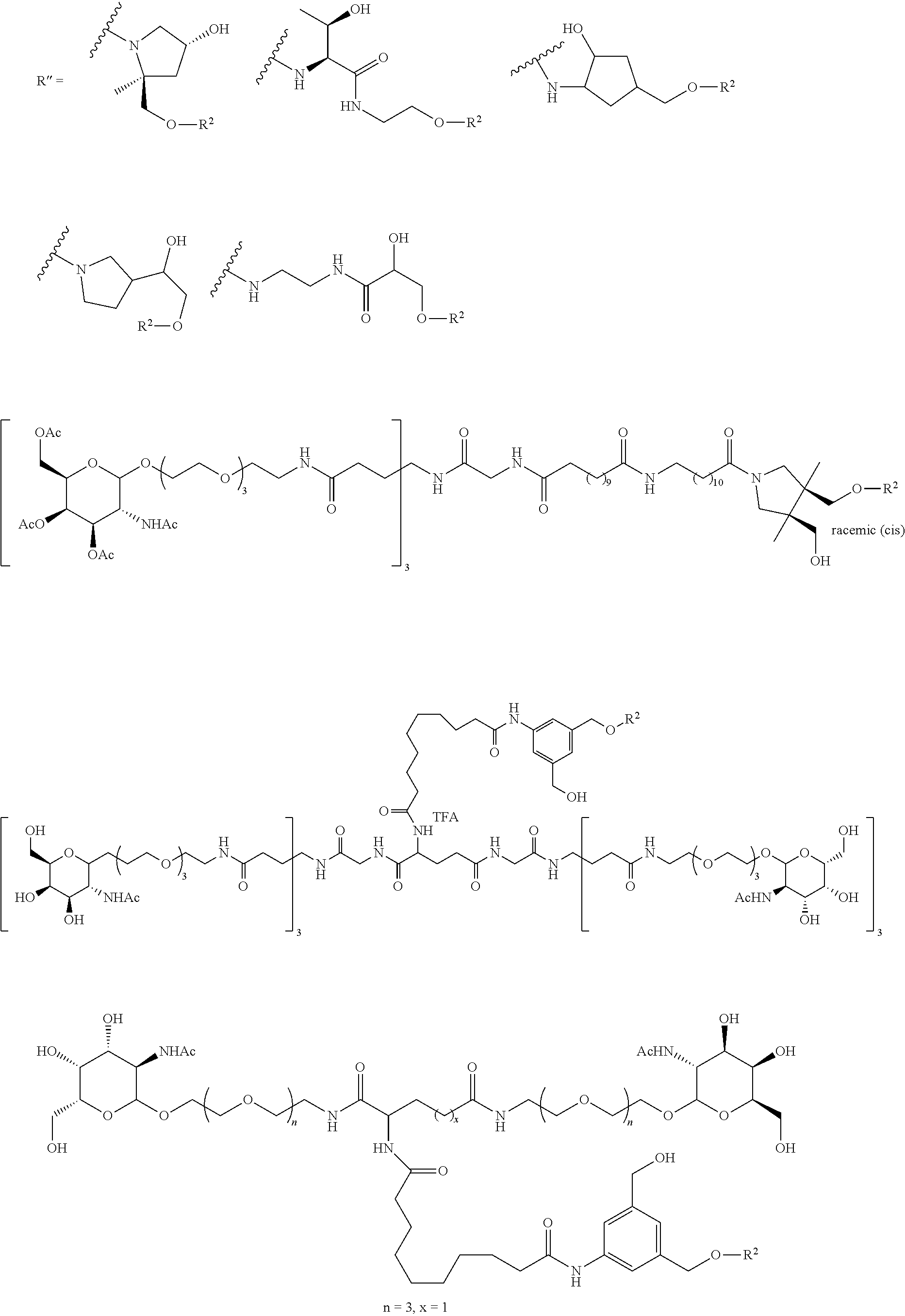

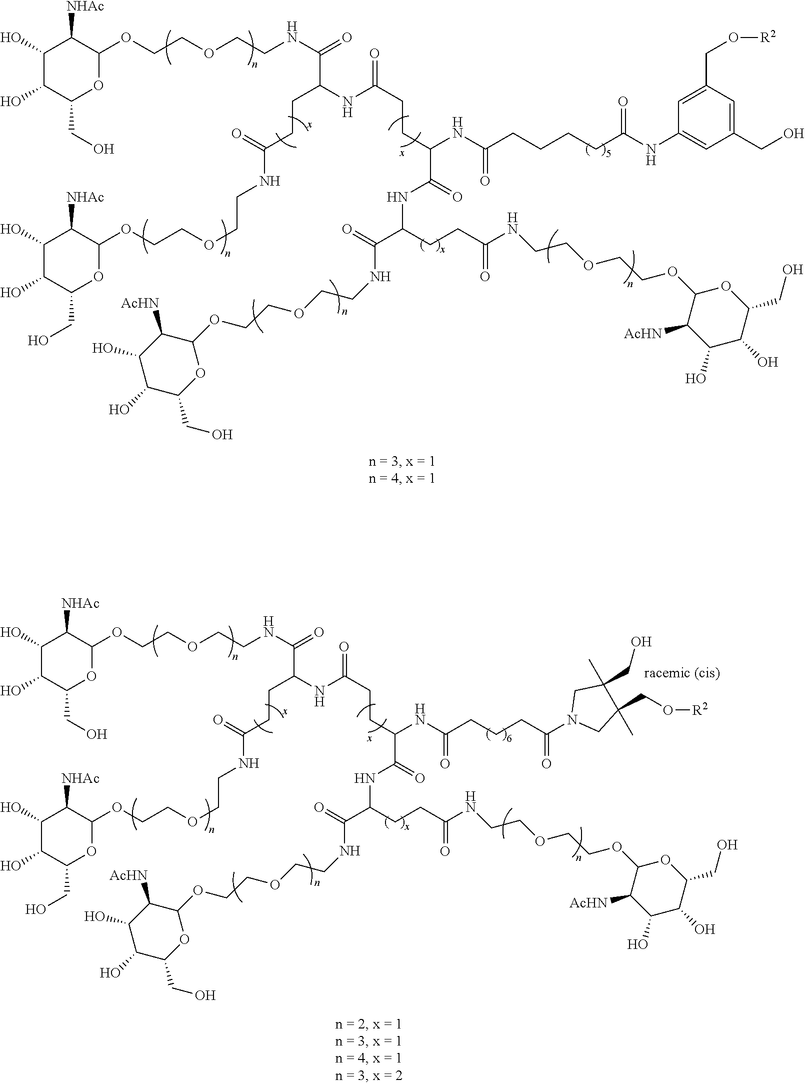

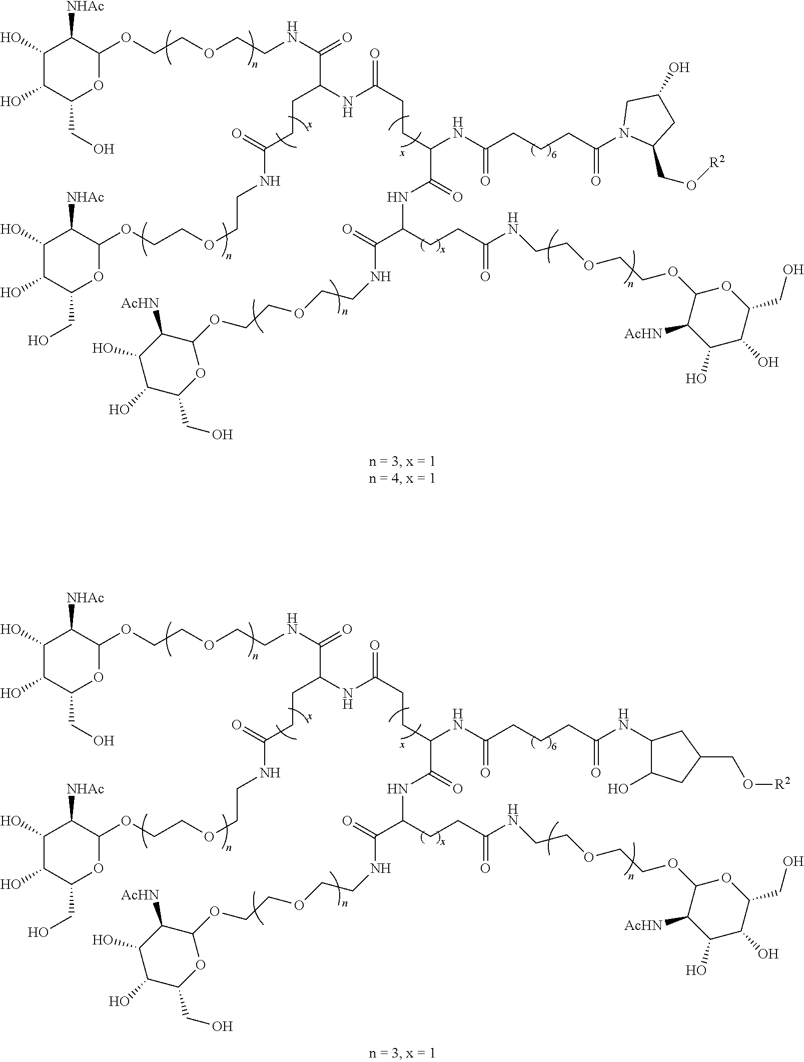

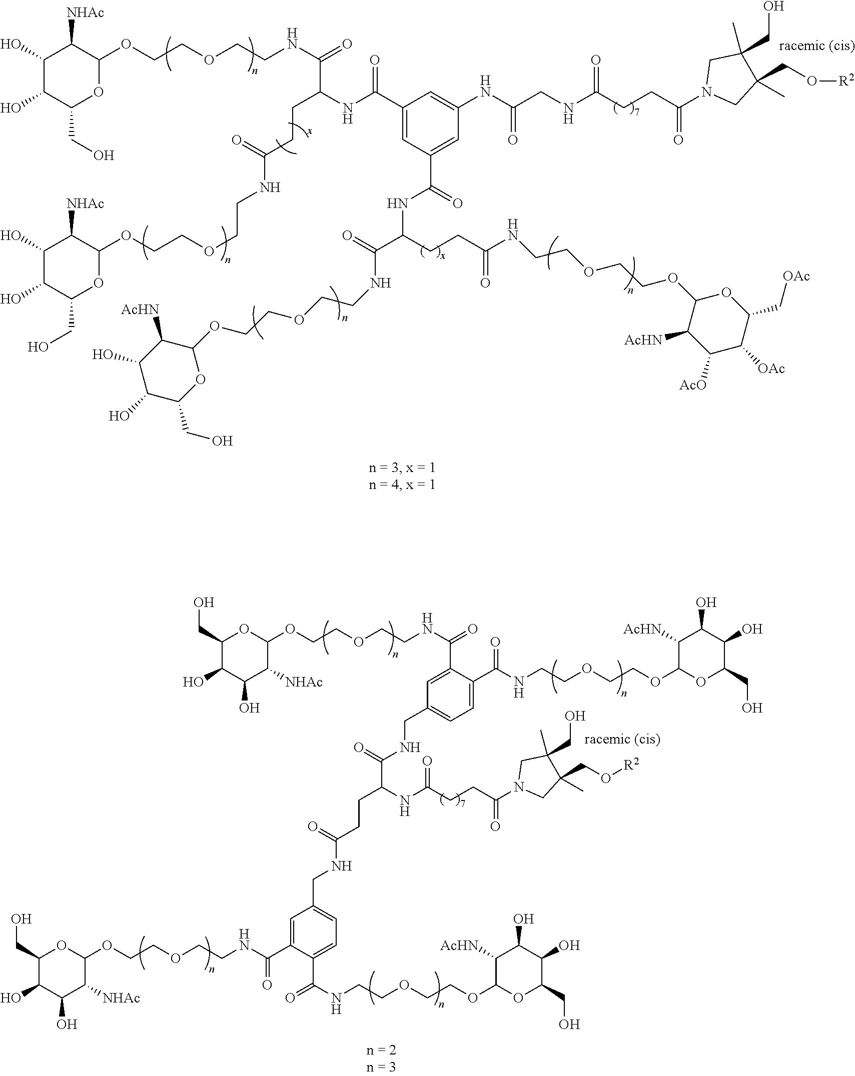

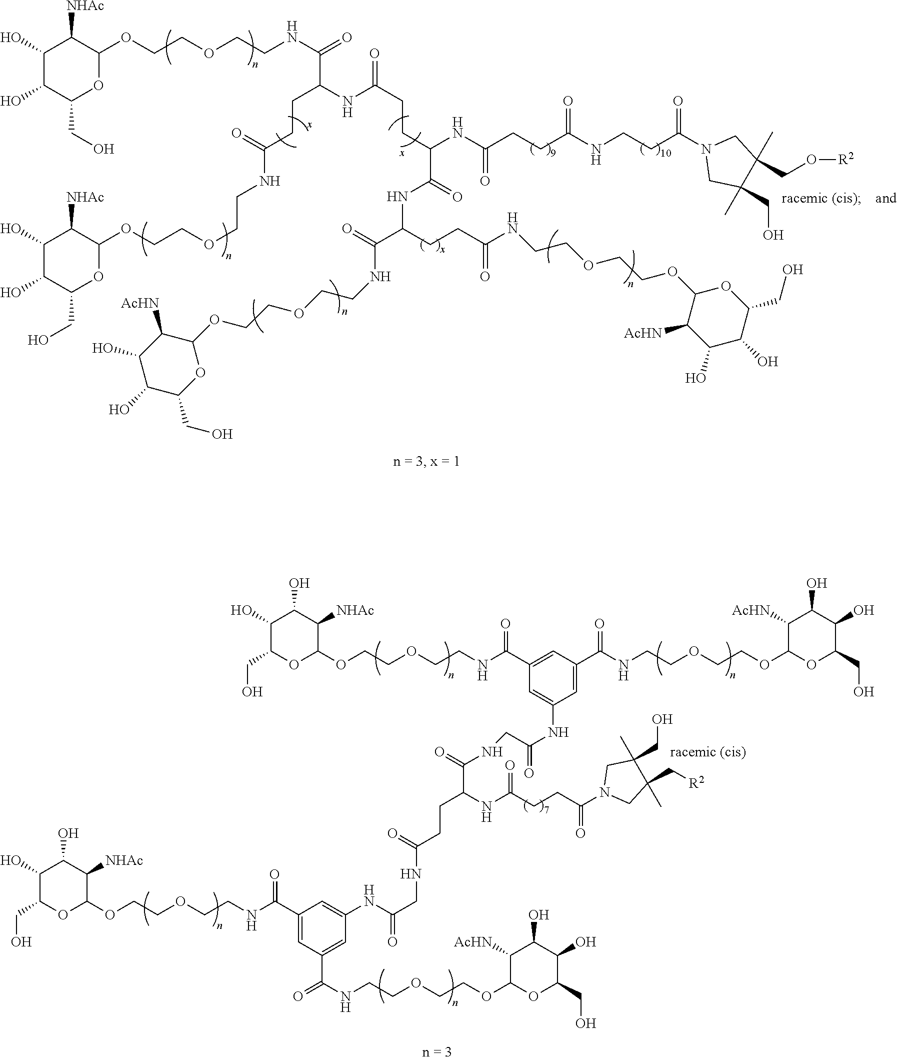

or pharmaceutically acceptable salts thereof.

In one embodiment the compound of formula I is:

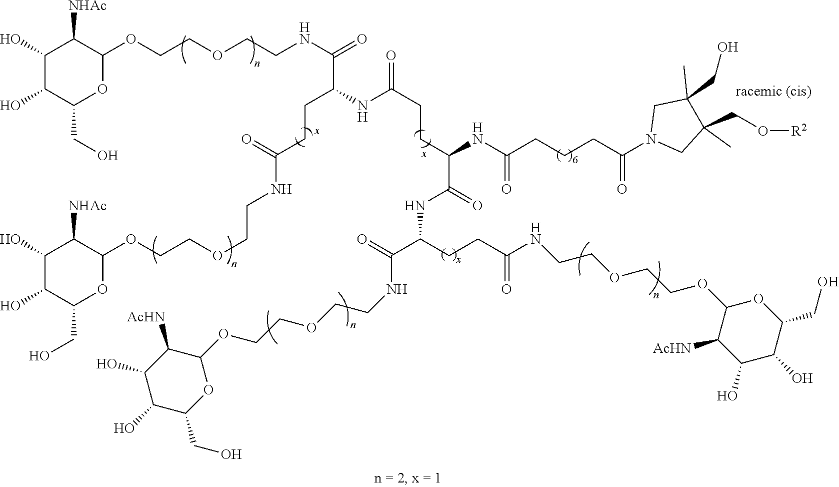

or a pharmaceutically acceptable salt thereof

In one embodiment the compound of formula I is:

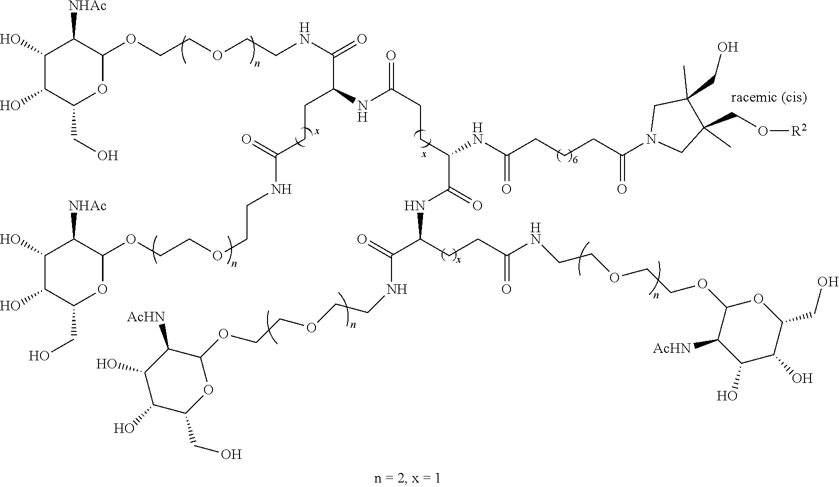

or a pharmaceutically acceptable salt thereof.

In one embodiment the compound of formula is:

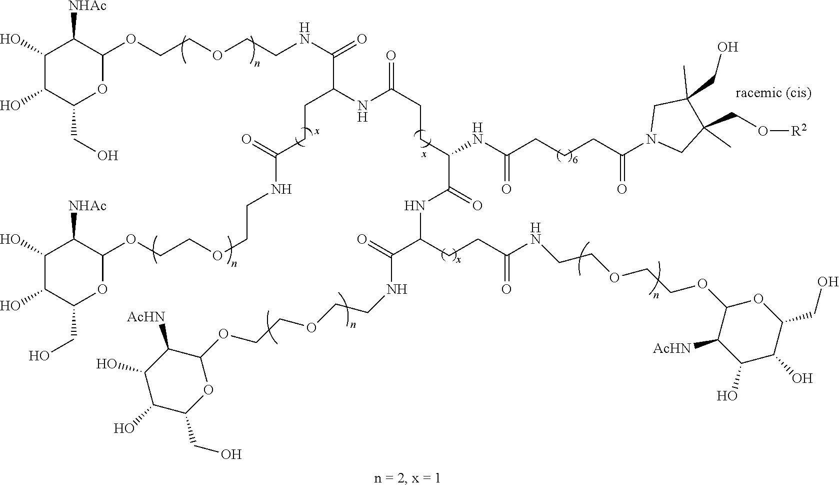

or a pharmaceutically acceptable salt thereof.

In one embodiment the compound of formula I is:

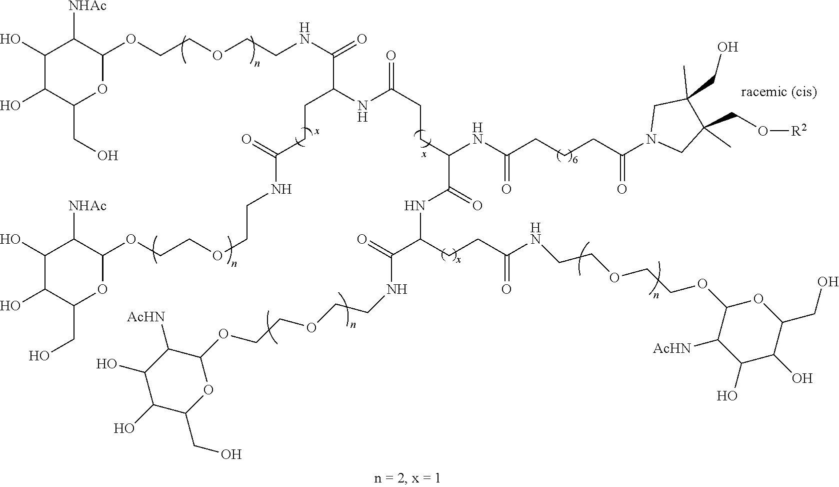

or a pharmaceutically acceptable salt.

In one embodiment the compound of formula I is:

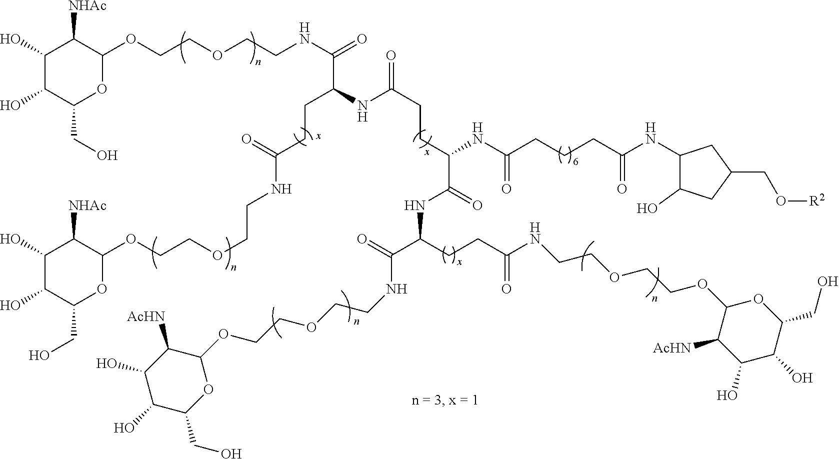

or a pharmaceutically acceptable salt thereof.

In one embodiment the compound of formula I is:

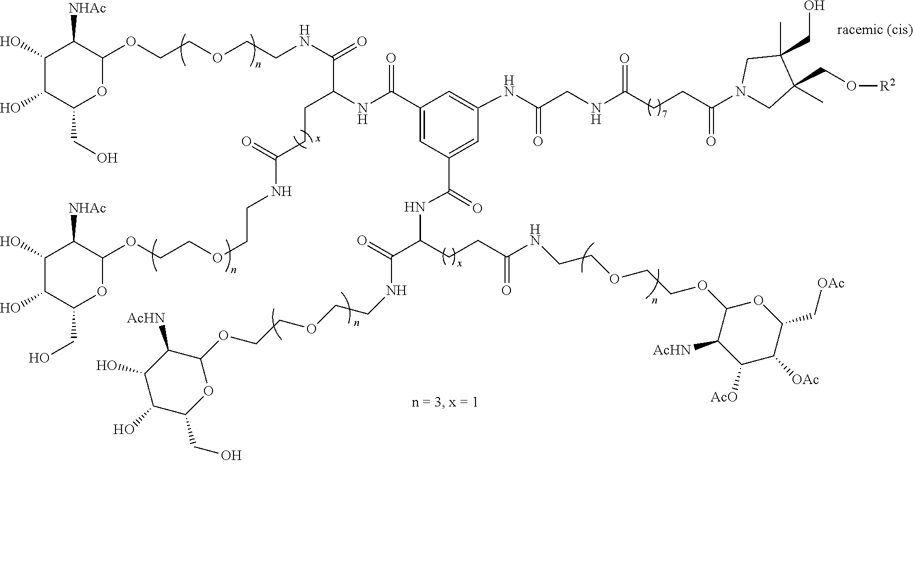

or a pharmaceutically acceptable salt thereof.

In one embodiment the compound of formula I is:

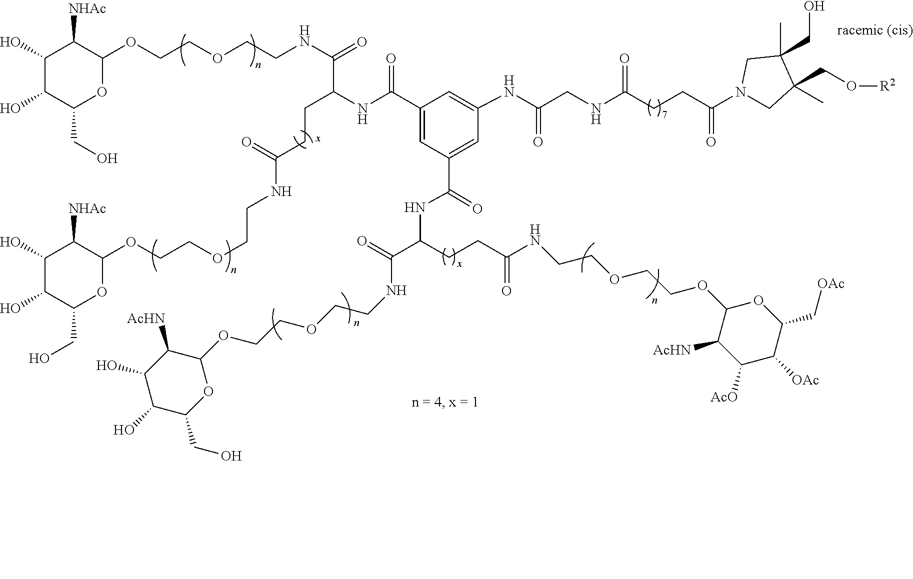

or a pharmaceutically acceptable salt thereof.

In one embodiment the compound of formula I is:

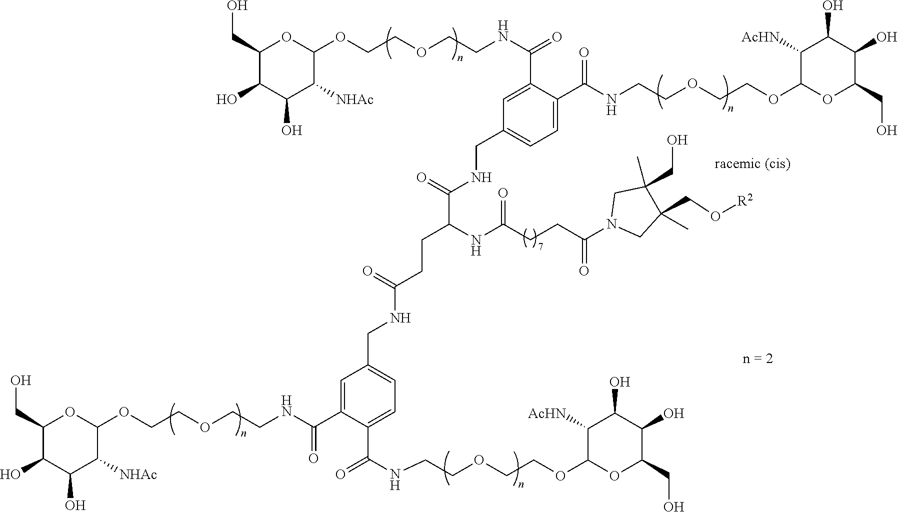

or a pharmaceutically acceptable salt thereof.

In one embodiment the compound of formula I is:

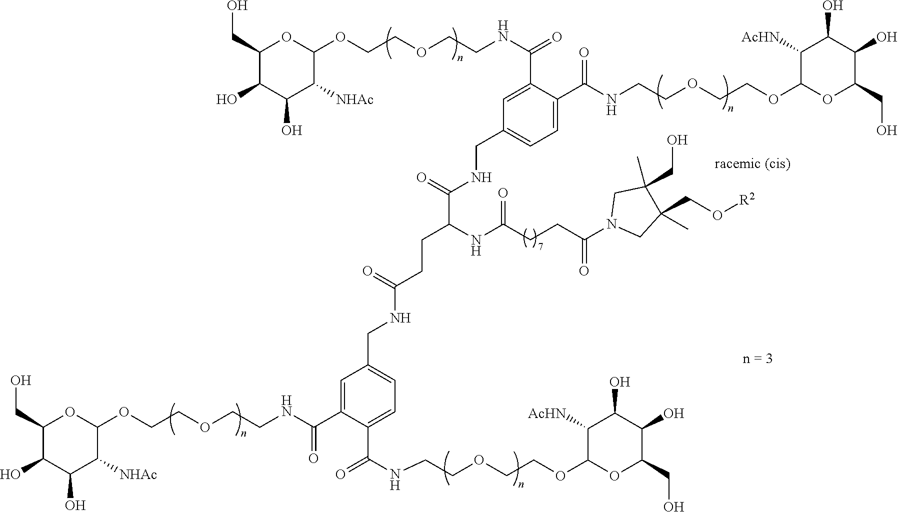

or a pharmaceutically acceptable salt thereof.

In one embodiment the compound of formula I is:

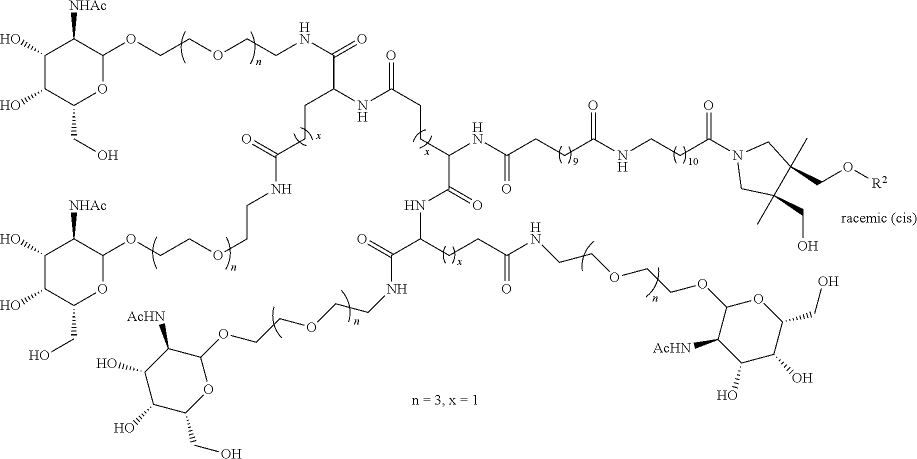

or a pharmaceutically acceptable salt thereof.

In one embodiment the compound of formula I is:

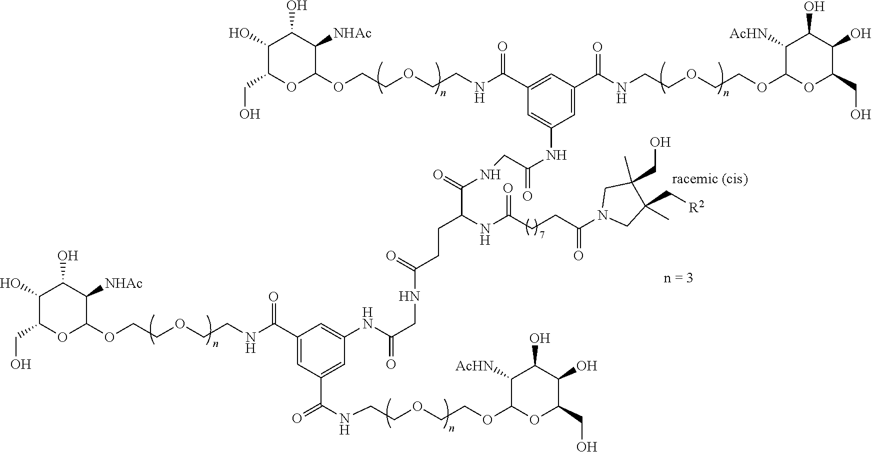

or a pharmaceutically acceptable salt thereof.

In one embodiment the compound of formula I is:

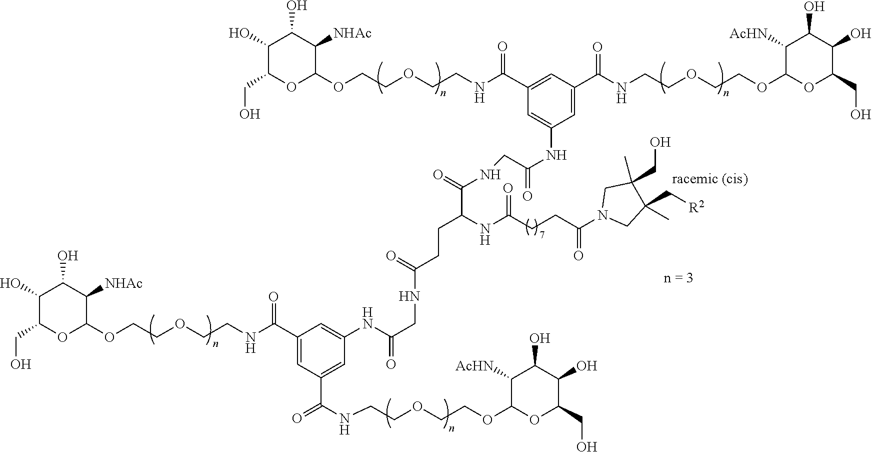

or a pharmaceutically acceptable salt thereof.

In one embodiment the compound of formula I is:

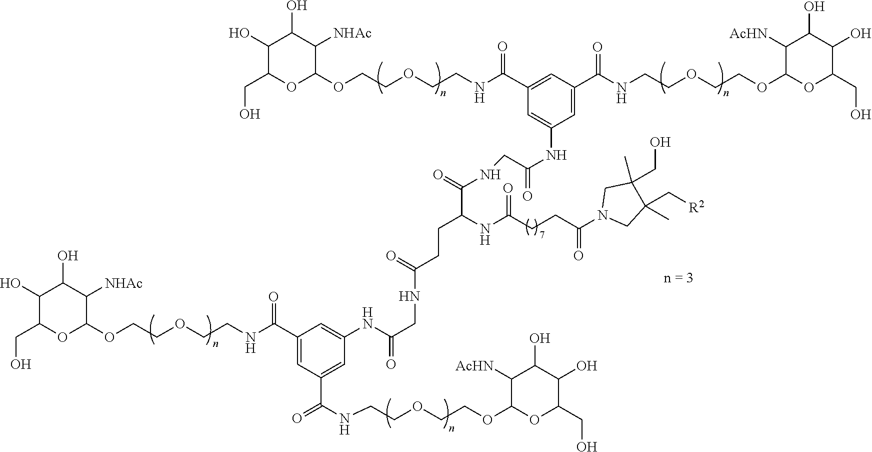

or a pharmaceutically acceptable salt thereof.

In one embodiment the invention provides a compound of formula:

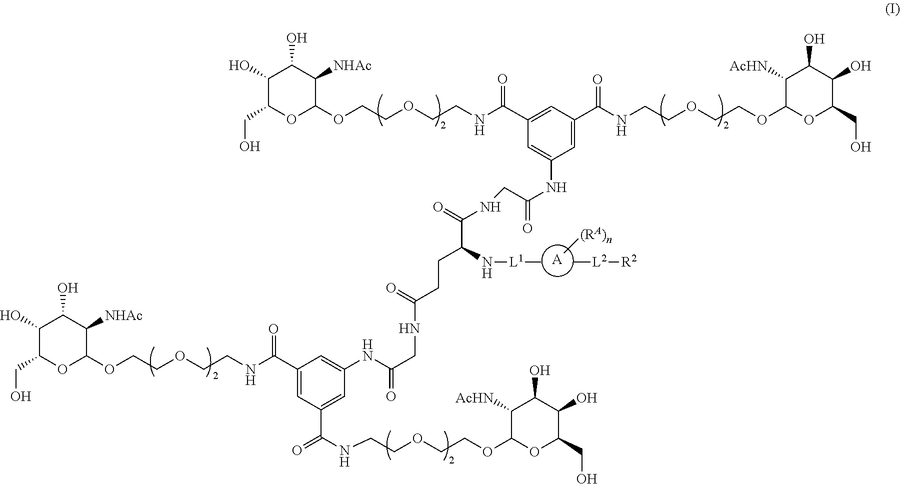

or a salt thereof.

In one embodiment the invention provides a compound of formula:

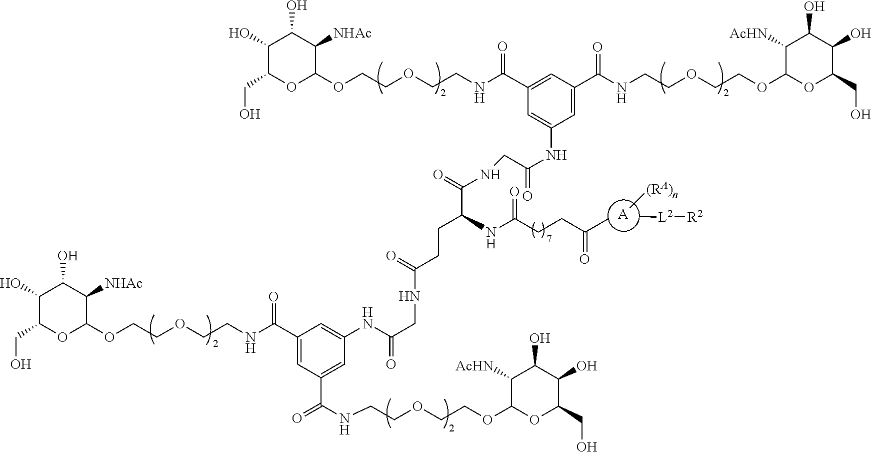

or a salt thereof.

In one embodiment the invention provides a compound of formula:

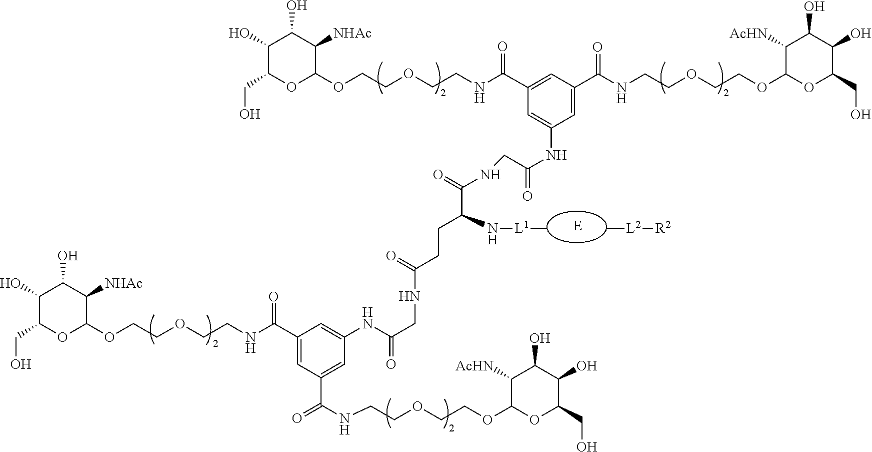

wherein:
L¹ is absent or a linking group;
L² is absent or a linking group;
R² is an siRNA molecule that comprises at least one unlocked nucleic acid (UNA) of the following formula:

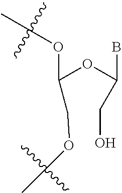

wherein B is a nucleobase:
ring E is divalent and is selected from the group consisting of:

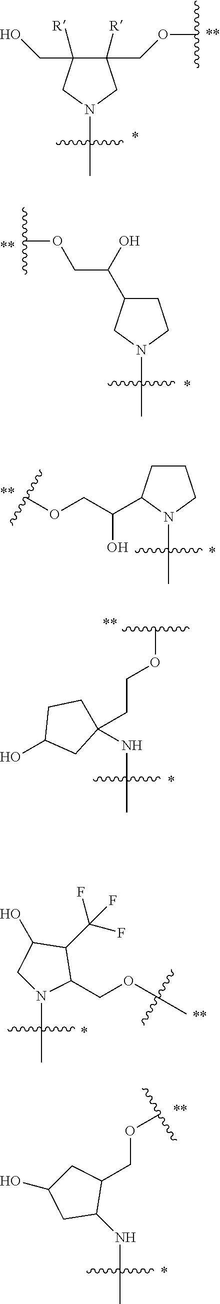

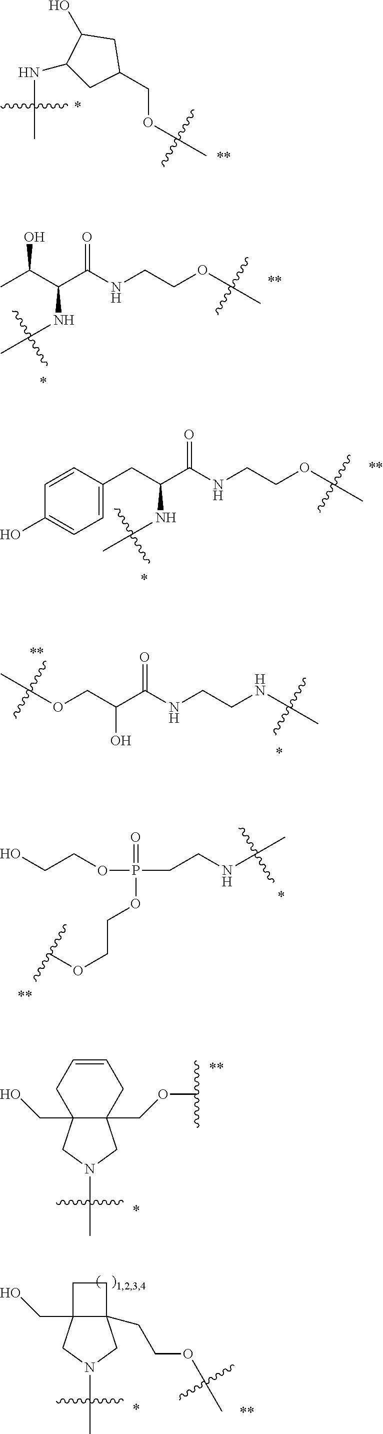

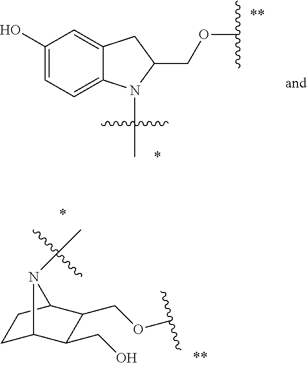

wherein:
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
the valence marked with * is attached to L¹ or is attached to R¹ if L¹ is absent; and
the valence marked with ** is attached to L² or is attached to R² if L² is absent;
or a salt thereof.

In one embodiment L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment L¹ is selected from the group consisting of:

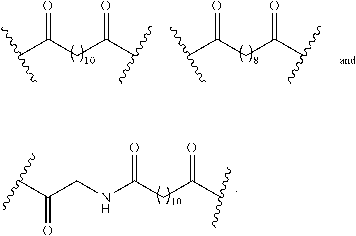

or a salt thereof.

In one embodiment L¹ is connected to B¹ through a linkage selected from the group consisting of —O—, —S—, —(C═O)—, —(C═O)—NH—, —NH—(C═O), —(C═O)—O—, —NH—(C═O)—NH—, or —NH—(SO₂)—.

In one embodiment L¹ is selected from the group consisting of:

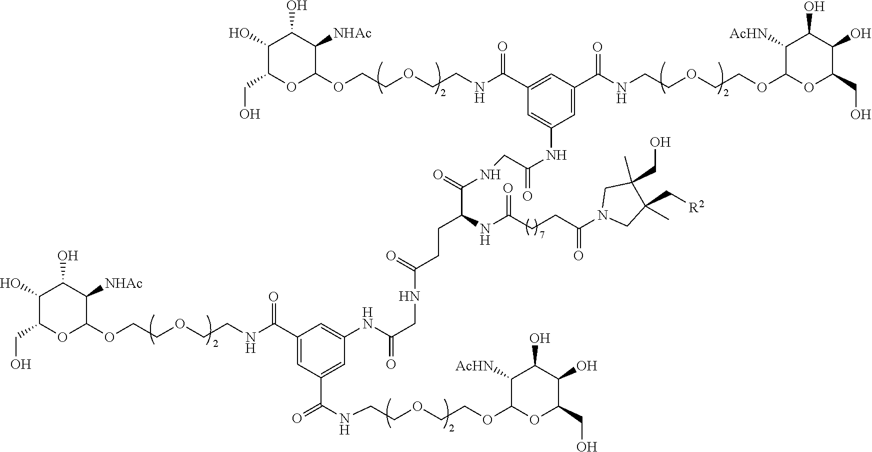

In one embodiment L² is connected to R² through —O—.

In one embodiment L² is C₁₋₄ alkylene-O— that is optionally substituted with hydroxy.

In one embodiment L² is absent.

In one embodiment the invention provides a compound,

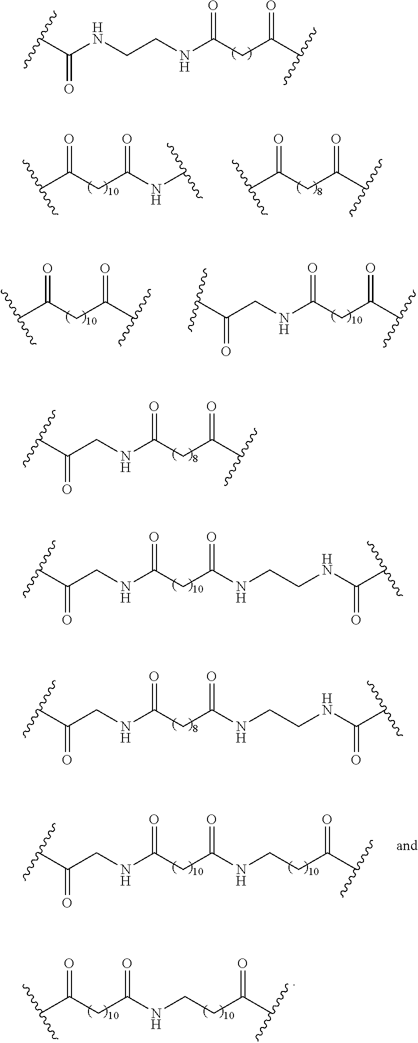

or a salt thereof wherein R² is a nucleic acid.

One aspect of this invention is pharmaceutical composition comprising a compound of formula I, and a pharmaceutically acceptable carrier.

Another aspect of this invention is a method to deliver a double stranded siRNA to the liver of an animal comprising administering a compound of formula I or a pharmaceutically acceptable salt thereof, to the animal.

Another aspect of this invention is a method to treat a disease or disorder (e.g., a liver disease or a viral infection, such as a hepatitis B viral infection) in an animal comprising administering a compound of formula I or a pharmaceutically acceptable salt thereof, to the animal.

Certain embodiments of the invention provide a compound of formula (I) or a pharmaceutically acceptable salt thereof for use in medical therapy.

Certain embodiments of the invention provide a compound of formula (I) or a pharmaceutically acceptable salt thereof for the prophylactic or therapeutic treatment of a disease or disorder (e.g., a liver disease or a viral infection, such as a hepatitis B virus infection) in an animal.

Certain embodiments of the invention provide the use of a compound of formula (I) or a pharmaceutically acceptable salt thereof to prepare a medicament for treating a disease or disorder (e.g., a liver disease or a viral infection, such as a hepatitis B virus infection) in an animal.

In certain embodiments, the animal is a mammal, such as a human (e.g., an HBV infected patient).

In one embodiment a compound of formula has the following formula (Id):

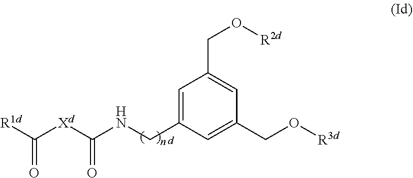

wherein:
R^{1d} is selected from:

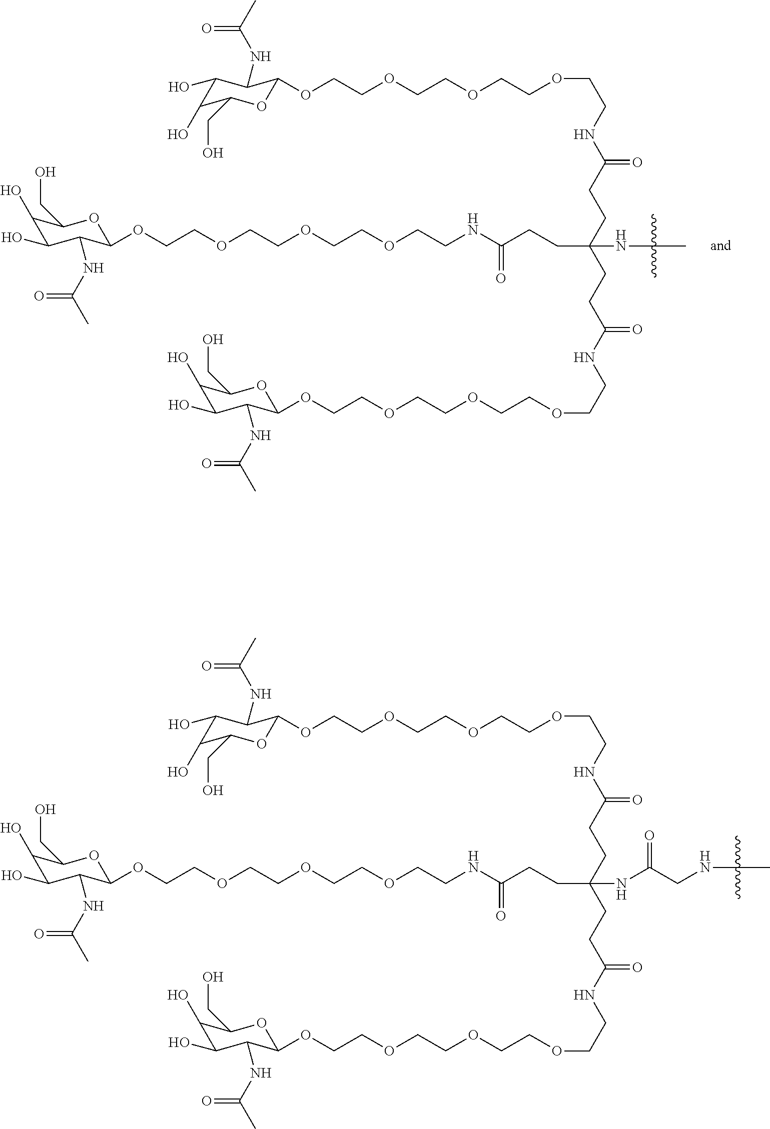 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₁₀ alkylene;
n^{d} is 0 or 1;
R^{2d} is an siRNA molecule that comprises at least one unlocked nucleic acid (UNA) of the following formula:

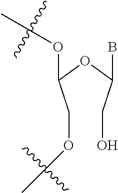

wherein B is a nucleobase, selected from the double stranded siRNA of Table 1; and
R^{3d} is H, a protecting group, a covalent bond to a solid support, or a bond to a linking group that is bound to a solid support.

In one embodiment R^{3d} includes a linking group that joins the remainder of the compound of formula Id to a solid support. The nature of the linking group is not critical provided the compound is a suitable intermediate for preparing a compound of formula Id wherein R^{2d} is an siRNA that comprises at least one unlocked nucleic acid (UNA) of the following formula:

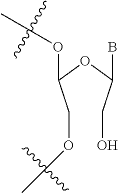

wherein B is a nucleobase.

In one embodiment the linker in R^{3d} has a molecular weight of from about 20 daltons to about 1,000 daltons.

In one embodiment the linker in R^{3d} has a molecular weight of from about 20 daltons to about 500 daltons.

In one embodiment the linker in R^{3d} separates the solid support from the remainder of the compound of formula I by about 5 angstroms to about 40 angstroms, inclusive, in length.

In one embodiment the linker in R^{3d} is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment the linker in R^{3d} is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment the linker in R^{3d} is —C(═O)CH₂CH₂C(═O)N(H)—.

In one embodiment R^{1d} is:

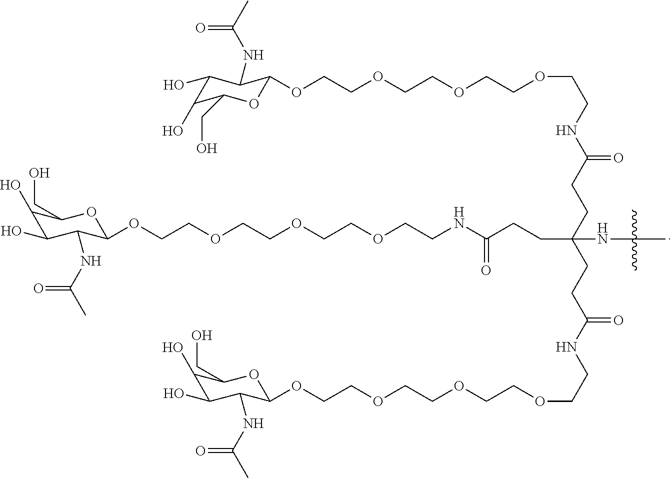 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In one embodiment R^{1d} is:

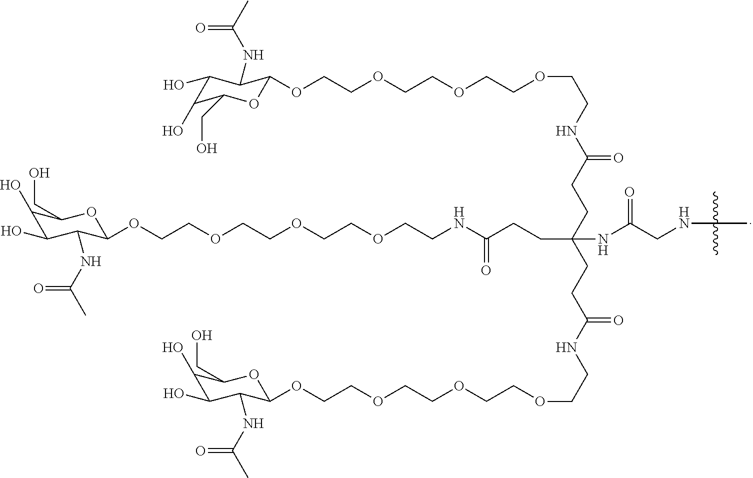 [SMILES: *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In one embodiment X^{d} is C₈alkylene.

In one embodiment n^{d} is 0.

In one embodiment R^{3d} is H.

In another embodiment a compound of (Id) or the salt thereof is selected from the group consisting of:

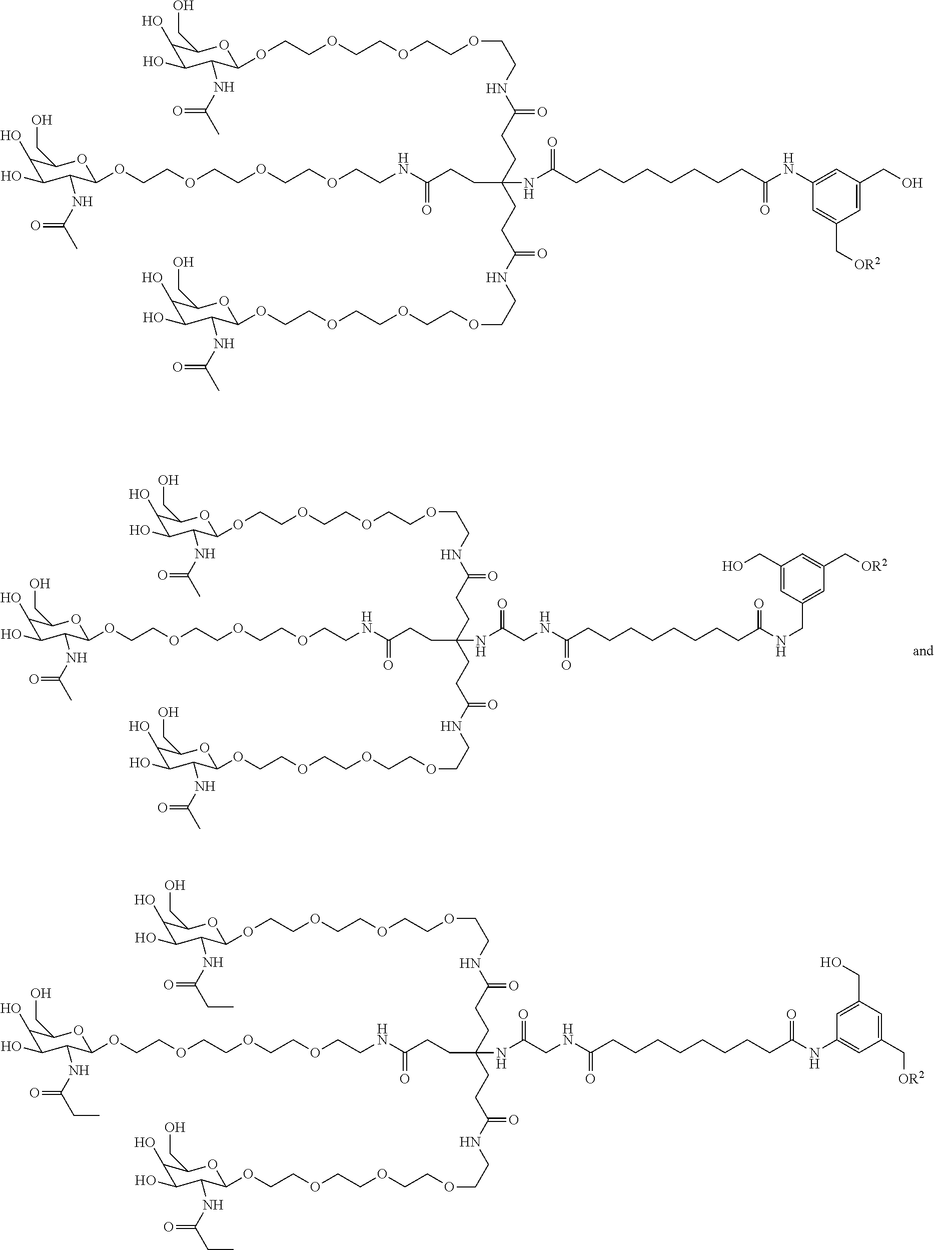

and salts thereof.

Another aspect of this invention is a method to treat a disease or disorder (e.g., a viral infection, such as a hepatitis B viral infection) in an animal comprising administering a compound of formula (Id) or a pharmaceutically acceptable salt thereof, to the animal.

Certain embodiments of the invention provide a compound of formula (Id) or a pharmaceutically acceptable salt thereof for use in medical therapy.

Certain embodiments of the invention provide a compound of formula (Id) or a pharmaceutically acceptable salt thereof for the prophylactic or therapeutic treatment of a disease or disorder (e.g., a viral infection, such as a hepatitis B virus infection) in an animal.

Certain embodiments of the invention provide the use of a compound of formula (Id) or a pharmaceutically acceptable salt thereof to prepare a medicament for treating a disease or disorder (e.g., a viral infection, such as a hepatitis B virus infection) in an animal.

In certain embodiments, the animal is a mammal, such as a human (e.g., an HBV infected patient).

The invention also provides synthetic intermediates and methods disclosed herein that are useful to prepare compounds of formula (Id). For example, the invention includes an intermediate compound of formula Ie:

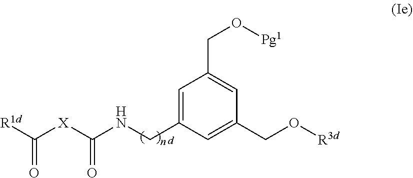

or a salt thereof, wherein:
R^{1d} is selected from:

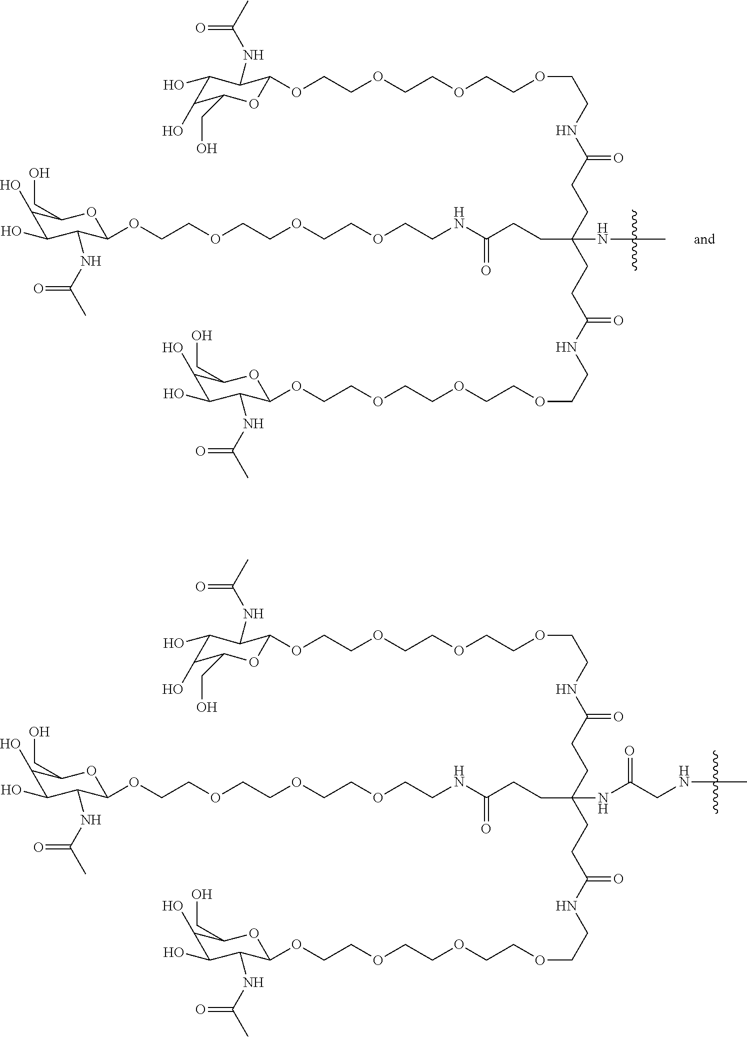 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₈ alkylene;
n^{d} is 0 or 1;
Pg¹ is H or a suitable protecting group; and
R^{3d} is H, a protecting group, a covalent bond to a solid support, or a bond to a linking group that is bound to a solid support.

In one embodiment Pg¹ is TMTr (Trimethoxytrityl), DMTr (Dimethoxytrityl), MMTr (Monomethoxytrityl), or Tr (Trityl).

The invention also provides a method to prepare a compound of formula (Id) as described herein comprising subjecting a corresponding compound of formula (Ie):

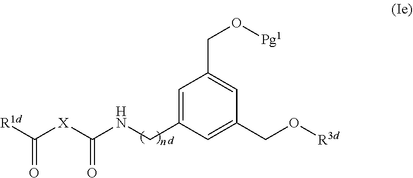

wherein:
X^{d} is C₂₋₈ alkylene;
n^{d} is 0 or 1;
Pg¹ is H; and
R^{3d} is a covalent bond to a solid support or a bond to a linking group that is bound to a solid support, to solid phase nucleic acid synthesis conditions to provide a corresponding compound of formula Id wherein R^{2d} is an siRNA molecule that comprises at least one unlocked nucleic acid (UNA) of the following formula:

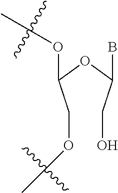

wherein B is a nucleobase.

In one embodiment the method further comprises removing the compound from the solid support to provide the corresponding compound of formula Id wherein R^{3d} is H.

In one embodiment the compound is not a compound formula Id:

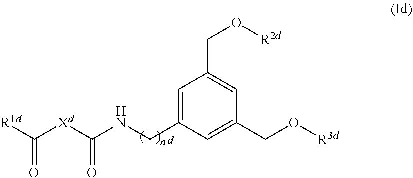

or a salt thereof, wherein:
R^{1d} is selected from:

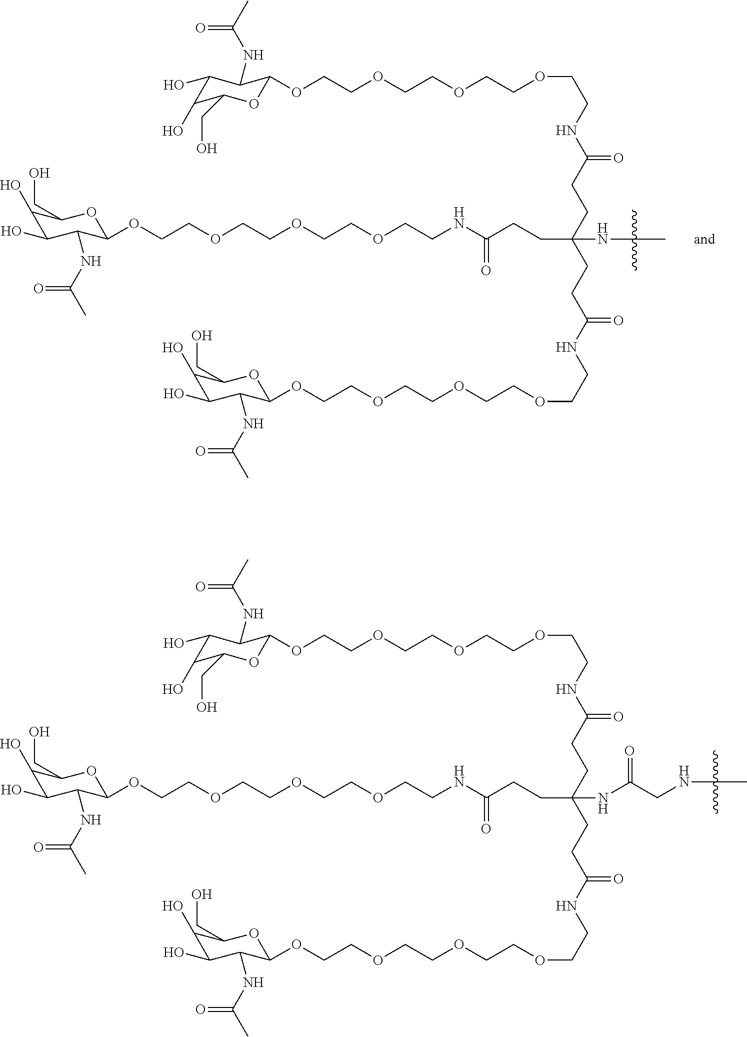 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₁₀ alkylene;
N^{d} is 0 or 1;
R^{2d} is an siRNA molecule that comprises at least one unlocked nucleic acid of the following formula:

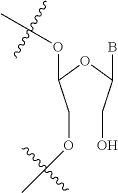

wherein B is a nucleobase; and
R^{3d} is H, a protecting group, a covalent bond to a solid support, or a bond to a linking group that is bound to a solid support.

In one embodiment the compound is not a compound formula Ie:

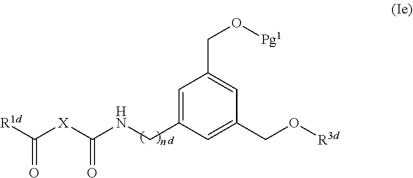

or a salt thereof, wherein:
R^{1d} is selected from:

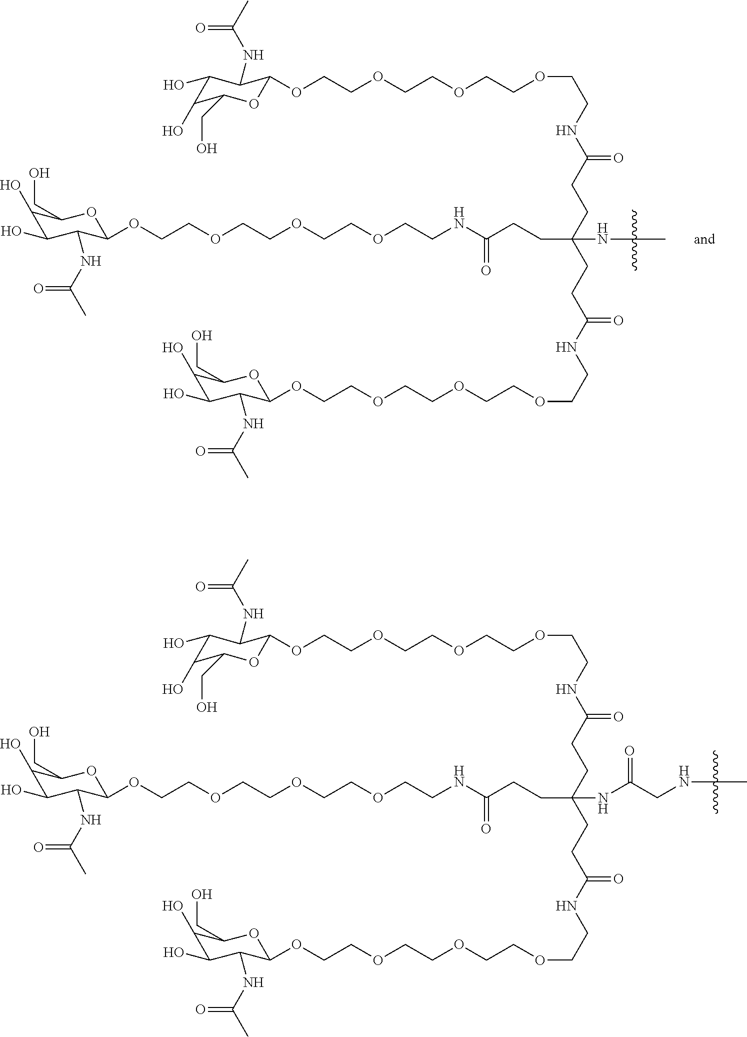 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₈ alkylene;
n^{d} is 0 or 1;
Pg¹ is H or a suitable protecting group; and
R^{3d} is H, a protecting group, a covalent bond to a solid support, or a bond to a linking group that is bound to a solid support.

In one embodiment R^{1d} is H.

In one embodiment R^{3d} is a covalent bond to a solid support.

In one embodiment R^{3d} is a bond to a linking group that is bound to a solid support, wherein the linking group is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment R^{3d} is a bond to a linking group that is bound to a solid support, wherein the linking group is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment R^{3d} is a bond to a linking group that is bound to a solid support, wherein the linking group is —C(═O)CH₂CH₂C(═O)N(H)—.

In one embodiment the invention provides a compound of formula (I):

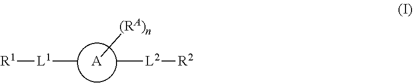

wherein:
R¹ is H or a synthetic activating group:
L¹ is absent or a linking group;
L² is absent or a linking group;
R² is an siRNA molecule that comprises at least one unlocked nucleic acid of the following formula:

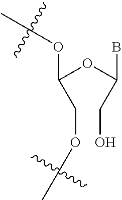

wherein B is a nucleobase;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR⁸, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
R^{B} is hydrogen, a protecting group, a covalent bond to a solid support, or a bond to a linking group that is bound to a solid support; and
n is 0, 1, 2, 3, 4, 5, 6, 7, 8, 9, or 10;
or a salt thereof.

In one embodiment the invention provides a compound of formula (II):

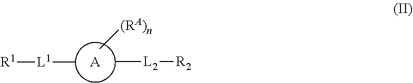

wherein:
R¹ a is targeting ligand:
L¹ is absent or a linking group;
L² is absent or a linking group;
R² is H or a synthetic activating group;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR⁸, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
R^{B} is hydrogen, a protecting group, a covalent bond to a solid support, or a bond to a linking group that is bound to a solid support; and
n is 0, 1, 2, 3, 4, 5, 6, 7, 8, 9, or 10;
or a salt thereof.

In one embodiment the invention provides a compound of formula (IIg):

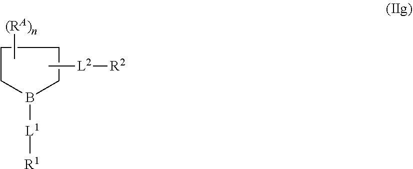

wherein:
B is —N— or —CH—;
L² is C₁₋₄ alkylene-O— that is optionally substituted with hydroxyl or halo; and
n is 0, 1, 2, 3, 4, 5, 6, or 7;
or a salt thereof.

In one embodiment the invention provides a compound selected from the group consisting of:

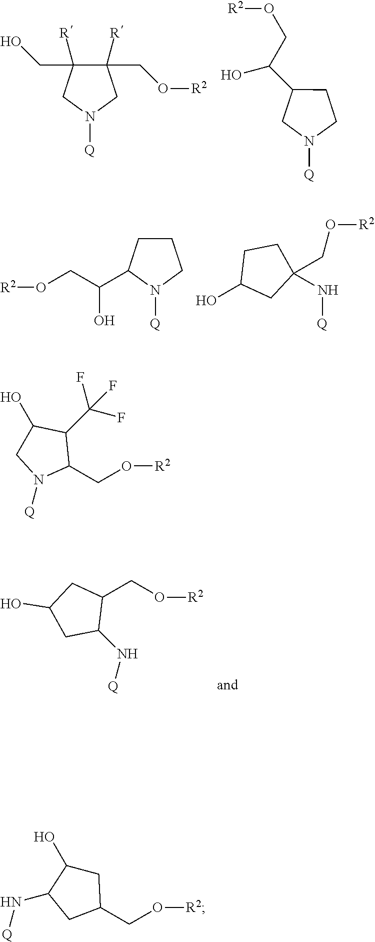

wherein:
Q is -L¹-R¹; and
R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
and salts thereof.

In one embodiment the invention provides a compound selected from the group consisting of:

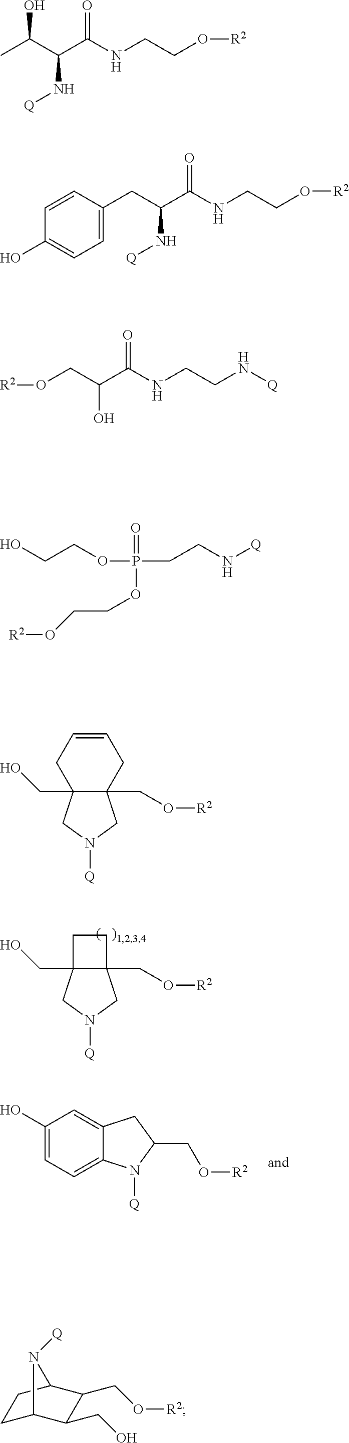

wherein: Q is -L¹-R¹; and salts thereof.

In one embodiment the invention provides a compound of formula (IIg):

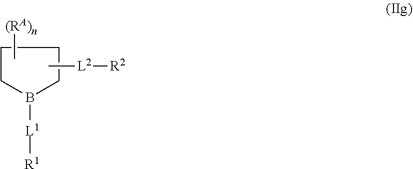

wherein:
B is —N— or —CH—;
L¹ is absent or a linking group;
L² is C₁₋₄ alkylene-O— that is optionally substituted with hydroxyl or halo;
n is 0, 1, 2, 3, 4, 5, 6, or 7:
R¹ is H or a synthetic activating group; and
R² is H or a synthetic activating group;
or a salt thereof.

In one embodiment the invention provides a compound selected from the group consisting of:

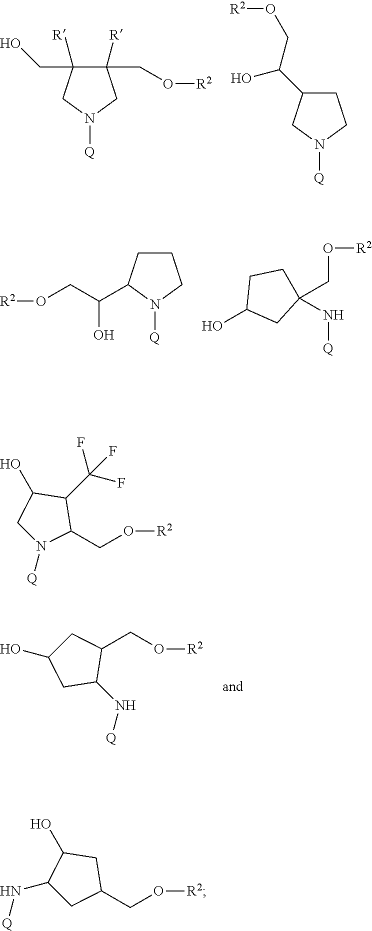

wherein Q is -L¹-R¹;
L¹ is absent or a linking group:
R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
R¹ is H or a synthetic activating group; and
R² is H or a synthetic activating group;
or a salt thereof.

In one embodiment the invention provides a compound selected from the group consisting of:

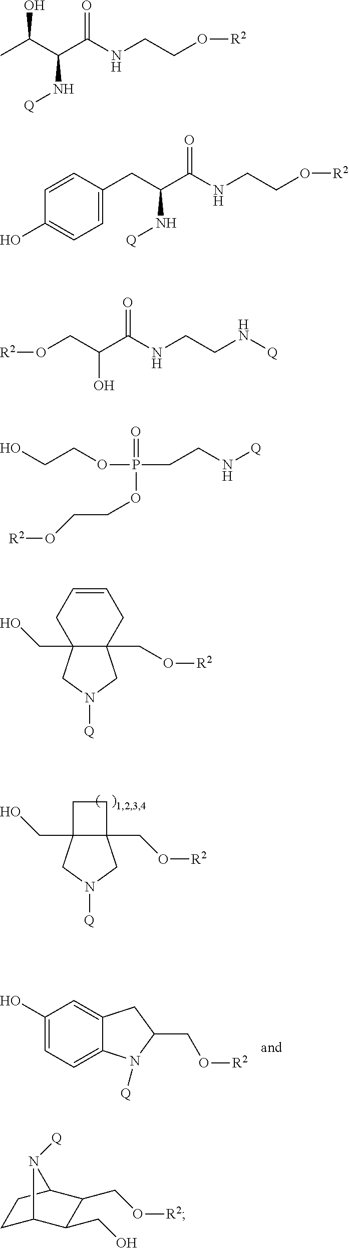

wherein:
Q is -L¹R¹;
L¹ is absent or a linking group:
R¹ is H or a synthetic activating group; and
R² is H or a synthetic activating group:
or a salt thereof.

In one embodiment R¹ is H or a synthetic activating group derivable from DCC, HOBt, EDC, BOP, PyBOP or HBTU.

In one embodiment R² is H, acetate, triflate, mesylate or succinate.

In one embodiment R¹ is a synthetic activating group derivable from DCC, HOBt, EDC, BOP, PyBOP or HBTU.

In one embodiment R² is acetate, triflate, mesylate or succinate.

In one embodiment L¹ is a divalent, branched or unbranched, saturated or unsaturated, hydrocarbon chain, having from 5 to 20 carbon atoms, wherein one or more (e.g. 1, 2, 3, or 4) of the carbon atoms in the hydrocarbon chain is optionally replaced —O—, —NH—, —NH—C(═O)—, C(═O)—NH— or —S—.

In one embodiment the invention provides a compound of formula (III):

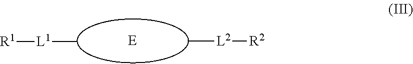

wherein:
R¹ is a targeting ligand that comprises one or more saccharide groups:
L¹ is absent or a linking group:
L² is absent or a linking group;
R² is an siRNA molecule that comprises at least one unlocked nucleic acid of the following formula:

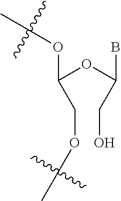

wherein B is a nucleobase;
ring E is divalent and is selected from the group consisting of:

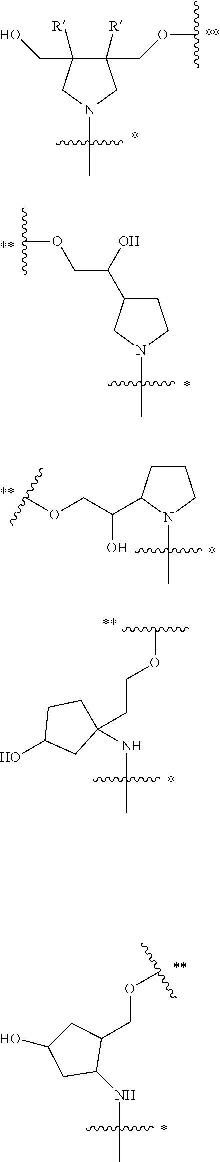

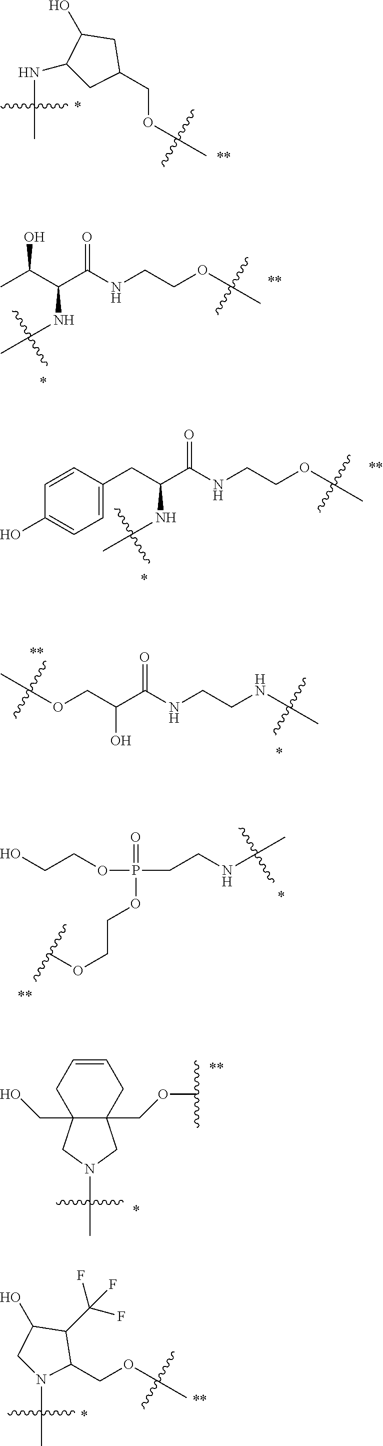

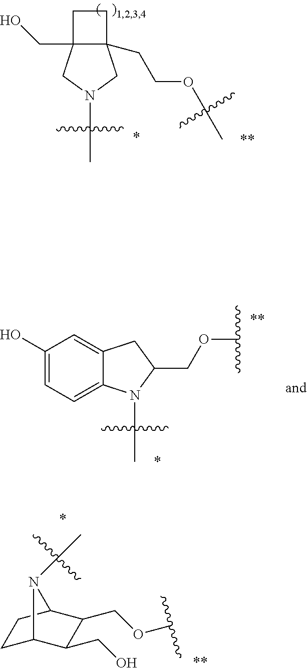

wherein:
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
the valence marked with * is attached to L¹ or is attached to R¹ if L¹ is absent; and
the valence marked with ** is attached to 12 or is attached to R² if L² is absent;
or a salt thereof.

In one embodiment R¹ comprises 2-8 saccharides.

In one embodiment R¹ comprises 2-6 saccharides.

In one embodiment R¹ comprises 2-4 saccharides.

In one embodiment R¹ comprises 3-8 saccharides.

In one embodiment R¹ comprises 3-6 saccharides.

In one embodiment R¹ comprises 3-4 saccharides.

In one embodiment R¹ comprises 3 saccharides.

In one embodiment R¹ comprises 4 saccharides.

In one embodiment R¹ has the following formula:

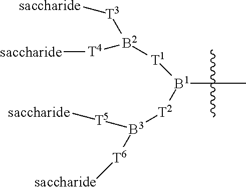

wherein:
B¹ is a trivalent group comprising about 1 to about 20 atoms and is covalently bonded to L¹, T¹, and T².
B² is a trivalent group comprising about 1 to about 20 atoms and is covalently bonded to T¹, T³, and T⁴;
B³ is a trivalent group comprising about 1 to about 20 atoms and is covalently bonded to T², T⁵, and T⁶;
T¹ is absent or a linking group:
T² is absent or a linking group:
T³ is absent or a linking group:
T⁴ is absent or a linking group;
T⁵ is absent or a linking group; and
T⁶ is absent or a linking group

In one embodiment each saccharide is independently selected from:

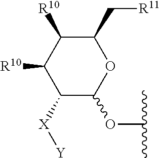

wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In one embodiment each saccharide is independently selected from the group consisting of:

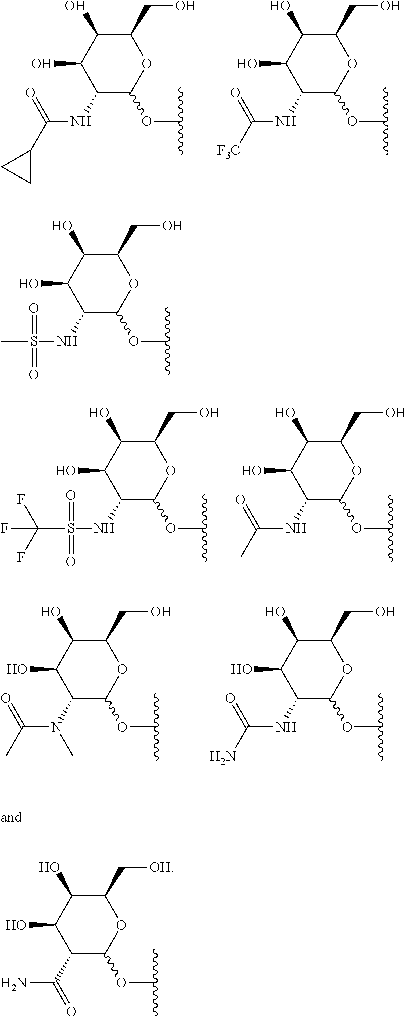

In one embodiment each saccharide is independently:

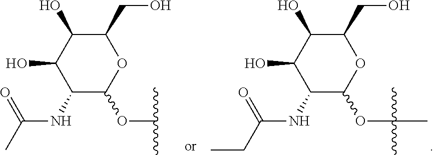 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment one of T¹ and T² is absent.

In one embodiment both T¹ and T² are absent.

In one embodiment each of T¹, T², T³, T⁴, T⁵, and T⁶ is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment each of T¹, T², T³, T⁴, T⁵, and T⁶ is independently absent or a branched or unbranched, saturated or unsaturated, hydrocarbon chain, having from 1 to 50 carbon atoms, or a salt thereof, wherein one or more (e.g. 1, 2, 3, or 4) of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In one embodiment at least one of T³, T⁴, T⁵, and T⁶ is:

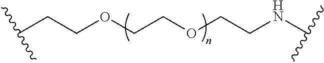

wherein:
n=1, 2, 3.

In one embodiment each of T³, T⁴, T⁵, and T⁶ is independently selected from the group consisting of:

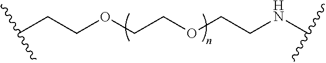

wherein:
n=1, 2, 3.

In one embodiment at least one of T¹ and T² is glycine

In one embodiment each of T¹ and T² is glycine.

In one embodiment B¹ is a trivalent group comprising 1 to 15 atoms and is covalently bonded to L¹, T¹, and T².

In one embodiment B¹ is a trivalent group comprising 1 to 10 atoms and is covalently bonded to L¹, T¹, and T².

In one embodiment B¹ comprises a (C₁-C₆)alkyl.

In one embodiment B¹ comprises a C₃₋₈ cycloalkyl.

In one embodiment B¹ comprises a silyl group.

In one embodiment B¹ comprises a D- or L-amino acid.

In one embodiment B¹ comprises a saccharide.

In one embodiment B¹ comprises a phosphate group.

In one embodiment B¹ comprises a phosphonate group.

In one embodiment B¹ comprises an aryl.

In one embodiment B¹ comprises a phenyl ring.

In one embodiment B¹ is a phenyl ring.

In one embodiment B¹ is CH.

In one embodiment B¹ comprises a heteroaryl.

In one embodiment B¹ is selected from the group consisting of:

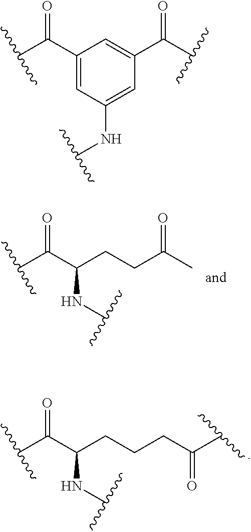 [SMILES: *Nc1cc(C(*)=O)cc(C(*)=O)c1 ; *N[C@H](CCC(C)=O)C(*)=O ; *N[C@H](CCCC(*)=O)C(*)=O]

In one embodiment B¹ is selected from the group consisting of:

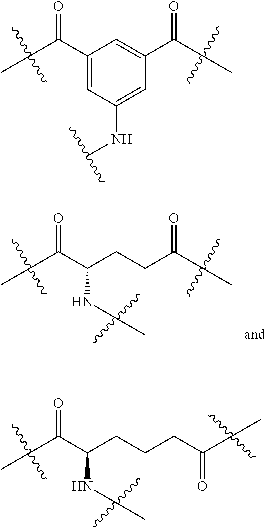 [SMILES: *Nc1cc(C(*)=O)cc(C(*)=O)c1 ; *N[C@@H](CCC(*)=O)C(*)=O ; *N[C@H](CCCC(*)=O)C(*)=O]

In one embodiment B² is a trivalent group comprising 1 to 15 atoms and is covalently bonded to L¹, T¹, and T².

In one embodiment B² is a trivalent group comprising 1 to 10 atoms and is covalently bonded to L¹, T¹, and T².

In one embodiment B² comprises a (C₁-C₆)alkyl.

In one embodiment B² comprises a C₃₋₈ cycloalkyl.

In one embodiment B² comprises a silyl group.

In one embodiment B² comprises a D- or L-amino acid.

In one embodiment B² comprises a saccharide.

In one embodiment B² comprises a phosphate group.

In one embodiment B² comprises a phosphonate group.

In one embodiment B² comprises an aryl.

In one embodiment B² comprises a phenyl ring.

In one embodiment B² is a phenyl ring.

In one embodiment B² is CH.

In one embodiment B² comprises a heteroaryl.

In one embodiment B² is selected from the group consisting of:

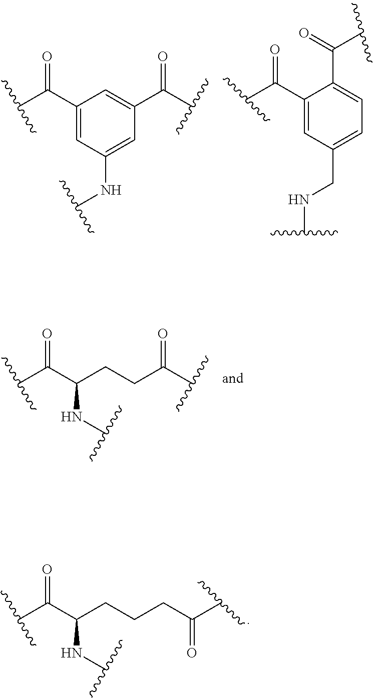 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

In one embodiment B² is selected from the group consisting of:

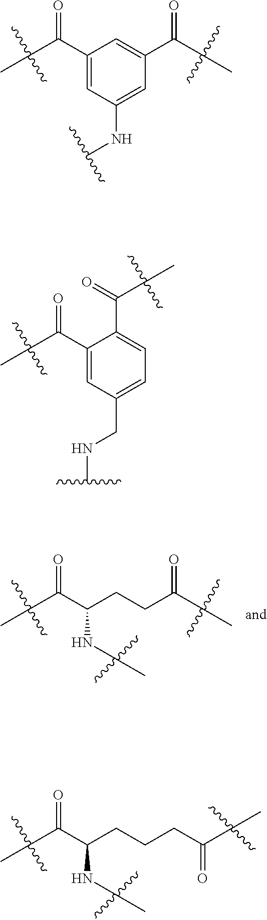 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

or a salt thereof.

In one embodiment B³ is a trivalent group comprising 1 to 15 atoms and is covalently bonded to L¹, T¹, and T².

In one embodiment B³ is a trivalent group comprising 1 to 10 atoms and is covalently bonded to L¹, T¹, and T².

In one embodiment B³ comprises a (C₁-C₆)alkyl.

In one embodiment B³ comprises a C₃₋₈ cycloalkyl.

In one embodiment B³ comprises a silyl group.

In one embodiment B³ comprises a D- or L-amino acid.

In one embodiment B³ comprises a saccharide.

In one embodiment B³ comprises a phosphate group.

In one embodiment B³ comprises a phosphonate group.

In one embodiment B³ comprises an aryl.

In one embodiment B³ comprises a phenyl ring.

In one embodiment B³ is a phenyl ring.

In one embodiment B³ is CH.

In one embodiment B³ comprises a heteroaryl.

In one embodiment B³ is selected from the group consisting of:

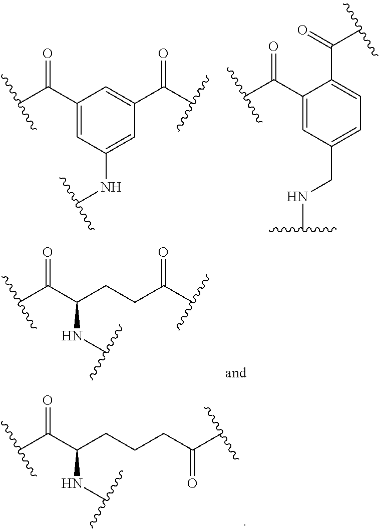 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

In one embodiment B³ is selected from the group consisting of:

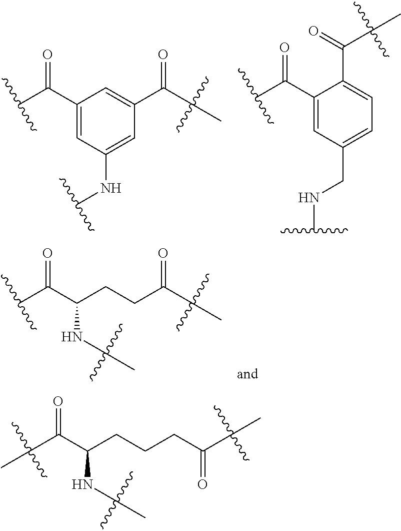 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

or a salt thereof.

In one embodiment L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment L¹ is selected from the group consisting of:

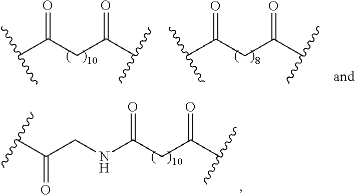

or a salt thereof.

In one embodiment L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In one embodiment L¹ is selected from the group consisting of:

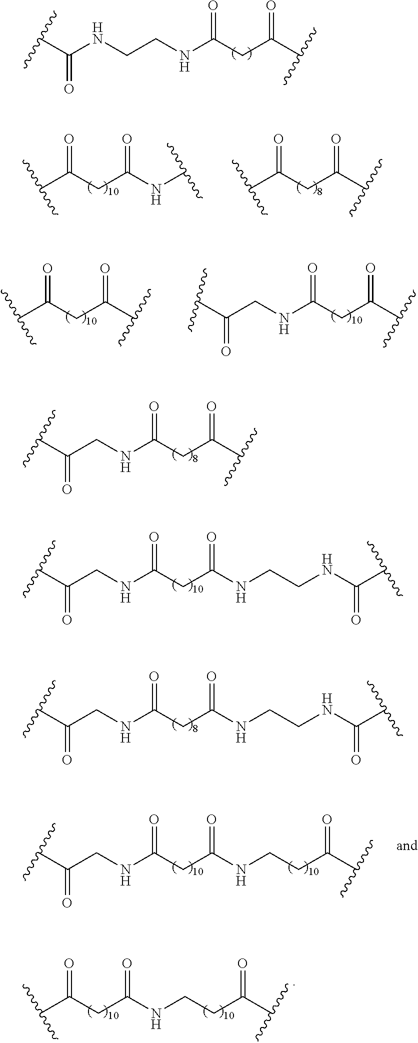

In one embodiment L² is connected to R² through —O—.

In one embodiment L² is C₁₋₄ alkylene-O— that is optionally substituted with hydroxy.

In one embodiment L² is connected to R² through —O—.

In one embodiment L² is absent.

In one embodiment the invention provides a compound or salt selected from the group consisting of:

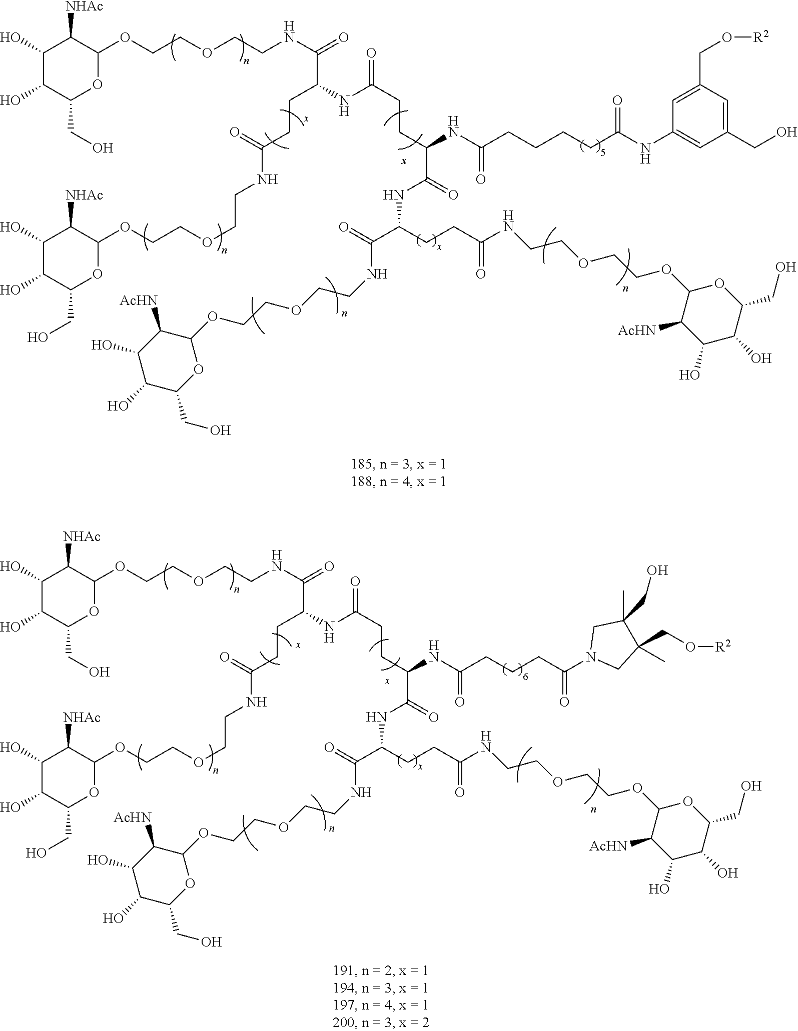

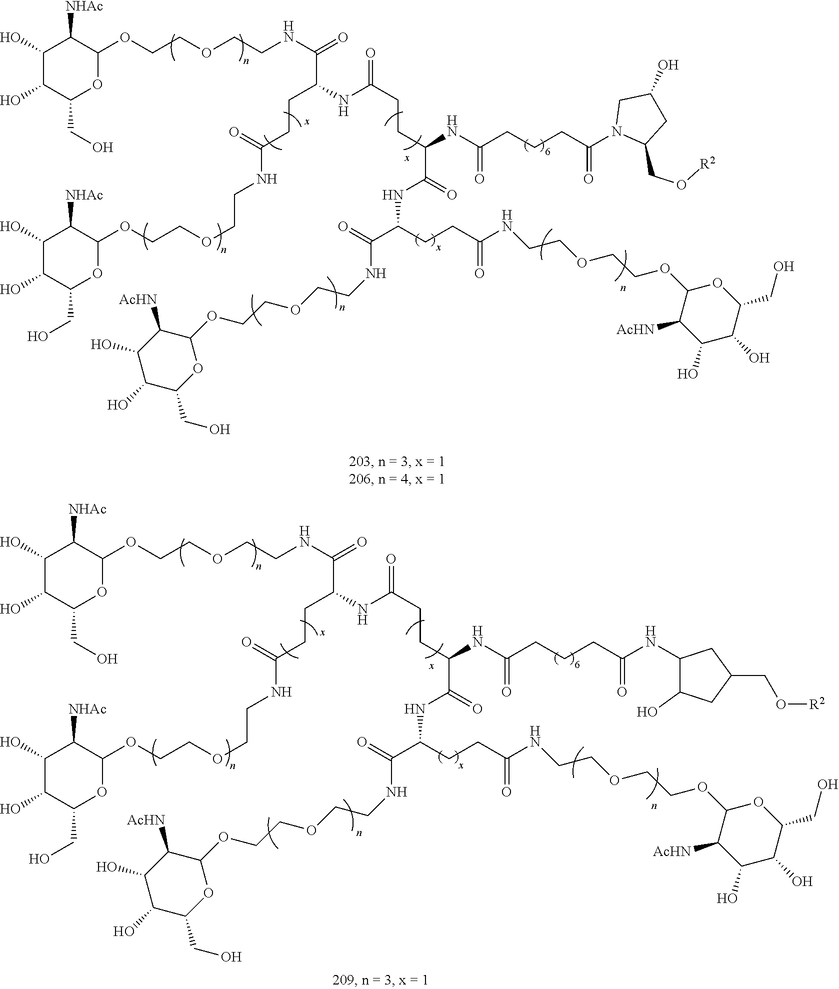

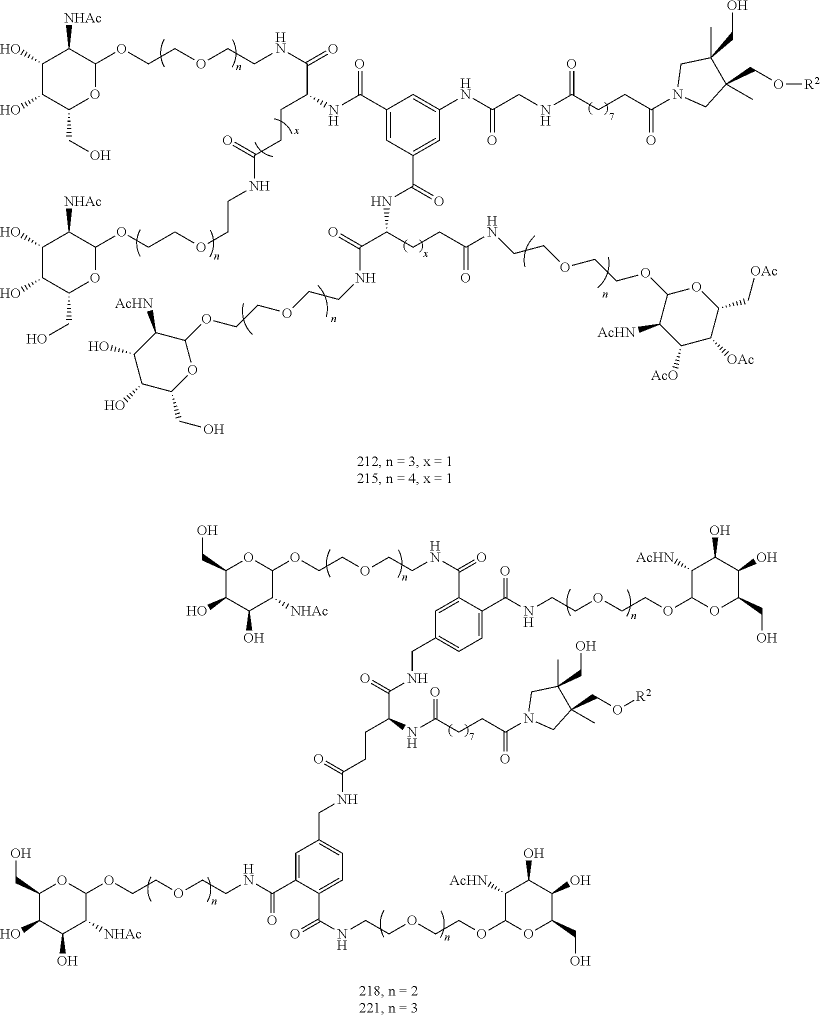

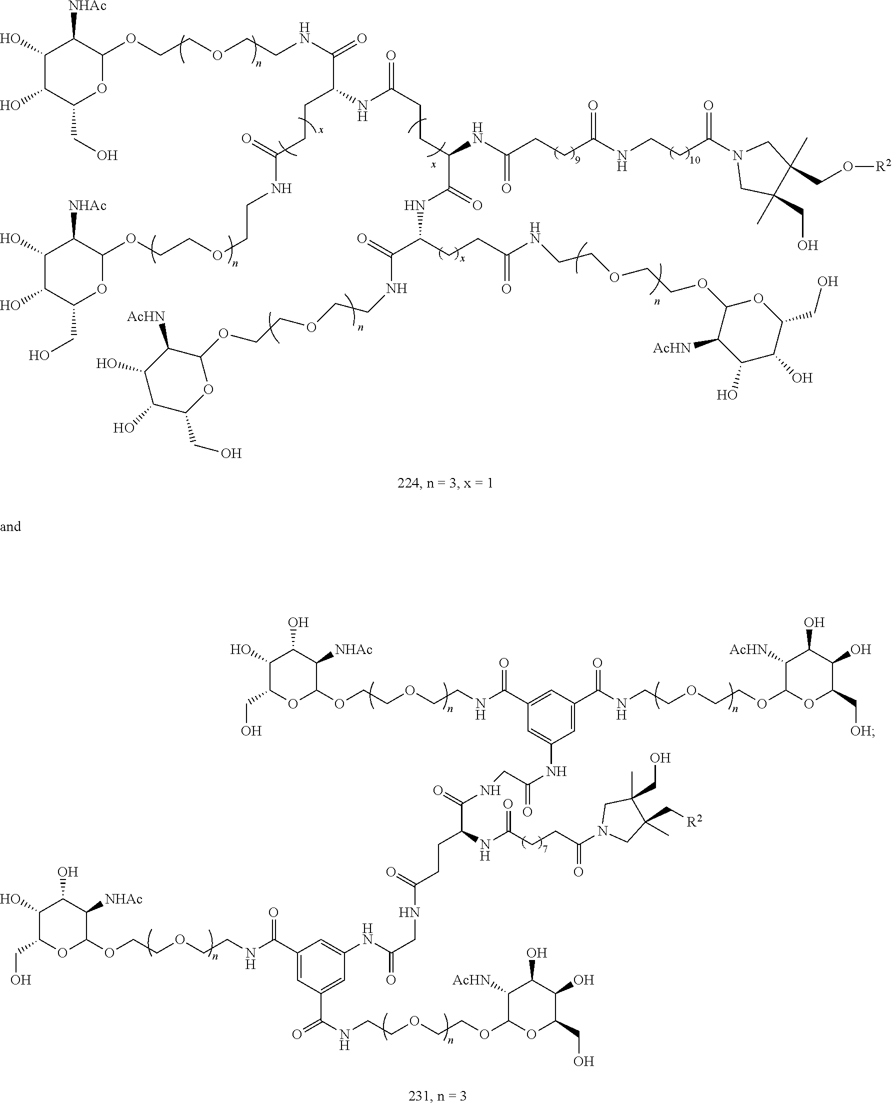

and pharmaceutically acceptable salts thereof, wherein R² is an siRNA that comprises at least one unlocked nucleic acid of the following formula:

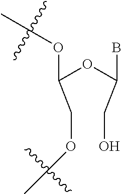

wherein B is a nucleobase.

In one embodiment the invention provides a compound of formula:

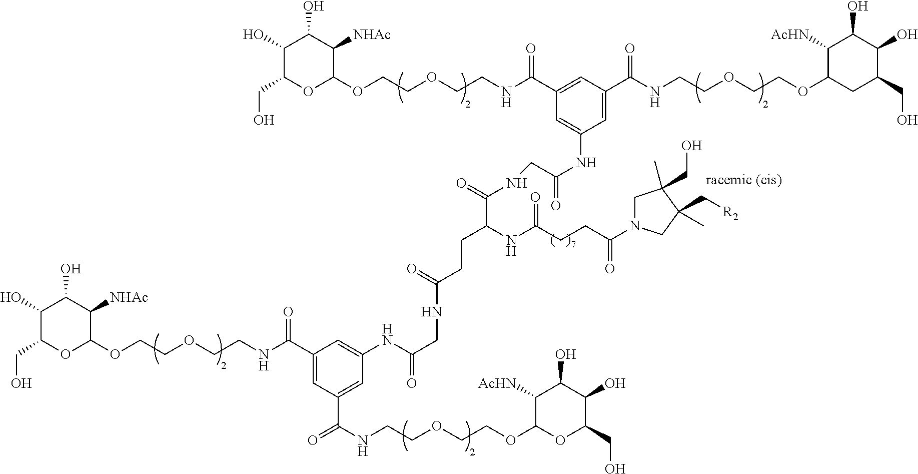

or a salt thereof wherein R² is a nucleic acid.

In one embodiment the invention provides a compound of formula

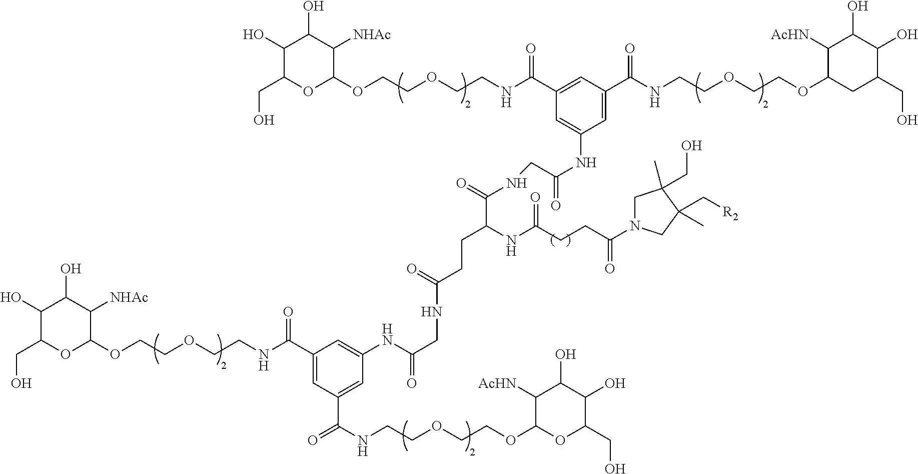

or a salt thereof wherein R² is a nucleic acid.

In one embodiment, the nucleic acid molecule (e.g., siRNA) is attached to the reminder of the compound through the oxygen of a phosphate at the 3′-end of the sense strand.

In one embodiment the compound or salt is administered subcutaneously.

When a compound comprises a group of the following formula:

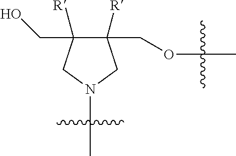

there are four stereoisomers possible on the ring, two cis and two trans. Unless otherwise noted, the compounds of the invention include all four stereoisomers about such a ring. In one embodiment, the two R′ groups are in a cis conformation. In one embodiment, the two R′ groups are in a trans conformation.

In certain embodiments, an additional therapeutic agent useful, e.g., to treat hepatitis B can be administered in combination with the conjugate described herein. Certain additional therapeutic agents are described hereinbelow. For example, the methods can comprise further administering to the subject at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome.

### Reverse Transcriptase Inhibitors

In certain embodiments, the reverse transcriptase inhibitor is a nucleoside analog.

In certain embodiments, the reverse transcriptase inhibitor is a nucleoside analog reverse-transcriptase inhibitor (NARTI or NRTI).

In certain embodiments, the reverse transcriptase inhibitor is a nucleoside analog inhibitor of HBV polymerase.

In certain embodiments, the reverse transcriptase inhibitor is a nucleotide analog reverse-transcriptase inhibitor (NtARTI or NtRTI).

In certain embodiments, the reverse transcriptase inhibitor is a nucleotide analog inhibitor of HBV polymerase.

The term reverse transcriptase inhibitor includes, but is not limited to: entecavir (ETV), clevudine, telbivudine, lamivudine, adefovir, tenofovir, tenofovir disoproxil, tenofovir alafenamide (TAF), tenofovir disoproxil fumarate (TDF), adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074), emtricitabine, abacavir, elvucitabine, ganciclovir, lobucavir, famciclovir, penciclovir, and amdoxovir.

The term reverse transcriptase inhibitor includes, but is not limited to: the reverse transcriptase inhibitor is entecavir (ETV), tenofovir disoproxil fumarate (TDF) or tenofovir alafenamide (TAF).

The term reverse transcriptase inhibitor includes, but is not limited to, entecavir, lamivudine, and (1R,2R,3R,5R)-3-(6-amino-9H-9-purinyl)-2-fluoro-5-(hydroxymethyl)-4-methylenecyclopentan-1-ol.

The term reverse transcriptase inhibitor includes, but is not limited to a covalently bound phosphoramidate or phosphonamidate moiety of the above-mentioned reverse transcriptase inhibitors, or as described in, for example, U.S. Pat. No. 8,816,074, US 2011/0245484 A1, and US 2008/0286230A1.

The term reverse transcriptase inhibitor includes, but is not limited to, nucleotide analogs that comprise a phosphoramidate moiety, such as, methyl ((((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy)phosphoryl)-(D or L)-alaninate. Also included are the individual diastereomers thereof, which includes, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S) (((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate.

The term reverse transcriptase inhibitor includes, but is not limited to a phosphonamidate moiety, such as, tenofovir alafenamide, as well as those described in US 2008/0286230 A1. Methods for preparing stereoselective phosphoramidate or phosphonamidate containing actives are described in, for example, U.S. Pat. No. 8,816,074, as well as US 2011/0245484 A1 and US 2008/0286230 A1.

### Capsid Inhibitors

As described herein the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA. Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

The term capsid inhibitor includes compounds described in WO 2018/172852, which patent document is specifically incorporated by reference in its entirety.

The term capsid inhibitor also includes compounds described in International Patent Applications Publication Numbers WO2013006394, WO2014106019, and WO2014089296, including the following compounds:

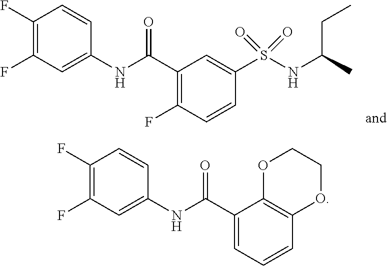

The term capsid inhibitor also includes the compounds Bay-41-4109 (see International Patent Application Publication Number WO/2013/144129), AT-61 (see International Patent Application Publication Number WO/1998/33501; and King, R W, et al., Antimicrob Agents Chemother., 1998, 42, 12, 3179-3186), DVR-01 and DVR-23 (see International Patent Application Publication Number WO 2013/006394; and Campagna, M R, et al., J. of Virology, 2013, 87, 12, 6931, and pharmaceutically acceptable salts thereof:

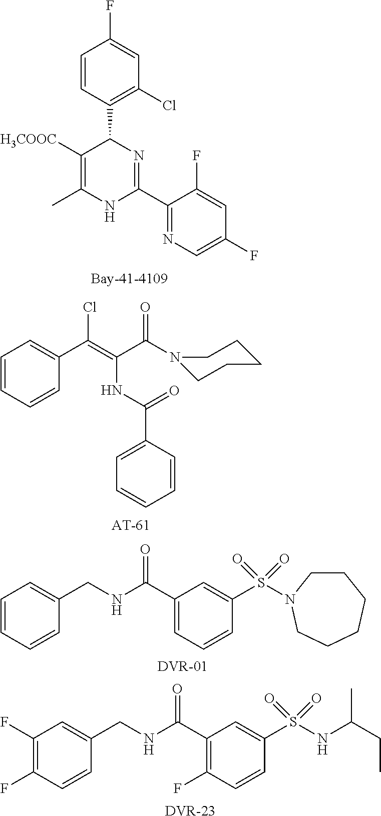

The term capsid inhibitor also includes the compound:

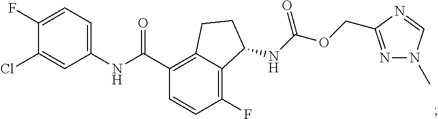 [SMILES: Cn1cnc(COC(=O)N[C@H]2CCc3c(C(=O)Nc4ccc(F)c(Cl)c4)ccc(F)c32)n1]

and pharmaceutically acceptable salts thereof (see WO 2018/172852).

In certain embodiments, a capsid inhibitor is a compound of the following formula, or a salt thereof:

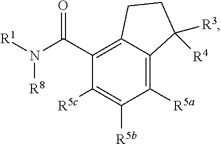

wherein the following definitions apply:
R¹ is selected from the group consisting of optionally substituted phenyl, optionally substituted benzyl, optionally substituted heteroaryl, and —(CH₂) (optionally substituted heteroaryl);
each occurrence of R² is independently selected from the group consisting of H and C₁-C₆ alkyl;
R³ is selected from the group consisting of —N(R²)C(═O)OR⁶, H, —OH, —OR⁶, —NH₂, —NHR⁶, —NR⁶R⁶, —OC(═O)OR⁶, —OC(═O)N(R²)R⁶, —NR⁷C(O)N(R⁶)(R⁷), —N(R²)C(═O)R⁶, —NR²S(═O)₁₋₂R⁶, optionally substituted aryl, optionally substituted heteroaryl. —CH₂C(═O)OH, —CH₂C(═O)NR⁶R⁶, —N(R²)C(═O)(CH₂)₁₋₂R⁶, NR²S(═O)₂N(R⁶)(R⁷), and —NR²C(═O)C(═O)N(R⁶)(R⁷);
R⁴ is H or C₁-C₆ alkyl, or
   R³ and R⁴ combine to form ═O or —C(═O)NR^{6a}—C(═O)—NR^{6a}—;
R^{5a} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;
R^{5b} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;
R^{5c} is independently selected from the group consisting of H, halo, C₁-C₆ alkyl, C₁-C₆ alkoxy, C₁-C₆ aminoalkyl, C₁-C₆ haloalkoxy, and C₁-C₆ haloalkyl;
each occurrence of R⁶ is independently selected from the group consisting of optionally substituted C₁-C₆ alkyl, optionally substituted C₃-C₈ cycloalkyl, optionally substituted phenyl, and optionally substituted hetereoaryl;
each occurrence of R^{6a} is independently selected from the group consisting of H, optionally substituted C₁-C₆ alkyl, optionally substituted C₃-C₈ cycloalkyl, optionally substituted phenyl, and optionally substituted hetereoaryl;
each occurrence of R⁷ is independently selected from the group consisting of H and optionally substituted C₁-C₆ alkyl;
   or, if R⁶ and R⁷ are bound to the same N atom, R⁶ and R⁷ optionally combine with the N atom to which both are bound to form optionally substituted 3-7 membered heterocyclyl; and
R⁸ is selected from the group consisting of H and C₁-C₆ alkyl.

In certain embodiments, each occurrence of R⁶ or R^{6a} is independently selected from the group consisting of —(CH₂)₁₋₃-(optionally substituted heteroaryl), —(CH₂)₁₋₃-(optionally substituted heterocyclyl), and —(CH₂)₁₋₃-(optionally substituted aryl).

In certain embodiments, each occurrence of optionally substituted alkyl, optionally substituted heterocyclyl, or optionally substituted cycloalkyl is independently optionally substituted with at least one substituent selected from the group consisting of C₁-C₆ alkyl, halo, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of optionally substituted aryl or optionally substituted heteroaryl is independently optionally substituted with at least one substituent selected from the group consisting of C₁-C₆ alkyl, C₁-C₆ haloalkyl, C₁-C₆ haloalkoxy, halo, —CN, OR^{b}, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

In certain embodiments, each occurrence of optionally substituted aryl or optionally substituted heteroaryl is independently optionally substituted with at least one substituent selected from the group consisting of C₁-C₆ alkyl, C₁-C₆ haloalkyl, C₁-C₆ haloalkoxy, halo, —CN, —OR^{c}, —N(R^{c})(R^{c}), and C₁-C₆ alkoxycarbonyl, wherein each occurrence of R is independently H, C₁-C₆ alkyl, or C₃-C₈ cycloalkyl.

In certain embodiments, R¹ is selected from the group consisting of optionally substituted phenyl, optionally substituted benzyl, and —(CH₂)(optionally substituted heteroaryl), wherein the phenyl, benzyl, or heteroaryl is optionally substituted with at least one selected from the group consisting of C₁-C₆ alkyl, halo, C₁-C₃ haloalkyl, and —CN.

In certain embodiments, R¹ is selected from the group consisting of 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, phenyl, 3-chlorophenyl, 4-chlorophenyl, 3-fluorophenyl, 4-fluorophenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyan-4-fluorophenyl, 4-cyano-3-fluorophenyl, 3-difluoromethyl-4-fluorophenyl, 4-difluoromethyl-3-fluorophenyl, benzo[d][1,3]dioxol-5-yl, 2,3-dihydrobenzo[b][1,4]dioxin-6-yl, benzyl, 3-fluorobenzyl, 4-fluorobenzyl, 3-chlorobenzyl, 4-chlorobenzyl, 2-pyridyl, 4-methyl-2-pyridyl, 5-methyl-2-pyridyl, 6-methyl-2-pyridyl, 3-pyridyl, 2-methyl-3-pyridyl, 3-methyl-3-pyridyl, 4-pyridyl, 2-methyl-4-pyridyl, and 6-methyl-4-pyridyl.

In certain embodiments, each occurrence of R² is independently selected from the group consisting of H and methyl.

In certain embodiments, R³ is selected from the group consisting of: —NH₂; —OH; —NH(pyridinyl); —NH(pyrimidinyl); —NH(piridinyl-pyrimidinyl); —NH(pyrrolo[2,3-d]pyrimidinyl); —NHS(═O)₂(C₁-C₆ alkyl); —NHS(═O)₂(C₃-C₆ cycloalkyl); —NHS(═O)₂(CH₂)₀₋₃pyridinyl: —NHS(═O)₂(benzyl); —NHS(═O)₂(pyrazolyl); —NHS(═O)₂(morpholinyl): —NHS(═O)₂NH(C₁-C₆ alkyl); —NHS(═O)₂NH(C₃-C₆ cycloalkyl); —NHS(═O)₂NH(CH₂)₀₋₃pyridinyl; —NHS(═O)₂NH(benzyl); —NHS(═O)₂NH(pyrazolyl); —NHS(═O)₂NH(morpholinyl); —NHC(═O)(C₁-C₆ alkyl); —NHC(═O)(C₃-C₈ cycloalkyl); —NHC(═O)(C₁-C₆ haloalkyl); —NHC(═O)(pyrazolyl); —NHC(═O)(thiazolyl): —NHC(═O)(oxazolyl); —NHC(═O)(pyridinyl); —NHC(═O)(CH₂)₁₋₃(pyridinyl); —NHC(═O)(CH₂)₁₋₃(pyrazinyl); —NHC(═O)(CH₂)₁₋₃(pyrimidinyl); —NHC(═O)(CH₂)₁₋₃(quinolinyl); —NHC(═O)(CH₂)₁₋₃(isoxazolyl); —NHC(═O)(CH₂)₁₋₃(oxazolyl); —NHC(═O)(CH₂)₁₋₃(oxadiazolyl); —NHC(═O)(CH₂)₁₋₃(triazolyl); —NHC(═O)(CH₂)₁₋₃(thiazolyl); —NHC(═O)(CH₂)₁₋₃(imidazolyl); —NHC(═O)(CH₂)₁₋₃(pyrazolyl); —NHC(═O)(CH₂)₁₋₃(piperidinyl); —NHC(═O)(CH₂)₁₋₃(oxopiperidinyl); —NHC(═O)(CH₂)₁₋₃(pyrrolidinyl); —NHC(═O)(CH₂)₁₋₃(oxopyrrolidinyl); —NHC(═O))(CH₂)₁₋₃(tetrahydrofuryl); —NHC(═O)(CH₂)₁₋₃(tetrahydropyranyl); —NHC(═O)(CH₂)₁₋₃(2-oxooxazolidinyl); —NHC(═O)(CH₂)₁₋₃(morpholinyl): —NHC(═O)(CH₂)₁₋₃(thiomorpholinyl); —NHC(═O)(CH₂)₁₋₃(1-oxido-thiomorpholinyl); —NHC(═O)(CH₂)₁₋₃(1,1-dioxido-thiomorpholinyl); —NHC(═O)(CH₂)₁₋₃(oxoazetidinyl); —NHC(═O)(CH₂)₁₋₃(imidazo[1,2-a]pyridin-2-yl); —NHC(═O)(CH₂)₁₋₃C(═O)-(pyrrolidin-1-yl); —NHC(═O)O(C₁-C₆ alkyl); —NHC(═O)O(C₃-C₈ cycloalkyl); —NHC(═O)O(C₁-C₆ haloalkyl); —NHC(═O))O(CH₂)₁₋₃(pyridinyl); —NHC(═O)O(CH₂)₁₋₃(pyrazinyl); —NHC(═O)O(CH₂)₁₋₃(pyrimidinyl); —NHC(═O)O(CH₂)₁₋₃(quinolinyl); —NHC(═O)O(CH₂)₁₋₃(isoxazolyl); —NHC(═O)O(CH₂)₁₋₃(oxazolyl); —NHC(═O)O(CH₂)₁₋₃(oxadiazolyl); —NHC(═O)O(CH₂)₁₋₃(triazolyl); —NHC(═O)O(CH₂)₁₋₃(thiazolyl); —NHC(═O)O(CH₂)₁₋₃(imidazolyl); —NHC(═O)O(CH₂)₁₋₃(pyrazolyl); —NHC(═O)O(CH₂)₁₋₃(piperidinyl): —NHC(═O)O(CH₂)₁₋₃(oxopiperidinyl): —NHC(═O)O(CH₂)₁₋₃(pyrrolidinyl); —NHC(═O)O(CH₂)₁₋₃(oxopyrrolidinyl); —NHC(═O)O(CH₂)₁₋₃(tetrahydrofuryl); —NHC(═O)O(CH₂)₁₋₃(tetrahydropyranyl); —NHC(═O)O(CH₂)₁₋₃(2-oxooxazolidinyl); —NHC(═O)O(CH₂)₁₋₃(morpholinyl); —NHC(═O)O(CH₂)₁₋₃(thiomorpholinyl): —NHC(═O)O(CH₂)₁₋₃(1-oxido-thiomorpholinyl); —NHC(═O)O(CH₂)₁₋₃(1,1-dioxido-thiomorpholinyl); —NHC(═O)O(CH₂)₁₋₃(oxoazetidinyl); —NHC(═O)O(CH₂)₁₋₃(imidazo[1,2-a]pyridin-2-yl); —NHC(═O)O(CH₂)₁₋₃C(═O)-(pyrrolidin-1-yl); —NHC(═O)NH(C₁-C₆ alkyl); —NHC(═O)NH(C₃-C₈ cycloalkyl); —NHC(═O)NH(C₁-C₆ haloalkyl); —NHC(═O)NH(CH₂)₁₋₃(pyridinyl); —NHC(═O)NH(CH₂)₁₋₃(pyrazinyl); —NHC(═O)NH(CH₂)₁₋₃(pyrimidinyl); —NHC(═O)NH(CH₂)₁₋₃(quinolinyl); —NHC(═O)NH(CH₂)₁₋₃(isoxazolyl); —NHC(═O)NH(CH₂)₁₋₃(oxazolyl); —NHC(═O)NH(CH₂)₁₋₃(oxadiazolyl); —NHC(═O)NH(CH₂)₁₋₃(triazolyl); —NHC(═O)NH(CH₂)₁₋₃(thiazolyl); —NHC(═O)NH(CH₂)₁₋₃(imidazolyl); —NHC(═O)NH(CH₂)₁₋₃(pyrazolyl); —NHC(═O)NH(CH₂)₁₋₃(piperidinyl); —NHC(═O))NH(CH₂)₁₋₃(oxopiperidinyl); —NHC(═O)NH(CH₂)₁₋₃(pyrrolidinyl); —NHC(═O)NH(CH₂)₁₋₃(oxopyrrolidinyl); —NHC(═O))NH(CH₂)₁₋₃(tetrahydrofuryl); —NHC(═O)NH(CH₂)₁₋₃(tetrahydropyranyl); —NHC(═O)NH(CH₂)₁₋₃(2-oxooxazolidinyl); —NHC(═O)NH(CH₂)₁₋₃(morpholinyl); —NHC(═O)NH(CH₂)₁₋₃(thiomorpholinyl); —NHC(═O)NH(CH₂)₁₋₃(1-oxido-thiomorpholinyl); —NHC(═O)NH(CH₂)₁₋₃(1,1-dioxido-thiomorpholinyl); —NHC(═O)NH(CH₂)₁₋₃(oxoazetidinyl); —NHC(═O)NH(CH₂)₁₋₃(imidazo[1,2-a]pyridin-2-yl); —NHC(═O)NH(CH₂)₁₋₃C(═O)-(pyrrolidin-1-yl); —C(═O)NHC(═O)NH—; —C(═O)N(C₁-C₆ alkyl)C(═O)NH—; —C(═O)N((CH₂)₁₋₃pyridinyl)CONH—; wherein the alkyl, cycloalkyl, heteroaryl, heterocyclyl, aryl, or benzyl group is optionally independently substituted with at least one group selected from the group consisting of C₁-C₆ alkyl; C₁-C₆ alkoxy; C₁-C₆ haloalkyl; C₁-C₆haloalkoxy; —NH₂, —NH(C₁-C₆ alkyl), —N(C₁-C₆ alkyl)(C₁-C₆ alkyl), halogen, —OH; —CN; phenoxy, —NHC(═O)H, —NHC(═O)C₁-C₆ alkyl, —C(═O)NH₂, —C(═O)NHC₁-C₆ alkyl, —C(═O)N(C₁-C₆ alkyl)(C₁-C₆ alkyl), tetrahydropyranyl, morpholinyl, —C(═O)CH₃, —C(═O)CH₂OH, —C(═O)NHCH₃, —C(═O)CH₂OMe, or an N-oxide thereof.

In certain embodiments, R₄ is H or CH₃.

In certain embodiments, R^{5a}, R^{5b}, and R^{5c} are independently selected from the group consisting of H, F, and Cl.

In certain embodiments, one of R^{5a}, R^{5b}, and R^{5c} is F, and the two remaining are H.

In certain embodiments, the compound is selected from the group consisting of:

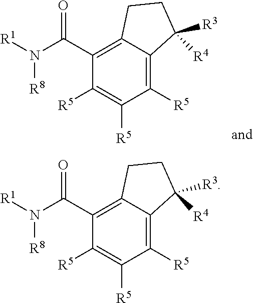

In certain embodiments, the compound is selected from the group consisting of

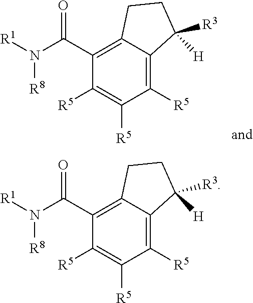

In certain embodiments, the compound is selected from the group consisting of:
O-methyl, N—(S)-(4-((3,4-difluorophenyl)carbamoyl)-2,3-dihydro-1H-inden-1-yl) carbamate;
(S)—N-(3,4-difluorophenyl)-1-(3-methylureido)-2,3-dihydro-1H-indene-4-carboxamide;
O-pyridin-2-ylmethyl, N—(S)-(4-((3,4-difluorophenyl)carbamoyl)-2,3-dihydro-1H-inden-1-yl) carbamate;
O—((R)-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-tert-butyl, N—(S)-(4-((3-chloro-4-fluorophenyl)carbamoyl)-7-fluoro-2,3-dihydro-1H-inden-1-yl) carbamate;
O-methyl, N—(S)-(7-fluoro-4-((4-fluoro-3-methylphenyl)carbamoyl)-2,3-dihydro-1H-inden-1-yl) carbamate;
(S)-7-fluoro-N-(4-fluoro-3-methylphenyl)-1-(3-methylureido)-2,3-dihydro-1H-indene-4-carboxamide;
(S)-1-amino-N-(3-chloro-4-fluorophenyl)-7-fluoro-2,3-dihydro-1H-indene-4-carboxamide;
O-2-(2-oxopyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-chloro-4-fluorophenyl)carbamoyl)-2,3-dihydro-1H-inden-1-yl) carbamate;
O—((S)-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-methylureido)-2,3-dihydro-1H-indene-4-carboxamide;
O—((R)-5-oxopyrrolidin-2-yl)methyl, N—((S)-4-((3-chloro-4-fluorophenyl)carbamoyl)-2,3-dihydro-1H-inden-1-yl) carbamate;
O—((S)-5-oxopyrrolidin-2-yl)methyl, N—((S)-4-((3-chloro-4-fluorophenyl)carbamoyl)-2,3-dihydro-1H-inden-1-yl) carbamate;
O-pyridin-2-ylmethyl, N—(S)-(4-((4-fluoro-3-methylphenyl)carbamoyl)-2,3-dihydro-1H-inden-1-yl) carbamate;
O—((R)-5-oxopyrrolidin-2-yl)methyl, N—((S)-4-((4-fluoro-3-methylphenyl)carbamoyl)-2,3-dihydro-1H-inden-1-yl)carbamate;
O—((S)-5-oxopyrrolidin-2-yl)methyl, N—((S)-4-((4-fluoro-3-methylphenyl)carbamoyl)-2,3-dihydro-1H-inden-1-yl) carbamate;
O-2-oxo-2-(pyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-chloro-4-fluorophenyl)carbamoyl)-7-fluoro-2,3-dihydro-1H-inden-1-yl) carbamate;
O—((S)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-pyridin-2-ylmethyl, N—(S)-(7-fluoro-4-((4-fluoro-3-methylphenyl)carbamoyl)-2,3-dihydro-1H-inden-1-yl) carbamate;
O-imidazo[1,2-a]pyridin-2-ylmethyl, N—(S)-(4-((3-chloro-4-fluorophenyl)carbamoyl)-7-fluoro-2,3-dihydro-1H-inden-1-yl) carbamate;
O-(6-morpholinopyridin-2-yl)methyl, N—(S)-(4-((3-chloro-4-fluorophenyl)carbamoyl)-7-fluoro-2,3-dihydro-1H-inden-1-yl) carbamate;
O—((R)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-(6-methoxypyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-2-ylamino)-2,3-dihydro-1H-indene-4-carboxamide;
O-(6-(dimethylamino) pyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-methoxypyrimidin-2-yl)amino)-2,3-dihydro-1H-indene-4-carboxamide;
(S)—N-(3-chloro-4-fluorophenyl)-7-fluoro-1-((4-(pyridin-2-yl)pyrimidin-2-yl)amino)-2,3-dihydro-1H-indene-4-carboxamide;
tert-butyl 2-(((((S)-4-((3-chloro-4-fluorophenyl)carbamoyl)-7-fluoro-2,3-dihydro-1H-inden-1-yl)carbamoyl)oxy)methyl)-4,4-difluoropyrrolidine-1-carboxylate;
O-(4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-(1-acetyl-4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-(1-(2,2,2-trifluoroethyl)piperidin-4-yl)methyl, N—(S)-(4-((3-chloro-4-fluorophenyl)carbamoyl)-7-fluoro-2,3-dihydro-1H-inden-1-yl) carbamate;
(S)-2-((((4-((3-chloro-4-fluorophenyl)carbamoyl)-7-fluoro-2,3-dihydro-1H-inden-1-yl)carbamoyl)oxy)methyl)pyridine 1-oxide;
O—(S)-1-(pyridin-2-yl)ethyl, N—((S)-4-((3-chloro-4-fluorophenyl)carbamoyl)-7-fluoro-2,3-dihydro-1H-inden-1-yl) carbamate;
O—(S)-pyrrolidin-2-ylmethyl, N—((S)-4-((3-chloro-4-fluorophen 1)carbamoyl)-7-fluoro-2,3-dihydro-1H-inden-1-yl) carbamate;
O-3,3,3-trifluoropropyl, N—(S)-(4-((3-chloro-4-fluorophenyl)carbamoyl)-7-fluoro-2,3-dihydro-1H-inden-1-yl) carbamate;
O-(1-methyl-1H-pyrazol-3-yl)methyl, N—(S)-(4-((3-chloro-4-fluorophenyl)carbamoyl)-7-fluoro-2,3-dihydro-1H-inden-1-yl) carbamate;
O—(R)-5-oxopyrrolidin-3-yl, N—((S)-4-((3-chloro-4-fluorophenyl)carbamoyl)-7-fluoro-2,3-dihydro-1H-inden-1-yl) carbamate;
O-(6-methylpyridin-2-yl)methyl, N—(S)-(4-((3-chloro-4-fluorophenyl)carbamoyl)-7-fluoro-2,3-dihydro-1H-inden-1-yl) carbamate;
N—(S)-4-((3-chloro-4-fluorophenyl)carbamoyl)-7-fluoro-2,3-dihydro-1H-inden-1-yl, 0-(pyridin-2-ylmethyl) carbamate;
(S)—N-(3-chloro-4-fluorophenyl)-7-fluoro-1-(2-methoxyacetamido)-2,3-dihydro-1H-indene-4-carboxamide;
(S)—N-(3-chloro-4-fluorophenyl)-7-fluoro-1-(3-fluoropropanamido)-2,3-dihydro-1H-indene-4-carboxamide;
(S)-1-acetamido-N-(3-chloro-4-fluorophenyl)-7-fluoro-2,3-dihydro-1H-indene-4-carboxamide;
O-pyrazin-2-ylmethyl, N—(S)-(4-((3-chloro-4-fluorophenyl)carbamoyl)-7-fluoro-2,3-dihydro-1H-inden-1-yl) carbamate;
O-pyrimidin-2-ylmethyl, N—(S)-(4-((3-chloro-4-fluorophenyl)carbamoyl)-7-fluoro-2,3-dihydro-1H-inden-1-yl) carbamate;
O-(4-chloropyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-hydroxy-2,3-dihydro-1H-indene-4-carboxamide;
O-isoxazol-3-ylmethyl, N—(S)-(4-((3-chloro-4-fluorophenyl)carbamoyl)-7-fluoro-2,3-dihydro-1H-inden-1-yl) carbamate;
O-2-(pyridin-2-yl)ethyl, N—(S)-(4-((3-chloro-4-fluorophenyl)carbamoyl)-7-fluoro-2,3-dihydro-1H-inden-1-yl) carbamate;
O-2,2-difluoroethyl, N—(S)-(4-((3-chloro-4-fluorophenyl)carbamoyl)-7-fluoro-2,3-dihydro-1H-inden-1-yl) carbamate;
O-pyrimidin-4-ylmethyl, N—(S)-(4-((3-chloro-4-fluorophenyl)carbamoyl)-7-fluoro-2,3-dihydro-1H-inden-1-yl) carbamate;
O-3-(2-oxopyrrolidin-1-yl)propyl, N-(8)-(4-((3-chloro-4-fluorophenyl)carbamoyl)-7-fluoro-2,3-dihydro-1H-inden-1-yl) carbamate;
O-(8-methylimidazo[1,2-a]pyridin-2-yl)methyl, N—(S)-(4-((3-chloro-4-fluorophenyl)carbamoyl)-7-fluoro-2,3-dihydro-1H-inden-1-yl) carbamate;
O-2,2,2-trifluoroethyl, N—(S)-(4-((3-chloro-4-fluorophenyl)carbamoyl)-7-fluoro-2,3-dihydro-1H-inden-1-yl) carbamate;
O—(S)-4-((3-chloro-4-fluorophenyl)carbamoyl)-7-fluoro-2,3-dihydro-1H-inden-1-yl, N-methylcarbamate;
N—(S)-4-((3-chloro-4-fluorophenyl)carbamoyl)-7-fluoro-2,3-dihydro-1H-inden-1-yl, O-(pyridin-2-ylmethyl) carbonate;
O-thiazol-5-ylmethyl, N—(S)-(4-((3-chloro-4-fluorophenyl)carbamoyl)-7-fluoro-2,3-dihydro-1H-inden-1-yl) carbamate;
O-thiazol-2-ylmethyl, N—(S)-(4-((3-chloro-4-fluorophenyl)carbamoyl)-7-fluoro-2,3-dihydro-1H-inden-1-yl) carbamate;
O-oxazol-4-ylmethyl, N—(S)-(4-((3-chloro-4-fluorophenyl)carbamoyl)-7-fluoro-2,3-dihydro-1H-inden-1-yl) carbamate;
O-oxazol-2-ylmethyl, N—(S)-(4-((3-chloro-4-fluorophenyl)carbamoyl)-7-fluoro-2,3-dihydro-1H-inden-1-yl) carbamate;
O-oxazol-5-ylmethyl, N—(S)-(4-((3-chloro-4-fluorophenyl)carbamoyl)-7-fluoro-2,3-dihydro-1H-inden-1-yl) carbamate;
O-2-(1H-imidazol-1-yl)ethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyridin-2-ylamino)-2,3-dihydro-1H-indene-4-carboxamide;
(S)—N-(4-((3-chloro-4-fluorophenyl)carbamoyl)-7-fluoro-2,3-dihydro-1H-inden-1-yl)-1-methyl-1H-pyrazole-3-carboxamide;
O-2-phenoxyethyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)-1-methyl-1H-pyrazole-5-carboxamide;
(S)—N-(3-chloro-4-fluorophenyl)-7-fluoro-1-((1-methyl-1H-pyrazole)-3-sulfonamido)-2,3-dihydro-1H-indene-4-carboxamide;
O-(1-methyl-1H-1,2,4-triazol-3-yl)methyl, N—(S)-(4-((3-chloro-4-fluorophenyl)carbamoyl)-7-fluoro-2,3-dihydro-1H-inden-1-yl) carbamate;
O-(1-methyl-1H-pyrazol-5-yl)methyl, N—(S)-(4-((3-chloro-4-fluorophenyl)carbamoyl)-7-fluoro-2,3-dihydro-1H-inden-1-yl) carbamate;
(S)-2-((4-((3-chloro-4-fluorophenyl)carbamoyl)-7-fluoro-2,3-dihydro-1H-inden-1-yl)amino)pyrimidine-4-carboxamide;
O-2-(4-methylthiazol-5-yl)ethyl, N—(S)-(4-((3-chloro-4-fluorophenyl)carbamoyl)-7-fluoro-2,3-dihydro-1H-inden-1-yl) carbamate;
O-(1-isopropyl-1H-pyrazol-3-yl)methyl, N—(S)-(4-((3-chloro-4-fluorophenyl)carbamoyl)-7-fluoro-2,3-dihydro-1H-inden-1-yl) carbamate;
O-(5-methoxypyridin-2-yl)methyl, N-(5)-(4-((3-chloro-4-fluorophenyl)carbamoyl)-7-fluoro-2,3-dihydro-1H-inden-1-yl) carbamate;
O—((S)-1-(2,2,2-trifluoroethyl)pyrrolidin-2-yl)methyl, N—((S)-4-((3-chloro-4-fluorophenyl)carbamoyl)-7-fluoro-2,3-dihydro-1H-inden-1-yl) carbamate;
O-(5-fluoropyridin-2-yl)methyl, N—(S)-(4-((3-chloro-4-fluorophenyl)carbamoyl)-7-fluoro-2,3-dihydro-1H-inden-1-yl) carbamate;
O-2-(1H-pyrazol-4-yl)ethyl, N—(S)-(4-((3-chloro-4-fluorophenyl)carbamoyl)-7-fluoro-2,3-dihydro-1H-inden-1-yl) carbamate;
O-2-methoxyethyl, N—(S)-(4-((3-chloro-4-fluorophenyl)carbamoyl)-7-fluoro-2,3-dihydro-1H-inden-1-yl) carbamate;
O—((R)-tetrahydrofuran-2-yl)methyl, N—((S)-4-((3-chloro-4-fluorophenyl)carbamoyl)-7-fluoro-2,3-dihydro-1H-inden-1-yl) carbamate;
O-tetrahydro-2H-pyran-4-yl, N—(S)-(4-((3-chloro-4-fluorophenyl)carbamoyl)-7-fluoro-2,3-dihydro-1H-inden-1-yl) carbamate; O-3-methoxypropyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)picolinamide;
(S)—N-(4-((3-chloro-4-fluorophenyl)carbamoyl)-7-fluoro-2,3-dihydro-1H-inden-1-yl)thiazole-5-carboxamide;
(S)—N-(3-chloro-4-fluorophenyl)-7-fluoro-1-(methylsulfonamido)-2,3-dihydro-1H-indene-4-carboxamide;
(S)—N-(3-chloro-4-fluorophenyl)-7-fluoro-1-(2-morpholinoacetamido)-2,3-dihydro-1H-indene-4-carboxamide;
(S)—N-(4-((3-chloro-4-fluorophenyl)carbamoyl)-7-fluoro-2,3-dihydro-1H-inden-1-yl)nicotinamide;
(S)—N-(4-((3-chloro-4-fluorophenyl)carbamoyl)-7-fluoro-2,3-dihydro-1H-inden-1-yl)isonicotinamide;
(S)-4-((3-chloro-4-fluorophenyl)carbamoyl)-7-fluoro-2,3-dihydro-1H-inden-1-yl methyl carbonate;
O-thiazol-4-ylmethyl. N—(S)-(4-((3-chloro-4-fluorophenyl)carbamoyl)-7-fluoro-2,3-dihydro-1H-inden-1-yl) carbamate;
O-3-(1H-imidazol-1-yl)propyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-cyano-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)thiazole-2-carboxamide;
(S)—N-(3-chloro-4-fluorophenyl)-1-(cyclopropanesulfonamido)-7-fluoro-2,3-dihydro-1H-indene-4-carboxamide;
(S)—N-(4-((3-chloro-4-fluorophenyl)carbamoyl)-7-fluoro-2,3-dihydro-1H-inden-1-yl)oxazole-5-carboxamide;
O-cyclopentyl, N—(S)-(4-((3-chloro-4-fluorophenyl)carbamoyl)-7-fluoro-2,3-dihydro-1H-inden-1-yl)carbamate;
O-(2-oxo-oxazolidin-5-yl)methyl, N—((S)-4-((3-chloro-4-fluorophenyl)carbamoyl)-7-fluoro-2,3-dihydro-1H-inden-1-yl) carbamate;
O-2-(1H-pyrazol-1-yl)ethyl, N—(S)-(4-((3-chloro-4-fluorophenyl)carbamoyl)-7-fluoro-2,3-dihydro-1H-inden-1-yl) carbamate;
O-(1-methyl-1H-imidazol-2-yl)methyl, N—(S)-(4-((3-chloro-4-fluorophenyl)carbamoyl)-7-fluoro-2,3-dihydro-1H-inden-1-yl) carbamate;
O-(3-fluoropyridin-2-yl)methyl, N—(S)-(4-((3-chloro-4-fluorophenyl)carbamoyl)-7-fluoro-2,3-dihydro-1H-inden-1-yl) carbamate;
O—((R)-morpholin-3-yl)methyl, N—((S)-4-((3-chloro-4-fluorophenyl)carbamoyl)-7-fluoro-2,3-dihydro-1H-inden-1-yl) carbamate;
O-(4-methoxypyridin-2-yl)methyl, N—(S)-(4-((3-chloro-4-fluorophenyl)carbamoyl)-7-fluoro-2,3-dihydro-1H-inden-1-yl) carbamate;
O-2-hydroxyethyl, N—(S)-(4-((3-chloro-4-fluorophenyl)carbamoyl)-7-fluoro-2,3-dihydro-1H-inden-1-yl) carbamate;
O—((S)-tetrahydrofuran-2-yl)methyl, N-((5)-4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-(2-hydroxyacetamido)-2,3-dihydro-1H-indene-4-carboxamide;
(S)—N-(3-chloro-4-fluorophenyl)-7-fluoro-1-(3-(pyridin-3-yl)ureido)-2,3-dihydro-1H-indene-4-carboxamide;
(S)—N-(3-chloro-4-fluorophenyl)-7-fluoro-1-(3-(pyridin-4-yl)ureido)-2,3-dihydro-1H-indene-4-carboxamide;
(S)—N-(3-chloro-4-fluorophenyl)-7-fluoro-1-(thiazol-2-ylamino)-2,3-dihydro-1H-indene-4-carboxamide;
O-2-(piperidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-(difluoromethyl)-4-fluorophenyl)carbamoyl)-7-fluoro-2,3-dihydro-1H-inden-1-yl) carbamate;
(S)—N-(3-chloro-4-fluorophenyl)-7-fluoro-1-(3-(pyridin-2-ylmethyl)ureido)-2,3-dihydro-1H-indene-4-carboxamide;
O-(6-cyanopyridin-2-yl)methyl, N—(S)-(4-((3-chloro-4-fluorophenyl)carbamoyl)-7-fluoro-2,3-dihydro-1H-inden-1-yl) carbamate;
O-quinolin-2-ylmethyl, N—(S)-(4-((3-chloro-4-fluorophenyl)carbamoyl)-7-fluoro-2,3-dihydro-1H-inden-1-yl) carbamate;
O-(5-methylpyrazin-2-yl)methyl, N—(S)-(4-((3-chloro-4-fluorophenyl)carbamoyl)-7-fluoro-2,3-dihydro-1H-inden-1-yl) carbamate;
O-2-morpholinoethyl-N—(S)-(4-((3-chloro-4-fluorophenyl)carbamoyl)-7-fluoro-2,3-dihydro-1H-inden-1-yl) carbamate;
O-[cis-4-hydroxycyclohexyl]-N—((S)-4-((3-chloro-4-fluorophenyl)carbamoyl)-7-fluoro-2,3-dihydro-1H-inden-1-yl)carbamate;
O-3-hydroxypropyl, N—(S)-(4-((3-chloro-4-fluorophenyl)carbamoyl)-7-fluoro-2,3-dihydro-1H-inden-1-yl) carbamate;
O-[trans-4-hydroxycyclohexyl]-N—((S)-4-((3-chloro-4-fluorophenyl)carbamoyl)-7-fluoro-2,3-dihydro-1H-inden-1-yl) carbamate;
O-2-acetamidoethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-propionamido-2,3-dihydro-1H-indene-4-carboxamide;
(S)—N-(3-chloro-4-fluorophenyl)-7-fluoro-1-((4-methoxypyrimidin-2-yl)amino)-2,3-dihydro-1H-indene-4-carboxamide;
(S)—N-(3-chloro-4-fluorophenyl)-7-fluoro-1-((4-methylpyrimidin-2-yl)amino)-2,3-dihydro-1H-indene-4-carboxamide;
(S)—N-(3-chloro-4-fluorophenyl)-7-fluoro-1-((2-methoxypyrimidin-4-yl)amino)-2,3-dihydro-1H-indene-4-carboxamide;
(S)—N-(3-chloro-4-fluorophenyl)-7-fluoro-1-((5-methylpyrimidin-2-yl)amino)-2,3-dihydro-1H-indene-4-carboxamide;
(S)—N-(3-chloro-4-fluorophenyl)-7-fluoro-1-((6-methoxypyrimidin-4-yl)amino)-2,3-dihydro-1H-indene-4-carboxamide;
(S)—N-(3-chloro-4-fluorophenyl)-1-((4,6-dimethylpyrimidin-2-yl)amino)-7-fluoro-2,3-dihydro-1H-indene-4-carboxamide;
O—(S)-5-oxopyrrolidin-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-(pyridin-2-yl)ethyl)sulfonamido)-2,3-dihydro-1H-indene-4-carboxamide;
O-(6-(trifluoromethyl)pyridin-2-yl)methyl, N—((S)-(4-((3-chloro-4-fluorophenyl)carbamoyl)-7-fluoro-2,3-dihydro-1H-inden-1-yl) carbamate;
O-(5-(trifluoromethyl) pyridin-2-yl)methyl, N—(S)-(4-((3-chloro-4-fluorophenyl)carbamoyl)-7-fluoro-2,3-dihydro-1H-inden-1-yl) carbamate;
O—(R)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-(1-methyl-1H-pyrazol-3-yl)propanamido)-2,3-dihydro-1H-indene-4-carboxamide;
O-(5-cyanopyridin-2-yl)methyl, N—(S)-(4-((3-chloro-4-fluorophenyl)carbamoyl)-7-fluoro-2,3-dihydro-1H-inden-1-yl) carbamate;
O-(3-methylpyrazin-2-yl)methyl, N—(S)-(4-((3-chloro-4-fluorophenyl)carbamoyl)-7-fluoro-2,3-dihydro-1H-inden-1-yl) carbamate;
O-(1-acetylpiperidin-4-yl)methyl, N—(S)-(4-((3-chloro-4-fluorophenyl)carbamoyl)-7-fluoro-2,3-dihydro-1H-inden-1-yl) carbamate;
O-(1-(2-hydroxyacetyl)piperidin-4-yl)methyl, N—(S)-(4-((3-chloro-4-fluorophenyl)carbamoyl)-7-fluoro-2,3-dihydro-1H-inden-1-yl) carbamate;
O-(1-(methylcarbamoyl)piperidin-4-yl)methyl, N—(S)-(4-((3-chloro-4-fluorophenyl)carbamoyl)-7-fluoro-2,3-dihydro-1H-inden-1-yl) carbamate;
O-(1,1-dioxidothiomorpholin-3-yl)methyl-N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-1-(cyclopropanecarboxamido)-7-fluoro-2,3-dihydro-1H-indene-4-carboxamide;
O—((S)-morpholin-3-yl)methyl, N—((S)-4-((3-chloro-4-fluorophenyl)carbamoyl)-7-fluoro-2,3-dihydro-1H-inden-1-yl) carbamate;
O—(S)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-methoxyethyl) sulfonamido)-2,3-dihydro-1H-indene-4-carboxamide;
(S)—N-(3-chloro-4-fluorophenyl)-7-fluoro-1-(phenylsulfonamido)-2,3-dihydro-1H-indene-4-carboxamide;
(S)—N-(3-chloro-4-fluorophenyl)-7-fluoro-1-(pyridine-2-sulfonamido)-2,3-dihydro-1H-indene-4-carboxamide;
O-(1-(2-methoxyacetyl) piperidin-4-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-hydroxypyrimidin-2-yl)amino)-2,3-dihydro-1H-indene-4-carboxamide
O-(1H-pyrazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-((1-methyl-1H-pyrazol-3-yl)methyl)ureido)-2,3-dihydro-1H-indene-4-carboxamide;
O-(1H-1,2,4-triazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-4-ylamino)-2,3-dihydro-1H-indene-4-carboxamide;
(S)—N-(3-chloro-4-fluorophenyl)-7-fluoro-1-((7-(4-methoxybenzyl)-7H-pyrrolo[2,3-d]pyrimidin-2-yl)amino)-2,3-dihydro-1H-indene-4-carboxamide;
O—((R)-6-oxopiperidin-2-yl)methyl, N—((S)-4-((3-chloro-4-fluorophenyl)carbamoyl)-7-fluoro-2,3-dihydro-1H-inden-1-yl) carbamate;
O—(R)-6-oxopiperidin-3-yl, N—((S)-4-(3-chloro-4-fluorophenyl)carbamoyl)-7-fluoro-2,3-dihydro-1H-inden-1-yl) carbamate;
O—(S)-6-oxopiperidin-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-1-(3-cyclopropylureido)-7-fluoro-2,3-dihydro-1H-indene-4-carboxamide;
O—((S)-6-oxopiperidin-2-yl)methyl, N—((S)-4-((3-chloro-4-fluorophenyl)carbamoyl)-7-fluoro-2,3-dihydro-1H-inden-1-yl) carbamate;
O-(4-oxoazetidin-2-yl)methyl, N—((S)-4-((3-chloro-4-fluorophenyl)carbamoyl)-7-fluoro-2,3-dihydro-1H-inden-1-yl) carbamate;
O-methyl, N-(4-((3-chloro-4-fluorophenyl)carbamoyl)-7-fluoro-1-methyl-2,3-dihydro-1H-inden-1-yl) carbamate;
N-(3-chloro-4-fluorophenyl)-7-fluoro-1-methyl-1-(3-methylureido)-2,3-dihydro-1H-indene-4-carboxamide;
(S)—N-(3-chloro-4-fluorophenyl)-1-(cyclopropanesulfonamido)-2,3-dihydro-1H-indene-4-carboxamide;
O-pyridin-2-ylmethyl, N-(4-((3-chloro-4-fluorophenyl)carbamoyl)-7-fluoro-1-methyl-2,3-dihydro-1H-inden-1-yl) carbamate;
(S)—N-(3-chloro-4-fluorophenyl)-1-((cyclopropylmethyl)sulfonamido)-7-fluoro-2,3-dihydro-1H-indene-4-carboxamide;
(S)—N-(3-chloro-4-fluorophenyl)-7-fluoro-1-((phenylmethyl)sulfonamido)-2,3-dihydro-1H-indene-4-carboxamide;
O-cyclopropyl, N—(S)-(4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-((N-methylsulfamoyl)amino)-2,3-dihydro-1H-indene-4-carboxamide;
(S)—N-(3-chloro-4-fluorophenyl)-7-fluoro-1-(morpholine-4-sulfonamido)-2,3-dihydro-1H-indene-4-carboxamide;
O-cyclopropyl, N—(S)-(4-((3-chloro-4-fluorophenyl)carbamoyl)-2,3-dihydro-1H-inden-1-yl) carbamate;
(S)—N-(3-chloro-4-fluorophenyl)-1-((N-methylsulfamoyl)amino)-2,3-dihydro-1H-indene-4-carboxamide;
O-(1,3,4-oxadiazol-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-1-(ethylsulfonamido)-7-fluoro-2,3-dihydro-1H-indene-4-carboxamide;
(S)—N-(3-chloro-4-fluorophenyl)-7-fluoro-1-(propylsulfonamido)-2,3-dihydro-1H-indene-4-carboxamide;
(S)—N-(4-chloro-3-fluorophenyl)-7-fluoro-1-((2-methylpropyl)sulfonamido)-2,3-dihydro-1H-indene-4-carboxamide;
(S)—N-(3-chloro-4-fluorophenyl)-7-fluoro-1-((N-isopropylsulfamoyl)amino)-2,3-dihydro-1H-indene-4-carboxamide;
(S)—N-(3-chloro-4-fluorophenyl)-7-fluoro-1-((1-methylethyl)sulfonamido)-2,3-dihydro-1H-indene-4-carboxamide;
(S)—N-(3-chloro-4-fluorophenyl)-1-(cyclopentanesulfonamido)-7-fluoro-2,3-dihydro-1H-indene-4-carboxamide;
(S)—N-(3-chloro-4-fluorophenyl)-1-(cyclohexanesulfonamido)-7-fluoro-2,3-dihydro-1H-indene-4-carboxamide;
(S)—N-(3-chloro-4-fluorophenyl)-1-((N-cyclopropylsulfamoyl)amino)-2,3-dihydro-1H-indene-4-carboxamide;
(S)—N-(3-chloro-4-fluorophenyl)-1-((N-cyclopropylsulfamoyl)amino)-7-fluoro-2,3-dihydro-1H-indene-4-carboxamide;
O-(1-(tetrahydro-2H-pyran-2-yl)-1H-1,2,4-triazol-3-yl)methyl, N—((S)-4-((3-chloro-4-fluorophenyl)carbamoyl)-7-fluoro-2,3-dihydro-1H-inden-1-yl) carbamate;
N-(3-Chloro-4-fluorophenyl)-7-fluoro-1-oxo-2,3-dihydro-1H-indene-4-carboxamide;
((1-(methyl-d₃)-1H-1,2,4-triazol-3-yl)methyl-d₂(S)-(4-((3-chloro-4-fluorophenyl)carbamoyl)-7-fluoro-2,3-dihydro-1H-inden-1-yl)carbamate;
(S)-(3-((((4-((3-chloro-4-fluorophenyl)carbamoyl)-7-fluoro-2,3-dihydro-1H-inden-1-yl)carbamoyl)oxy)methyl)-1H-1,2,4-triazol-1-yl)methyl phosphoric acid;
(S)-(3-((((4-((3-chloro-4-fluorophenyl)carbamoyl)-7-fluoro-2,3-dihydro-1H-inden-1-yl)carbamoyl)oxy)methyl)-1H-pyrazol-1-yl)methyl phosphoric acid;
O—(S)-2-cyanoethyl, N-4-(3-chloro-4-fluorophenylcarbamoyl)-7-fluoro-2,3-dihydro-1H-inden-1-yl carbamate;
O—(S)-3-cyanopropyl, N-4-(3-chloro-4-fluorophenylcarbamoyl)-7-fluoro-2,3-dihydro-1H-inden-1-yl carbamate;
N-(3-chloro-4-fluorophenyl)-7′-fluoro-2,5-dioxo-2′,3′-dihydrospiro[imidazolidine-4,1′-indene]-4′-carboxamide;
N-(3-chloro-4-fluorophenyl)-7′-fluoro-2,5-dioxo-1-(pyridin-2-ylmethyl)-2′,3′-dihydrospiro[imidazolidine-4,1′-indene]-4′-carboxamide;
N-(3-chloro-4-fluoro-phenyl)-7′-fluoro-1-methyl-2,5-dioxo-spiro[imidazolidine-4,1′-indane]-4′-carboxamide;
(S)-1-(((S)-tert-butylsulfinyl)amino)-N-(3-chloro-4-fluorophenyl)-7-fluoro-2,3-dihydro-1H-indene-4-carboxamide;
(S)-1-(((R)-tert-butylsulfinyl)amino)-N-(3-chloro-4-fluorophenyl)-7-fluoro-2,3-dihydro-1H-indene-4-carboxamide;
   or a salt thereof.

In certain embodiments, a capsid inhibitor is a compound of the following formula, or a salt thereof:

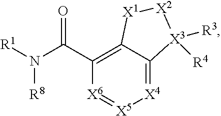

wherein the following definitions apply:
—X¹—X²— is selected from the group consisting of —CH₂CH₂—*, —CH₂CH(CH₃)—*, —CH₂C(CH₃)₂—*, —CH(CH₃)CH₂—*, —C(CH₃)₂CH₂—*, —CH₂CHF—*, —CH₂CF₂—*, —OCH₂—*, —SCH₂—*, —CH₂NR^{6a}—*, and —CH₂CH(OR^{6a})—*, wherein the single bond marked as “*” is between —X¹—X²— and X³;
X³ is C, or X³ combines with R³ and R⁴ to form —S(═O)₂—;
X⁴ is N or C(R^{5a}),
X⁵ is N or C(R^{5b}),
X⁶ is N or C(R^{5c}),
   wherein 0-1 of X⁴, X⁵, and X⁶ is N;
R¹ is selected from the group consisting of optionally substituted phenyl, optionally substituted benzyl, optionally substituted heteroaryl, and —(CH₂)(optionally substituted heteroaryl);
each occurrence of R² is independently selected from the group consisting of H and C₁-C₆ alkyl;
R³ is selected from the group consisting of —N(R)C(═O)OR⁶, H, —OH, —OR⁶, —NH₂, —NHR⁶, —NR⁶R⁶, —OC(═O)OR⁶, —OC(═O)N(R²)R⁶, —NR⁷C(═O)N(R⁶)(R⁷), —N(R²)C(═O)R⁶, —NR²S(═O)₁₋₂R⁶, optionally substituted aryl, optionally substituted heteroaryl, —CH₂C(O)OH, —CH₂C(═O)NR⁶R⁶, —N(R²)C(═O)(CH₂)₁₋₂R⁶, NR²S(═O)₂N(R⁶)(R⁷), and —NR²C(═O)C(═O)N(R⁶)(R⁷);
R⁴ is H or C₁-C₆ alkyl,
   or R³ and R⁴ combine to form ═O or —C(═O)NR^{6a}—C(═O)—NR^{6a}—;
R^{5a} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;
R^{5b} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;
R^{5c} is independently selected from the group consisting of H, halo, C₁-C₆ alkyl, C₁-C₆ alkoxy, C₁-C₆ aminoalkyl, C₁-C₆ haloalkoxy, and C₁-C₆ haloalkyl;
each occurrence of R⁶ is independently selected from the group consisting of optionally substituted C₁-C₆ alkyl, optionally substituted C₃-C₈ cycloalkyl, optionally substituted phenyl, and optionally substituted hetereoaryl;
each occurrence of R^{6a} is independently selected from the group consisting of H, optionally substituted C₁-C₆ alkyl, optionally substituted C₃-C₈ cycloalkyl, optionally substituted phenyl, and optionally substituted hetereoaryl;
each occurrence of R⁷ is independently selected from the group consisting of H and optionally substituted C₁-C₆ alkyl;
   or, if R⁶ and R⁷ are bound to the same N atom, R⁶ and R⁷ optionally combine with the N atom to which both are bound to form an optionally substituted 3-7 membered heterocycle:
R⁸ is selected from the group consisting of H and C₁-C₆ alkyl.

In certain embodiments, a capsid inhibitor is a compound of the following formula, or a salt thereof:

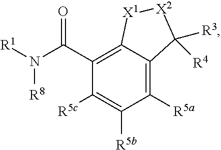

wherein the following definitions apply:
—X¹—X²— is selected from the group consisting of —CH₂CH₂—*, —CH₂CH(CH₃)—*, —CH₂C(CH₃)₂—*, —CH(CH₃)CH₂—*, —C(CH₃)₂CH₂—*, —CH₂CHF—*, —CH₂CF₂—*, —OCH₂—*, —SCH₂—*, and —CH₂CH(OR²)—*, wherein the single bond marked as “*” is between —X¹—X²— and —CR³R⁴—;
R¹ is selected from the group consisting of optionally substituted phenyl, optionally substituted benzyl, optionally substituted heteroaryl, and —(CH₂)(optionally substituted heteroaryl);
each occurrence of R² is independently selected from the group consisting of H and C₁-C₆ alkyl;
R³ is selected from the group consisting of H, —OH, —OR⁶, —NH₂, —NHR⁶, —NR⁶R⁶, —OC(═O)OR⁶, —OC(═O)N(R²)R⁶, —N(R²)C(═O)OR⁶—NR⁷C(O)N(R⁶)(R⁷), —N(R²)C(═O)R⁶, —NR²S(═O)₂R⁶, optionally substituted aryl, optionally substituted heteroaryl, —CH₂C(═O)OH, —CH₂C(O)NR⁶R⁶, —N(R²)C(═O)(CH₂)₀₋₂R⁶, NR²S(═O)₂N(R⁶)(R⁷), and —NR²C(═O)C(═O)N(R⁶)(R⁷);
R⁴ is H or C₁-C₆ alkyl, or R³ and R⁴ combine to form ═O;
R^{5a} is selected from the group consisting of H, halo. C₁-C₆ alkyl, C₁-C₆ alkoxy, C₁-C₆ aminoalkyl, C₁-C₆ haloalkoxy, and C₁-C₆ haloalkyl;
R^{5b} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl:
R^{5c} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;
each occurrence of R⁶ is independently selected from the group consisting of optionally substituted C₁-C₆ alkyl, optionally substituted C₃-C₈ cycloalkyl, optionally substituted phenyl, and optionally substituted hetereoaryl;
each occurrence of R⁷ is independently selected from the group consisting of H and optionally substituted C₁-C₆ alkyl;
   or, if R⁶ and R⁷ are bound to the same N atom, R⁶ and R⁷ optionally combine with the N atom to which both are bound to form an optionally substituted 3-7 membered heterocycle;
R⁸ is selected from the group consisting of H and C₁-C₆ alkyl.

In certain embodiments, at least one of R^{5a}, R^{5b}, and R^{5c} is H.

In certain embodiments, is a compound is:

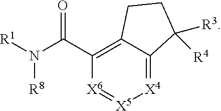

In certain embodiments, is a compound is selected from the group consisting of:

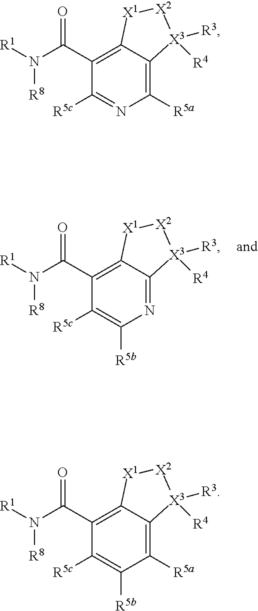

In certain embodiments, the compound is at least partially deuterated.

In certain embodiments, the compound is a prodrug.

In certain embodiments, the compound comprises a —(CRR)—O—P(═O)(OR)₂ group, or a salt thereof, which is attached to a heteroatom, wherein each occurrence of R is independently H and C₁-C₆ alkyl.

In certain embodiments, the compound is selected from the group consisting of:
O-methyl, N—(S)-(4-((3,4-difluorophenyl)carbamoyl)-2,3-dihydro-1H-inden-1-yl) carbamate;
(S)—N-(3,4-difluorophenyl)-1-(3-methylureido)-2,3-dihydro-1H-indene-4-carboxamide;
O-pyridin-2-ylmethyl, N—(S)-(4-((3,4-difluorophenyl)carbamoyl)-2,3-dihydro-1H-inden-1-yl) carbamate;
O-methyl, N-(7-((3,4-difluorophenyl)carbamoyl)-2,3-dihydrobenzofuran-3-yl) carbamate;
N-(3,4-difluorophenyl)-3-(3-methylureido)-2,3-dihydrobenzofuran-7-carboxamide;
O—((R)-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-tert-butyl, N—(S)-(4-((3-chloro-4-fluorophenyl)carbamoyl)-7-fluoro-2,3-dihydro-1H-inden-1-yl) carbamate;
O-methyl, N—(S)-(7-fluoro-4-((4-fluoro-3-methylphenyl)carbamoyl)-2,3-dihydro-1H-inden-1-yl) carbamate;
(S)-7-fluoro-N-(4-fluoro-3-methylphenyl)-1-(3-methylureido)-2,3-dihydro-1H-indene-4-carboxamide;
(S)-1-amino-N-(3-chloro-4-fluorophenyl)-7-fluoro-2,3-dihydro-1H-indene-4-carboxamide;
O-2-(2-oxopyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N-(7-((3,4-difluorophenyl)carbamoyl)-2,3-dihydrobenzofuran-3-yl) carbamate;
O-pyridin-2-ylmethyl, N—(S)-(4-((3-chloro-4-fluorophenyl)carbamoyl)-2,3-dihydro-1H-inden-1-yl) carbamate; O—((S)-5-oxopyrrolidin-211)methyl, N—((S)-4-((3-chloro-4-fluorophenyl)carbamoyl)-7-fluoro-2,3-dihydro-1H-inden-1-yl) carbamate;
O-methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-methylureido)-2,3-dihydro-1H-indene-4-carboxamide;
O—((R)-5-oxopyrrolidin-2-yl)methyl, N—((S)-4-((3-chloro-4-fluorophenyl)carbamoyl)-2,3-dihydro-1H-inden-1-yl) carbamate;
O—((S)-5-oxopyrrolidin-2-yl)methyl, N—((S)-4-((3-chloro-4-fluorophenyl)carbamoyl)-2,3-dihydro-1H-inden-1-yl) carbamate;
O-pyridin-2-ylmethyl, N—(S)-(4-((4-fluoro-3-methylphenyl)carbamoyl)-2,3-dihydro-1H-inden-1-yl) carbamate;
O—((R)-5-oxopyrrolidin-2-yl)methyl, N—((S)-4-((4-fluoro-3-methylphenyl)carbamoyl)-2,3-dihydro-1H-inden-1-yl)carbamate;
O—((S)-5-oxopyrrolidin-211)methyl, N—((S)-4-((4-fluoro-3-methylphenyl)carbamoyl)-2,3-dihydro-1H-inden-1-yl) carbamate;
O-2-oxo-2-(pyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N-(7-((3,4-difluorophenyl)carbamoyl)-2,3-dihydrobenzo[b]thiophen-3-yl) carbamate;
O-pyridin-2-ylmethyl, N-(7-((3-chloro-4-fluorophenyl)carbamoyl)-2,3-dihydrobenzo[b]thiophen-3-yl) carbamate;
O-methyl, N-(7-((3-chloro-4-fluorophenyl)carbamoyl)-2,3-dihydrobenzo[b]thiophen-3-yl) carbamate;
O-methyl, N-(7-((3,4-difluorophenyl)carbamoyl)-2,3-dihydrobenzo[b]thiophen-3-yl) carbamate;
O-pyridin-2-ylmethyl, N—(S)-(4-((3-chloro-4-fluorophenyl)carbamoyl)-7-fluoro-2,3-dihydro-1H-inden-1-yl) carbamate;
O-methyl, N-(7-((3-chloro-4-fluorophenyl)carbamoyl)-2,3-dihydrobenzo[b]thiophen-3-yl) carbamate;
O—((S)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-pyridin-2-ylmethyl, N—(S)-(7-fluoro-4-((4-fluoro-3-methylphenyl)carbamoyl)-2,3-dihydro-1H-inden-1-yl) carbamate;
O-imidazo[1,2-a]pyridin-2-ylmethyl, N—(S)-(4-((3-chloro-4-fluorophenyl)carbamoyl)-7-fluoro-2,3-dihydro-1H-inden-1-yl) carbamate;
O-(6-morpholinopyridin-2-yl)methyl, N—(S)-(4-((3-chloro-4-fluorophenyl)carbamoyl)-7-fluoro-2,3-dihydro-1H-inden-1-yl) carbamate;
O—((R)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-(6-methoxypyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-2-ylamino)-2,3-dihydro-1H-indene-4-carboxamide;
O-methyl, N-((1R,2R)-4-((3-chloro-4-fluorophenyl)carbamoyl)-2-hydroxy-2,3-dihydro-1H-inden-1-yl) carbamate;
N-(3-chloro-4-fluorophenyl)-2-hydroxy-1-(3-methylureido)-2,3-dihydro-1H-indene-4-carboxamide;
O-(6-(dimethylamino) pyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-methoxypyrimidin-2-yl)amino)-2,3-dihydro-1H-indene-4-carboxamide;
(S)—N-(3-chloro-4-fluorophenyl)-7-fluoro-1-((4-(pyridin-2-yl)pyrimidin-2-yl)amino)-2,3-dihydro-1H-indene-4-carboxamide;
O-pyridin-2-ylmethyl, N-((1R,2R)-4-((3-chloro-4-fluorophenyl)carbamoyl)-2-hydroxy-2,3-dihydro-1H-inden-1-yl) carbamate;
O-methyl, N-(4-((3,4-difluorophenyl)carbamoyl)-2-hydroxy-2,3-dihydro-1H-inden-1-yl) carbamate;
N-(3,4-difluorophenyl)-2-hydroxy-1-(3-methylureido)-2,3-dihydro-1H-indene-4-carboxamide;
tert-butyl 2-(((((S)-4-((3-chloro-4-fluorophenyl)carbamoyl)-7-fluoro-2,3-dihydro-1H-inden-1-yl)carbamoyl)oxy)methyl)-4,4-difluoropyrrolidine-1-carboxylate;
O-methyl, N-(7-((3,4-difluorophenyl)carbamoyl)-4-fluoro-2,3-dihydrobenzo[b]thiophen-3-yl) carbamate;
O-(4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-methyl, N-(7-((3-chloro-4-fluorophenyl)carbamoyl)-4-fluoro-2,3-dihydrobenzo[b]thiophen-3-yl) carbamate;
O-pyridin-2-ylmethyl, N-((1R,2R)-4-((3,4-difluorophenyl)carbamoyl)-2-hydroxy-2,3-dihydro-1H-inden-1-yl) carbamate;
O-(1-acetyl-4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-(1-(2,2,2-trifluoroethyl)piperidin-4-yl)methyl, N—(S)-(4-((3-chloro-4-fluorophenyl)carbamoyl)-7-fluoro-2,3-dihydro-1H-inden-1-yl) carbamate;
O-pyridin-2-ylmethyl, N-(7-((3,4-difluorophenyl)carbamoyl)-4-fluoro-2,3-dihydrobenzo[b]thiophen-3-yl) carbamate;
O-pyridin-2-ylmethyl, N-(7-((3-chloro-4-fluorophenyl)carbamoyl)-4-fluoro-2,3-dihydrobenzo[b]thiophen-3-yl) carbamate;
(S)-2-((((4-((3-chloro-4-fluorophenyl)carbamoyl)-7-fluoro-2,3-dihydro-1H-inden-1-yl)carbamoyl)oxy)methyl)pyridine 1-oxide;
O—(S)-1-(pyridin-2-yl)ethyl, N—((S)-4-((3-chloro-4-fluorophenyl)carbamoyl)-7-fluoro-2,3-dihydro-1H-inden-1-yl) carbamate;
O—(S)-pyrrolidin-2-ylmethyl, N—((S)-4-((3-chloro-4-fluorophenyl)carbamoyl)-7-fluoro-2,3-dihydro-1H-inden-1-yl) carbamate;
O-3,3,3-trifluoropropyl, N—(S)-(4-((3-chloro-4-fluorophenyl)carbamoyl)-7-fluoro-2,3-dihydro-1H-inden-1-yl) carbamate;
O-(1-methyl-1H-pyrazol-3-yl)methyl, N—(S)-(4-((3-chloro-4-fluorophenyl)carbamoyl)-7-fluoro-2,3-dihydro-1H-inden-1-yl) carbamate;
O—(R)-5-oxopyrrolidin-3-yl, N—((S)-4-((3-chloro-4-fluorophenyl)carbamoyl)-7-fluoro-2,3-dihydro-1H-inden-1-yl) carbamate;
O-(6-methylpyridin-2-yl)methyl, N—(S)-(4-((3-chloro-4-fluorophenyl)carbamoyl)-7-fluoro-2,3-dihydro-1H-inden-1-yl) carbamate;
N—(S)-4-((3-chloro-4-fluorophenyl)carbamoyl)-7-fluoro-2,3-dihydro-1H-inden-1-yl, O-(pyridin-2-ylmethyl) carbamate;
(S)—N-(3-chloro-4-fluorophenyl)-7-fluoro-1-(2-methoxyacetamido)-2,3-dihydro-1H-indene-4-carboxamide;
(S)—N-(3-chloro-4-fluorophenyl)-7-fluoro-1-(3-fluoropropanamido)-2,3-dihydro-1H-indene-4-carboxamide;
(S)-1-acetamido-N-(3-chloro-4-fluorophenyl)-7-fluoro-2,3-dihydro-1H-indene-4-carboxamide;
O-pyrazin-2-ylmethyl, N—(S)-(4-((3-chloro-4-fluorophenyl)carbamoyl)-7-fluoro-2,3-dihydro-1H-inden-1-yl) carbamate;
O-pyrimidin-2-ylmethyl, N—(S)-(4-((3-chloro-4-fluorophenyl)carbamoyl)-7-fluoro-2,3-dihydro-1H-inden-1-yl) carbamate;
O-(4-chloropyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-hydroxy-2,3-dihydro-1H-indene-4-carboxamide;
O-isoxazol-3-ylmethyl, N—(S)-(4-((3-chloro-4-fluorophenyl)carbamoyl)-7-fluoro-2,3-dihydro-1H-inden-1-yl) carbamate;
O-2-(pyridin-2-yl)ethyl, N—(S)-(4-((3-chloro-4-fluorophenyl)carbamoyl)-7-fluoro-2,3-dihydro-1H-inden-1-yl) carbamate;
O-2,2-difluoroethyl, N—(S)-(4-((3-chloro-4-fluorophenyl)carbamoyl)-7-fluoro-2,3-dihydro-1H-inden-1-yl) carbamate;
O-pyrimidin-4-ylmethyl, N—(S)-(4-((3-chloro-4-fluorophenyl)carbamoyl)-7-fluoro-2,3-dihydro-1H-inden-1-yl) carbamate;
O-3-(2-oxopyrrolidin-1-yl)propyl, N—(S)-(4-((3-chloro-4-fluorophenyl)carbamoyl)-7-fluoro-2,3-dihydro-1H-inden-1-yl) carbamate;
O-(8-methylimidazo[1,2-a]pyridin-2-yl)methyl, N—(S)-(4-((3-chloro-4-fluorophenyl)carbamoyl)-7-fluoro-2,3-dihydro-1H-inden-1-yl) carbamate;
O-2,2,2-trifluoroethyl, N—(S)-(4-((3-chloro-4-fluorophenyl)carbamoyl)-7-fluoro-2,3-dihydro-1H-inden-1-yl) carbamate;
O—(S)-4-((3-chloro-4-fluorophenyl)carbamoyl)-7-fluoro-2,3-dihydro-1H-inden-1-yl, N-methylcarbamate;
N—(S)-4-((3-chloro-4-fluorophenyl)carbamoyl)-7-fluoro-2,3-dihydro-1H-inden-1-yl, O-(pyridin-2-ylmethyl) carbonate;
O-thiazol-5-ylmethyl, N—(S)-(4-((3-chloro-4-fluorophenyl)carbamoyl)-7-fluoro-2,3-dihydro-1H-inden-1-yl) carbamate;
O-thiazol-2-ylmethyl. N—(S)-(4-((3-chloro-4-fluorophenyl)carbamoyl)-7-fluoro-2,3-dihydro-1H-inden-1-yl) carbamate;
O-oxazol-4-ylmethyl, N—(S)-(4-((3-chloro-4-fluorophenyl)carbamoyl)-7-fluoro-2,3-dihydro-1H-inden-1-yl) carbamate;
O-oxazol-2-ylmethyl, N-(8)-(4-((3-chloro-4-fluorophenyl)carbamoyl)-7-fluoro-2,3-dihydro-1H-inden-1-yl) carbamate;
O-oxazol-5-ylmethyl, N—(S)-(4-((3-chloro-4-fluorophenyl)carbamoyl)-7-fluoro-2,3-dihydro-1H-inden-1-yl) carbamate;
O-2-(1H-imidazol-1-yl)ethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyridin-2-ylamino)-2,3-dihydro-1H-indene-4-carboxamide;
(S)—N-(4-((3-chloro-4-fluorophenyl)carbamoyl)-7-fluoro-2,3-dihydro-1H-inden-1-yl)-1-methyl-1H-pyrazole-3-carboxamide;
O-2-phenoxyethyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)-1-methyl-1H-pyrazole-5-carboxamide;
(S)—N-(3-chloro-4-fluorophenyl)-7-fluoro-1-((1-methyl-1H-pyrazole)-3-sulfonamido)-2,3-dihydro-1H-indene-4-carboxamide;
O-(1-methyl-1H-1,2,4-triazol-3-yl)methyl, N—(S)-(4-((3-chloro-4-fluorophenyl)carbamoyl)-7-fluoro-2,3-dihydro-1H-inden-1-yl) carbamate;
O-(1-methyl-1H-pyrazol-5-yl)methyl, N—(S)-(4-((3-chloro-4-fluorophenyl)carbamoyl)-7-fluoro-2,3-dihydro-1H-inden-1-yl) carbamate;
(S)-2-((4-((3-chloro-4-fluorophenyl)carbamoyl)-7-fluoro-2,3-dihydro-1H-inden-1-yl)amino)pyrimidine-4-carboxamide;
O-2-(4-methylthiazol-5-yl)ethyl, N—(S)-(4-((3-chloro-4-fluorophenyl)carbamoyl)-7-fluoro-2,3-dihydro-1H-inden-1-yl) carbamate;
O-(1-isopropyl-1H-pyrazol-3-yl)methyl, N—(S)-(4-((3-chloro-4-fluorophenyl)carbamoyl)-7-fluoro-2,3-dihydro-1H-inden-1-yl) carbamate;
O-(5-methoxypyridin-2-yl)methyl, N—(S)-(4-((3-chloro-4-fluorophenyl)carbamoyl)-7-fluoro-2,3-dihydro-1H-inden-1-yl) carbamate;
O—((S)-1-(2,2,2-trifluoroethyl)pyrrolidin-2-yl)methyl, N—((S)-4-((3-chloro-4-fluorophenyl)carbamoyl)-7-fluoro-2,3-dihydro-1H-inden-1-yl) carbamate;
O-(5-fluoropyridin-2-yl)methyl, N—(S)-(4-((3-chloro-4-fluorophenyl)carbamoyl)-7-fluoro-2,3-dihydro-1H-inden-1-yl) carbamate;
O-2-(1H-pyrazol-4-yl)ethyl, N—(S)-(4-((3-chloro-4-fluorophenyl)carbamoyl-7-fluoro-2,3-dihydro-1H-inden-1-yl) carbamate;
O-2-methoxyethyl, N—(S)-(4-((3-chloro-4-fluorophenyl)carbamoyl)-7-fluoro-2,3-dihydro-1H-inden-1-yl) carbamate;
O—((R)-tetrahydrofuran-2-yl)methyl, N—((S)-4-((3-chloro-4-fluorophenyl)carbamoyl)-7-fluoro-2,3-dihydro-1H-inden-1-yl) carbamate;
O-tetrahydro-2H-pyran-4-yl, N—(S)-(4-((3-chloro-4-fluorophenyl)carbamoyl)-7-fluoro-2,3-dihydro-1H-inden-1-yl) carbamate;
O-3-methoxypropyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)picolinamide;
(S)—N-(4-((3-chloro-4-fluorophenyl)carbamoyl)-7-fluoro-2,3-dihydro-1H-inden-1-yl)thiazole-5-carboxamide;
(S)—N-(3-chloro-4-fluorophenyl)-7-fluoro-1-(methylsulfonamido)-2,3-dihydro-1H-indene-4-carboxamide;
(S)—N-(3-chloro-4-fluorophenyl)-7-fluoro-1-(2-morpholinoacetamido)-2,3-dihydro-1H-indene-4-carboxamide;
(S)—N-(4-((3-chloro-4-fluorophenyl)carbamoyl)-7-fluoro-2,3-dihydro-1H-inden-1-yl)nicotinamide;
(S)—N-(4-((3-chloro-4-fluorophenyl)carbamoyl)-7-fluoro-2,3-dihydro-1H-inden-1-yl)isonicotinamide:
O-methyl, N-(4-((3-chloro-4-fluorophenyl)carbamoyl)-7-fluoro-2,2-dimethyl-2,3-dihydro-1H-inden-1-yl)carbamate;
(S)-4-((3-chloro-4-fluorophenyl)carbamoyl)-7-fluoro-2,3-dihydro-1H-inden-1-yl methyl carbonate;
O-thiazol-4-ylmethyl, N—(S)-(4-((3-chloro-4-fluorophenyl)carbamoyl)-7-fluoro-2,3-dihydro-1H-inden-1-yl) carbamate;
O-3-(1H-imidazol-1-yl)propyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-cyano-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)thiazole-2-carboxamide;
(S)—N-(3-chloro-4-fluorophenyl 1-(cyclopropanesulfonamido)-7-fluoro-2,3-dihydro-1H-indene-4-carboxamide;
(S)—N-(4-((3-chloro-4-fluorophenyl)carbamoyl)-7-fluoro-2,3-dihydro-1H-inden-1-yl)oxazole-5-carboxamide;
O-methyl, N-((1R,2R)-4-((3-chloro-4-fluorophenyl)carbamoyl)-7-fluoro-2-methoxy-2,3-dihydro-1H-inden-1-yl) carbamate;
O-cyclopentyl, N—(S)-(4-((3-chloro-4-fluorophenyl)carbamoyl)-7-fluoro-2,3-dihydro-1H-inden-1-yl)carbamate;
O-(2-oxo-oxazolidin-5-yl)methyl, N—((S)-4-((3-chloro-4-fluorophenyl)carbamoyl)-7-fluoro-2,3-dihydro-1H-inden-1-yl) carbamate;
O-2-(1H-pyrazol-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N-(4-((3-chloro-4-fluorophenyl)carbamoyl)-7-fluoro-2,2-dimethyl-2,3-dihydro-1H-inden-1-yl) carbamate;
O-(1-methyl-1H-imidazol-2-yl)methyl, N—(S)-(4-((3-chloro-4-fluorophenyl)carbamoyl)-7-fluoro-2,3-dihydro-1H-inden-1-yl) carbamate;
O-(3-fluoropyridin-2-yl)methyl, N—(S)-(4-((3-chloro-4-fluorophenyl)carbamoyl)-7-fluoro-2,3-dihydro-1H-inden-1-yl) carbamate;
O—((R)-morpholin-3-yl)methyl, N—((S)-4-((3-chloro-4-fluorophenyl)carbamoyl)-7-fluoro-2,3-dihydro-1H-inden-1-yl) carbamate;
O-(4-methoxypyridin-2-yl)methyl, N—(S)-(4-((3-chloro-4-fluorophenyl)carbamoyl)-7-fluoro-2,3-dihydro-1H-inden-1-yl) carbamate;
O-2-hydroxyethyl, N—(S)-(4-((3-chloro-4-fluorophenyl)carbamoyl)-7-fluoro-2,3-dihydro-1H-inden-1-yl) carbamate;
O—((S)-tetrahydrofuran-2-yl)methyl, N—((S)-4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-(2-hydroxyacetamido)-2,3-dihydro-1H-indene-4-carboxamide;
(S)—N-(3-chloro-4-fluorophenyl)-7-fluoro-1-(3-(pyridin-3-yl)ureido)-2,3-dihydro-1H-indene-4-carboxamide;
(S)—N-(3-chloro-4-fluorophenyl)-7-fluoro-1-(3-(pyridin-4-yl)ureido)-2,3-dihydro-1H-indene-4-carboxamide;
(S)—N-(3-chloro-4-fluorophenyl)-7-fluoro-1-(thiazol-2-ylamino)-2,3-dihydro-1H-indene-4-carboxamide;
O-2-(piperidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-(difluoromethyl)-4-fluorophenyl)carbamoyl)-7-fluoro-2,3-dihydro-1H-inden-1-yl) carbamate;
(S)—N-(3-chloro-4-fluorophenyl)-7-fluoro-1-(3-(pyridin-2-ylmethyl)ureido)-2,3-dihydro-1H-indene-4-carboxamide;
O-(6-cyanopyridin-2-yl)methyl, N—(S)-(4-((3-chloro-4-fluorophenyl)carbamoyl)-7-fluoro-2,3-dihydro-1H-inden-1-yl) carbamate;
O-quinolin-2-ylmethyl, N—(S)-(4-((3-chloro-4-fluorophenyl)carbamoyl)-7-fluoro-2,3-dihydro-1H-inden-1-yl) carbamate;
O-(5-methylpyrazin-2-yl)methyl, N—(S)-(4-((3-chloro-4-fluorophenyl)carbamoyl)-7-fluoro-2,3-dihydro-1H-inden-1-yl) carbamate;
O-2-morpholinoethyl-N—(S)-(4-((3-chloro-4-fluorophenyl)carbamoyl)-7-fluoro-2,3-dihydro-1H-inden-1-yl) carbamate;
O-[cis-4-hydroxycyclohexyl]-N—((S)-4-((3-chloro-4-fluorophenyl)carbamoyl)-7-fluoro-2,3-dihydro-1H-inden-1-yl)carbamate;
O-3-hydroxypropyl, N—(S)-(4-((3-chloro-4-fluorophenyl)carbamoyl)-7-fluoro-2,3-dihydro-1H-inden-1-yl) carbamate;
O-[trans-4-hydroxycyclohexyl]-N—((S)-4-((3-chloro-4-fluorophenyl)carbamoyl)-7-fluoro-2,3-dihydro-1H-inden-1-yl) carbamate;
O-2-acetamidoethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-propionamido-2,3-dihydro-1H-indene-4-carboxamide;
(S)—N-(3-chloro-4-fluorophenyl)-7-fluoro-1-((4-methoxypyrimidin-2-yl)amino)-2,3-dihydro-1H-indene-4-carboxamide;
(S)—N-(3-chloro-4-fluorophenyl)-7-fluoro-1-((4-methylpyrimidin-2-yl)amino)-2,3-dihydro-1H-indene-4-carboxamide;
(S)—N-(3-chloro-4-fluorophenyl)-7-fluoro-1-((2-methoxypyrimidin-4-yl)amino)-2,3-dihydro-1H-indene-4-carboxamide;
(S)—N-(3-chloro-4-fluorophenyl)-7-fluoro-1-((5-methylpyrimidin-2-yl)amino)-2,3-dihydro-1H-indene-4-carboxamide;
(S)—N-(3-chloro-4-fluorophenyl)-7-fluoro-1-((6-methoxypyrimidin-4-yl)amino)-2,3-dihydro-1H-indene-4-carboxamide;
(S)—N-(3-chloro-4-fluorophenyl)-1-((4,6-dimethylpyrimidin-2-yl)amino)-7-fluoro-2,3-dihydro-1H-indene-4-carboxamide;
(1R,2R)—N-(3-chloro-4-fluorophenyl)-2-methoxy-1-(3-methylureido)-2,3-dihydro-1H-indene-4-carboxamide;
O—(S)-5-oxopyrrolidin-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-(pyridin-2-yl)ethyl)sulfonamido)-2,3-dihydro-1H-indene-4-carboxamide;
O-(6-(trifluoromethyl)pyridin-2-yl)methyl, N—(S)-(4-((3-chloro-4-fluorophenyl)carbamoyl)-7-fluoro-2,3-dihydro-1H-inden-1-yl) carbamate;
O-(5-(trifluoromethyl) pyridin-2-yl)methyl, N—(S)-(4-((3-chloro-4-fluorophenyl)carbamoyl)-7-fluoro-2,3-dihydro-1H-inden-1-yl) carbamate;
O—(R)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-(1-methyl-1H-pyrazol-3-yl)propanamido)-2,3-dihydro-1H-indene-4-carboxamide;
O-(5-cyanopyridin-2-yl)methyl, N—(S)-(4-((3-chloro-4-fluorophenyl)carbamoyl)-7-fluoro-2,3-dihydro-1H-inden-1-yl) carbamate;
O-(3-methylpyrazin-2-yl)methyl, N—(S)-(4-((3-chloro-4-fluorophenyl)carbamoyl)-7-fluoro-2,3-dihydro-1H-inden-1-yl) carbamate;
O-(1-acetylpiperidin-4-yl)methyl, N—(S)-(4-((3-chloro-4-fluorophenyl)carbamoyl)-7-fluoro-2,3-dihydro-1H-inden-1-yl) carbamate;
O-(1-(2-hydroxyacetyl)piperidin-4-yl)methyl, N—(S)-(4-((3-chloro-4-fluorophenyl)carbamoyl)-7-fluoro-2,3-dihydro-1H-inden-1-yl) carbamate;
O-(1-(methylcarbamoyl)(piperidin-4-yl)methyl, N—(S)-(4-((3-chloro-4-fluorophenyl)carbamoyl)-7-fluoro-2,3-dihydro-1H-inden-1-yl) carbamate;
O-(1,1-dioxidothiomorpholin-3-yl)methyl-N—((S)-4-((3-chloro-4-fluorophenyl)carbamoyl)-7-fluoro-2,3-dihydro-1H-inden-1-yl) carbamate;
O-pyridin-2-ylmethyl, N-((1R,2R)-4-((3-chloro-4-fluorophenyl)carbamoyl)-2-methoxy-2,3-dihydro-1H-inden-1-yl) carbamate;
(S)—N-(3-chloro-4-fluorophenyl)-1-(cyclopropanecarboxamido)-7-fluoro-2,3-dihydro-1H-indene-4-carboxamide;
O—((S)-morpholin-3-yl)methyl, N—((S)-4-((3-chloro-4-fluorophenyl)carbamoyl)-7-fluoro-2,3-dihydro-1H-inden-1-yl) carbamate;
O—(S)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-methoxyethyl) sulfonamido)-2,3-dihydro-1H-indene-4-carboxamide;
(S)—N-(3-chloro-4-fluorophenyl)-7-fluoro-1-(phenylsulfonamido)-2,3-dihydro-1H-indene-4-carboxamide;
(S)—N-(3-chloro-4-fluorophenyl)-7-fluoro-1-(pyridine-2-sulfonamido)-2,3-dihydro-1H-indene-4-carboxamide;
O-(1-(2-methoxyacetyl) pi pert din-4-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-hydroxypyrimidin-2-yl)amino)-2,3-dihydro-1H-indene-4-carboxamide
O-methyl, N-(7-((3-chloro-4-fluorophenyl)carbamoyl)-4-fluoro-2,3-dihydrobenzofuran-3-yl) carbamate;
N-(3-chloro-4-fluorophenyl)-4-fluoro-3-(3-methylureido)-2,3-dihydrobenzofuran-7-carboxamide;
O-pyridin-2-ylmethyl, N-(7-((3-chloro-4-fluorophenyl)carbamoyl)-4-fluoro-2,3-dihydrobenzofuran-3-yl) carbamate;
O-(1H-pyrazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-((1-methyl-1H-pyrazol-3-yl)methyl)ureido)-2,3-dihydro-1H-indene-4-carboxamide;
O-(1H-1,2,4-triazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-4-ylamino)-2,3-dihydro-1H-indene-4-carboxamide;
(S)—N-(3-chloro-4-fluorophenyl)-7-fluoro-1-((7-(4-methoxybenzyl)-7H-pyrrolo[2,3-d]pyrimidin-2-yl)amino)-2,3-dihydro-1H-indene-4-carboxamide;
O—((R)-6-oxopiperidin-2-yl)methyl, N—((S)-4-((3-chloro-4-fluorophenyl)carbamoyl)-7-fluoro-2,3-dihydro-1H-inden-1-yl) carbamate;
O—(R)-6-oxopiperidin-3-yl, N—((S)-4-((3-chloro-4-fluorophenyl)carbamoyl)-7-fluoro-2,3-dihydro-1H-inden-1-yl) carbamate;
O—(S)-6-oxopiperidin-3-yl, N—((S)-4-((3-chloro-4-fluorophenyl)carbamoyl)-7-fluoro-2,3-dihydro-1H-inden-1-yl) carbamate;
O-methyl, N-(4-fluoro-7-((4-fluoro-3-methylphenyl)carbamoyl)-2,3-dihydrobenzofuran-3-yl) carbamate;
4-fluoro-N-(4-fluoro-3-methylphenyl)-3-(3-methylureido)-2,3-dihydrobenzofuran-7-carboxamide;
O-pyridin-2-ylmethyl, N-(4-fluoro-7-((4-fluoro-3-methylphenyl)carbamoyl)-2,3-dihydrobenzofuran-3-yl) carbamate;
N-(3-chloro-4-fluorophenyl)-3-(cyclopropanesulfonamido)-4-fluoro-2,3-dihydrobenzofuran-7-carboxamide;
(S)—N-(3-chloro-4-fluorophenyl)-1-(3-cyclopropylureido)-7-fluoro-2,3-dihydro-1H-indene-4-carboxamide;
O-methyl, N-(4-((3-chloro-4-fluorophenyl)carbamoyl-2,2,7-trifluoro-2,3-dihydro-1H-inden-1-yl) carbamate;
N-(3-chloro-4-fluorophenyl)-2,2,7-trifluoro-1-(3-methylureido)-2,3-dihydro-1H-indene-4-carboxamide;
O—((S)-6-oxopiperidin-2-yl)methyl, N—((S)-4-((3-chloro-4-fluorophenyl)carbamoyl)-7-fluoro-2,3-dihydro-1H-inden-1-yl) carbamate;
O-(4-oxoazetidin-2-yl)methyl, N—((S)-4-((3-chloro-4-fluorophenyl)carbamoyl)-7-fluoro-2,3-dihydro-1H-inden-1-yl) carbamate;
O-methyl, N-(4-((3-chloro-4-fluorophenyl)carbamoyl)-7-fluoro-1-methyl-2,3-dihydro-1H-inden-1-yl) carbamate;
N-(3-chloro-4-fluorophenyl)-7-fluoro-1-methyl-1-(3-methylureido)-2,3-dihydro-1H-indene-4-carboxamide;
(S)—N-(3-chloro-4-fluorophenyl)-1-(cyclopropanesulfonamido)-2,3-dihydro-1H-indene-4-carboxamide;
O-pyridin-2-ylmethyl, N-(4-((3-chloro-4-fluorophenyl)carbamoyl)-7-fluoro-1-methyl-2,3-dihydro-1H-inden-1-yl) carbamate;
O-pyridin-2-ylmethyl, N-(4-((3-chloro-4-fluorophenyl)carbamoyl)-2,2,7-trifluoro-2,3-dihydro-1H-inden-1-yl) carbamate;
(S)—N-(3-chloro-4-fluorophenyl)-1-((cyclopropylmethyl)sulfonamido)-7-fluoro-2,3-dihydro-1H-indene-4-carboxamide;
(S)—N-(3-chloro-4-fluorophenyl)-7-fluoro-1-((phenylmethyl)sulfonamido)-2,3-dihydro-1H-indene-4-carboxamide;
O-cyclopropyl, N—(S)-(4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-((N-methylsulfamoyl)amino)-2,3-dihydro-1H-indene-4-carboxamide;
(S)—N-(3-chloro-4-fluorophenyl)-7-fluoro-1-(morpholine-4-sulfonamido)-2,3-dihydro-1H-indene-4-carboxamide;
O-cyclopropyl, N—(S)-(4-((3-chloro-4-fluorophenyl)carbamoyl)-2,3-dihydro-1H-inden-1-yl) carbamate;
(S)—N-(3-chloro-4-fluorophenyl)-1-((N-methylsulfamoyl)amino)-2,3-dihydro-1H-indene-4-carboxamide;
O-(1,3,4-oxadiazol-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-1-(ethylsulfonamido)-7-fluoro-2,3-dihydro-1H-indene-4-carboxamide;
(S)—N-(3-chloro-4-fluorophenyl)-7-fluoro-1-(propylsulfonamido)-2,3-dihydro-1H-indene-4-carboxamide;
(S)—N-(4-chloro-3-fluorophenyl)-7-fluoro-1-((2-methylpropyl)sulfonamido)-2,3-dihydro-1H-indene-4-carboxamide;
N-(3-chloro-4-fluorophenyl)-7-fluoro-2-methoxy-1-(3-methylureido)-2,3-dihydro-1H-indene-4-carboxamide;
O-methyl, N-(4-((3-chloro-4-fluorophenyl)carbamoyl)-7-fluoro-2-methoxy-2,3-dihydro-1H-inden-1-yl)carbamate;
(S)—N-(3-chloro-4-fluorophenyl)-7-fluoro-1-((N-isopropylsulfamoyl)amino)-2,3-dihydro-1H-indene-4-carboxamide;
(S)—N-(3-chloro-4-fluorophenyl)-7-fluoro-1-((1-methylethyl)sulfonamido)-2,3-dihydro-1H-indene-4-carboxamide;
(S)—N-(3-chloro-4-fluorophenyl)-1-(cyclopentanesulfonamido)-7-fluoro-2,3-dihydro-1H-indene-4-carboxamide;
(S)—N-(3-chloro-4-fluorophenyl)-1-(cyclohexanesulfonamido)-7-fluoro-2,3-dihydro-1H-indene-4-carboxamide;
N-(3-chloro-4-fluorophenyl)-7-fluoro-3,3-dimethyl-1-(3-methylureido)-2,3-dihydro-1H-indene-4-carboxamide;
(S)—N-(3-chloro-4-fluorophenyl)-1-((N-cyclopropylsulfamoyl)amino)-2,3-dihydro-1H-indene-4-carboxamide;
(S)—N-(3-chloro-4-fluorophenyl)-1-((N-cyclopropylsulfamoyl)amino)-7-fluoro-2,3-dihydro-1H-indene-4-carboxamide;
O-methyl, N-(4-((3,4-difluorophenyl)carbamoyl)-7-fluoro-2-methoxy-2,3-dihydro-1H-inden-1-yl) carbamate;
N-(3,4-difluorophenyl)-7-fluoro-2-methoxy-1-(3-methylureido)-2,3-dihydro-1H-indene-4-carboxamide;
O-pyridin-2-ylmethyl, N-(4-((3,4-difluorophenyl)carbamoyl)-7-fluoro-2-methoxy-2,3-dihydro-1H-inden-1-yl)carbamate
O-pyridin-2-ylmethyl, N-(4-((3-chloro-4-fluorophenyl)carbamoyl)-7-fluoro-2-methoxy-2,3-dihydro-1H-inden-1-yl)carbamate;
O-(1-(tetrahydro-2H-pyran-2-yl)-1H-1,2,4-triazol-3-yl)methyl, N—((S)-4-((3-chloro-4-fluorophenyl)carbamoyl)-7-fluoro-2,3-dihydro-1H-inden-1-yl) carbamate;
N-(3-chloro-4-fluorophenyl)-7-fluoro-2,2-dimethyl-1-(3-methylureido)-2,3-dihydro-1H-indene-4-carboxamide;
N-(3-Chloro-4-fluorophenyl)-7-fluoro-1-oxo-2,3-dihydro-1H-indene-4-carboxamide;
((1-(methyl-d₃)-1H-1,2,4-triazol-3-yl)methyl-d₂ (S)-(4-((3-chloro-4-fluorophenyl)carbamoyl)-7-fluoro-2,3-dihydro-1H-inden-1-yl)carbamate;
(S)-(3-((((4-((3-chloro-4-fluorophenyl)carbamoyl)-7-fluoro-2,3-dihydro-1H-inden-1-yl)carbamoyl)oxy)methyl)-1H-1,2,4-triazol-1-yl)methyl phosphoric acid;
(S)-(3-((((4-((3-chloro-4-fluorophenyl)carbamoyl)-7-fluoro-2,3-dihydro-1H-inden-1-yl)carbamoyl)oxy)methyl)-1H-pyrazol-1-yl)methyl phosphoric acid:
O—(S)-2-cyanoethyl, N-4-(3-chloro-4-fluorophenylcarbamoyl)-7-fluoro-2,3-dihydro-1H-inden-1-yl carbamate;
O—(S)-3-cyanopropyl, N-4-(3-chloro-4-fluorophenylcarbamoyl)-7-fluoro-2,3-dihydro-1H-inden-1-yl carbamate;
N-(3-chloro-4-fluorophenyl)-7′-fluoro-2,5-dioxo-2′,3′-dihydrospiro[imidazolidine-4,1′-indene-]-4′-carboxamide;
N-(3-chloro-4-fluorophenyl)-7′-fluoro-2,5-dioxo-1-(pyridin-2-ylmethyl)-2′,3′-dihydrospiro[imidazolidine-4,1′-indene]-4′-carboxamide;
N-(3-chloro-4-fluoro-phenyl)-7′-fluoro-1-methyl-2,5-dioxo-spiro[imidazolidine-4,1′-indane]-4′-carboxamide;
N-(3-chloro-4-fluorophenyl)-7-(3-methylureido)-6,7-dihydro-5H-cyclopenta[b]pyridine-4-carboxamide;
O-methyl, N-(4-((3-chloro-4-fluorophenyl)carbamoyl)-6,7-dihydro-5H-cyclopenta[b]pyridin-7-yl)carbamate;
O-pyridin-2-ylmethyl, N-(4-((3-chloro-4-fluorophenyl)carbamoyl)-6,7-dihydro-5H-cyclopenta[b]pyridin-7-yl) carbamate;
N-(3-chloro-4-fluorophenyl)-7-(cyclopropanesulfonamido)-6,7-dihydro-5H-cyclopenta[c]pyridine-4-carboxamide;
O-(pyridin-2-ylmethyl)-N-[(4-((3-chloro-4-fluorophenyl)carbamoyl)-6,7-dihydro-5H-cyclopenta[c]pyridin-7-yl)] carbamate;
N-(3-chloro-4-fluorophenyl)-7-fluoro-2,3-dihydrobenzo[b]thiophene-4-carboxamide 1,1-dioxide;
N-(3-chloro-4-fluorophenyl)-2,3-dihydrobenzo[b]thiophene-4-carboxamide 1,1-dioxide;
2-(tert-butyl)-N-(3-chloro-4-fluorophenyl)-2,3-dihydrobenzo[d]isothiazole-4-carboxamide 1,1-dioxide;
N-(3-chloro-4-fluorophenyl)-2,3-dihydrobenzo[d]isothiazole-4-carboxamide-1,1-dioxide;
N-(3-chloro-4-fluorophenyl)-2-(2-hydroxyethyl)-2,3-dihydrobenzo[d]isothiazole-4-carboxamide 1,1-dioxide;
N-(3-chloro-4-fluorophenyl)-2-methyl-2,3-dihydrobenzo[d]isothiazole-4-carboxamide 1,1-dioxide;
N-(3-chloro-4-fluorophenyl)-2-isopropyl-2,3-dihydrobenzo[d]isothiazole-4-carboxamide 1,1-dioxide′
N-(3-chloro-4-fluorophenyl)-2-cyclopropyl-2,3-dihydrobenzo[d]isothiazole-4-carboxamide 1,1-dioxide;
(S)-1-(((S)-tert-butylsulfinyl)amino)-N-(3-chloro-4-fluorophenyl)-7-fluoro-2,3-dihydro-1H-indene-4-carboxamide;
(S)-1-(((R)-tert-butylsulfinyl)amino)-N-(3-chloro-4-fluorophenyl)-7-fluoro-2,3-dihydro-1H-indene-4-carboxamide;
O-methyl, N-(4-((3-chloro-4-fluorophenyl)carbamoyl)-7-fluoro-3,3-dimethyl-2,3-dihydro-1H-inden-1-yl) carbamate;
   or a salt thereof.

cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

The term cccDNA formation inhibitor includes compounds described in International Patent Application Publication Number WO2013130703, including the following compound:

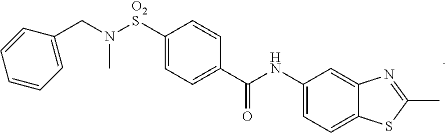 [SMILES: Cc1nc2cc(NC(=O)c3ccc(S(=O)(=O)N(C)Cc4ccccc4)cc3)ccc2s1]

The term cccDNA formation inhibitor includes, but is not limited to, those generally and specifically described in United States Patent Application Publication Number US 2015/0038515 A1. The term cccDNA formation inhibitor includes, but is not limited to, 1-(phenylsulfonyl)-N-(pyridin-4-ylmethyl)-1H-indole-2-carboxamide; 1-Benzenesulfonyl-pyrrolidine-2-carboxylic acid (pyridin-4-ylmethyl)-amide; 2-(2-chloro-N-(2-chloro-5-(trifluoromethyl)phenyl)-4-(trifluoromethyl)phenylsulfonamido)-N-(pyridin-4-ylmethyl)acetamide; 2-(4-chloro-N-(2-chloro-5-(trifluoromethyl)phenyl)phenylsulfonamido)-N-(pyridin-4-ylmethyl)acetamide; 2-(N-(2-chloro-5-(trifluoromethyl)phenyl)-4-(trifluoromethyl)phenylsulfonamido)-N-(pyridin-4-ylmethyl)acetamide; 2-(N-(2-chloro-5-(trifluoromethyl)phenyl)-4-methoxyphenylsulfonamido)-N-(pyridin-4-ylmethyl)acetamide; 2-(N-(2-chloro-5-(trifluoromethyl)phenyl)phenylsulfonamido)-N-((1-methylpiperidin-4-yl)methyl)acetamide; 2-(N-(2-chloro-5-(trifluoromethyl)phenyl)phenylsulfonamido)-N-(piperidin-4-ylmethyl)acetamide; 2-(N-(2-chloro-5-(trifluoromethyl)phenyl)phenylsulfonamido)-N-(pyridin-4-ylmethyl)propanamide; 2-(N-(2-chloro-5-(trifluoromethyl)phenyl)phenylsulfonamido)-N-(pyridin-3-ylmethyl)acetamide; 2-(N-(2-chloro-5-(trifluoromethyl)phenyl)phenylsulfonamido)-N-(pyrimidin-5-ylmethyl)acetamide; 2-(N-(2-chloro-5-(trifluoromethyl)phenyl)phenylsulfonamido)-N-(pyrimidin-4-ylmethyl)acetamide; 2-(N-(5-chloro-2-fluorophenyl)phenylsulfonamido)-N-(pyridin-4-ylmethyl)acetamide; 2-[(2-chloro-5-trifluoromethyl-phenyl)-(4-fluoro-benzenesulfonyl)-amino]-N-pyridin-4-ylmethyl-acetamide; 2-[(2-chloro-5-trifluoromethyl-phenyl)-(toluene-4-sulfonyl)-amino]-N-pyridin-4-ylmethyl-acetamide; 2-[benzenesulfonyl-(2-bromo-5-trifluoromethyl-phenyl)-amino]-N-pyridin-4-ylmethyl-acetamide; 2-[benzenesulfonyl-(2-chloro-5-trifluoromethyl-phenyl)-amino]-N-(2-methyl-benzothiazol-5-yl)-acetamide; 2-[benzenesulfonyl-(2-chloro-5-trifluoromethyl-phenyl)-amino]-N-[4-(4-methyl-piperazin-1-yl)-benzyl]-acetamide; 2-[benzenesulfonyl-(2-chloro-5-trifluoromethyl-phenyl)-amino]-N-[3-(4-methyl-piperazin-1-yl)-benzyl]-acetamide; 2-[benzenesulfonyl-(2-chloro-5-trifluoromethyl-phenyl)-amino]-N-benzyl-acetamide; 2-[benzenesulfonyl-(2-chloro-5-trifluoromethyl-phenyl)-amino]-N-pyridin-4-ylmethyl-acetamide; 2-[benzenesulfonyl-(2-chloro-5-trifluoromethyl-phenyl)-amino]-N-pyridin-4-ylmethyl-propionamide; 2-[benzenesulfonyl-(2-fluoro-5-trifluoromethyl-phenyl)-amino]-N-pyridin-4-ylmethyl-acetamide; 4 (N-(2-chloro-5-(trifluoromethyl)phenyl)phenylsulfonamido)-N-(pyridin-4-yl-methyl)butanamide; 4-((2-(N-(2 chloro-5-(trifluoromethyl)phenyl)phenylsulfonamido)-acetamido)-methyl)-1,1-dimethylpiperidin-1-ium chloride; 4-(benzyl-methyl-sulfamoyl)-N-(2-chloro-5-trifluoromethyl-phenyl)-benzamide; 4-(benzyl-methyl-sulfamoyl)-N-(2-methyl-1H-indol-5-yl)-benzamide; 4-(benzyl-methyl-sulfamoyl)-N-(2-methyl-1H-indol-5-yl)-benzamide; 4-(benzyl-methyl-sulfamoyl)-N-(2-methyl-benzothiazol-5-yl)-benzamide; 4-(benzyl-methyl-sulfamoyl)-N-(2-methyl-benzothiazol-6-yl)-benzamide; 4-(benzyl-methyl-sulfamoyl)-N-(2-methyl-benzothiazol-6-yl)-benzamide; 4-(benzyl-methyl-sulfamoyl)-N-pyridin-4-ylmethyl-benzamide; N-(2-aminoethyl)-2-(N-(2-chloro-5-(trifluoromethyl)phenyl)phenylsulfonamido)-acetamide; N-(2-chloro-5-(trifluoromethyl)phenyl)-N-(2-(3,4-dihydro-2,6-naphthyridin-2(1H)-yl)-2-oxoethyl)benzenesulfonamide: N-benzothiazol-6-yl-4-(benzyl-methyl-sulfamoyl)-benzamide; N-benzothiazol-6-yl-4-(benzyl-methyl-sulfamoyl)-benzamide; tert-butyl (2-(2-(N-(2-chloro-5-(trifluoromethyl)phenyl)phenylsulfonamido)acetamido)-ethyl)carbamate; and tert-butyl 4-((2-(N-(2-chloro-5-(trifluoromethyl)phenyl)phenylsulfonamido)-acetamido)-methyl)piperidine-1-carboxylate, and optionally, combinations thereof.

### sAg Secretion Inhibitors/RNA Destabilizers

As described herein the term “sAg secretion inhibitor” includes compounds that are capable of inhibiting, either directly or indirectly, the secretion of sAg (S. M and/or L surface antigens) bearing subviral particles and/or DNA containing viral particles from HBV-infected cells. As used herein, “sAg secretion inhibitors” are also known as “RNA destabilizers”, and these terms are used interchangeably. For example, in certain embodiments, the inhibitor detectably inhibits the secretion of sAg as measured, e.g., using assays known in the art or described herein, e.g., ELISA assay or by Western Blot. In certain embodiments, the inhibitor inhibits the secretion of sAg by at least 5%, at least 10%, at least 20%, at least 50%, at least 75%, or at least 90%. In certain embodiments, the inhibitor reduces serum levels of sAg in a patient by at least 5%, at least 10%, at least 20%, at least 50%, at least 75%, or at least 90%.

The term RNA destabilizer includes compounds described in WO 2018/085619, which patent document is specifically incorporated by reference in its entirety.

The term sAg secretion inhibitor includes compounds described in U.S. Pat. No. 8,921,381, as well as compounds described in United States Patent Application Publication Numbers 2015/0087659 and 2013/0303552. For example, the term includes the compounds PBHBV-001 and PBHBV-2-15, and pharmaceutically acceptable salts thereof:

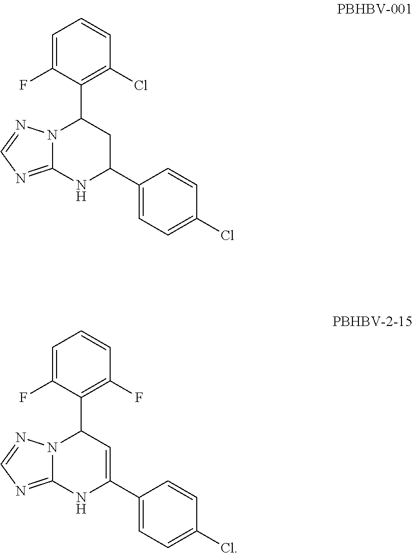 [SMILES: Fc1cccc(Cl)c1C1CC(c2ccc(Cl)cc2)Nc2ncnn21 ; Fc1cccc(F)c1C1C=C(c2ccc(Cl)cc2)Nc2ncnn21]

The term sAg secretion inhibitor/RNA destabilizer also includes the compound:

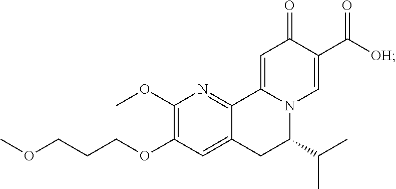 [SMILES: COCCCOc1cc2c(nc1OC)-c1cc(=O)c(C(=O)O)cn1[C@H](C(C)C)C2]

and pharmaceutically acceptable salts thereof (see WO 2018/085619).

In certain embodiments, a sAg secretion inhibitor/RNA destabilizer is a compound of the following formula, or a salt thereof:

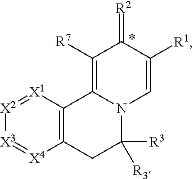

wherein the following definitions apply:
R¹ is selected from the group consisting of H; halo; —OR⁸; —C(R⁹)(R⁹)OR⁸; —C(═O)R⁸; —C(═O)OR⁸; —C(═O)NH—OR⁸; —C(═O)NHNHR⁸; —C(═O)NHNHC(═O)R⁸; —C(═O)NHS(═O)₂R⁸; —CH₂C(═O)OR⁸; —CN; —NH₂; —N(R⁸)C(═O)H; —N(R⁸)C(═O)R¹⁰; —N(R⁸)C(═O)OR¹⁰; —N(R⁸)C(═O)NHR⁸; —NR⁹S(═O)₂R¹⁰: —P(═O)(OR⁸)₂; —B(OR⁸)₂; 2,5-dioxo-pyrrolidin-1-yl; 2H-tetrazol-5-yl; 3-hydroxy-isoxazol-5-yl; 1,4-dihydro-5-oxo-5H-tetrazol-1-yl; pyridin-2-yl optionally substituted with C₁-C₆ alkyl; pyrimidin-2-yl optionally substituted with C₁-C₆ alkyl; (pyridin-2-yl)methyl; (pyrimidin-2-yl)methyl; (pyrimidin-2-yl)amino; bis-(pyrimidin-2-yl)-amino; 5-R⁸-1,3,4,-thiadiazol-2-yl; 5-thioxo-4,5-dihydro-1H-1,2,4-triazol-3-yl; 1H-1,2,4-triazol-5-yl: 1,3,4-oxadiazol-2-yl; 1,2,4-oxadiazol-5-yl, and 3-R¹⁰-1,2,4-oxadiazol-5-yl;
R² is selected from the group consisting of ═O, ═NR⁹, ═N(OR⁹), and ═N(NR⁹R⁹);
   or R¹ and R² combine to form ═N—O—C(O)— or ═N—N(R⁹)—C(O)—, wherein the ═N group is bound to the ring carbon atom marked “*”:
X¹ is selected from the group consisting of CR^{6I} and N, X² is selected from the group consisting of CR^{6II} and N, X³ is selected from the group consisting of CR^{6III} and N, X⁴ is selected from the group consisting of CR^{6IV} and N, or either X³ and X⁴, or X¹ and X², combine to form —S—;
   wherein 1-2 substituents selected from the group consisting of X¹, X², X³ and X⁴ are N; each of which, if present, is optionally alkylated with C₁-C₆ alkyl if the adjacent carbon atom in the ring is substituted with —OH;
R^{6I}, R^{6II}, R^{6III} and R^{6IV} are independently selected from the group consisting of H, halo, —CN, pyrrolidinyl, optionally substituted C₁-C₆ alkyl, optionally substituted C₁-C₆ alkenyl, optionally substituted C₃-C₈ cycloalkyl, optionally substituted heterocyclyl, —OR, C₁-C₆ haloalkoxy, —N(R)(R), —NO₂, —S(═O)₂N(R)(R), acyl, and C₁-C₆ alkoxycarbonyl,
   wherein each occurrence of R is independently selected from the group consisting of H, C₁-C₆ alkyl, R′-substituted C₁-C₆ alkyl, C₁-C₆ hydroxyalkyl, optionally substituted (C₁-C₆ alkoxy)-C₁-C₆ alkyl, and optionally substituted C₃-C₈ cycloalkyl,
   wherein each occurrence of R′ is independently selected from the group consisting of —NH₂, —NH(C₁-C₆ alkyl), —N(C₁-C₆ alkyl)(C₁-C₆ alkyl), —NHC(═O)O^{t}Bu, —N(C₁-C₆ alkyl)C(═O)O^{t}Bu, or a 5- or 6-membered heterocyclic group, which is optionally N-linked;
   or X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—;
R⁷ is selected from the group consisting of H, OH, halo, C₁-C₆ alkoxy, and optionally substituted C₁-C₆ alkyl;
R⁸ is selected from the group consisting of H, optionally substituted C₁-C₆ alkyl, and optionally substituted C₃-C₈ cycloalkyl;
each occurrence of R⁹ is independently selected from the group consisting of H and C₁-C₆ alkyl;
R¹⁰ is selected from the group consisting of optionally substituted C₁-C₆ alkyl and optionally substituted phenyl; and,
each occurrence of R¹¹ is independently selected from the group consisting of H, OH, C₁-C₆ alkyl, C₁-C₆ alkoxy, alkoxy-C₁-C₆ alkyl and alkoxy-C₁-C₆ alkoxy, wherein two R¹¹ groups bound to the same carbon atom are not simultaneously OH; or two R¹¹ groups combine with the carbon atom to which they are bound to form a moiety selected from the group consisting of C═O, C═CH₂ and oxetane-3,3-diyl.

In certain embodiments, each occurrence of alkyl or cycloalkyl is independently optionally substituted with at least one substituent selected from the group consisting of C₁-C₆ alkyl, halo, —OR″, phenyl and —N(R″)(R″), wherein each occurrence of R″ is independently H, C₁-C₆ alkyl or C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo. —CN, —OR, —N(R″)(R″), —NO₂, —S(═O)₂N(R″)(R″), acyl, and C₁-C₆ alkoxycarbonyl, wherein each occurrence of R″ is independently H, C₁-C₆ alkyl or C₃-C₈ cycloalkyl.

In certain embodiments, the compound is selected from the group consisting of:

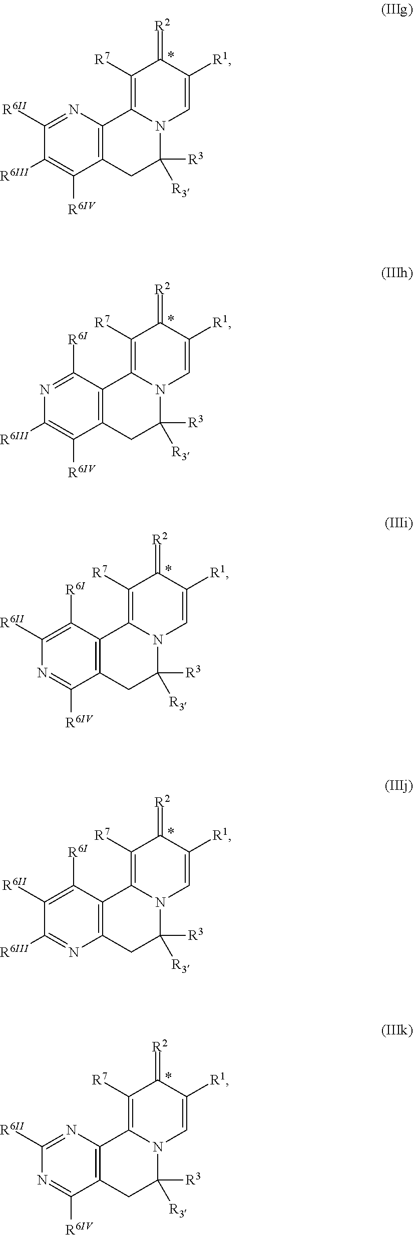

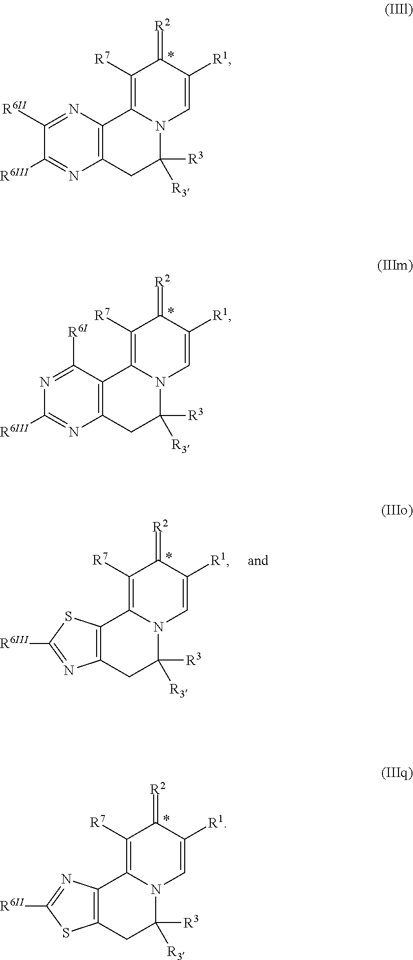

In certain embodiments, R¹ is selected from the group consisting of optionally substituted triazolyl, optionally substituted oxadiazolyl, —C(═O)OH, —C(═O)OMe, —C(═O)OEt. —C(═O)O-nPr, —C(═O)O-iPr, —C(═O)O-cyclopentyl, and —C(═O)O-cyclohexyl.

In certain embodiments, R² is selected from the group consisting of O, N(OH), N(Me), N(OMe), and N(NH₂).

In certain embodiments, R³ and R^{3′} are each independently selected from the group consisting of H, methyl, ethyl, n-propyl, isopropyl, n-butyl, isobutyl, sec-butyl, t-butyl, hydroxymethyl, 2-hydroxy-ethyl, 2-methoxy-ethyl, methoxymethyl, and 2-methyl-1-methoxy-prop-2-yl.

In certain embodiments, at least one applies: R³ is H, R^{3′} is isopropyl; R³ is H, R^{3′} is tert-butyl; R³ is methyl, R^{3′} is isopropyl; R³ is methyl, R^{3′} is tert-butyl; R³ is methyl, R^{3′} is methyl; R³ is methyl, R^{3′} is ethyl; and R³ is ethyl, R^{3′} is ethyl.

In certain embodiments, R³ and R³ are not H.

In certain embodiments, R³/R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and wherein each divalent group is optionally substituted with at least one C₁-C₆ alkyl or halo.

In certain embodiments, when present, R^{6I}, R^{6II}, R^{6III} and R^{6IV} are independently selected from the group consisting of H, F, Cl, Br, I, CN, amino, methylamino, dimethylamino, methoxyethylamino, pyrrolidinyl, methoxy, ethoxy, n-propoxy, isopropoxyl, n-butoxy, sec-butoxy, isobutoxy, t-butoxy, 2-methoxy-ethoxy, 2-hydroxy-ethoxy, 3-methoxy-prop-1-yl, 3-hydroxy-prop-1-yl, 3-methoxy-prop-1-oxy, 3-hydroxy-prop-1-oxy, 4-methoxy-but-1-yl, 4-hydroxy-but-1-yl, 4-methoxy-but-1-oxy, 4-hydroxy-but-1-oxy, 2-hydroxy-ethoxy, 3-hydroxy-prop-1-yl, 4-hydroxy-but-1-yl, 3-hydroxy-2,2-dimethyl-prop-1-oxy, cyclopropylmethoxy, 2,2,2-trifluoroethoxy, 2-(2-haloethoxy)-ethoxy, 2-(N-morpholino)-ethyl, 2-(N-morpholino)-ethoxy, 3-(N-morpholino)-prop-1-yl, 3-(N-morpholino)-prop-1-oxy, 4-(N-morpholino)-but-1-yl, 4-(N-morpholino)-butl-oxy, 2-amino-ethyl, 2-(NHC(═O)O^{t}Bu)-ethyl, 2-amino-ethoxy, 2-(NHC(═O)O^{t}Bu)-ethoxy, 3-amino-prop-1-yl, 3-(NHC(═O)O^{t}Bu)-prop-1-yl, 3-amino-prop-1-oxy, 3-(NHC(═O)O^{t}Bu)-prop-1-oxy, 4-amino-but-1-yl, 4-(NHC(═O)O^{t}Bu)-but-1-yl, 4-amino-but-1-oxy, and 4-(NHC(═O)O^{t}Bu)-but-1-oxy.

In certain embodiments, X¹ is CH or N.

In certain embodiments, X⁴ is CH.

In certain embodiments, X² is CR^{6II}, CR^{6II} is not H, X³ is CR^{6III} and R^{6III} is not H.

In certain embodiments, X¹ is N, X² is CR^{6II}, X³ is CR^{6III}, and X⁴ is CH, and one of the following applies: R^{6II} is methoxy, R^{6III} is 3-methoxy-propoxy; R^{6II} is chloro, R^{6III} is 3-methoxy-propoxy; R^{6II} is cyclopropyl, R^{6III} is 3-methoxy-propoxy; R^{6II} is methoxy, R^{6III} is methoxy; R^{6II} is chloro, R^{6III} is methoxy; and R^{6II} is cyclopropyl, R^{6III} is methoxy.

In certain embodiments, X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O—, and —O(CH₂)(CR¹¹R¹¹)(CH₂)O.

In certain embodiments, R⁷ is selected from the group consisting of H, methyl, ethyl, and fluoro.

In certain embodiments, a sAg secretion inhibitor/RNA destabilizer is a compound of the following formula, or a salt thereof:

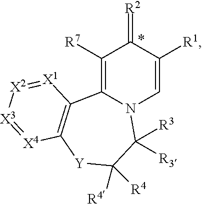

wherein the following definitions apply:
Y is selected from the group consisting of CHR⁵ and O;
each occurrence of R⁵ is independently selected from the group consisting of H, optionally substituted C₁-C₆ alkyl, and optionally substituted C₃-C₈ cycloalkyl;
R¹ is selected from the group consisting of H; halo; —OR⁸; —C(R⁹)(R⁹)OR⁸; —C(═O)R⁸; —C(═O)OR⁸; —C(═O)NH—OR⁸; —C(═O)NHNHR⁸; —C(═O)NHNHC(═O)R⁸; —C(═O)NHS(═O)₂R⁸; —CH₂C(═O)OR⁸; —CN; —NH₂; —N(R⁸)C(═O)H; —N(R⁸)C(═O)R¹⁰; —N(R⁸)C(═O)OR¹⁰; —N(R⁸)C(═O)NHR⁸; —NR⁹S(═O)₂R¹⁰; —P(═O)(OR⁸)₂; —B(OR⁸)₂: 2,5-dioxo-pyrrolidin-1-yl; 2H-tetrazol-5-yl; 3-hydroxy-isoxazol-5-yl; 1,4-dihydro-5-oxo-5H-tetrazol-1-yl; pyridin-2-yl optionally substituted with C₁-C₆ alkyl; pyrimidin-2-yl optionally substituted with C₁-C₆ alkyl; (pyridin-2-yl)methyl; (pyrimidin-2-yl)methyl; (pyrimidin-2-yl)amino; bis-(pyrimidin-2-yl)-amino; 5-R⁸-1,3,4,-thiadiazol-2-yl; 5-thioxo-4,5-dihydro-1H-1,2,4-triazol-3-yl; 1H-1,2,4-triazol-5-yl; 1,3,4-oxadiazol-2-yl; 1,2,4-oxadiazol-5-yl, and 3-R¹⁰-1,2,4-oxadiazol-5-yl;
R² is selected from the group consisting of ═O, ═NR⁹, ═N(OR⁹), and ═N(NR⁹R⁹);
   or R¹ and R² combine to form ═N—O—C(═O)— or ═N—N(R⁹)—C(═O)—, wherein the ═N group is bound to the ring carbon atom marked “*”;
R³, R^{3′}, R⁴ and R^{4′} are each independently selected from the group consisting of H, alkyl-substituted oxetanyl, optionally substituted C₁-C₆ alkyl and optionally substituted C₃-C₈ cycloalkyl;
   or one pair selected from the group consisting of R³/R^{3′}, R⁴/R^{4′}, and R³/R⁴ combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;
X¹ is selected from the group consisting of CR^{6I} and N, X² is selected from the group consisting of CR^{6II} and N, X³ is selected from the group consisting of CR^{6III} and N, X⁴ is selected from the group consisting of CR^{6IV} and N, or either X³ and X⁴, or X¹ and X², combine to form —S—;
   wherein 0-2 substituents selected from the group consisting of X¹, X², X³ and X⁴ are N, each of which, if present, is optionally alkylated with C₁-C₆ alkyl if the adjacent carbon atom in the ring is substituted with —OH;
R^{6I}, R^{6II}, R^{6III} and R^{6IV} are independently selected from the group consisting of H, halo, —CN, pyrrolidinyl, optionally substituted C₁-C₆ alkyl, optionally substituted C₁-C₆ alkenyl, optionally substituted C₃-C₈ cycloalkyl, optionally substituted heterocyclyl, —OR, C₁-C₆ haloalkoxy, —N(R)(R), —NO₂, —S(═O)₂N(R)(R), acyl, and C₁-C₆ alkoxycarbonyl,
   wherein each occurrence of R is independently selected from the group consisting of H, C₁-C₆ alkyl, R′-substituted C₁-C₆ alkyl, C₁-C₆ hydroxyalkyl, optionally substituted (C₁-C₆ alkoxy)-C₁-C₆ alkyl, and optionally substituted C₃-C₈ cycloalkyl,
   wherein each occurrence of R′ is independently selected from the group consisting of —NH₂, —NH(C₁-C₆ alkyl), —N(C₁-C₆ alkyl)(C₁-C₆ alkyl), —NHC(═O)O^{t}Bu, —N(C₁-C₆ alkyl)C(O)O^{t}Bu, or a 5- or 6-membered heterocyclic group, which is optionally N-linked;
   or X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—;
R⁷ is selected from the group consisting of H, OH, halo, C₁-C₆ alkoxy, and optionally substituted C₁-C₆ alkyl.
R⁸ is selected from the group consisting of H, optionally substituted C₁-C₆ alkyl, and optionally substituted C₃-C₈ cycloalkyl;
each occurrence of R⁹ is independently selected from the group consisting of H and C₁-C₆ alkyl;
R¹⁰ is selected from the group consisting of optionally substituted C₁-C₆ alkyl and optionally substituted phenyl; and,
each occurrence of R¹¹ is independently selected from the group consisting of H, OH, C₁-C₆ alkyl, C₁-C₆ alkoxy, alkoxy-C₁-C₆ alkyl and alkoxy-C₁-C₆ alkoxy, wherein two R¹¹ groups bound to the same carbon atom are not simultaneously OH; or two R¹¹ groups combine with the carbon atom to which they are bound to form a moiety selected from the group consisting of C═O, C═CH₂ and oxetane-3,3-diyl.

In certain embodiments, each occurrence of alkyl or cycloalkyl is independently optionally substituted with at least one substituent selected from the group consisting of C₁-C₆ alkyl, halo, —OR″, phenyl and —N(R″)(R″), wherein each occurrence of R″ is independently H, C₁-C₆ alkyl Or C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl. C₁-C₆ haloalkoxy, halo, —CN, —OR, —N(R″)(R″), —NO₂, —S(═O)₂N(R″)(R″), acyl, and C₁-C₆ alkoxycarbonyl, wherein each occurrence of R″ is independently H, C₁-C₆ alkyl or C₃-C₈ cycloalkyl.

In certain embodiments, the compound is selected from the group consisting of:

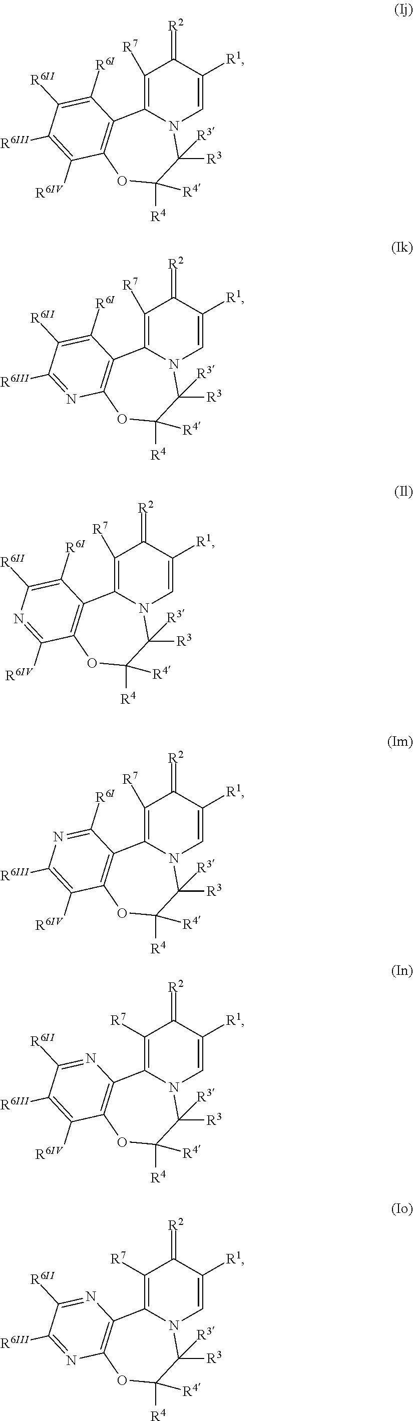

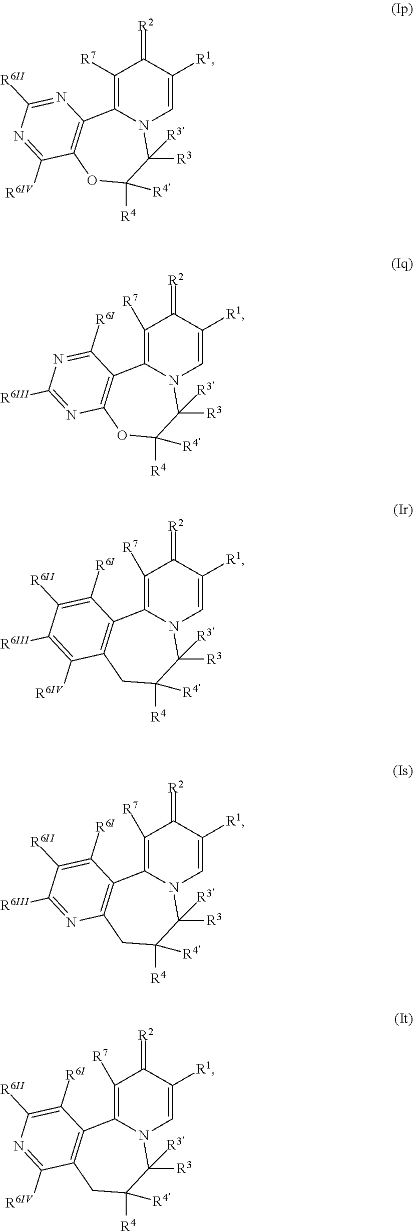

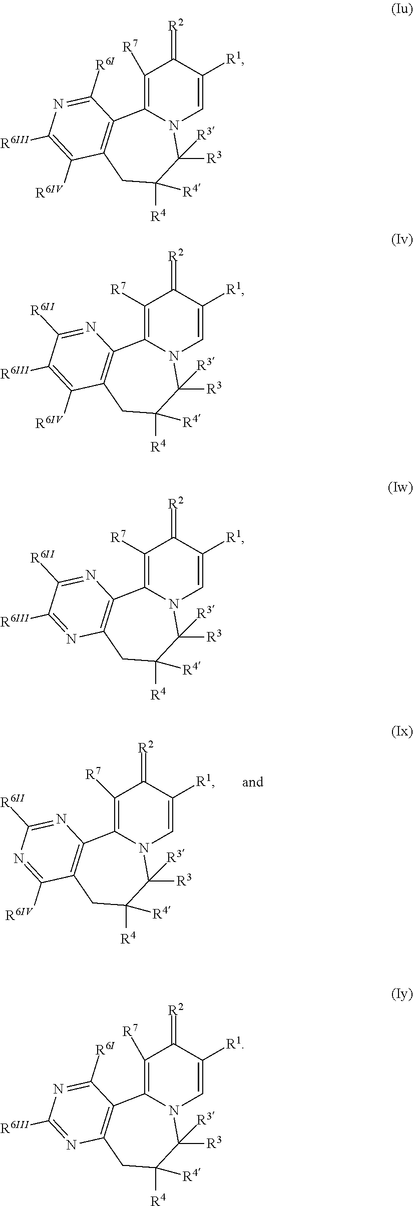

In certain embodiments, R¹ is selected from the group consisting of optionally substituted triazolyl, optionally substituted oxadiazolyl, —C(═O)OH, —C(═O)OMe, —C(═O)OEt, —C(═O)O-nPr, —C(═O)O-iPr, —C(═O)O-cyclopentyl, and —C(═O)O-cyclohexyl.

In certain embodiments, R² is selected from the group consisting of O, N(OH), N(Me), N(OMe), and N(NH₂).

In certain embodiments, R³ and R^{3′}, and R⁴ and R^{4′}, are each independently selected from the group consisting of H, methyl, ethyl, n-propyl, isopropyl, n-butyl, isobutyl, sec-butyl, t-butyl, hydroxymethyl, 2-hydroxy-ethyl, 2-methoxy-ethyl, methoxymethyl, and 2-methyl-1-methoxy-prop-2-yl.

In certain embodiments, at least one applies: R³ is H, R^{3′} is isopropyl; R³ is H, R^{3′} is tert-butyl; R³ is methyl, R^{3′} is isopropyl; R³ is methyl, R^{3′} is tert-butyl; R³ is methyl, R^{3′} is methyl; R³ is methyl, R^{3′} is ethyl; and R³ is ethyl, R^{3′} is ethyl.

In certain embodiments, R³ and R^{3′} are not H.

In certain embodiments, R⁴ and R^{4′} are H.

In certain embodiments, R³/R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and wherein each divalent group is optionally substituted with at least one C₁-C₆ alkyl or halo.

In certain embodiments, R^{6I}, R^{6II}, R^{6III} and R^{6IV}, when present, are independently selected from the group consisting of H, F, Cl, Br, I, CN, amino, methylamino, dimethylamino, methoxyethylamino, pyrrolidinyl, methoxy, ethoxy, n-propoxy, isopropoxyl, n-butoxy, sec-butoxy, isobutoxy, t-butoxy, 2-methoxy-ethoxy, 2-hydroxy-ethoxy, 3-methoxy-prop-1-yl, 3-hydroxy-prop-1-yl, 3-methoxy-prop-1-oxy, 3-hydroxy-prop-1-oxy, 4-methoxy-but-1-yl, 4-hydroxy-but-1-yl, 4-methoxy-but-1-oxy, 4-hydroxy-but-1-oxy, 2-hydroxy-ethoxy, 3-hydroxy-prop-1-yl, 4-hydroxy-but-1-yl, 3-hydroxy-2,2-dimethyl-prop-1-oxy, cyclopropylmethoxy, 2,2,2-trifluoroethoxy, 2-(2-haloethoxy)-ethoxy, 2-(N-morpholino)-ethyl, 2-(N-morpholino)-ethoxy, 3-(N-morpholino)-prop-1-yl, 3-(N-morpholino)-prop-1-oxy, 4-(N-morpholino)-but-1-yl, 4-(N-morpholino)-but 1-oxy, 2-amino-ethyl, 2-(NHC(═O)O^{t}Bu)-ethyl, 2-amino-ethoxy, 2-(NHC(═O)O^{t}Bu)-ethoxy, 3-amino-prop-1-yl, 3-(NHC(═O)O^{t}Bu)-prop-1-yl, 3-amino-prop-1-oxy, 3-(NHC(═O)O^{t}Bu)-prop-1-oxy, 4-amino-but-1-yl, 4-(NHC(═O)O^{t}Bu)-but-1-yl, 4-amino-but-1-oxy, and 4-(NHC(═O)O^{t}Bu)-but-1-oxy.

In certain embodiments, X¹ is CH or N.

In certain embodiments, X⁴ is CH.

In certain embodiments, X² is CR^{6II}, R^{6II} is not H, X³ is CR^{6III}, and R^{6III} is not H.

In certain embodiments, X¹ is CH, X² is CR^{6II}, X³ is CR^{6III}, and X⁴ is CH, and one of the following applies: R^{6II} is methoxy, R^{6III} is 3-methoxy-propoxy; R^{6II} is chloro, R^{6III} is 3-methoxy-propoxy; R^{6II} is isopropyl, R^{6III} is 3-methoxy-propoxy; R^{6II} is methoxy, R^{6III} is methoxy; R^{6II} is chloro, R^{6III} is methoxy; and R^{6II} is cyclopropyl, R^{6III} is methoxy.

In certain embodiments, X¹ is N, X² is CR^{6II}, X³ is CR^{6III}, and X⁴ is CH, and one of the following applies: R^{6II} is methoxy, R^{6III} is 3-methoxy-propoxy; R^{6II} is chloro, R^{6III} is 3-methoxy-propoxy; R^{6II} is cyclopropyl, R^{6III} is 3-methoxy-propoxy; R^{6II} is methoxy, R^{6III} is methoxy; R^{6II} is chloro, R^{6III} is methoxy; and R^{6II} is cyclopropyl, R^{6III} is methoxy.

In certain embodiments, X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O—, and —O(CH₂)(CR¹¹R¹¹)(CH₂)O.

In certain embodiments, R⁷ is selected from the group consisting of H, methyl, ethyl, and fluoro.

In certain embodiments, a sAg secretion inhibitor/RNA destabilizer is elected from the group consisting of compounds of formula (I), (II), and (III), or a salt thereof, wherein for the compounds of formulas (I), (II), and (III) the following definitions apply:
R¹ is selected from the group consisting of H; halo; —OR⁸; —C(R⁹)(R⁹)OR⁸; —C(═O)R⁸; —C(═O)OR⁸; —C(═O)NH—OR⁸; —C(═O)NHNHR⁸; —C(═O)NHNHC(═O)R⁸; —C(═O)NHS(═O)₂R⁸; —CH₂C(═O)OR⁸; —CN: —NH₂; —N(R⁸)C(═O)H; —N(R⁸)C(═O)R¹⁰; —N(R⁸)C(═O)OR¹⁰; —N(R⁸)C(═O)NHR⁸; —NR⁹S(═O)₂R¹⁰; —P(═O)(OR⁸)₂; —B(OR⁸)₂; 2,5-dioxo-pyrrolidin-1-yl; 2H-tetrazol-5-yl; 3-hydroxy-isoxazol-5-yl; 1,4-dihydro-5-oxo-5H-tetrazol-1-yl; pyridin-2-yl optionally substituted with C₁-C₆ alkyl; pyrimidin-2-yl optionally substituted with C₁-C₆ alkyl; (pyridin-2-yl)methyl; (pyrimidin-2-yl)methyl; (pyrimidin-2-yl)amino; bis-(pyrimidin-2-yl)-amino; 5-R⁸-1,3,4,-thiadiazol-2-yl; 5-thioxo-4,5-dihydro-1H-1,2,4-triazol-3-yl; 1H-1,2,4-triazol-5-yl; 1,3,4-oxadiazol-2-yl; 1,2,4-oxadiazol-5-yl, and 3-R¹⁰-1,2,4-oxadiazol-5-yl;
R² is selected from the group consisting of ═O, ═NR⁹, ═N(OR⁹), and ═N(NR⁹R⁹);
   or R¹ and R² combine to form ═N—O—C(═O)— or ═N—N(R⁹)—C(═O)—, wherein the ═N group is bound to the ring carbon atom marked “*”;
X¹ is selected from the group consisting of CR^{6I} and N, X² is selected from the group consisting of CR^{6II} and N, X³ is selected from the group consisting of CR^{6III} and N, X⁴ is selected from the group consisting of CR^{6IV} and N, or either X³ and X⁴, or X¹ and X², combine to form —S—;
   wherein 0-2 substituents selected from the group consisting of X¹, X², X³ and X⁴ are N, each of which, if present, is optionally alkylated with C₁-C₆ alkyl if the adjacent carbon atom in the ring is substituted with —OH;
R^{6I}, R^{6II}, R^{6III} and R^{6IV} are independently selected from the group consisting of H, halo, —CN, pyrrolidinyl, optionally substituted C₁-C₆ alkyl, optionally substituted C₁-C₆ alkenyl, optionally substituted C₃-C₈ cycloalkyl, optionally substituted heterocyclyl, —OR, C₁-C₆ haloalkoxy, —N(R)(R), —NO₂, —S(═O)₂N(R)(R), acyl, and C₁-C₆ alkoxycarbonyl,
   wherein each occurrence of R is independently selected from the group consisting of H, C₁-C₆ alkyl, R′-substituted C₁-C₆ alkyl, C₁-C₆ hydroxyalkyl, optionally substituted (C₁-C₆ alkoxy)-C₁-C₆ alkyl, and optionally substituted C₃-C₈ cycloalkyl,
   wherein each occurrence of R′ is independently selected from the group consisting of —NH₂, —NH(C₁-C₆ alkyl), —N(C₁-C₆ alkyl)(C₁-C₆ alkyl), —NHC(═O))O^{t}Bu, —N(C₁-C₆ alkyl)C(═O)O^{t}Bu, or a 5- or 6-membered heterocyclic group, which is optionally N-linked;
   or X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—;
R⁷ is selected from the group consisting of H, OH, halo, C₁-C₆ alkoxy, optionally substituted C₁-C₆ alkyl, and optionally substituted C₃-C₈ cycloalkyl;
R⁸ is selected from the group consisting of H, optionally substituted C₁-C₆ alkyl, and optionally substituted C₃-C₈ cycloalkyl;
each occurrence of R⁹ is independently selected from the group consisting of H and C₁-C₆ alkyl;
R¹⁰ is selected from the group consisting of optionally substituted C₁-C₆ alkyl and optionally substituted phenyl; and,
each occurrence of R¹¹ is independently selected from the group consisting of H, OH, C₁-C₆ alkyl, C₁-C₆ alkoxy, alkoxy-C₁-C₆ alkyl and alkoxy-C₁-C₆ alkoxy, wherein two R¹¹ groups bound to the same carbon atom are not simultaneously OH; or two R¹¹ groups combine with the carbon atom to which they are bound to form a moiety selected from the group consisting of C═O, C═CH₂ and oxetane-3,3-diyl:
(a) wherein the compound of formula (I) is

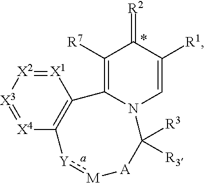

wherein in (I):
bond a is a single or double bond, wherein:
(i) if bond a is a single bond, then:
   Y is C(═O), and M is selected from the group consisting of C(R⁴)(R^{4′}) and NR⁸, or
   Y is selected from the group consisting of CHR⁵, O, S, S(═O), S(═O)₂, and NR⁵, M is C(R⁴)(R^{4′}),
      wherein, if Y is selected from the group consisting of CHR⁵, O, and NR⁵, R⁴ and R^{4′} optionally combine with each other to form ═O; or
      Y is CH, M is C(R⁴)(R^{4′}), R^{4′} is CH₂, and Y and R^{4′} form a single bond to generate cyclopropyl;
(ii) if bond a is a double bond, then Y is selected from the group consisting of CR and N, M is C(R⁴)(R^{4′}), and R^{4′} is absent;
R³, R^{3′}, R⁴ and R^{4′} are each independently selected from the group consisting of H, alkyl-substituted oxetanyl, optionally substituted C₁-C₆ alkyl and optionally substituted C₃-C₈ cycloalkyl;
   or one pair selected from the group consisting of R³/R^{3′}, R⁴/R^{4′}, and R³/R⁴ combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;
each occurrence of R⁵ is independently selected from the group consisting of H, optionally substituted C₁-C₆ alkyl, and optionally substituted C₃-C₈ cycloalkyl;
(b) wherein the compound of formula (II) is

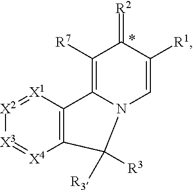

wherein in (II):
R³ and R^{3′} are each independently selected from the group consisting of H, alkyl-substituted oxetanyl, optionally substituted C₁-C₆ alkyl, and optionally substituted C₃-C₈ cycloalkyl;
   or R³ and R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙ(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;
(c) a compound of formula (III) is:

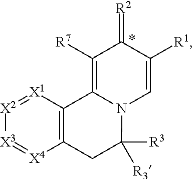

wherein in (III):
R³ and R^{3′} are each independently selected from the group consisting of H, alkyl-substituted oxetanyl, optionally substituted C₁-C₆ alkyl, and optionally substituted C₃-C₈ cycloalkyl;
   or R³ and R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;
and
the compound of formula (III) is selected from the group consisting of
a compound of formula (IIIa)

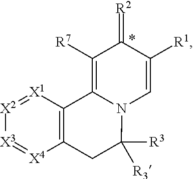

wherein 1-2 substituents selected from the group consisting of X¹, X², X³ and X⁴ are N;
a compound of formula (IIIb)

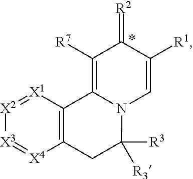

wherein at least one applies: R¹ is not —C(═O)OR⁸, R² is not ═O;
a compound of formula (IIIc)

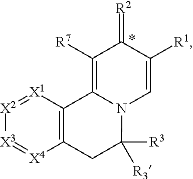

wherein X³ and X⁴, or X¹ and X², combine to form —S—;
a compound of formula (IIId)

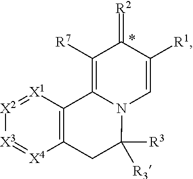

wherein X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—; and
a compound of formula (IIIe)

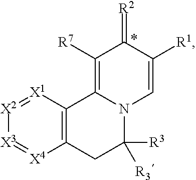

wherein R³ and R^{3′} are each independently selected from the group consisting of H, alkyl-substituted oxetanyl, optionally substituted C₁-C₆ alkyl, and optionally substituted C₃-C₈cycloalkyl, or R³ and R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2, and each divalent group is optionally substituted with at least one C₁-C₆ alkyl or halo.

In certain embodiments, the compound of formula (I) is a compound of formula (Ia):

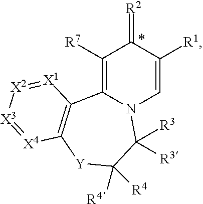

wherein in (Ia):
Y is selected from the group consisting of CHR⁵ and O; and
R³, R^{3′}, R⁴ and R^{4′} are each independently selected from the group consisting of H, alkyl-substituted oxetanyl, optionally substituted C₁-C₆ alkyl and optionally substituted C₃-C₈ cycloalkyl;
   or one pair selected from the group consisting of R³/R^{3′}, R⁴/R^{4′}, and R³/R⁴ combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo.

In certain embodiments, the compound of formula (I) is selected from the group consisting of:

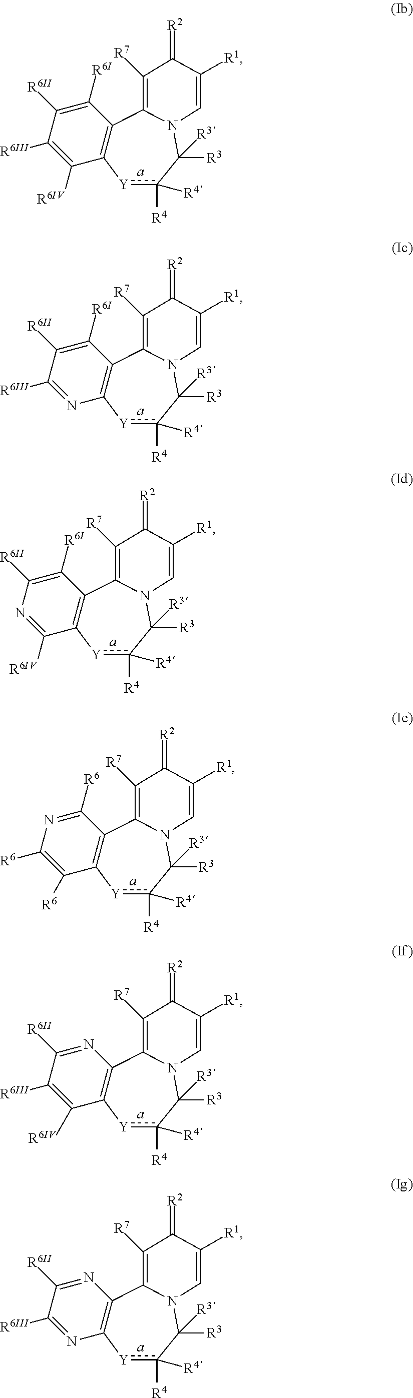

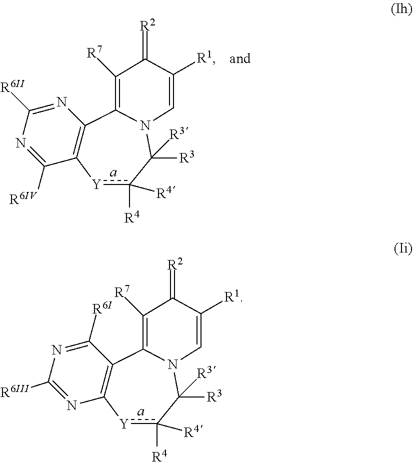

In certain embodiments, the compound of formula (Ia) is selected from the group consisting of:

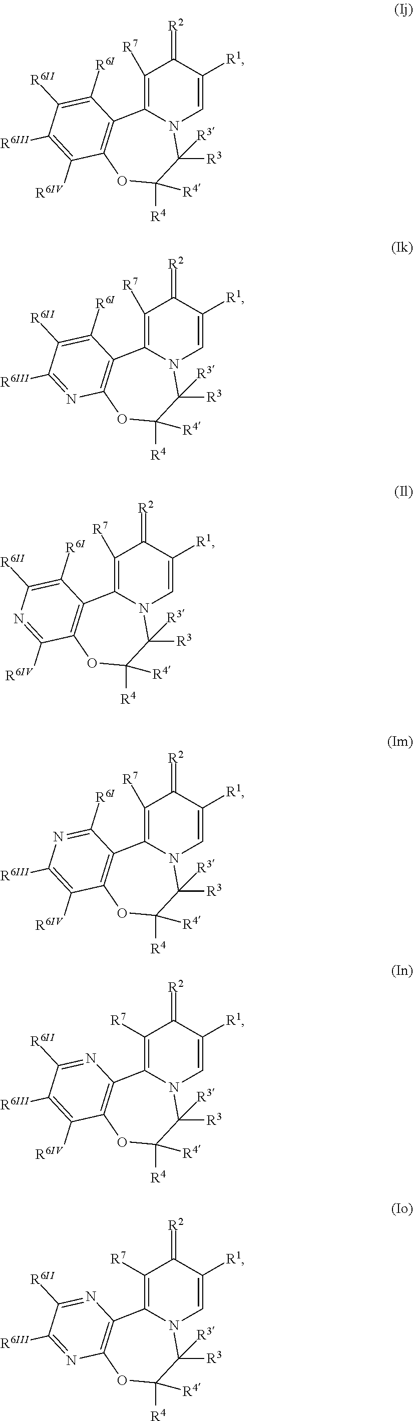

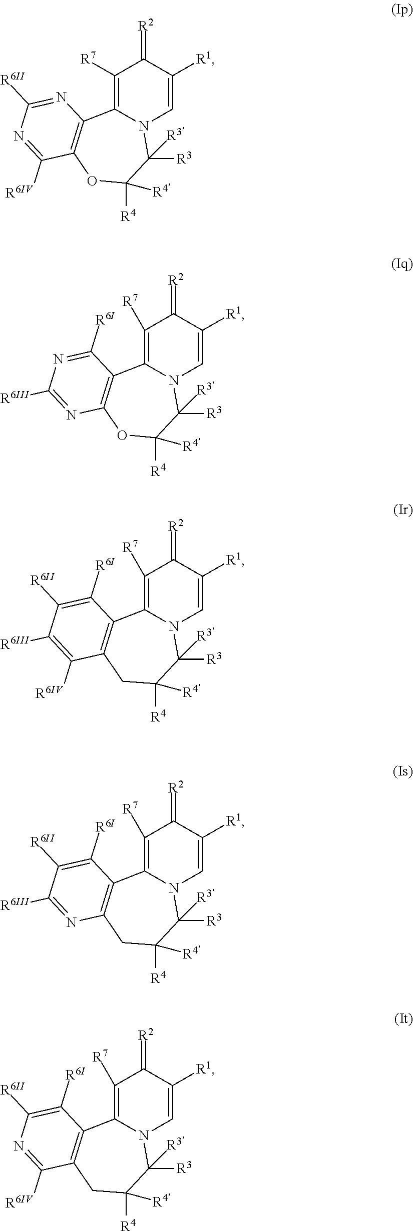

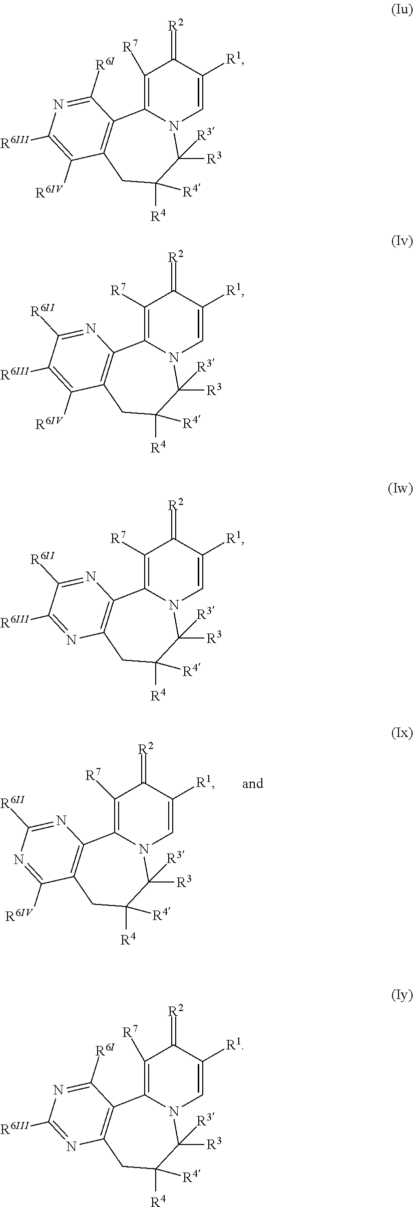

In certain embodiments, the compound of formula (II) is selected from the group consisting of:

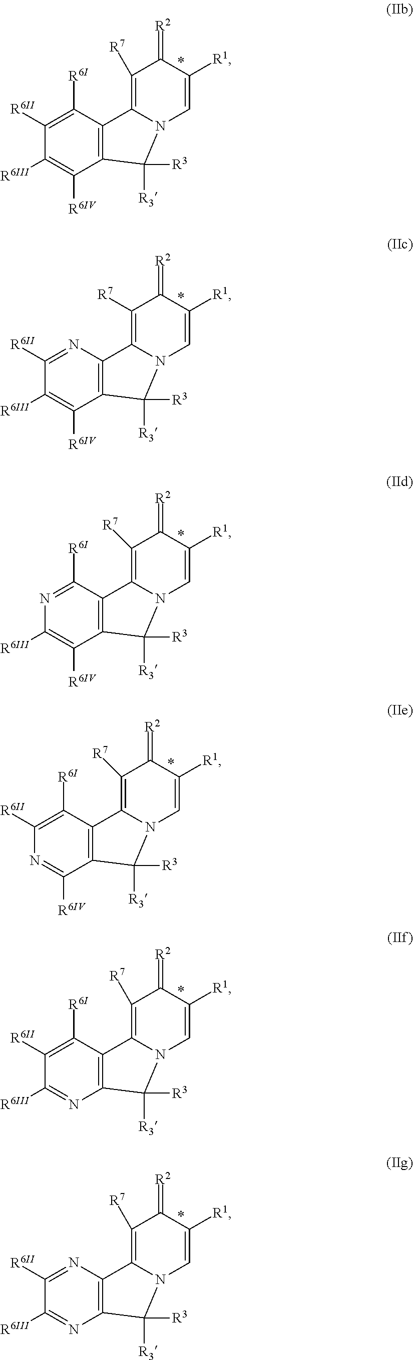

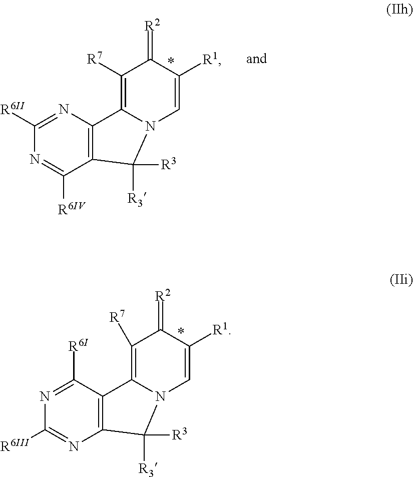

in certain embodiments, the compound of formula (III) is selected from the group consisting of:

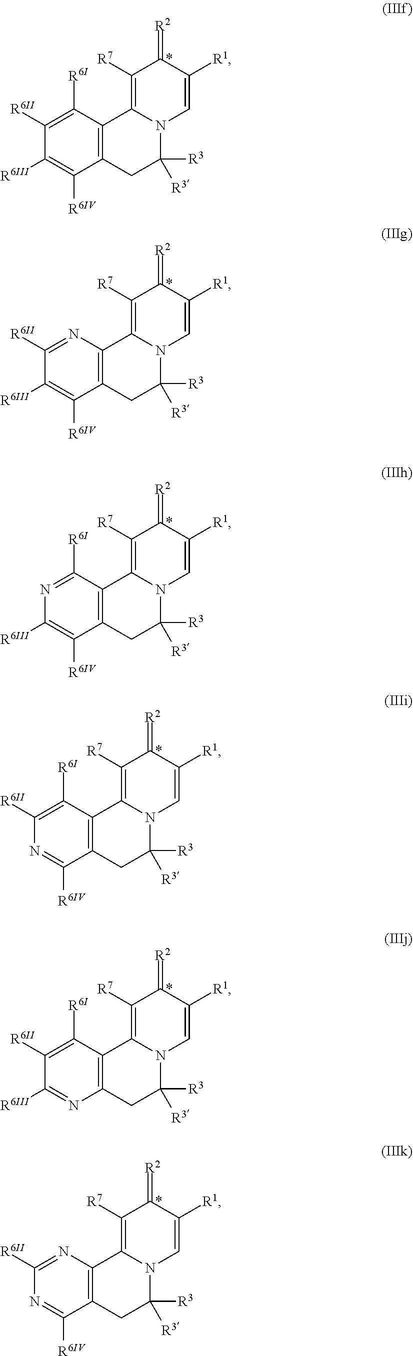

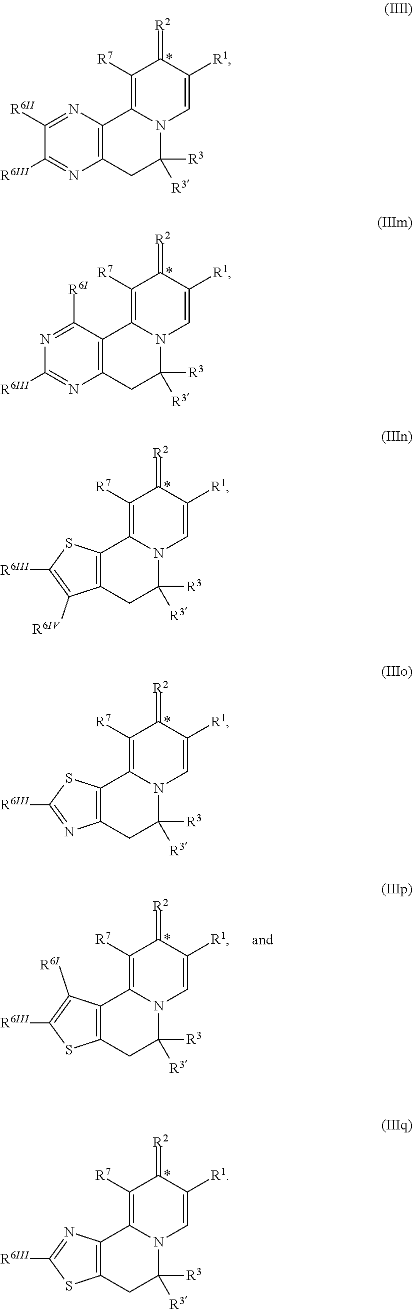

In certain embodiments, a sAg secretion inhibitor/RNA destabilizer is elected from the following compounds, or salts thereof.

| Structure | Nomenclature |
|---|---|
| 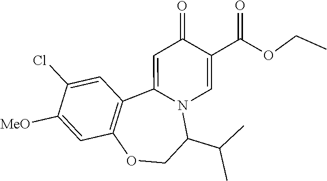 [SMILES: CCOC(=O)c1cn2c(cc1=O)-c1cc(Cl)c(OC)cc1OCC2C(C)C] | ethyl 2-chloro-7-isopropyl-3-methoxy-11-oxo- 6,7-dihydro-11H-benzo[f]pyrido[1,2- d][1,4]oxazepine-10-carboxylate |
| 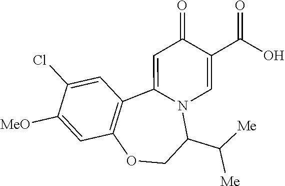 [SMILES: COc1cc2c(cc1Cl)-c1cc(=O)c(C(=O)O)cn1C(C(C)C)CO2] | 2-chloro-7-isopropyl-3-methoxy-11-oxo-6,7- dihydro-11H-benzo[f]pyrido[1,2- d][1,4]oxazepine-10-carboxylic acid |
| 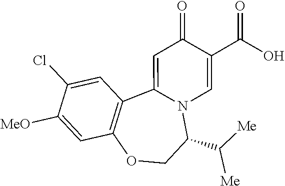 [SMILES: COc1cc2c(cc1Cl)-c1cc(=O)c(C(=O)O)cn1[C@H](C(C)C)CO2] | (R)-2-chloro-7-isopropyl-3-methoxy-11-oxo-6,7- dihydro-11H-benzo[f]pyrido[1,2- d][1,4]oxazepine-10-carboxylic acid |
| 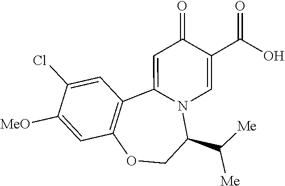 [SMILES: COc1cc2c(cc1Cl)-c1cc(=O)c(C(=O)O)cn1[C@@H](C(C)C)CO2] | (S)-2-chloro-7-isopropyl-3-methoxy-11-oxo-6,7- dihydro-11H-benzo[f]pyrido[1,2- d][1,4]oxazepine-10-carboxylic acid |
| 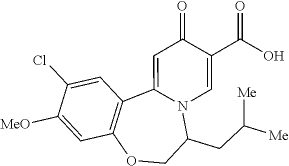 [SMILES: COc1cc2c(cc1Cl)-c1cc(=O)c(C(=O)O)cn1C(CC(C)C)CO2] | 2-chloro-7-isobutyl-3-methoxy-11-oxo-6,7- dihydro-11H-benzo[f]pyrido[1,2- d][1,4]oxazepine-10-carboxylic acid |
| 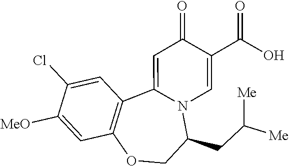 [SMILES: COc1cc2c(cc1Cl)-c1cc(=O)c(C(=O)O)cn1[C@@H](CC(C)C)CO2] | (S)-2-chloro-7-isobutyl-3-methoxy-11-oxo-6,7- dihydro-11H-benzo[f]pyrido[1,2- d][1,4]oxazepine-10-carboxylic acid |
| 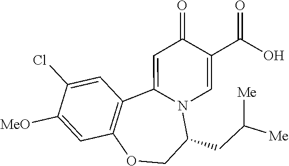 [SMILES: COc1cc2c(cc1Cl)-c1cc(=O)c(C(=O)O)cn1[C@H](CC(C)C)CO2] | (R)-2-chloro-7-isobutyl-3-methoxy-11-oxo-6,7- dihydro-11H-benzo[f]pyrido[1,2- d][1,4]oxazepine-10-carboxylic acid |
| 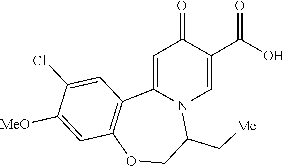 [SMILES: CCC1COc2cc(OC)c(Cl)cc2-c2cc(=O)c(C(=O)O)cn21] | 2-chloro-7-ethyl-3-methoxy-11-oxo-6,7-dihydro- 11H-benzo[f]pyrido[1,2-d][1,4]oxazepine-10- carboxylic acid |
| 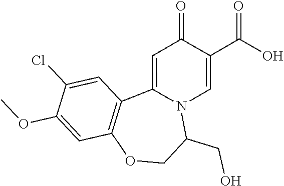 [SMILES: COc1cc2c(cc1Cl)-c1cc(=O)c(C(=O)O)cn1C(CO)CO2] | 2-chloro-7-(hydroxymethyl)-3-methoxy-11-oxo- 6,7-dihydro-11H-benzo[f]pyrido[1,2- d][1,4]oxazepine-10-carboxylic acid |
| 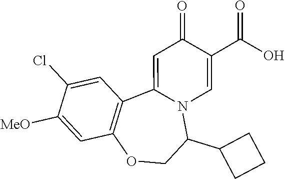 [SMILES: COc1cc2c(cc1Cl)-c1cc(=O)c(C(=O)O)cn1C(C1CCC1)CO2] | 2-chloro-7-cyclobutyl-3-methoxy-11-oxo-6,7- dihydro-11H-benzo[f]pyrido[1,2- d][1,4]oxazepine-10-carboxylic acid |
| 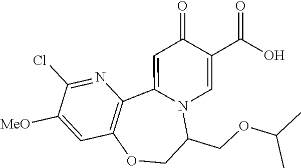 [SMILES: COc1cc2c(nc1Cl)-c1cc(=O)c(C(=O)O)cn1C(COC(C)C)CO2] | 2-chloro-7-(isopropoxymethyl)-3-methoxy-11- oxo-6,7-dihydro-11H-dipyrido[1,2-d:2′,3′- f][1,4]oxazepine-10-carboxylic acid |
| 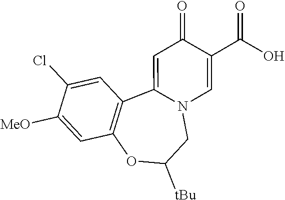 [SMILES: COc1cc2c(cc1Cl)-c1cc(=O)c(C(=O)O)cn1CC(C(C)(C)C)O2] | 6-(tert-butyl)-2-chloro-3-methoxy-11-oxo-6,7- dihydro-11H-benzo[f]pyrido[1,2- d][1,4]oxazepine-10-carboxylic acid |
| 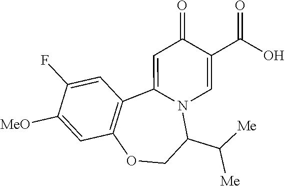 [SMILES: COc1cc2c(cc1F)-c1cc(=O)c(C(=O)O)cn1C(C(C)C)CO2] | 2-fluoro-7-isopropyl-3-methoxy-11-oxo-6,7- dihydro-11H-benzo[f]pyrido[1,2- d][1,4]oxazepine-10-carboxylic acid |
| 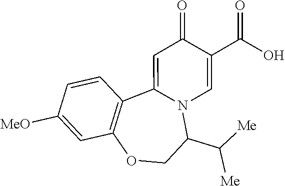 [SMILES: COc1ccc2c(c1)OCC(C(C)C)n1cc(C(=O)O)c(=O)cc1-2] | 7-isopropyl-3-methoxy-11-oxo-6,7-dihydro-11H- benzo[f]pyrido[1,2-d][1,4]oxazepine-10- carboxylic acid |
| 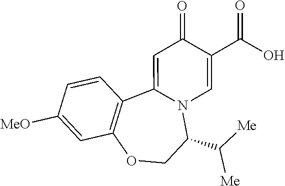 [SMILES: COc1ccc2c(c1)OC[C@@H](C(C)C)n1cc(C(=O)O)c(=O)cc1-2] | (R)-7-isopropyl-3-methoxy-11-oxo-6,7-dihydro- 11H-benzo[f]pyrido[1,2-d][1,4]oxazepine-10- carboxylic acid |
| 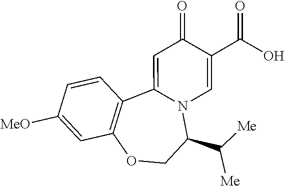 [SMILES: COc1ccc2c(c1)OC[C@H](C(C)C)n1cc(C(=O)O)c(=O)cc1-2] | (S)-7-isopropyl-3-methoxy-11-oxo-6,7-dihydro- 11H-benzo[f]pyrido[1,2-d][1,4]oxazepine-10- carboxylic acid |
| 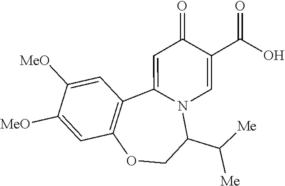 [SMILES: COc1cc2c(cc1OC)-c1cc(=O)c(C(=O)O)cn1C(C(C)C)CO2] | 6-isopropyl-10,11-dimethoxy-2-oxo-2,6,7,8- tetrahydrobenzo[c]pyrido[1,2-a]azepine-3- carboxylic acid |
| 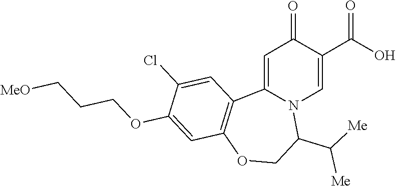 [SMILES: COCCCOc1cc2c(cc1Cl)-c1cc(=O)c(C(=O)O)cn1C(C(C)C)CO2] | 2-chloro-7-isopropyl-3-(3-methoxy propoxy)-11- oxo-6,7-dihydro-11H-benzo[f]pyrido[1,2- d][1,4]oxazepine-10-carboxylic acid |
| 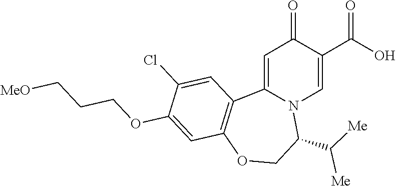 [SMILES: COCCCOc1cc2c(cc1Cl)-c1cc(=O)c(C(=O)O)cn1[C@H](C(C)C)CO2] | (R)-2-chloro-7-isopropyl-3-(3-methoxypropoxy)- 11-oxo-6,7-dihydro-11H-benzo[f]pyrido[1,2- d][1,4]oxazepine-10-carboxylic acid |
| 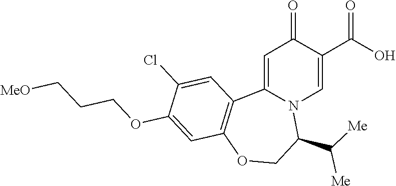 [SMILES: COCCCOc1cc2c(cc1Cl)-c1cc(=O)c(C(=O)O)cn1[C@@H](C(C)C)CO2] | (S)-2-chloro-7-isopropyl-3-(3-methoxypropoxy)- 11-oxo-6,7-dihydro-11H-benzo[f]pyrido[1,2- d][1,4]oxazepine-10-carboxylic acid |
| 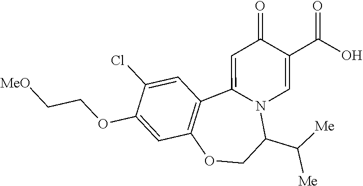 [SMILES: COCCOc1cc2c(cc1Cl)-c1cc(=O)c(C(=O)O)cn1C(C(C)C)CO2] | 2-chloro-7-isopropyl-3-(2-methoxy ethoxy)-11- oxo-6,7-dihydro-11H-benzo[f]pyrido[1,2- d][1,4]oxazepine-10-carboxylic acid |
| 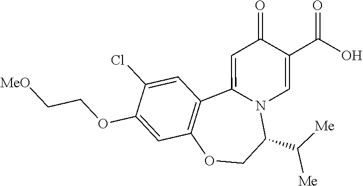 [SMILES: COCCOc1cc2c(cc1Cl)-c1cc(=O)c(C(=O)O)cn1[C@H](C(C)C)CO2] | (R)-2-chloro-7-isopropyl-3-(2-methoxyethoxy)- 11-oxo-6,7-dihydro-11H-benzo[f]pyrido[1,2- d][1,4]oxazepine-10-carboxylic acid |
| 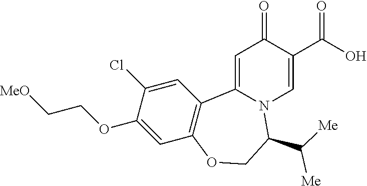 [SMILES: COCCOc1cc2c(cc1Cl)-c1cc(=O)c(C(=O)O)cn1[C@@H](C(C)C)CO2] | (S)-2-chloro-7-isopropyl-3-(2-methoxyethoxy)- 11-oxo-6,7-dihydro-11H-benzo[f]pyrido[1,2- d][1,4]oxazepine-10-carboxylic acid |
| 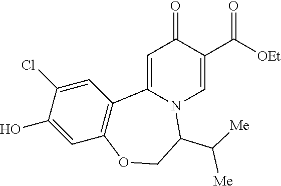 [SMILES: CCOC(=O)c1cn2c(cc1=O)-c1cc(Cl)c(O)cc1OCC2C(C)C] | ethyl 2-chloro-3-hydroxy-7-isopropyl-11-oxo-6,7- dihydro-11H-benzo[f]pyrido[1,2- d][1,4]oxazepine-10-carboxylate |
| 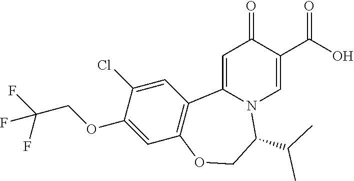 [SMILES: CC(C)[C@@H]1COc2cc(OCC(F)(F)F)c(Cl)cc2-c2cc(=O)c(C(=O)O)cn21] | (R)-2-chloro-7-isopropyl-11-oxo-3-(2,2,2- trifluoroethoxy)-6,7-dihydro-11H- benzo[f]pyrido[1,2-d][1,4]oxazepine-10- carboxylic acid |
| 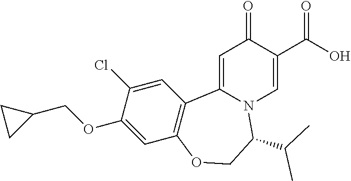 [SMILES: CC(C)[C@@H]1COc2cc(OCC3CC3)c(Cl)cc2-c2cc(=O)c(C(=O)O)cn21] | (R)-2-chloro-3-(cyclopropylmethoxy)-7- isopropyl-11-oxo-6,7-dihydro-11H- benzo[f]pyrido[1,2-d][1,4]oxazepine-10- carboxylic acid |
| 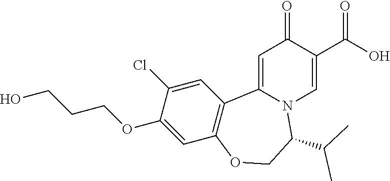 [SMILES: CC(C)[C@@H]1COc2cc(OCCCO)c(Cl)cc2-c2cc(=O)c(C(=O)O)cn21] | (R)-2-chloro-3-(3-hydroxypropoxy)-7-isopropyl- 11-oxo-6,7-dihydro-11H-benzo[f]pyrido[1,2- d][1,4]oxazepine-10-carboxylic acid |
| 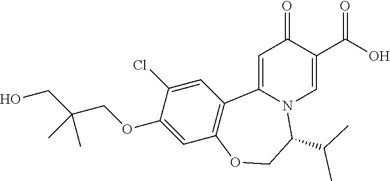 [SMILES: CC(C)[C@@H]1COc2cc(OCC(C)(C)CO)c(Cl)cc2-c2cc(=O)c(C(=O)O)cn21] | (R)-2-chloro-3-(3-hydroxy-2,2-dimethylpropoxy)- 7-isopropyl-11-oxo-6,7-dihydro-11H- benzo[f]pyrido[1,2-d][1,4]oxazepine-10- carboxylic acid |
| 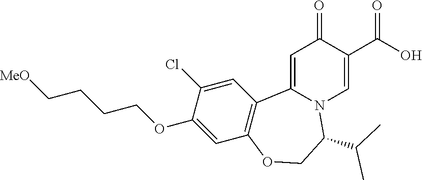 [SMILES: COCCCCOc1cc2c(cc1Cl)-c1cc(=O)c(C(=O)O)cn1[C@H](C(C)C)CO2] | (R)-2-chloro-7-isopropyl-3-(4-methoxybutoxy)- 11-oxo-6,7-dihydro-11H-benzo[f]pyrido[1,2- d][1,4]oxazepine-10-carboxylic acid |
| 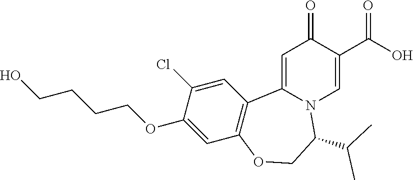 [SMILES: CC(C)[C@@H]1COc2cc(OCCCCO)c(Cl)cc2-c2cc(=O)c(C(=O)O)cn21] | (R)-2-chloro-3-(4-hydroxybutoxy)-7-isopropyl- 11-oxo-6,7-dihydro-11H-benzo[f]pyrido[1,2- d][1,4]oxazepine-10-carboxylic acid |
| 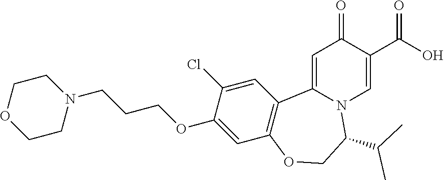 [SMILES: CC(C)[C@@H]1COc2cc(OCCCN3CCOCC3)c(Cl)cc2-c2cc(=O)c(C(=O)O)cn21] | (R)-2-chloro-7-isopropyl-3-(3- morpholinopropoxy)-11-oxo-6,7-dihydro-11H- benzo[f]pyrido[1,2-d][1,4]oxazepine-10- carboxylic acid |
| 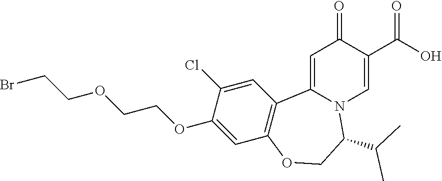 [SMILES: CC(C)[C@@H]1COc2cc(OCCOCCBr)c(Cl)cc2-c2cc(=O)c(C(=O)O)cn21] | (R)-3-(2-(2-bromoethoxy)ethoxy)-2-chloro-7- isopropyl-11-oxo-6,7-dihydro-11H- benzo[f]pyrido[1,2-d][1,4]oxazepine-10- carboxylic acid |
| 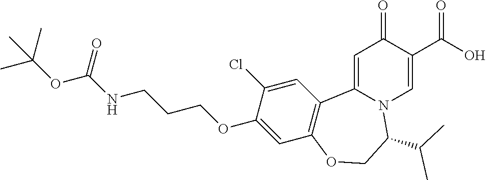 [SMILES: CC(C)[C@@H]1COc2cc(OCCCNC(=O)OC(C)(C)C)c(Cl)cc2-c2cc(=O)c(C(=O)O)cn21] | (R)-3-(3-((tert-butoxycarbonyl) amino)propoxy)- 2-chloro-7-isopropyl-11-oxo-6,7-dihydro-11H- benzo[f]pyrido[1,2-d][1,4]oxazepine-10- carboxylic acid |
| 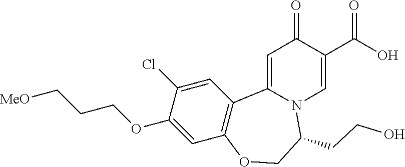 [SMILES: COCCCOc1cc2c(cc1Cl)-c1cc(=O)c(C(=O)O)cn1[C@H](CCO)CO2] | (R)-2-chloro-7-(2-hydroxyethyl)-3-(3- methoxypropoxy)-11-oxo-6,7-dihydro-11H- benzo[f]pyrido[1,2-d][1,4]oxazepine-10- carboxylic acid |
| 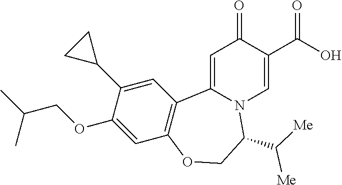 [SMILES: CC(C)COc1cc2c(cc1C1CC1)-c1cc(=O)c(C(=O)O)cn1[C@H](C(C)C)CO2] | (R)-2-cyclopropyl-3-isobutoxy-7-isopropyl-11- oxo-6,7-dihydro-11H-benzo[f]pyrido[1,2- d][1,4]oxazepine-10-carboxylic acid |
| 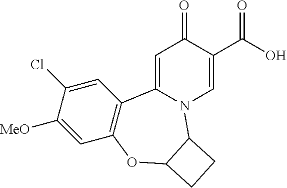 [SMILES: COc1cc2c(cc1Cl)-c1cc(=O)c(C(=O)O)cn1C1CCC1O2] | 11-chloro-10-methoxy-2-oxo-5a,6,7,7a- tetrahydro-2H-benzo[f]cyclobuta[b]pyrido[1,2- d][1,4]oxazepine-3-carboxylic acid |
| 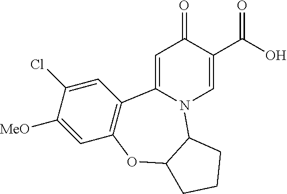 [SMILES: COc1cc2c(cc1Cl)-c1cc(=O)c(C(=O)O)cn1C1CCCC1O2] | 12-chloro-11-methoxy-2-oxo-5a,7,8,8a- tetrahydro-2H,6H- benzo[f]cyclopenta[b]pyrido[1,2- d][1,4]oxazepine-3-carboxylic acid |
| 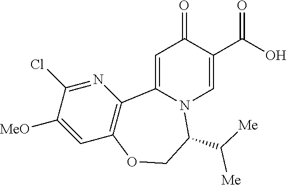 [SMILES: COc1cc2c(nc1Cl)-c1cc(=O)c(C(=O)O)cn1[C@H](C(C)C)CO2] | (R)-2-chloro-7-isopropyl-3-methoxy-11-oxo-6,7- dihydro-11H-dipyrido[1,2-d:2′,3′- f][1,4]oxazepine-10-carboxylic acid |
| 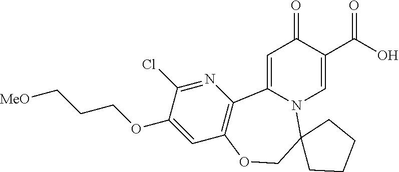 [SMILES: COCCCOc1cc2c(nc1Cl)-c1cc(=O)c(C(=O)O)cn1C1(CCCC1)CO2] | 2′-chloro-3′-(3-methoxypropoxy)-11′-oxo- 6′H, 11′H-spiro[cyclopentane-1,7′-dipyrido[1,2- d:2′,3′-f][1,4]oxazepine]-10′-carboxylic acid |
| 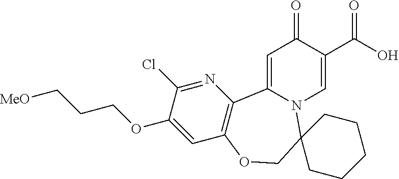 [SMILES: COCCCOc1cc2c(nc1Cl)-c1cc(=O)c(C(=O)O)cn1C1(CCCCC1)CO2] | 2′-chloro-3′-(3-methoxypropoxy)-11′-oxo- 6′H, 11′H-spiro[cyclohexane-1,7′-dipyrido[1,2- d:2′,3′-f][1,4]oxazepine]-10′-carboxylic acid |
| 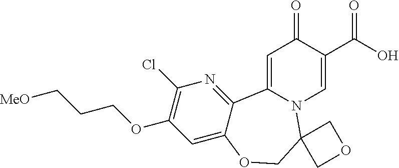 [SMILES: COCCCOc1cc2c(nc1Cl)-c1cc(=O)c(C(=O)O)cn1C1(COC1)CO2] | 2-chloro-3-(3-methoxypropoxy)-11-oxo-6H,11H- spiro[dipyrido[1,2-d:2′,3′-f][1,4]oxazepine-7,3′- oxetane]-10-carboxylic acid |
| 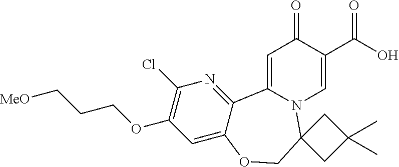 [SMILES: COCCCOc1cc2c(nc1Cl)-c1cc(=O)c(C(=O)O)cn1C1(CO2)CC(C)(C)C1] | 2′-chloro-3′-(3-methoxypropoxy)-3,3-dimethyl- 11′-oxo-6′H, 11′H-spiro[cyclobutane-1,7′- dipyrido[1,2-d:2′,3′-f][1,4]oxazepine]-10′- carboxylic acid |
| 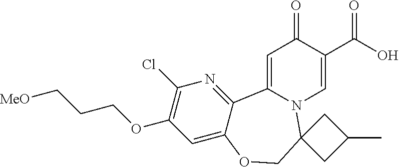 [SMILES: COCCCOc1cc2c(nc1Cl)-c1cc(=O)c(C(=O)O)cn1C1(CO2)CC(C)C1] | 2′-chloro-3′-(3-methoxypropoxy)-3-methyl-11′- oxo-6′H,11′H-spiro[cyclobutane-1,7′- dipyrido[1,2-d:2′,3′-f][1,4]oxazepine]-10′- carboxylic acid |
| 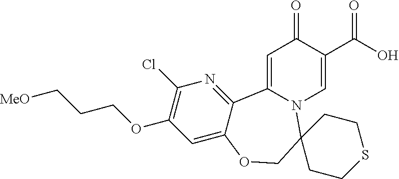 [SMILES: COCCCOc1cc2c(nc1Cl)-c1cc(=O)c(C(=O)O)cn1C1(CCSCC1)CO2] | 2-chloro-3-(3-methoxypropoxy)-11-oxo- 2′,3′,5′,6′-tetrahydro-6H,11H-spiro[dipyrido[1,2- d:2′,3′-f][1,4]oxazepine-7,4′-thiopyran]-10- carboxylic acid |
| 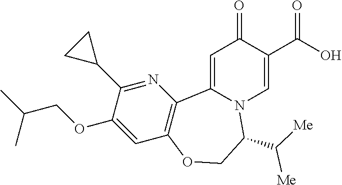 [SMILES: CC(C)COc1cc2c(nc1C1CC1)-c1cc(=O)c(C(=O)O)cn1[C@H](C(C)C)CO2] | (R)-2-cyclopropyl-3-isobutoxy-7-isopropyl-11- oxo-6,7-dihydro-11H-dipyrido[1,2-d:2′,3′- f][1,4]oxazepine-10-carboxylic acid |
| 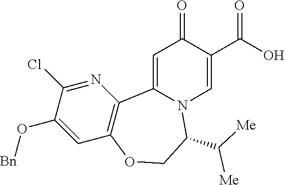 [SMILES: CC(C)[C@@H]1COc2cc(OCc3ccccc3)c(Cl)nc2-c2cc(=O)c(C(=O)O)cn21] | (R)-3-(benzyloxy)-2-chloro-7-isopropyl-11-oxo- 6,7-dihydro-11H-dipyrido[1,2-d:2′,3′- f][1,4]oxazepine-10-carboxylic acid |
| 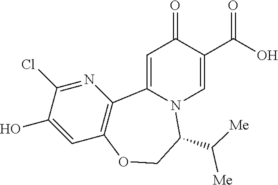 [SMILES: CC(C)[C@@H]1COc2cc(O)c(Cl)nc2-c2cc(=O)c(C(=O)O)cn21] | (R)-2-chloro-3-hydroxy-7-isopropyl-11-oxo-6,7- dihydro-11H-dipyrido[1,2-d:2′,3′- f][1,4]oxazepine-10-carboxylic acid |
| 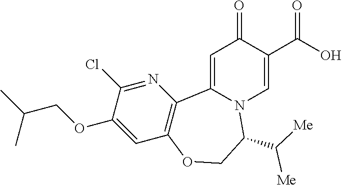 [SMILES: CC(C)COc1cc2c(nc1Cl)-c1cc(=O)c(C(=O)O)cn1[C@H](C(C)C)CO2] | (R)-2-chloro-3-isobutoxy-7-isopropyl-11-oxo-6,7- dihydro-11H-dipyrido[1,2-d:2′,3′- f][1,4]oxazepine-10-carboxylic acid |
| 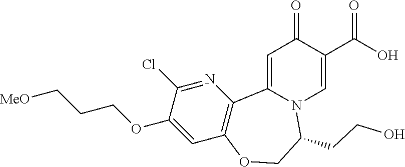 [SMILES: COCCCOc1cc2c(nc1Cl)-c1cc(=O)c(C(=O)O)cn1[C@H](CCO)CO2] | (R)-2-chloro-7-(2-hydroxyethyl)-3-(3- methoxypropoxy)-11-oxo-6,7-dihydro-11H- dipyrido[1,2-d:2′,3′-f][1,4]oxazepine-10- carboxylic acid |
| 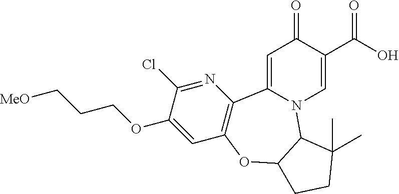 [SMILES: COCCCOc1cc2c(nc1Cl)-c1cc(=O)c(C(=O)O)cn1C1C(CCC1(C)C)O2] | 6-chloro-7-(3-methoxypropoxy)-12,12-dimethyl- 3-oxo-9a,11,12,12a-tetrahydro-3H,10H- cyclopenta[b]dipyrido[1,2-d:2′,3′- f][1,4]oxazepine-2-carboxylic acid |
| 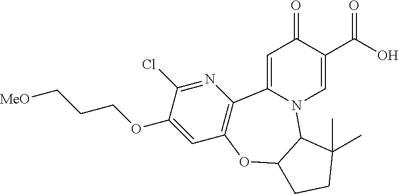 [SMILES: COCCCOc1cc2c(nc1Cl)-c1cc(=O)c(C(=O)O)cn1C1C(CCC1(C)C)O2] | 6-chloro-7-(3-methoxypropoxy)-12,12-dimethyl- 3-oxo-9a,11,12,12a-tetrahydro-3H,10H- cyclopenta[b]dipyrido[1,2-d:2′,3′- f][1,4]oxazepine-2-carboxylic acid (single enantiomer I) |
| 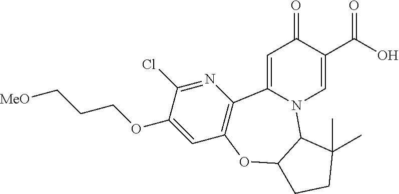 [SMILES: COCCCOc1cc2c(nc1Cl)-c1cc(=O)c(C(=O)O)cn1C1C(CCC1(C)C)O2] | 6-chloro-7-(3-methoxypropoxy)-12,12-dimethyl- 3-oxo-9a,11,12,12a-tetrahydro-3H,10H- cyclopenta[b]dipyrido[1,2-d:2′,3′- f][1,4]oxazepine-2-carboxylic acid (single enantiomer II) |
| 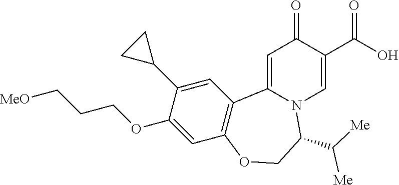 [SMILES: COCCCOc1cc2c(cc1C1CC1)-c1cc(=O)c(C(=O)O)cn1[C@H](C(C)C)CO2] | (R)-2-cyclopropyl-7-isopropyl-3-(3- methoxypropoxy)-11-oxo-6,7-dihydro-11H- benzo[f]pyrido[1,2-d][1,4]oxazepine-10- carboxylic acid |
| 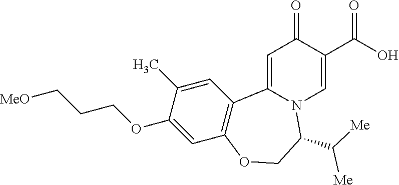 [SMILES: COCCCOc1cc2c(cc1C)-c1cc(=O)c(C(=O)O)cn1[C@H](C(C)C)CO2] | (R)-7-isopropyl-3-(3-methoxypropoxy)-2-methyl- 11-oxo-6,7-dihydro-11H-benzo[f]pyrido[1,2- d][1,4]oxazepine-10-carboxylic acid |
| 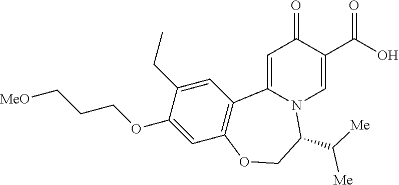 [SMILES: CCc1cc2c(cc1OCCCOC)OC[C@@H](C(C)C)n1cc(C(=O)O)c(=O)cc1-2] | (R)-2-ethyl-7-isopropyl-3-(3-methoxypropoxy)- 11-oxo-6,7-dihydro-11H-benzo[f]pyrido[1,2- d][1,4]oxazepine-10-carboxylic acid |
| 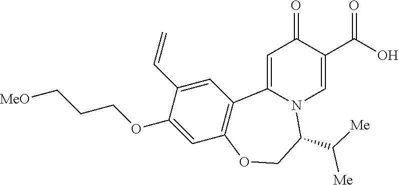 [SMILES: C=Cc1cc2c(cc1OCCCOC)OC[C@@H](C(C)C)n1cc(C(=O)O)c(=O)cc1-2] | (R)-7-isopropyl-3-(3-methoxypropoxy)-11-oxo-2- vinyl-6,7-dihydro-11H-benzo[f]pyrido[1,2- d][1,4]oxazepine-10-carboxylic acid |
| 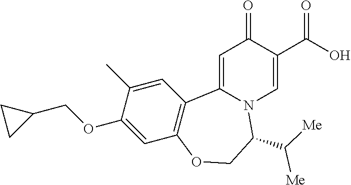 [SMILES: Cc1cc2c(cc1OCC1CC1)OC[C@@H](C(C)C)n1cc(C(=O)O)c(=O)cc1-2] | (R)-3-(cyclopropylmethoxy)-7-isopropyl-2- methyl-11-oxo-6,7-dihydro-11H- benzo[f]pyrido[1,2-d][1,4]oxazepine-10- carboxylic acid |
| 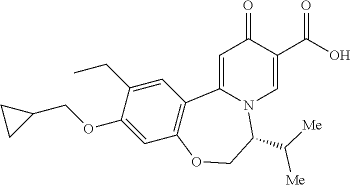 [SMILES: CCc1cc2c(cc1OCC1CC1)OC[C@@H](C(C)C)n1cc(C(=O)O)c(=O)cc1-2] | (R)-3-(cyclopropylmethoxy)-2-ethyl-7-isopropyl- 11-oxo-6,7-dihydro-11H-benzo[f]pyrido[1,2- d][1,4]oxazepine-10-carboxylic acid |
| 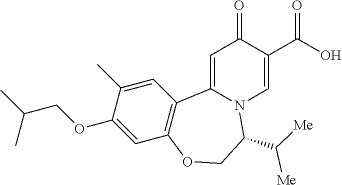 [SMILES: Cc1cc2c(cc1OCC(C)C)OC[C@@H](C(C)C)n1cc(C(=O)O)c(=O)cc1-2] | (R)-3-isobutoxy-7-isopropyl-2-methyl-11-oxo- 6,7-dihydro-11H-benzo[f]pyrido[1,2- d][1,4]oxazepine-10-carboxylic acid |
| 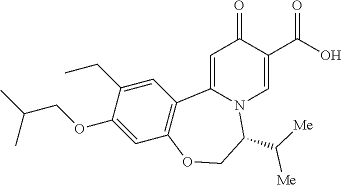 [SMILES: CCc1cc2c(cc1OCC(C)C)OC[C@@H](C(C)C)n1cc(C(=O)O)c(=O)cc1-2] | (R)-2-ethyl-3-isobutoxy-7-isopropyl-11-oxo-6,7- dihydro-11H-benzo[f]pyrido[1,2- d][1,4]oxazepine-10-carboxylic acid |
| 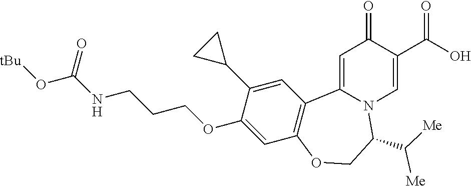 [SMILES: CC(C)[C@@H]1COc2cc(OCCCNC(=O)OC(C)(C)C)c(C3CC3)cc2-c2cc(=O)c(C(=O)O)cn21] | (R)-3-(3-((tert-butoxycarbonyl)amino)propoxy)- 2-cyclopropyl-7-isopropyl-11-oxo-6,7-dihydro- 11H-benzo[f]pyrido[1,2-d][1,4]oxazepine-10- carboxylic acid |
| 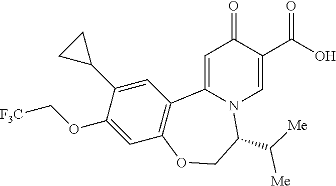 [SMILES: CC(C)[C@@H]1COc2cc(OCC(F)(F)F)c(C3CC3)cc2-c2cc(=O)c(C(=O)O)cn21] | (R)-2-cyclopropyl-7-isopropyl-11-oxo-3-(2,2,2- trifluoroethoxy)-6,7-dihydro-11H- benzo[f]pyrido[1,2-d][1,4]oxazepine-10- carboxylic acid |
| 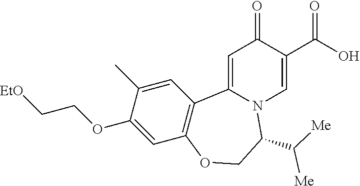 [SMILES: CCOCCOc1cc2c(cc1C)-c1cc(=O)c(C(=O)O)cn1[C@H](C(C)C)CO2] | (R)-3-(2-ethoxyethoxy)-7-isopropyl-2-methyl-11- oxo-6,7-dihydro-11H-benzo[f]pyrido[1,2- d][1,4]oxazepine-10-carboxylic acid |
| 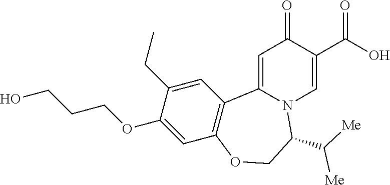 [SMILES: CCc1cc2c(cc1OCCCO)OC[C@@H](C(C)C)n1cc(C(=O)O)c(=O)cc1-2] | (R)-2-ethyl-3-(3-hydroxypropoxy)-7-isopropyl- 11-oxo-6,7-dihydro-11H-benzo[f]pyrido[1,2- d][1,4]oxazepine-10-carboxylic acid |
| 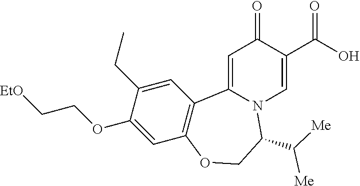 [SMILES: CCOCCOc1cc2c(cc1CC)-c1cc(=O)c(C(=O)O)cn1[C@H](C(C)C)CO2] | (R)-3-(2-ethoxyethoxy)-2-ethyl-7-isopropyl-11- oxo-6,7-dihydro-11H-benzo[f]pyrido[1,2- d][1,4]oxazepine-10-carboxylic acid |
| 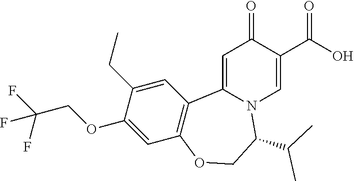 [SMILES: CCc1cc2c(cc1OCC(F)(F)F)OC[C@@H](C(C)C)n1cc(C(=O)O)c(=O)cc1-2] | (R)-2-ethyl-7-isopropyl-11-oxo-3-(2,2,2- trifluoroethoxy)-6,7-dihydro-11H- benzo[f]pyrido[1,2-d][1,4]oxazepine-10- carboxylic acid |
| 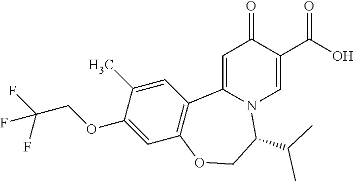 [SMILES: Cc1cc2c(cc1OCC(F)(F)F)OC[C@@H](C(C)C)n1cc(C(=O)O)c(=O)cc1-2] | (R)-7-isopropyl-2-methyl-11-oxo-3-(2,2,2- trifluoroethoxy)-6,7-dihydro-11H- benzo[f]pyrido[1,2-d][1,4]oxazepine-10- carboxylic acid |
| 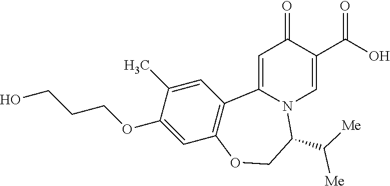 [SMILES: Cc1cc2c(cc1OCCCO)OC[C@@H](C(C)C)n1cc(C(=O)O)c(=O)cc1-2] | (R)-3-(3-hydroxypropoxy)-7-isopropyl-2-methyl- 11-oxo-6,7-dihydro-11H-benzo[f]pyrido[1,2- d][1,4]oxazepine-10-carboxylic acid |
| 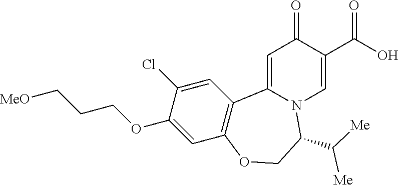 [SMILES: COCCCOc1cc2c(cc1Cl)-c1cc(=O)c(C(=O)O)cn1[C@H](C(C)C)CO2] | (R)-2-chloro-7-isopropyl-3-((3- methoxypropyl)amino)-11-oxo-6,7-dihydro-11H- benzo[f]pyrido[1,2-d][1,4]oxazepine-10- carboxylic acid |
| 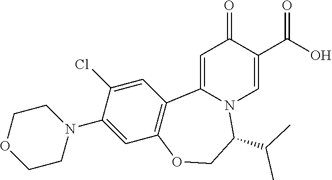 [SMILES: CC(C)[C@@H]1COc2cc(N3CCOCC3)c(Cl)cc2-c2cc(=O)c(C(=O)O)cn21] | (R)-2-chloro-7-isopropyl-3-morpholino-11-oxo- 6,7-dihydro-11H-benzo[f]pyrido[1,2- d][1,4]oxazepine-10-carboxylic acid |
| 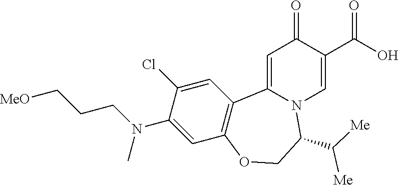 [SMILES: COCCCN(C)c1cc2c(cc1Cl)-c1cc(=O)c(C(=O)O)cn1[C@H](C(C)C)CO2] | (R)-2-chloro-7-isopropyl-3-((3- methoxypropyl)(methyl)amino)-11-oxo-6,7- dihydro-11H-benzo[f]pyrido[1,2- d][1,4]oxazepine-10-carboxylic acid |
| 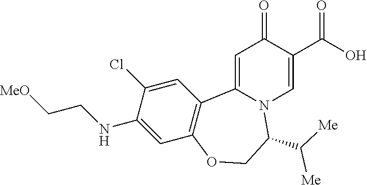 [SMILES: COCCNc1cc2c(cc1Cl)-c1cc(=O)c(C(=O)O)cn1[C@H](C(C)C)CO2] | (R)-2-chloro-7-isopropyl-3-((2- methoxyethyl)amino)-11-oxo-6,7-dihydro-11H- benzo[f]pyrido[1,2-d][1,4]oxazepine-10- carboxylic acid |
| 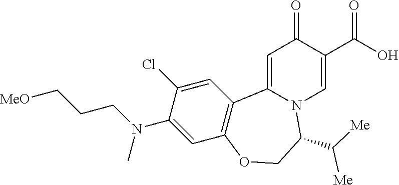 [SMILES: COCCCN(C)c1cc2c(cc1Cl)-c1cc(=O)c(C(=O)O)cn1[C@H](C(C)C)CO2] | (R)-2-chloro-7-isopropyl-3-((2- methoxyethyl)(methyl)amino)-11-oxo-6,7- dihydro-11H-benzo[f]pyrido[1,2- d][1,4]oxazepine-10-carboxylic acid |
| 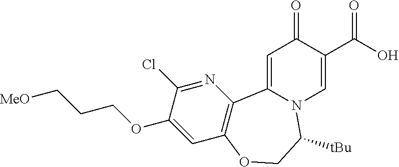 [SMILES: COCCCOc1cc2c(nc1Cl)-c1cc(=O)c(C(=O)O)cn1[C@H](C(C)(C)C)CO2] | (R)-7-(tert-butyl)-2-chloro-3-(3- methoxypropoxy)-11-oxo-6,7-dihydro-11H- dipyrido[1,2-d:2′,3′-f][1,4]oxazepine-10- carboxylic acid |
| 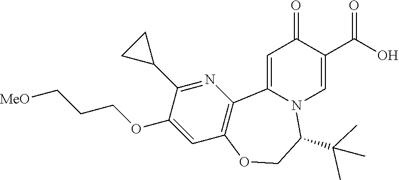 [SMILES: COCCCOc1cc2c(nc1C1CC1)-c1cc(=O)c(C(=O)O)cn1[C@H](C(C)(C)C)CO2] | (R)-7-(tert-butyl)-2-cyclopropyl-3-(3- methoxypropoxy)-11-oxo-6,7-dihydro-11H- dipyrido[1,2-d:2′,3′-f][1,4]oxazepine-10- carboxylic acid |
| 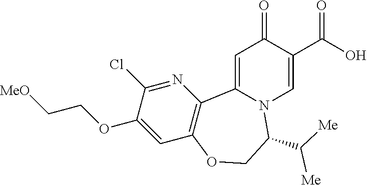 [SMILES: COCCOc1cc2c(nc1Cl)-c1cc(=O)c(C(=O)O)cn1[C@H](C(C)C)CO2] | (R)-2-chloro-7-isopropyl-3-(3-methoxypropoxy)- 11-oxo-6,7-dihydro-11H-dipyrido[1,2-d:2′,3′- f][1,4]oxazepine-10-carboxylic acid |
| 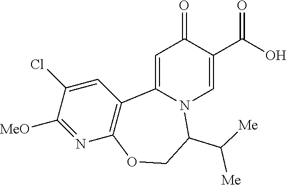 [SMILES: COc1nc2c(cc1Cl)-c1cc(=O)c(C(=O)O)cn1C(C(C)C)CO2] | 2-chloro-7-isopropyl-3-methoxy-11-oxo-6,7- dihydro-11H-dipyrido[1,2-d:3′,2′- f][1,4]oxazepine-10-carboxylic acid |
| 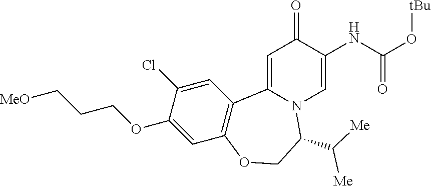 [SMILES: COCCCOc1cc2c(cc1Cl)-c1cc(=O)c(NC(=O)OC(C)(C)C)cn1[C@H](C(C)C)CO2] | tert-butyl (R)-(2-chloro-7-isopropyl-3-(3- methoxypropoxy)-11-oxo-6,7-dihydro-11H- benzo[f]pyrido[1,2-d][1,4]oxazepin-10- yl)carbamate |
| 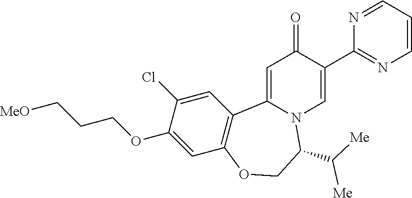 [SMILES: COCCCOc1cc2c(cc1Cl)-c1cc(=O)c(-c3ncccn3)cn1[C@H](C(C)C)CO2] | (R)-2-chloro-7-isopropyl-3-(3-methoxypropoxy)- 10-(pyrimidin-2-yl)-6,7-dihydro-11H- benzo[f]pyrido[1,2-d][1,4]oxazepin-11-one |
| 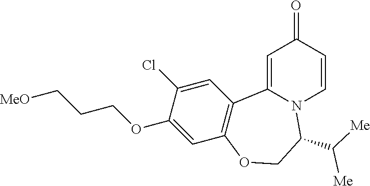 [SMILES: COCCCOc1cc2c(cc1Cl)-c1cc(=O)ccn1[C@H](C(C)C)CO2] | (R)-2-chloro-7-isopropyl-3-(3-methoxypropoxy)- 6,7-dihydro-11H-benzo[f]pyrido[1,2- d][1,4]oxazepin-11-one |
| 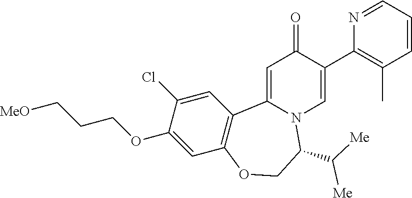 [SMILES: COCCCOc1cc2c(cc1Cl)-c1cc(=O)c(-c3ncccc3C)cn1[C@H](C(C)C)CO2] | (R)-2-chloro-7-isopropyl-3-(3-methoxypropoxy)- 10-(3-methylpyridin-2-yl)-6,7-dihydro-11H- benzo[f]pyrido[1,2-d][1,4]oxazepin-11-one |
| 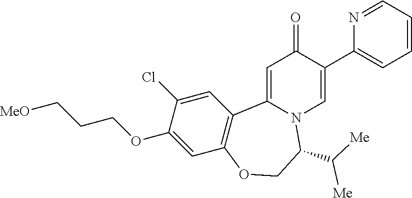 [SMILES: COCCCOc1cc2c(cc1Cl)-c1cc(=O)c(-c3ccccn3)cn1[C@H](C(C)C)CO2] | (R)-2-chloro-7-isopropyl-3-(3-methoxypropoxy)- 10-(pyridin-2-yl)-6,7-dihydro-11H- benzo[f]pyrido[1,2-d][1,4]oxazepin-11-one |
| 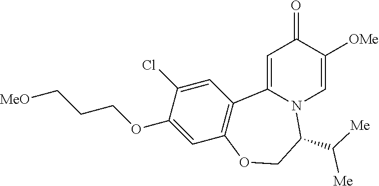 [SMILES: COCCCOc1cc2c(cc1Cl)-c1cc(=O)c(OC)cn1[C@H](C(C)C)CO2] | (R)-2-chloro-7-isopropyl-10-methoxy-3-(3- methoxypropoxy)-6,7-dihydro-11H- benzo[f]pyrido[1,2-d][1,4]oxazepin-11-one |
| 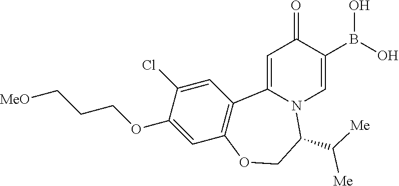 [SMILES: COCCCOc1cc2c(cc1Cl)-c1cc(=O)c(B(O)O)cn1[C@H](C(C)C)CO2] | (R)-(2-chloro-7-isopropyl-3-(3-methoxypropoxy)- 11-oxo-6,7-dihydro-11H-benzo[f]pyrido[1,2- d][1,4]oxazepin-10-yl)boronic acid |
| 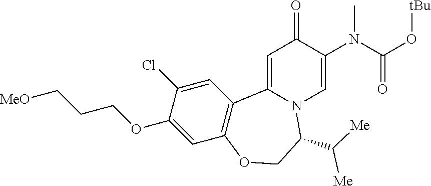 [SMILES: COCCCOc1cc2c(cc1Cl)-c1cc(=O)c(N(C)C(=O)OC(C)(C)C)cn1[C@H](C(C)C)CO2] | tert-butyl (R)-(2-chloro-7-isopropyl-3-(3- methoxypropoxy)-11-oxo-6,7-dihydro-11H- benzo[f]pyrido[1,2-d][1,4]oxazepin-10- yl)(methyl)carbamate |
| 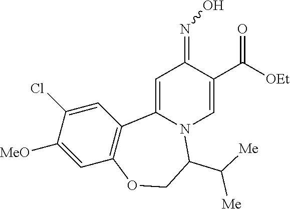 [SMILES: CCOC(=O)c1cn2c(cc1=NO)-c1cc(Cl)c(OC)cc1OCC2C(C)C] | ethyl 2-chloro-11-(hydroxyimino)-7-isopropyl-3- methoxy-6,7-dihydro-11H-benzo[f]pyrido[1,2- d][1,4]oxazepine-10-carboxylate |
| 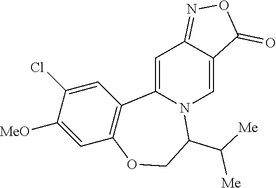 [SMILES: COc1cc2c(cc1Cl)-c1cc3noc(=O)c-3cn1C(C(C)C)CO2] | 2-chloro-7-isopropyl-3-methoxy-6,7-dihydro- 10H-benzo[f]isoxazolo[3′,4′:4,5]pyrido[1,2- d][1,4]oxazepin-10-one |
| 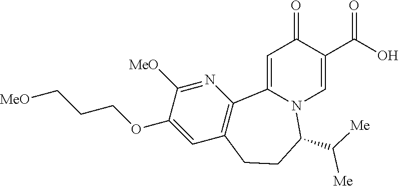 [SMILES: COCCCOc1cc2c(nc1OC)-c1cc(=O)c(C(=O)O)cn1[C@H](C(C)C)CC2] | (S)-7-isopropyl-2-methoxy-3-(3- methoxypropoxy)-11-oxo-5,6,7,11- tetrahydrodipyrido[1,2-a:2′,3′-c]azepine-10- carboxylic acid |
| 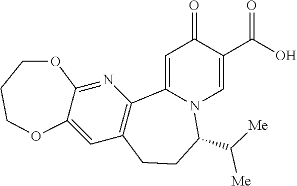 [SMILES: CC(C)[C@@H]1CCc2cc3c(nc2-c2cc(=O)c(C(=O)O)cn21)OCCCO3] | (S)-6-isopropyl-2-oxo-2,6,7,8,12,13-hexahydro- 11H-[1,4]dioxepino[2′,3′:5,6]pyrido[2,3- c]pyrido[1,2-a]azepine-3-carboxylic acid |
| 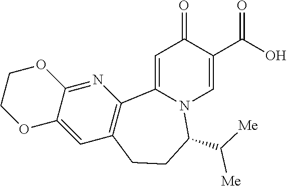 [SMILES: CC(C)[C@@H]1CCc2cc3c(nc2-c2cc(=O)c(C(=O)O)cn21)OCCO3] | (S)-6-isopropyl-2-oxo-2,6,7,8,11,12-hexahydro- [1,4]dioxino[2′,3′:5,6]pyrido[2,3-c]pyrido[1,2- a]azepine-3-carboxylic acid |
| 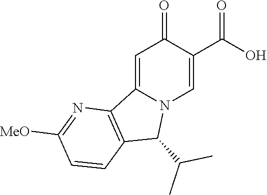 [SMILES: COc1ccc2c(n1)-c1cc(=O)c(C(=O)O)cn1[C@@H]2C(C)C] | (R)-5-isopropyl-2-methoxy-9-oxo-5,9- dihydropyrido[2,3-a]indolizine-8-carboxylic acid |
| 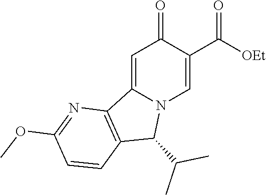 [SMILES: CCOC(=O)c1cn2c(cc1=O)-c1nc(OC)ccc1[C@H]2C(C)C] | ethyl (R)-5-isopropyl-2-methoxy-9-oxo-5,9- dihydropyrido[2,3-a]indolizine-8-carboxylate |
| 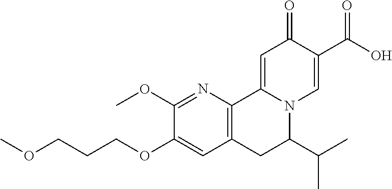 [SMILES: COCCCOc1cc2c(nc1OC)-c1cc(=O)c(C(=O)O)cn1C(C(C)C)C2] | 6-isopropyl-2-methoxy-3-(3-methoxypropoxy)- 10-oxo-5,10-dihydro-6H-pyrido[1,2- h][1,7]naphthyridine-9-carboxylic acid |
| 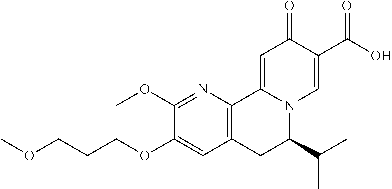 [SMILES: COCCCOc1cc2c(nc1OC)-c1cc(=O)c(C(=O)O)cn1[C@@H](C(C)C)C2] | (R)-6-isopropyl-2-methoxy-3-(3- methoxypropoxy)-10-oxo-5,10-dihydro-6H- pyrido[1,2-h][1,7]naphthyridine-9-carboxylic acid |
| 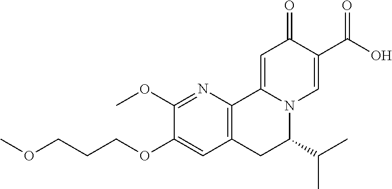 [SMILES: COCCCOc1cc2c(nc1OC)-c1cc(=O)c(C(=O)O)cn1[C@H](C(C)C)C2] | (S)-6-isopropyl-2-methoxy-3-(3- methoxypropoxy)-10-oxo-5,10-dihydro-6H- pyrido[1,2-h][1,7]naphthyridine-9-carboxylic acid |
| 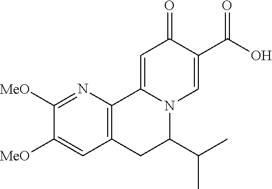 [SMILES: COc1cc2c(nc1OC)-c1cc(=O)c(C(=O)O)cn1C(C(C)C)C2] | 6-isopropyl-2,3-dimethoxy-10-oxo-5,10-dihydro- 6H-pyrido[1,2-h][1,7]naphthyridine-9-carboxylic acid |
| 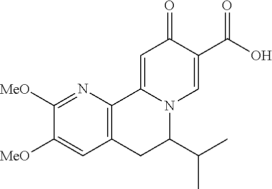 [SMILES: COc1cc2c(nc1OC)-c1cc(=O)c(C(=O)O)cn1C(C(C)C)C2] | 6-isopropyl-2,3-dimethoxy-10-oxo-5,10-dihydro- 6H-pyrido[1,2-h][1,7]naphthyridine-9-carboxylic acid (single enantiomer I) |
| 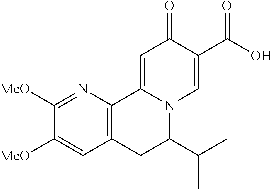 [SMILES: COc1cc2c(nc1OC)-c1cc(=O)c(C(=O)O)cn1C(C(C)C)C2] | 6-isopropyl-2,3-dimethoxy-10-oxo-5,10-dihydro- 6H-pyrido[1,2-h][1,7]naphthyridine-9-carboxylic acid (single enantiomer II) |
| 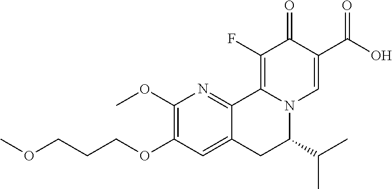 [SMILES: COCCCOc1cc2c(nc1OC)-c1c(F)c(=O)c(C(=O)O)cn1[C@H](C(C)C)C2] | (S)-11-fluoro-6-isopropyl-2-methoxy-3-(3- methoxypropoxy)-10-oxo-5,10-dihydro-6H- pyrido[1,2-h][1,7]naphthyridine-9-carboxylic acid |
| 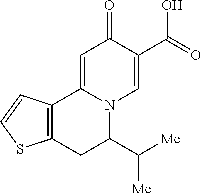 [SMILES: CC(C)C1Cc2sccc2-c2cc(=O)c(C(=O)O)cn21] | 5-isopropyl-9-oxo-4,9-dihydro-5H-thieno[3,2- a]quinolizine-8-carboxylic acid |
| 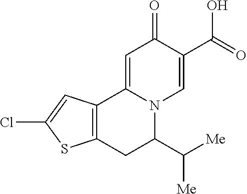 [SMILES: CC(C)C1Cc2sc(Cl)cc2-c2cc(=O)c(C(=O)O)cn21] | 2-chloro-5-isopropyl-9-oxo-4,9-dihydro-5H- thieno[3,2-a]quinolizine-8-carboxylic acid |
| 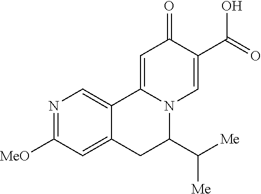 [SMILES: COc1cc2c(cn1)-c1cc(=O)c(C(=O)O)cn1C(C(C)C)C2] | 6-isopropyl-3-methoxy-10-oxo-5,10-dihydro-6H- pyrido[2,1-a][2,7]naphthyridine-9-carboxylic acid |
| 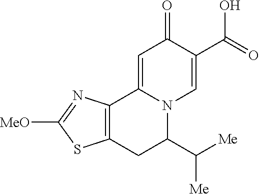 [SMILES: COc1nc2c(s1)CC(C(C)C)n1cc(C(=O)O)c(=O)cc1-2] | 5-isopropyl-2-methoxy-9-oxo-4,9-dihydro-5H- thiazolo[4,5-a]quinolizine-8-carboxylic acid |
| 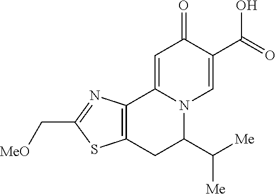 [SMILES: COCc1nc2c(s1)CC(C(C)C)n1cc(C(=O)O)c(=O)cc1-2] | 5-isopropyl-2-(methoxymethyl)-9-oxo-4,9- dihydro-5H-thiazolo[4,5-a]quinolizine-8- carboxylic acid |
| 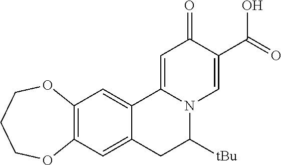 [SMILES: CC(C)(C)C1Cc2cc3c(cc2-c2cc(=O)c(C(=O)O)cn21)OCCCO3] | 6-(tert-butyl)-2-oxo-6,7,11,12-tetrahydro- 2H,10H-[1,4]dioxepino[2,3-g]pyrido[2,1- a]isoquinoline-3-carboxylic acid |
| 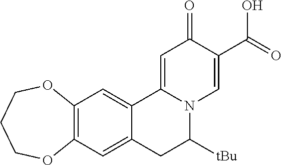 [SMILES: CC(C)(C)C1Cc2cc3c(cc2-c2cc(=O)c(C(=O)O)cn21)OCCCO3] | 6-(tert-butyl)-2-oxo-6,7,11,12-tetrahydro- 2H,10H-[1,4]dioxepino[2,3-g]pyrido[2,1- a]isoquinoline-3-carboxylic acid (single enantiomer I) |
| 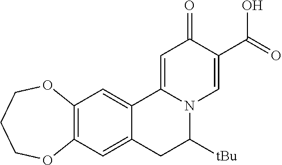 [SMILES: CC(C)(C)C1Cc2cc3c(cc2-c2cc(=O)c(C(=O)O)cn21)OCCCO3] | 6-(tert-butyl)-2-oxo-6,7,11,12-tetrahydro- 2H,10H-[1,4]dioxepino[2,3-g]pyrido[2,1- a]isoquinoline-3-carboxylic acid (single enantiomer II) |
| 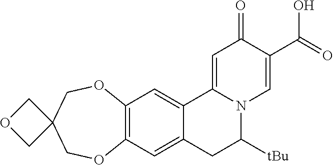 [SMILES: CC(C)(C)C1Cc2cc3c(cc2-c2cc(=O)c(C(=O)O)cn21)OCC1(COC1)CO3] | 6′-(tert-butyl)-2′-oxo-6′,7′-dihydro-2′H,10′H,12′H- spiro[oxetane-3,11′-[1,4]dioxepino[2,3- g]pyrido[2,1-a]isoquinoline]-3′-carboxylic acid |
| 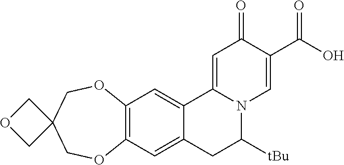 [SMILES: CC(C)(C)C1Cc2cc3c(cc2-c2cc(=O)c(C(=O)O)cn21)OCC1(COC1)CO3] | 6′-(tert-butyl)-2′-oxo-6′,7′-dihydro-2′H,10′H,12′H- spiro[oxetane-3,11′-[1,4]dioxepino[2,3- g]pyrido[2,1-a]isoquinoline]-3′-carboxylic acid (single enantiomer I) |
| 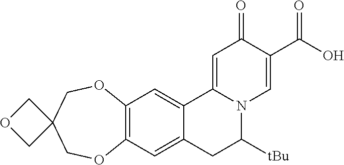 [SMILES: CC(C)(C)C1Cc2cc3c(cc2-c2cc(=O)c(C(=O)O)cn21)OCC1(COC1)CO3] | 6′-(tert-butyl)-2′-oxo-6′,7′-dihydro-2′H,10′H,12′H- spiro[oxetane-3,11′-[1,4]dioxepino[2,3- g]pyrido[2,1-a]isoquinoline]-3′-carboxylic acid (single enantiomer II) |
| 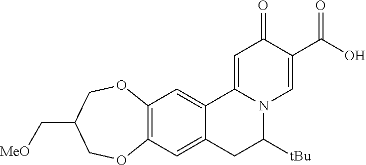 [SMILES: COCC1COc2cc3c(cc2OC1)-c1cc(=O)c(C(=O)O)cn1C(C(C)(C)C)C3] | 6-(tert-butyl)-11-(methoxymethyl)-2-oxo- 6,7,11,12-tetrahydro-2H,10H-[1,4]dioxepino[2,3- g]pyrido[2,1-a]isoquinoline-3-carboxylic acid |
| 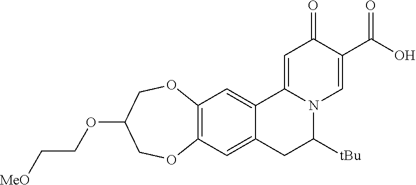 [SMILES: COCCOC1COc2cc3c(cc2OC1)-c1cc(=O)c(C(=O)O)cn1C(C(C)(C)C)C3] | 6-(tert-butyl)-11-(2-methoxyethoxy)-2-oxo- 6,7,11,12-tetrahydro-2H,10H-[1,4]dioxepino[2,3- g]pyrido[2,1-a]isoquinoline-3-carboxylic acid |
| 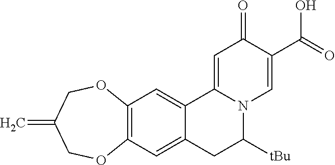 [SMILES: C=C1COc2cc3c(cc2OC1)-c1cc(=O)c(C(=O)O)cn1C(C(C)(C)C)C3] | 6-(tert-butyl)-11-methylene-2-oxo-6,7,11,12- tetrahydro-2H,10H-[1,4]dioxepino[2,3- g]pyrido[2,1-a]isoquinoline-3-carboxylic acid |
| 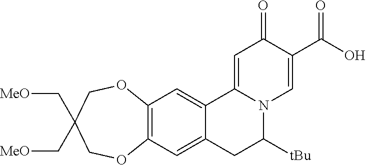 [SMILES: COCC1(COC)COc2cc3c(cc2OC1)-c1cc(=O)c(C(=O)O)cn1C(C(C)(C)C)C3] | 6-(tert-butyl)-11,11-bis(methoxymethyl)-2-oxo- 6,7,11,12-tetrahydro-2H,10H-[1,4]dioxepino[2,3- g]pyrido[2,1-a]isoquinoline-3-carboxylic acid |
| 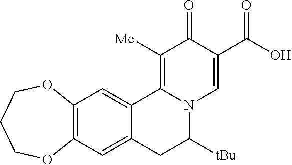 [SMILES: Cc1c2n(cc(C(=O)O)c1=O)C(C(C)(C)C)Cc1cc3c(cc1-2)OCCCO3] | 6-(tert-butyl)-1-methyl-2-oxo-6,7,11,12- tetrahydro-2H,10H-[1,4]dioxepino[2,3- g]pyrido[2,1-a]isoquinoline-3-carboxylic acid |
| 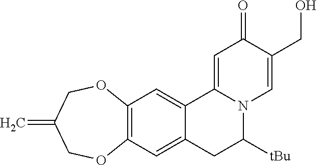 [SMILES: C=C1COc2cc3c(cc2OC1)-c1cc(=O)c(CO)cn1C(C(C)(C)C)C3] | 6-(tert-butyl)-3-(hydroxymethyl)-11-methylene- 6,7,11,12-tetrahydro-2H, 10H-[1,4]dioxepino[2,3- g]pyrido[2,1-a]isoquinolin-2-one |
| 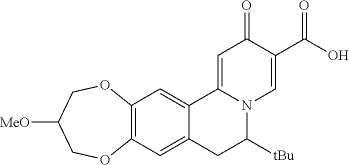 [SMILES: COC1COc2cc3c(cc2OC1)-c1cc(=O)c(C(=O)O)cn1C(C(C)(C)C)C3] | 6-(tert-butyl)-11-methoxy-2-oxo-6,7,11,12- tetrahydro-2H,10H-[1,4]dioxepino[2,3- g]pyrido[2,1-a]isoquinoline-3-carboxylic acid |
| 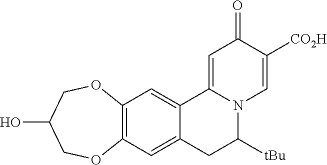 [SMILES: CC(C)(C)C1Cc2cc3c(cc2-c2cc(=O)c(C(=O)O)cn21)OCC(O)CO3] | 6-(tert-butyl)-11-hydroxy-2-oxo-6,7,11,12- tetrahydro-2H,10H-[1,4]dioxepino[2,3- g]pyrido[2,1-a]isoquinoline-3-carboxylic acid |
| 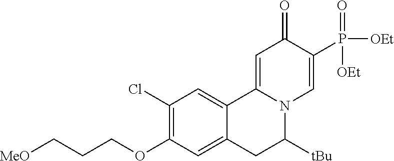 [SMILES: CCOP(=O)(OCC)c1cn2c(cc1=O)-c1cc(Cl)c(OCCCOC)cc1CC2C(C)(C)C] | diethyl (6-(tert-butyl)-10-chloro-9-(3- methoxypropoxy)-2-oxo-6,7-dihydro-2H- pyrido[2,1-a]isoquinolin-3-yl)phosphonate |
| 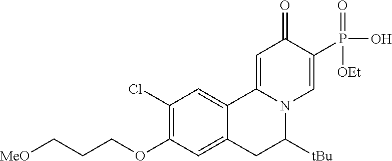 [SMILES: CCOP(=O)(O)c1cn2c(cc1=O)-c1cc(Cl)c(OCCCOC)cc1CC2C(C)(C)C] | ethyl hydrogen (6-(tert-butyl)-10-chloro-9-(3- methoxypropoxy)-2-oxo-6,7-dihydro-2H- pyrido[2,1-a]isoquinolin-3-yl)phosphonate |
| 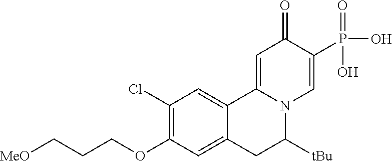 [SMILES: COCCCOc1cc2c(cc1Cl)-c1cc(=O)c(P(=O)(O)O)cn1C(C(C)(C)C)C2] | (6-(tert-butyl)-10-chloro-9-(3-methoxypropoxy)- 2-oxo-6,7-dihydro-2H-pyrido[2,1-a]isoquinolin- 3-yl)phosphonic acid |
| 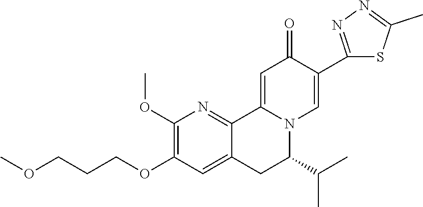 | (S)-6-isopropyl-2-methoxy-3-(3- methoxypropoxy)-9-(5-methyl-1,3,4-thiadiazol-2- yl)-5,6-dihydro-10H-pyrido[1,2- h][1,7]naphthyridin-10-one |
| 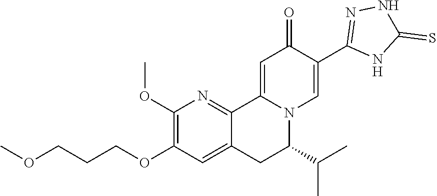 [SMILES: COCCCOc1cc2c(nc1OC)-c1cc(=O)c(-c3n[nH]c(=S)[nH]3)cn1[C@H](C(C)C)C2] | (S)-6-isopropyl-2-methoxy-3-(3- methoxypropoxy)-9-(5-thioxo-4,5-dihydro-1H- 1,2,4-triazol-3-yl)-5,6-dihydro-10H-pyrido[1,2- h][1,7]naphthyridin-10-one |
| 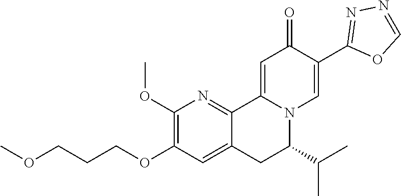 [SMILES: COCCCOc1cc2c(nc1OC)-c1cc(=O)c(-c3nnco3)cn1[C@H](C(C)C)C2] | (S)-6-isopropyl-2-methoxy-3-(3- methoxypropoxy)-9-(1,3,4-oxadiazol-2-yl)-5,6- dihydro-10H-pyrido[1,2-h][1,7]naphthyridin-10- one |
| 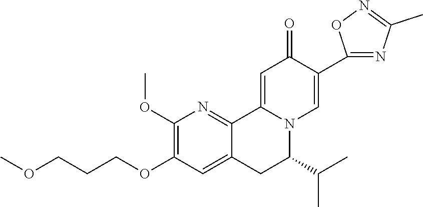 | (S)-6-isopropyl-2-methoxy-3-(3- methoxypropoxy)-9-(3-methyl-1,2,4-oxadiazol-5- yl)-5,6-dihydro-10H-pyrido[1,2- h][1,7]naphthyridin-10-one |
| 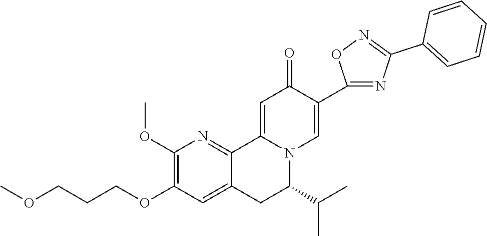 [SMILES: COCCCOc1cc2c(nc1OC)-c1cc(=O)c(-c3nc(-c4ccccc4)no3)cn1[C@H](C(C)C)C2] | (S)-6-isopropyl-2-methoxy-3-(3- methoxypropoxy)-9-(3-phenyl-1,2,4-oxadiazol-5- yl)-5,6-dihydro-10H-pyrido[1,2- h][1,7]naphthyridin-10-one |
| 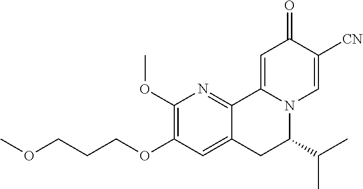 [SMILES: COCCCOc1cc2c(nc1OC)-c1cc(=O)c(C#N)cn1[C@H](C(C)C)C2] | (S)-6-isopropyl-2-methoxy-3-(3- methoxypropoxy)-10-oxo-5,10-dihydro-6H- pyrido[1,2-h][1,7]naphthyridine-9-carbonitrile |
| 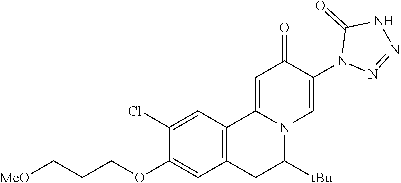 [SMILES: COCCCOc1cc2c(cc1Cl)-c1cc(=O)c(-n3nn[nH]c3=O)cn1C(C(C)(C)C)C2] | 6-(tert-butyl)-10-chloro-9-(3-methoxypropoxy)-3- (5-oxo-4,5-dihydro-1H-tetrazol-1-yl)-6,7- dihydro-2H-pyrido[2,1-a]isoquinolin-2-one |
| 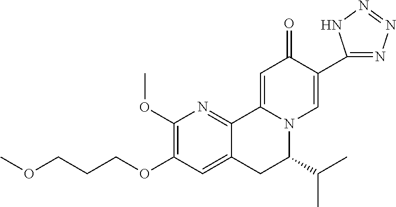 [SMILES: COCCCOc1cc2c(nc1OC)-c1cc(=O)c(-c3nnn[nH]3)cn1[C@H](C(C)C)C2] | (S)-6-isopropyl-2-methoxy-3-(3- methoxypropoxy)-9-(1H-tetrazol-5-yl)-5,6- dihydro-10H-pyrido[1,2-h][1,7]naphthyridin-10- one |
| 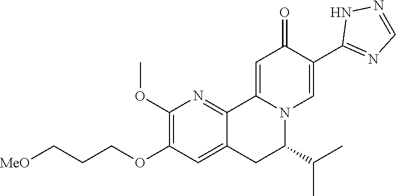 [SMILES: COCCCOc1cc2c(nc1OC)-c1cc(=O)c(-c3ncn[nH]3)cn1[C@H](C(C)C)C2] | (S)-6-isopropyl-2-methoxy-3-(3- methoxypropoxy)-9-(1H-1,2,4-triazol-5-yl)-5,6- dihydro-10H-pyrido[1,2-h][1,7]naphthyridin-10- one |
| 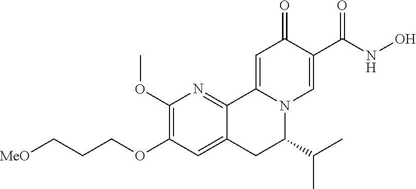 [SMILES: COCCCOc1cc2c(nc1OC)-c1cc(=O)c(C(=O)NO)cn1[C@H](C(C)C)C2] | (S)-N-hydroxy-6-isopropyl-2-methoxy-3-(3- methoxypropoxy)-10-oxo-5,10-dihydro-6H- pyrido[1,2-h][1,7]naphthyridine-9-carboxamide |
| 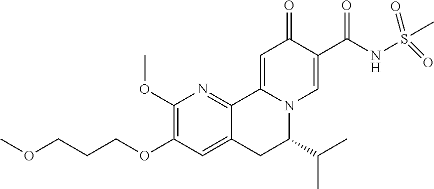 | (S)-6-isopropyl-2-methoxy-3-(3- methoxypropoxy)-N-(methylsulfonyl)-10-oxo- 5,10-dihydro-6H-pyrido[1,2- b][1,7]naphthyridine-9-carboxamide |
| 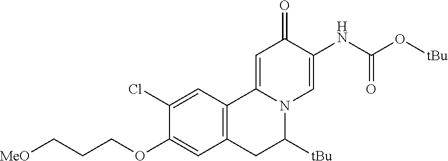 [SMILES: COCCCOc1cc2c(cc1Cl)-c1cc(=O)c(NC(=O)OC(C)(C)C)cn1C(C(C)(C)C)C2] | tert-butyl (6-(tert-butyl)-10-chloro-9-(3- methoxypropoxy)-2-oxo-6,7-dihydro-2H- pyrido[2,1-a]isoquinolin-3-yl)carbamate |
| 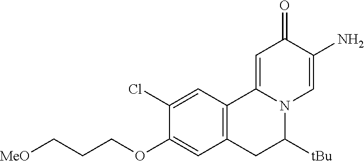 [SMILES: COCCCOc1cc2c(cc1Cl)-c1cc(=O)c(N)cn1C(C(C)(C)C)C2] | 3-amino-6-(tert-butyl)-10-chloro-9-(3- methoxypropoxy)-6,7-dihydro-2H-pyrido[2,1- a]isoquinolin-2-one |
| 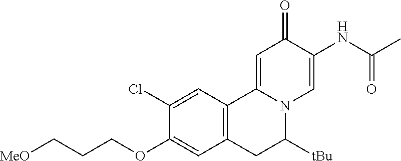 [SMILES: COCCCOc1cc2c(cc1Cl)-c1cc(=O)c(NC(C)=O)cn1C(C(C)(C)C)C2] | N-(6-(tert-butyl)-10-chloro-9-(3- methoxypropoxy)-2-oxo-6,7-dihydro-2H- pyrido[2,1-a]isoquinolin-3-yl)acetamide |
| 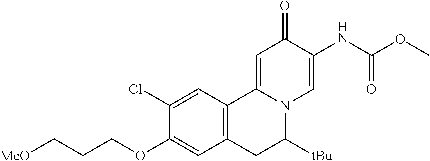 [SMILES: COCCCOc1cc2c(cc1Cl)-c1cc(=O)c(NC(=O)OC)cn1C(C(C)(C)C)C2] | methyl (6-(tert-butyl)-10-chloro-9-(3- methoxypropoxy)-2-oxo-6,7-dihydro-2H- pyrido[2,1-a]isoquinolin-3-yl)carbamate |
| 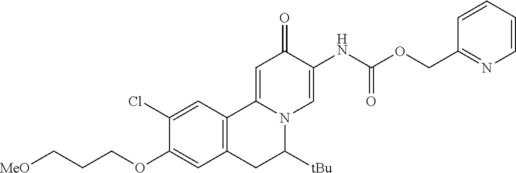 [SMILES: COCCCOc1cc2c(cc1Cl)-c1cc(=O)c(NC(=O)OCc3ccccn3)cn1C(C(C)(C)C)C2] | pyridin-2-ylmethyl (6-(tert-butyl)-10-chloro-9-(3- methoxypropoxy)-2-oxo-6,7-dihydro-2H- pyrido[2,1-a]isoquinolin-3-yl)carbamate |
| 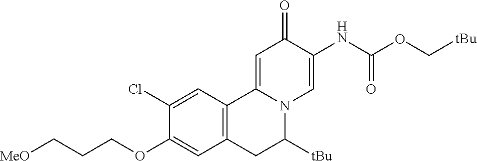 [SMILES: COCCCOc1cc2c(cc1Cl)-c1cc(=O)c(NC(=O)OCC(C)(C)C)cn1C(C(C)(C)C)C2] | neopentyl (6-(tert-butyl)-10-chloro-9-(3- methoxypropoxy)-2-oxo-6,7-dihydro-2H- pyrido[2,1-a]isoquinolin-3-yl)carbamate |
| 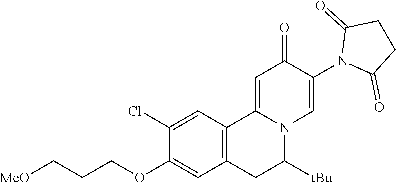 [SMILES: COCCCOc1cc2c(cc1Cl)-c1cc(=O)c(N3C(=O)CCC3=O)cn1C(C(C)(C)C)C2] | 1-(6-(tert-butyl)-10-chloro-9-(3- methoxypropoxy)-2-oxo-6,7-dihydro-2H- pyrido[2,1-a]isoquinolin-3-yl)pyrrolidine-2,5- dione |
| 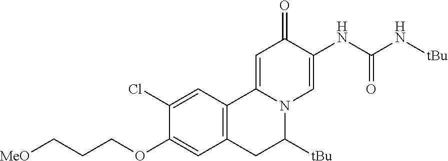 [SMILES: COCCCOc1cc2c(cc1Cl)-c1cc(=O)c(NC(=O)NC(C)(C)C)cn1C(C(C)(C)C)C2] | 1-(tert-butyl)-3-(6-(tert-butyl)-10-chloro-9-(3- methoxypropoxy)-2-oxo-6,7-dihydro-2H- pyrido[2,1-a]isoquinolin-3-yl)urea |
| 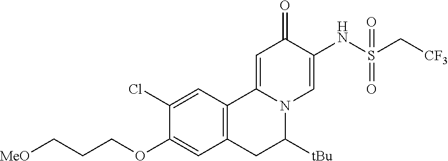 [SMILES: COCCCOc1cc2c(cc1Cl)-c1cc(=O)c(NS(=O)(=O)CC(F)(F)F)cn1C(C(C)(C)C)C2] | N-(6-(tert-butyl)-10-chloro-9-(3- methoxypropoxy)-2-oxo-6,7-dihydro-2H- pyrido[2,1-a]isoquinolin-3-yl)-2,2,2- trifluoroethane-1-sulfonamide |
| 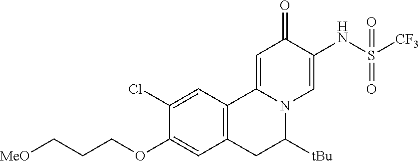 [SMILES: COCCCOc1cc2c(cc1Cl)-c1cc(=O)c(NS(=O)(=O)C(F)(F)F)cn1C(C(C)(C)C)C2] | N-(6-(tert-butyl)-10-chloro-9-(3- methoxypropoxy)-2-oxo-6,7-dihydro-2H- pyrido[2,1-a]isoquinolin-3-yl)-1,1,1- trifluoromethanesulfonamide |
| 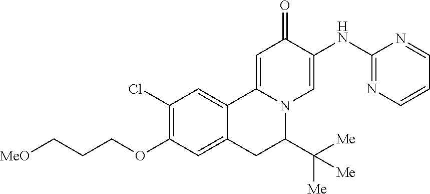 [SMILES: COCCCOc1cc2c(cc1Cl)-c1cc(=O)c(Nc3ncccn3)cn1C(C(C)(C)C)C2] | 6-(tert-butyl)-10-chloro-9-(3-methoxypropoxy)-3- (pyrimidin-2-ylamino)-6,7-dihydro-2H- pyrido[2,1-a]isoquinolin-2-one |
| 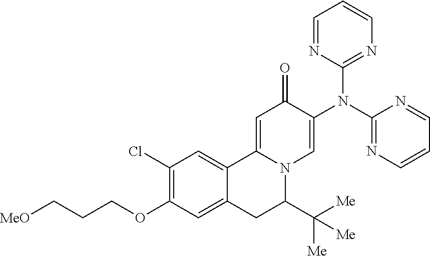 [SMILES: COCCCOc1cc2c(cc1Cl)-c1cc(=O)c(N(c3ncccn3)c3ncccn3)cn1C(C(C)(C)C)C2] | 6-(tert-butyl)-10-chloro-3-(di(pyrimidin-2- yl)amino)-9-(3-methoxypropoxy)-6,7-dihydro- 2H-pyrido[2,1-a]isoquinolin-2-one |
| 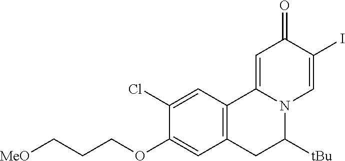 [SMILES: COCCCOc1cc2c(cc1Cl)-c1cc(=O)c(I)cn1C(C(C)(C)C)C2] | 6-(tert-butyl)-10-chloro-3-iodo-9-(3- methoxypropoxy)-6,7-dihydro-2H-pyrido[2,1- a]isoquinolin-2-one |
| 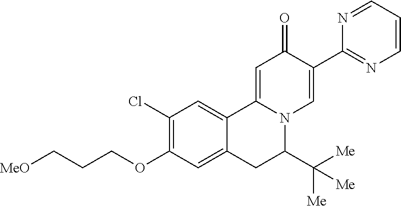 [SMILES: COCCCOc1cc2c(cc1Cl)-c1cc(=O)c(-c3ncccn3)cn1C(C(C)(C)C)C2] | 6-(tert-butyl)-10-chloro-9-(3-methoxypropoxy)-3- (pyrimidin-2-yl)-6,7-dihydro-2H-pyrido[2,1- a]isoquinolin-2-one |
| 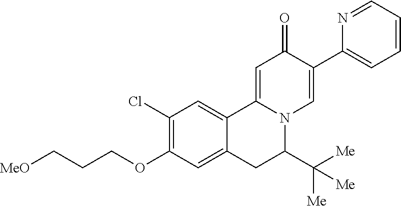 [SMILES: COCCCOc1cc2c(cc1Cl)-c1cc(=O)c(-c3ccccn3)cn1C(C(C)(C)C)C2] | 6-(tert-butyl)-10-chloro-9-(3-methoxypropoxy)-3- (pyridin-2-yl)-6,7-dihydro-2H-pyrido[2,1- a]isoquinolin-2-one |
| 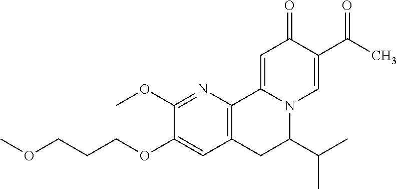 [SMILES: COCCCOc1cc2c(nc1OC)-c1cc(=O)c(C(C)=O)cn1C(C(C)C)C2] | 9-acetyl-6-isopropyl-2-methoxy-3-(3- methoxypropoxy)-5,6-dihydro-10H-pyrido[1,2- h][1,7]naphthyridin-10-one |
| 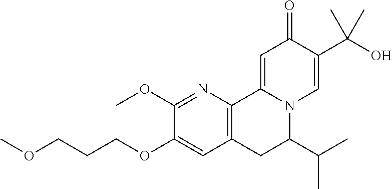 [SMILES: COCCCOc1cc2c(nc1OC)-c1cc(=O)c(C(C)(C)O)cn1C(C(C)C)C2] | 9-(2-hydroxypropan-2-yl)-6-isopropyl-2- methoxy-3-(3-methoxypropoxy)-5,6-dihydro- 10H-pyrido[1,2-h][1,7]naphthyridin-10-one |
| 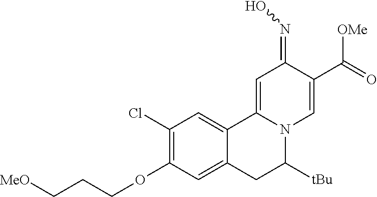 [SMILES: COCCCOc1cc2c(cc1Cl)-c1cc(=NO)c(C(=O)OC)cn1C(C(C)(C)C)C2] | methyl 6-(tert-butyl)-10-chloro-2- (hydroxyimino)-9-(3-methoxypropoxy)-6,7- dihydro-2H-pyrido[2,1-a]isoquinoline-3- carboxylate |
| 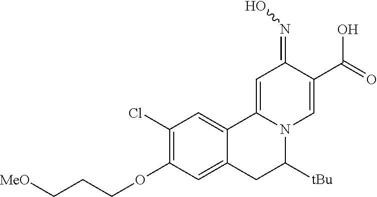 [SMILES: COCCCOc1cc2c(cc1Cl)-c1cc(=NO)c(C(=O)O)cn1C(C(C)(C)C)C2] | 6-(tert-butyl)-10-chloro-2-(hydroxyimino)-9-(3- methoxypropoxy)-6,7-dihydro-2H-pyrido[2,1- a]isoquinoline-3-carboxylic acid |
| 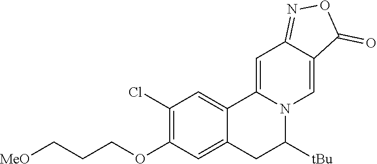 [SMILES: COCCCOc1cc2c(cc1Cl)-c1cc3noc(=O)c-3cn1C(C(C)(C)C)C2] | 6-(tert-butyl)-2-chloro-3-(3-methoxypropoxy)- 5,6-dihydro-9H-isoxazolo[3′,4′:4,5]pyrido[2,1- a]isoquinolin-9-one |
| 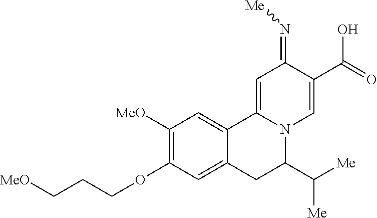 | 6-isopropyl-10-methoxy-9-(3-methoxypropoxy)- 2-(methylimino)-6,7-dihydro-2H-pyrido[2,1- a]isoquinoline-3-carboxylic acid |
| 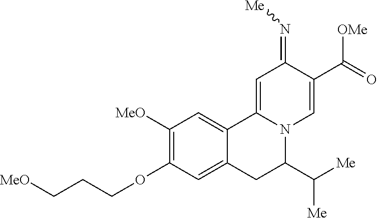 | methyl 6-isopropyl-10-methoxy-2- (methoxyimino)-9-(3-methoxypropoxy)-6,7- dihydro-2H-pyrido[2,1-a]isoquinoline-3- carboxylate |
| 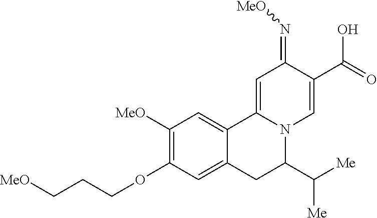 [SMILES: COCCCOc1cc2c(cc1OC)-c1cc(=NOC)c(C(=O)O)cn1C(C(C)C)C2] | 6-isopropyl-10-methoxy-2-(methoxyimino)-9-(3- methoxypropoxy)-6,7-dihydro-2H-pyrido[2,1- a]isoquinoline-3-carboxylic acid |
| 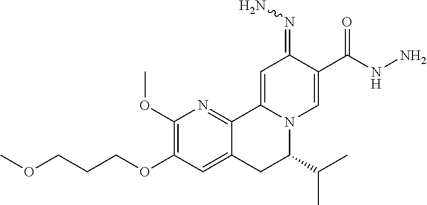 [SMILES: COCCCOc1cc2c(nc1OC)-c1cc(=NN)c(C(=O)NN)cn1[C@H](C(C)C)C2] | (S)-10-hydrazineylidene-6-isopropyl-2-methoxy- 3-(3-methoxypropoxy)-5,10-dihydro-6H- pyrido[1,2-h][1,7]naphthyridine-9-carbohydrazide |
| 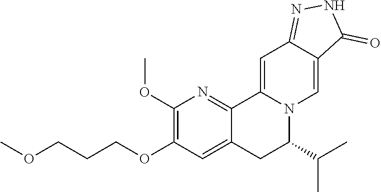 [SMILES: COCCCOc1cc2c(nc1OC)-c1cc3n[nH]c(=O)c-3cn1[C@H](C(C)C)C2] | (S)-6-isopropyl-2-methoxy-3-(3- methoxypropoxy)-5,10- dihydropyrazolo[3′,4′:4,5]pyrido[1,2- h][1,7]naphthyridin-9(6H)-one |
| 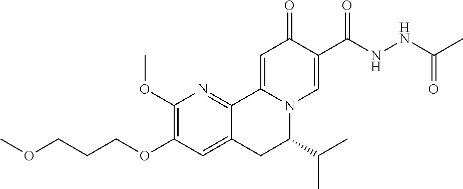 [SMILES: COCCCOc1cc2c(nc1OC)-c1cc(=O)c(C(=O)NNC(C)=O)cn1[C@H](C(C)C)C2] | (S)-N′-acetyl-6-isopropyl-2-methoxy-3-(3- methoxypropoxy)-10-oxo-5,10-dihydro-6H- pyrido[1,2-h][1,7]naphthyridine-9-carbohydrazide |
| 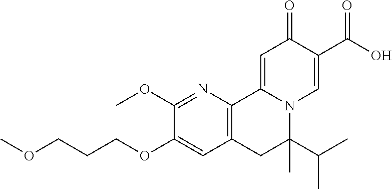 [SMILES: COCCCOc1cc2c(nc1OC)-c1cc(=O)c(C(=O)O)cn1C(C)(C(C)C)C2] | 6-isopropyl-2-methoxy-3-(3-methoxypropoxy)-6- methyl-10-oxo-5,10-dihydro-6H-pyrido[1,2- h][1,7]naphthyridine-9-carboxylic acid |
| 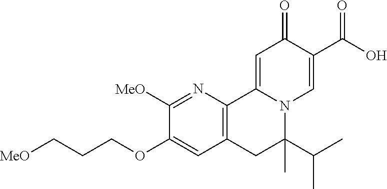 [SMILES: COCCCOc1cc2c(nc1OC)-c1cc(=O)c(C(=O)O)cn1C(C)(C(C)C)C2] | 6-isopropyl-2-methoxy-3-(3-methoxypropoxy)-6- methyl-10-oxo-5,10-dihydro-6H-pyrido[1,2- h][1,7]naphthyridine-9-carboxylic acid (single enantiomer I) |
| 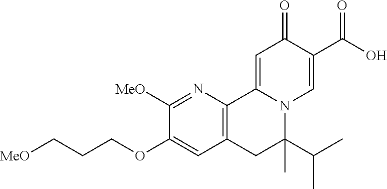 [SMILES: COCCCOc1cc2c(nc1OC)-c1cc(=O)c(C(=O)O)cn1C(C)(C(C)C)C2] | 6-isopropyl-2-methoxy-3-(3-methoxypropoxy)-6- methyl-10-oxo-5,10-dihydro-6H-pyrido[1,2- h][1,7]naphthyridine-9-carboxylic acid (single enantiomer II) |
| 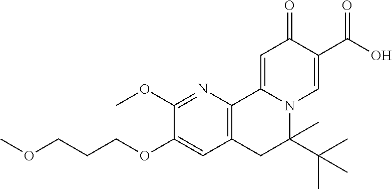 [SMILES: COCCCOc1cc2c(nc1OC)-c1cc(=O)c(C(=O)O)cn1C(C)(C(C)(C)C)C2] | 6-(tert-butyl)-2-methoxy-3-(3-methoxypropoxy)- 6-methyl-10-oxo-5,10-dihydro-6H-pyrido[1,2- h][1,7]naphthyridine-9-carboxylic acid |
| 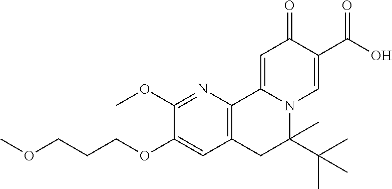 [SMILES: COCCCOc1cc2c(nc1OC)-c1cc(=O)c(C(=O)O)cn1C(C)(C(C)(C)C)C2] | 6-(tert-butyl)-2-methoxy-3-(3-methoxypropoxy)- 6-methyl-10-oxo-5,10-dihydro-6H-pyrido[1,2- h][1,7]naphthyridine-9-carboxylic acid (single enantiomer I) |
| 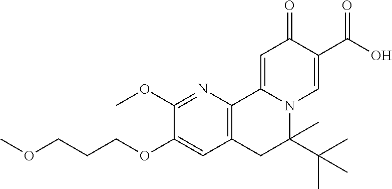 [SMILES: COCCCOc1cc2c(nc1OC)-c1cc(=O)c(C(=O)O)cn1C(C)(C(C)(C)C)C2] | 6-(tert-butyl)-2-methoxy-3-(3-methoxypropoxy)- 6-methyl-10-oxo-5,10-dihydro-6H-pyrido[1,2- h][1,7]naphthyridine-9-carboxylic acid (single enantiomer II) |
| 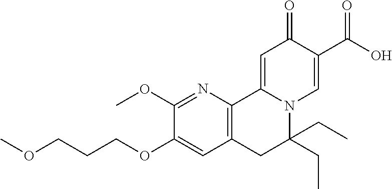 [SMILES: CCC1(CC)Cc2cc(OCCCOC)c(OC)nc2-c2cc(=O)c(C(=O)O)cn21] | 6,6-diethyl-2-methoxy-3-(3-methoxypropoxy)- 10-oxo-5,10-dihydro-6H-pyrido[1,2- h][1,7]naphthyridine-9-carboxylic acid |
| 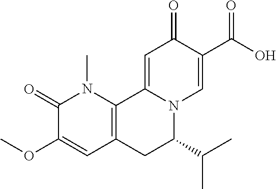 [SMILES: COc1cc2c(n(C)c1=O)-c1cc(=O)c(C(=O)O)cn1[C@H](C(C)C)C2] | (S)-6-isopropyl-3-methoxy-1-methyl-2,10-dioxo- 2,5,6,10-tetrahydro-1H-pyrido[1,2- h][1,7]naphthyridine-9-carboxylic acid |
| 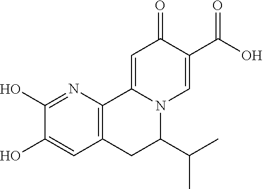 [SMILES: CC(C)C1Cc2cc(O)c(O)nc2-c2cc(=O)c(C(=O)O)cn21] | 2,3-dihydroxy-6-isopropyl-10-oxo-5,10-dihydro- 6H-pyrido[1,2-h][1,7]naphthyridine-9-carboxylic acid |
| 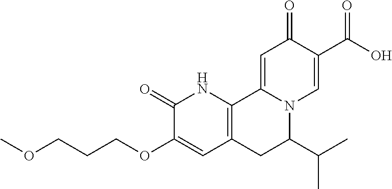 [SMILES: COCCCOc1cc2c([nH]c1=O)-c1cc(=O)c(C(=O)O)cn1C(C(C)C)C2] | 6-isopropyl-3-(3-methoxypropoxy)-2,10-dioxo- 2,5,6,10-tetrahydro-1H-pyrido[1,2- h][1,7]naphthyridine-9-carboxylic acid (single enantiomer I) |
| 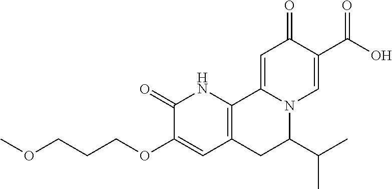 [SMILES: COCCCOc1cc2c([nH]c1=O)-c1cc(=O)c(C(=O)O)cn1C(C(C)C)C2] | 6-isopropyl-3-(3-methoxypropoxy)-2,10-dioxo- 2,5,6,10-tetrahydro-1H-pyrido[1,2- h][1,7]naphthyridine-9-carboxylic acid (single enantiomer II) |
| 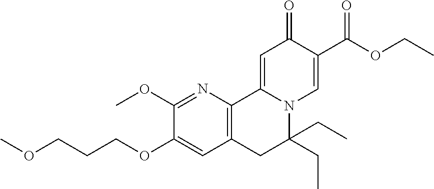 [SMILES: CCOC(=O)c1cn2c(cc1=O)-c1nc(OC)c(OCCCOC)cc1CC2(CC)CC] | ethyl 6,6-diethyl-2-methoxy-3-(3- methoxypropoxy)-10-oxo-5,10-dihydro-6H- pyrido[1,2-h][1,7]naphthyridine-9-carboxylate |
| 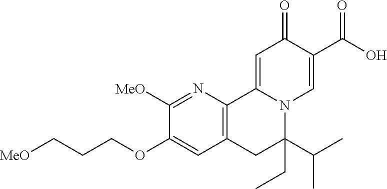 [SMILES: CCC1(C(C)C)Cc2cc(OCCCOC)c(OC)nc2-c2cc(=O)c(C(=O)O)cn21] | 6-ethyl-6-isopropyl-2-methoxy-3-(3- methoxypropoxy)-10-oxo-5,10-dihydro-6H- pyrido[1,2-h][1,7]naphthyridine-9-carboxylic acid |
| 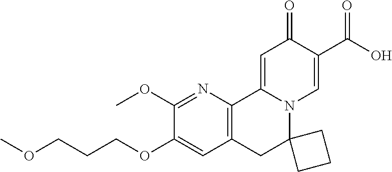 [SMILES: COCCCOc1cc2c(nc1OC)-c1cc(=O)c(C(=O)O)cn1C1(CCC1)C2] | 2′-methoxy-3′-(3-methoxypropoxy)-10′-oxo- 5′,10′-dihydrospiro[cyclobutane-1,6′-pyrido[1,2- h][1,7]naphthyridine]-9′-carboxylic acid |

### Immunostimulators

The term “immunostimulator” includes compounds that are capable of modulating an immune response (e.g., stimulate an immune response (e.g., an adjuvant)). The term immunostimulators includes polyinosinic:polycytidylic acid (poly I:C) and interferons.

The term immunostimulators includes agonists of stimulator of IFN genes (STING) and interleukins. The term also includes HBsAg release inhibitors, TLR-7 agonists (GS-9620, RG-7795), T-cell stimulators (GS-4774), RIG-1 inhibitors (SB-9200), and SMAC-mimetics (Birinapant). The term immunostimulators also includes anti-PD-1 antibodies, and fragments thereof.

### Examples

The present inventions will be described by way of specific examples. The following examples are offered for illustrative purposes and are not intended to limit the inventions in any manner. Those of skill in the art will readily recognize a variety of noncritical parameters which can be changed or modified to yield essentially the same results.

It should be understood that in one embodiment the oligonucleotide is an siRNA molecule that comprises a UNA. e.g., as described herein, e.g., in Table 1. Certain conjugates are depicted herein. Other conjugates and synthetic intermediates thereof, including methods of making, are described in International Publication Numbers WO 2017/177326 and WO 2018/191278, which are specifically incorporated by reference with respect to the conjugates and synthetic intermediates thereof. In certain embodiments herein, the nucleic acid of the conjugates and synthetic intermediates thereof (which may also have been referred to as an oligonucleotide or R²) is a siRNA molecule that comprises a UNA, e.g., as described herein, e.g., in Table 1 or Table A.

Specific siRNA molecules having a UNA used in the Examples herein are depicted in Table 1. Certain chemically modified siRNA sequences are also depected in Table A. Accordingly, certain embodiments of the invention are directed to any one of the siRNA described in Table 1, or to any one of the sense or antisense strands thereof. Certain embodiments of the invention are directed to any one of the siRNA from Table A that comprises a replacement of a nucleotide with a UNA, e.g., in the antisense strand, e.g., at position(s) 5 and or 6 of the antisense strand.

In certain embodiments, the siRNA of the conjugates described herein is selected from any one of the siRNA described in Table 1.

In certain embodiments, the siRNA of the conjugates described herein is selected from any one of the siRNA from Table A that comprises a replacement of a nucleotide with a UNA, e.g., in the antisense strand, e.g., at position(s) 5 and or 6 of the antisense strand.

The conjugate used in the Examples herein is depicted below.

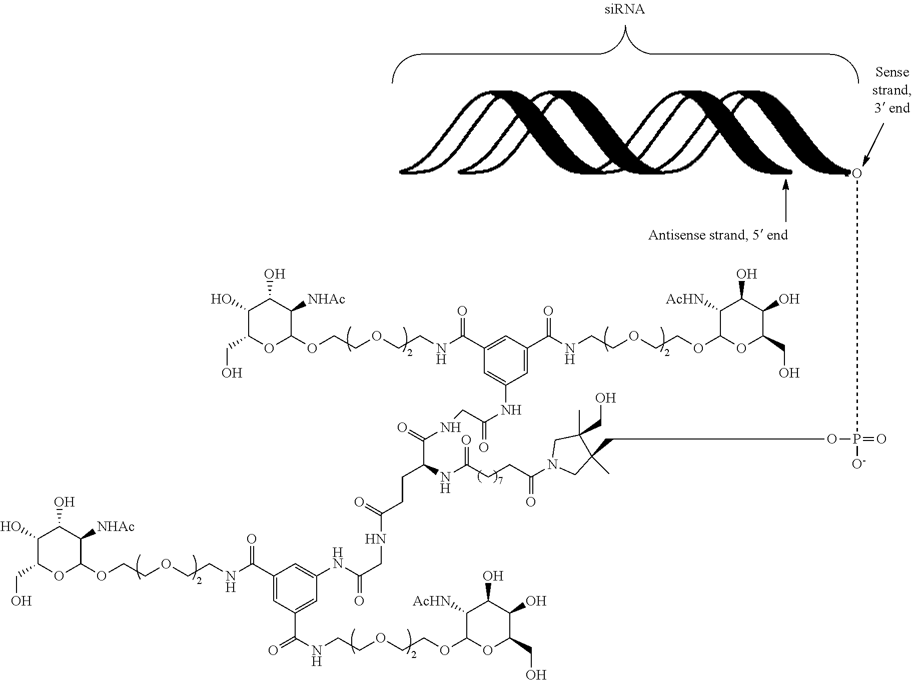

### Example 1. Synthesis of UNA Containing siRNA Conjugates

2′-Bz UNA phosphoramidites were purchased from ThermoFisher Scientific and used for the synthesis of UNA containing siRNAs. The UNA modified siRNA described in Table 1 were prepared. Table A provide siRNA sequences that can be further modified to contain a UNA, e.g., as a replacement for one of the nucleotides depicted.

TABLE 1
Specific Chemically Modified HBV siRNA Duplexes

| | Sense | | Antisense | |
|---|---|---|---|---|
| siRNA | strand SEQ | Sense strand | strand SEQ | Antisense strand |
| Number | ID NO | 5′-3′ | ID NO | 5′-3′ |
| 1 | SEQ ID NO: 1 | gsusgcACUucgcuucaca | SEQ ID NO: 2 | usGsugaagcgaaguGcAcacsgsgr(u) |
| 2 | SEQ ID NO: 1 | gsusgcACUucgcuucaca | SEQ ID NO: 3 | usU(g)sugaagcgaaguGcAcacsgsgr(u) |
| 3 | SEQ ID NO: 1 | gsusgcACUucgcuucaca | SEQ ID NO: 4 | usGsU(u)gaagcgaaguGcAcacsgsgr(u) |
| 4 | SEQ ID NO: 1 | gsusgcACUucgcuucaca | SEQ ID NO: 5 | usGsuU(g)aagcgaaguGcAcacsgsgr(u) |
| 5 | SEQ ID NO: 1 | gsusgcACUucgcuucaca | SEQ ID NO: 6 | usGsugU(a)agcgaaguGcAcacsgsgr(u) |
| 6 | SEQ ID NO: 1 | gsusgcACUucgcuucaca | SEQ ID NO: 7 | usGsugaU(a)gcgaaguGcAcacsgsgr(u) |
| 7 | SEQ ID NO: 1 | gsusgcACUucgcuucaca | SEQ ID NO: 8 | usGsugaaU(g)cgaaguGcAcacsgsgr(u) |
| 8 | SEQ ID NO: 9 | cscsgaUCCauacugcgga | SEQ ID NO: 10 | usCscgcaguauggaUcGgcasgsar(u) |
| 9 | SEQ ID NO: 9 | cscsgaUCCauacugcgga | SEQ ID NO: 11 | usCscgcU(a)guauggaUcGgcasgsar(u) |

2′-O-Methyl nucleotides = lower case; 2′-Fluoro nucleotides = UPPER CASE; Phosphorothioate linker = s; Unmodified = r(nucleotide), UNA = U(nucleotide)

TABLE A
Chemically Modified HBV siRNA Duplexes

| siRNA | Sense strand | Antisense strand |
|---|---|---|
| Number | 5′-3′ | 5′-3′ |
| 10 | csgsugugCaCUUcgcuucaccu | asGsgugAaGCgaagUgCacacgsgsuUU |
| 11 | usgsCaCUUcgcuucaccu | asGsgugAaGCgaagUgCacascsgU |
| 12 | usgscaCUUcgcuucaccu | asGsgugaagcgaagUgCacascsgU |
| 13 | usgscaCUUCgcuucaccu | asGsgugAagcgaagUgCacascsgU |
| 14 | CscsGuGuGcACUucGcuuCacc | gsGsUgAaGcgAaguGcAcAcGgsusc |
| 15 | cscsguguGcACUucgcuucacc | gsGsugaAgCGaaguGcAcacggsusc |
| 16 | cscsguGuGcAcUucgcuucacc | gsGsugaAgCGaaguGcAcacggsusc |
| 17 | cscsguguGcACUucgcuuCacc | gsGsugaAgCgaaguGcAcacGgsusc |
| 18 | cscsgugugcACUucgcuucacc | gsGsugaagcgaaguGcAcacggsusc |
| 19 | cscsguguGcacuucgcuucacc | gsgsugaAgCGaagugcacacggsusc |
| 20 | CscsGuGuGcACUucGcuuCacc | gsGsUgAaGcgAaguGcAcAcGgsuscUU |
| 21 | cscsguguGcACUucgcuucacc | gsGsugaAgCGaaguGcAcacggsuscUU |
| 22 | cscsguGuGcAcUucgcuucacc | gsGsugaAgCGaaguGcAcacggsuscUU |
| 23 | cscsguguGcACUucgcuuCacc | gsGsugaAgCgaaguGcAcacGgsuscUU |
| 24 | GsusGcACUucGcuuCacc | gsGsUgAaGcgAaguGcAcAcsGsgU |
| 25 | GsusGcACUucGcuuCacc | gsGsUgAaGcgAaguGcAcAcsGsg |
| 26 | GsusGcACUucGcuuCacc | gsGsUgAaGcgAaguGcAcsAscsGsg |
| 27 | CscsGuGuGcACUucGcuuCaca | usGsUgAaGcgAaguGcAcAcGgsusc |
| 28 | CscsGuGuGcACUucGcuuCaca | usGsUgAaGcgAaguGcAcAcGgsuscUU |
| 29 | cscsguguGcACUucgcuucaca | usGsugaAgCGaaguGcAcacggsuscUU |
| 30 | cscsguGuGcAcUucgcuucaca | usGsugaAgCGaaguGcAcacggsuscUU |
| 31 | cscsguguGcACUucgcuuCaca | usGsugaAgCgaaguGcAcacGgsuscUU |
| 32 | cscsgugugcACUucgcuucaca | usGsugaagcgaaguGcAcacggsuscUU |
| 33 | gsusGcACUucgcuucaca | usGsugaAgCGaaguGcAcacsgsgU |
| 34 | gsusgcACUucgcuucaca | usGsugaagcgaaguGcAcacsgsgU |
| 35 | gsusGcaCUucgcuucaca | usGsugaagcgaaguGcAcacsgsgU |
| 36 | GsusGcACUucGcuuCaca | usGsUgAaGcgAaguGcAcAcsGsg |
| 37 | uscsgcuuCaCCUcugcacgucg | csGsacgUgCAgaggUgAagcgasasgUU |
| 38 | uscsgcuuCaCCUcugcacguca | usGsacgUgCAgaggUgAagcgasasgUU |
| 39 | uscsgcUuCaCcUcugcacguca | usGsacgUgCAgaggUgAagcgasasgUU |
| 40 | ususCaCCUcugcacguca | usGsacgUgCAgaggUgAagcsgsaU |
| 41 | ususcaCCUcugcacguca | usGsacgugcagaggUgAagcsgsaU |
| 42 | ususCaCCUcugcacguca | usGsacgUgcagaggUgAagcsgsaU |
| 343 | ususuaCuAgUGCcaUuuguuca | usGsAaCaAauGgcaCuAgUaAascsu |
| 44 | ususuaCuAgUGCcaUuuguuca | usGsAaCaAauGgcaCuAgUaAascsuUU |
| 45 | ususuacuAgUGCcauuuguuca | usGsaacAaAUggcaCuAguaaascsuUU |
| 46 | ususuaCuAgUgCcauuuguuca | usGsaacAaAUggcaCuAguaaascsuUU |

2′-O-Methyl nucleotides = lower case; 2′-Fluoro nucleotides = UPPER CASE; Phosphorothioate linker = s; Unmodified = UPPER CASE

### Example 2. In Vitro Testing of HBV siRNA Modified with UNA at Varying Positions in a Dual Luciferase Reporter Cell Culture System

UNA modified HBV siRNA described in Table 1, siRNAs 1 to 7, were tested for in vitro activity in a dual luciferase reporter cell culture system. An HBV genomic sequence was edited to contain four sequence regions, one of which covered the target site for the non-UNA modified siRNA sequence. These HBV sequence regions were joined, in silico, including flanking regions and this synthetic consensus HBV target fragment was cloned between the stop codon and polyadenylation signal of Renilla luciferase on a reporter plasmid. The gene silencing activity of the non-UNA and UNA-containing siRNAs was tested by measuring reduction of Renilla luciferase (R-Luc) activity in relation to firefly luciferase (F-Luc) activity in the Dual-Glop Luciferase Assay System (Promega, Madison, WI, USA). Briefly, HepG2 cells were seeded at a density of 60.000 cells per well in 96-well plates and transfected with 80 ng reporter plasmid per well and HBV siRNAs at varying concentrations in duplicate using Lipofectamine 3000. After incubation for 24 hours at 37° C./5% CO2, media was replaced, and cells were incubated for another 72 hours at the conditions described above. Following the 72 hour the incubation, the cells were processed using the Dual-Glo® Luciferase kit. Expression of both luciferases was determined by luminescence detection. R-Luc/F-Luc expression of HBV-siRNA treated samples was normalized to the mean of R-Luc/F-Luc expression in non-siRNA treated cells. As a positive control, an siRNA against R-Luc was included. A non-HBV-targeting siRNA was included as a negative control.

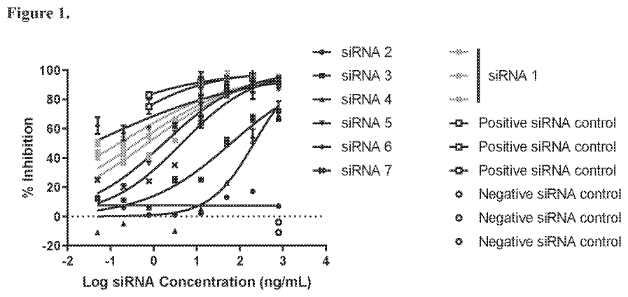

FIG. 1. depicts the activity data from the dual luciferase reporter cell culture experiment. A single UNA modification at antisense strand positions 5 and 6 retained similar activity as the non-UNA modified siRNA reference, confirming that UNA modifications at these positions on the antisense strand do not significantly impact siRNA activity.

### Example 3. In Vitro Testing of UNA Modified HBV siRNA in AAV-HBV Primary Mouse Hepatocytes

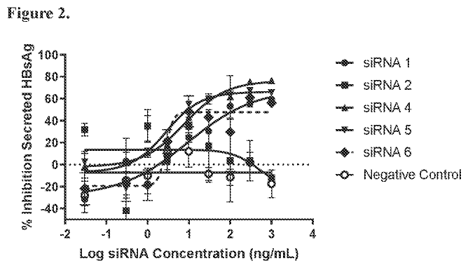

HBV siRNA modified with UNA at various positions within the antisense strand were tested for anti-HBV activity in primary mouse hepatocytes (PMHs) isolated from an adeno-associated virus (AAV) mouse model of HBV infection. PMHs were isolated from AAV-HBV mice, a well-established in vivo tool for assessing anti-HBV drug activity which involves intravenous delivery of recombinant AAV containing a transgene encompassing a 1.2× overlength sequence of the HBV genome to the mouse liver, resulting in the transduction of mouse hepatocytes and consequent expression of HBV RNA, protein, DNA, and viral particles (Dion, S., et al., Journal of Virology, 2013, 87(10): 5554-5563). Briefly, mouse hepatocytes were isolated from AAV-HBV mice in a similar manner as described in Severgnini, M., et al. (Cytotechnology, 2012, 64(2): 187-195) and were seeded at a density of 27,500 cells/well in collagen-coated 96-well plates. Cells were transfected with HBV siRNAs (siRNA Number 1, 2, 4, 5 and 6 in Table 1) or a non-HBV-targeting siRNA as a negative control at varying concentrations in triplicate using a lipid nanoparticle delivery process and incubated for 24 hours at 37° C./5% CO2, after which media was replaced and cells were incubated for another 24 hours at the conditions described above. HBsAg levels in cell supernatants were determined using the Bio-Rad EIA GS HBsAg 3.0 kit (Bio-Rad, catalog no. 32591) as per manufacturer's instructions. Data was analyzed and expressed as HBsAg levels relative to untreated cells. FIG. 2. depicts the anti-HBV activity of HBV siRNA modified with UNA in PMH from AAV-HBV mice. The half-maximal effective concentration (EC₅₀) value for each of the siRNAs tested are presented in the following Table 2.

TABLE 2
Anti-HBV activity EC₅₀ values in AAV-HBV PMHs treated
with UNA Chemically Modified HBV siRNA Duplexes

| siRNA Number | EC₅₀ (ng/mL) |
|---|---|
| 1 | 9.4 |
| 2 | Not assigned |
| 4 | 7.8 |
| 5 | 2.8 |
| 6 | 2.8 |

A single UNA modification at either antisense strand position 4, 5 or 6 retains anti-HBV activity as compared to the non-UNA modified siRNA.

### Example 4. In Vitro Testing of UNA Modified HBV siRNA Targeting Distinct Target Sites

UNA modified HBV siRNA described in Table 1, siRNAs 1 and 6, 8 and 9, were tested for in vitro activity in the dual luciferase reporter cell culture system described in Example 1. HepG2 cells were seeded at a density of 60,000 cells per well in 96-well plates and rested for 24 hours at 37° C./5% CO2. The cells were then transfected with 80 ng reporter plasmid per well and HBV siRNAs at varying concentrations in triplicate using Lipofectamine 3000. After incubation for 24 hours at 37° C./5% CO2, media was replaced, and cells were incubated for another 24 hours at the conditions described above. Following the second incubation, the cells were processed using the Dual-Glo® Luciferase kit. Expression of both luciferases was determined by luminescence detection. R-Luc/F-Luc expression of HBV-siRNA treated samples was normalized to the mean of R-Luc/F-Luc expression in non-siRNA treated cells. As a positive control, an siRNA against R-Luc was included. A non-HBV-targeting siRNA was included as a negative control.

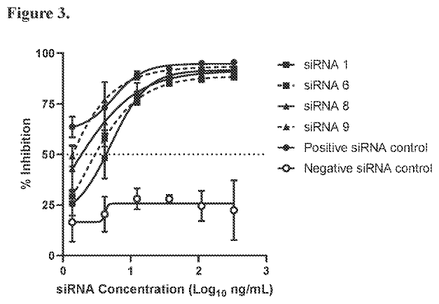

FIG. 3. depicts the activity data from the dual luciferase reporter cell culture experiment. A single UNA modification at antisense strand position 6 in two distinct siRNA sequences retained a similar degree of activity as the respective non-UNA modified siRNA reference, confirming that a UNA modification at this position on the antisense strand does not generally impact siRNA activity.

### Example 5. In Vivo Activity Testing of UNA HBV siRNA Conjugates

Compounds having siRNA described in Table 1 conjugated to GalNAc ligands were prepared as described in International Publication Number WO 2018/191278.

Chemically modified HBV siRNA described in Table 1 conjugated to GalNAc ligands were tested for in vivo activity in an established mouse model of HBV infection. In the AAV-HBV 1.2 C57BL/6 mouse model, stable and persistent HBV expression is achieved after injection of an adeno-associated virus (AAV) vector encoding an over-genomic length sequence of HBV, leading to hepatic expression of HBV RNA and proteins and the secretion of viral and sub-viral particles into the blood.

The AAV-HBV construct used in these studies was based on details provided in Dion, S., et al., Journal of Virology, 2013, 87(10): 5554-5563. All animal-related procedures were conducted according to written operating procedures, in accordance with Canadian Council on Animal Care (CCAC) Guidelines on Good Animal Practices and approved by the local Institutional Animal Care and Use Committee (IACUC). Each animal was inoculated with 1E11 vector genomes (VG) of AAV-HBV vector. Prior to treatment, all animals were test bled and serum HBsAg levels determined for individual animals to confirm established HBV expression.

siRNA treatment: Groups of mice (n=5) were administered a single 3 mg/kg dose of HBV siRNA conjugate once on Day 0 (1 dose per animal) via subcutaneous injection in the scapular region. One group of animals administered vehicle only (saline) served as controls.

Collections: All mice were test bled on Day 0, prior to treatment, and at defined time points after test article administration (on study days 0, 7, 14, 21 and 28) to determine maximum reductions in serum HBsAg levels and the duration of pharmacologic activity.

Analysis: HBsAg levels in serum samples were determined using the Bio-Rad EIA GS HBsAg 3.0 kit (Bio-Rad, catalog no. 32591) as per the manufacturer's instructions. Individual animal serum from each treatment group was used to determine the group mean HBsAg levels at individual time points. Data was analyzed and expressed as HBsAg levels relative to pre treatment baseline (% relative to Day 0).

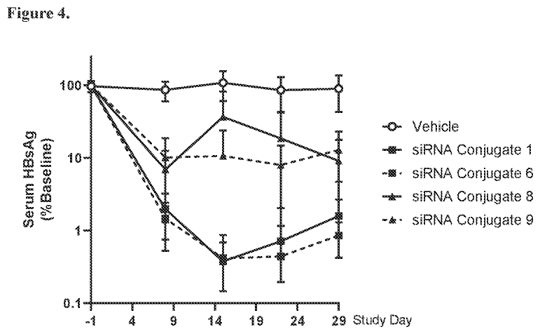

Results from testing siRNAs 1, 2, 8 and 9 described in Table 1 are presented in FIG. 4. Similar in vivo anti-HBV activity profiles were observed in animals treated with HBV siRNA conjugates containing a single UNA modification at antisense strand position 6 when compared to animals administered the respective siRNA conjugates lacking UNA modification, demonstrating that UNA modified siRNA conjugates retain an equivalent degree of activity as non-UNA modified siRNAs in a whole-body system.

### Example 6. Off-Target Effect of UNA HBV siRNA Conjugate

Incorporation of a thermally destabilizing chemical modification within siRNA antisense strand positions 2-7 (the “seed region”) may decrease the likelihood of siRNA seed-region-based pairing and silencing of unintended transcripts, which would otherwise result in so called “off target effects”. To assess whether UNA modification of siRNA conjugates is able to reduce the degree of siRNA-mediated off-target effects, an RNA sequencing analysis of global transcriptome changes present in the livers of AAV-HBV mice treated with HBV siRNA conjugates of siRNA Nos. 1 (non-UNA modified) and 6 (UNA modified) was undertaken.

Groups of AAV-HBV mice (n=5) as described in Example 4 were administered a single 3 mg/kg dose of HBV siRNA conjugate once on Day 0 (1 dose per animal) via subcutaneous injection in the scapular region. One group of animals administered vehicle only (saline) served as controls.

Collections and RNA sequencing: All mice were sacrificed at 14 days post-siRNA conjugate administration, and total RNA extracted from livers using the Qiagen RNeasy kit as per manufacturer's instructions (Qiagen, catalog no. 74136). Extracted total RNA was eluted in a total of 120 μL RNase-free water. Concentrations were assigned using Nanodrop spectrophotometric analysis. Ribosomal RNA depletion and library preparation was conducted as per manufacturer's instructions using the Illumina Ribo-Zero rRNA Removal kit (Illumina, catalog no. RZH1046) and the NEBNext Ultra 11 RNA Library Prep Kit (NEB, catalog no. E7770S). Samples were run on the Illumina HiSeq platform and differentially expressed genes were identified through comparisons with saline control.

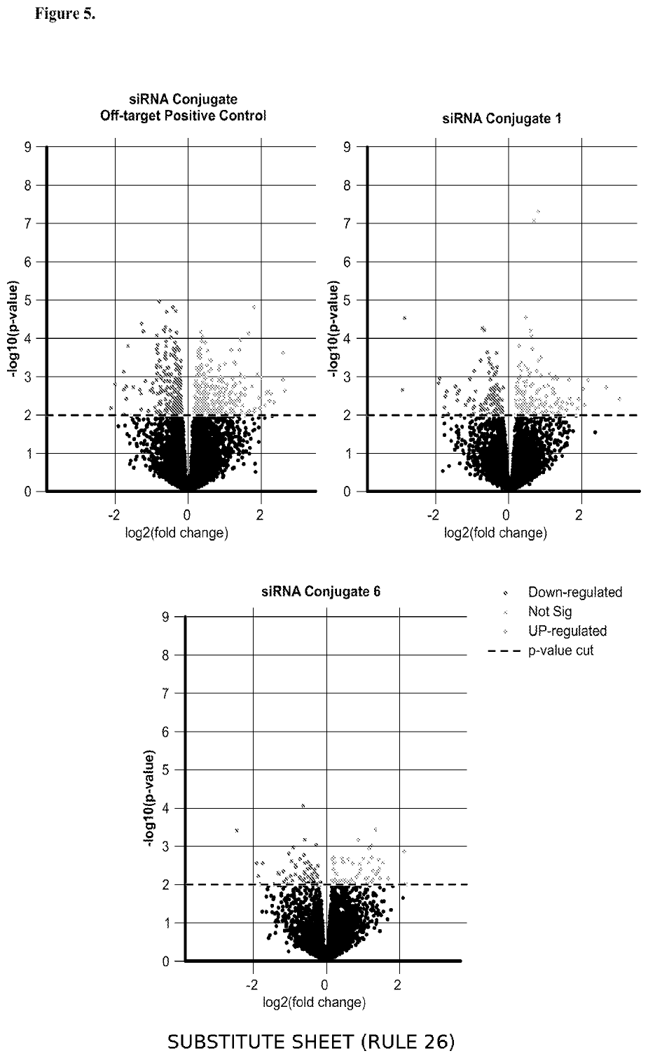

Volcano plots (FIG. 5) were prepared to compare the number of differentially expressed genes falling above the applied adjusted p-value threshold. In livers of mice treated with UNA-containing siRNA conjugate, fewer differentially expressed genes were observed when compared to the non-UNA modified siRNA parental sequence. Animals administered a non-UNA modified siRNA previously identified as eliciting off-target effects (positive control) displayed a larger degree of unintended transcriptional gene changes, as expected. These results demonstrate that a single UNA modification located at antisense strand position 6 is able to reduce the degree of siRNA off-target activity.

### Example 7. In Vivo Evaluation of Liver Toxicity of HBV siRNA Modified with UNA in a Humanized Liver Chimeric Mouse Model

UNA modified HBV siRNA described in Table 1 conjugated to GalNAc ligands, siRNA 1 and 6, were tested for the ability to induce liver toxicity in a humanized liver chimeric mouse model. All animal-related procedures were performed in accordance with the animal welfare bylaws of Shin Nippon Biomedical Laboratories, Ltd., which is accredited by AAALAC International. cDNA-uPA^{wild/+}/SCID mice were transplanted with human hepatocytes as described (Tateno, C., and Kojima, Y., Laboratory Animal Research, 2020; 36:2). 18 week-old animals with an estimated >70% human hepatocyte engraftment as determined by serum human albumin levels were randomized into siRNA treatment groups.

siRNA treatment: Groups of mice (n=5-6) were administered 5 total doses of either 36 or 100 mg/kg of a positive control siRNA conjugate previously reported to induce ALT elevations in this model (Gane, E. et al., SAT-424, International Liver Congress, 2020), or 5 total doses of 12, 36 or 100 mg/kg of siRNA conjugates 1 or 6. siRNA doses were administered on study Day 0, 21, 28, 35 and 42 via subcutaneous injection in the dorsal region. One group of animals administered vehicle only (saline) served as controls.

Collections: All mice were test bled on Day 0, prior to treatment, and at defined time points after test article administration (on study days −4, 6, 13, 20, 27, 34, 41 and 49) to determine levels of total alanine transaminase (ALT) and human alanine transaminase (hALT1). Livers of animals were collected on Day 49 to confirm levels of siRNA conjugates present.

Analysis: hALT1 levels in serum samples were determined using an enzyme immunoassay. Total ALT levels in serum samples were determined using a JCA-BM6070 automatic analyzer (JEOL Ltd.). Individual animal serum from each treatment group expressed as fold change over predose levels for that individual animal was used to determine the group mean hALT or total ALT levels at individual time points. siRNA conjugate levels present in liver was quantitated using LC-MS/MS.

TABLE 3
Total ALT levels in humanized liver chimeric mice administered siRNA
conjugates
Total ALT group mean data expressed as fold relative to Day −4 levels

| | | Day | Day | Day | Day | Day | Day | Day | Day |
|---|---|---|---|---|---|---|---|---|---|
| SiRNA Conjugate | Dose | −4 | 6 | 13 | 20 | 27 | 34 | 41 | 49 |
| Positive control | 36 mg/kg | 1.0 | 1.2 | 1.9 | 1.9 | 2.1 | 2.1 | 2.7 | 2.5 |
| siRNA conjugate | 100 mg/kg | 1.0 | 1.7 | 2.5 | 2.5 | 2.6 | 2.8 | 2.9 | 2.8 |
| SIRNA Conjugate 1 | 12 mg/kg | 1.0 | 1.0 | 1.5 | 1.7 | 2.0 | 2.1 | 2.1 | 2.2 |
| | 36 mg/kg | 1.0 | 1.5 | 1.8 | 2.0 | 2.5 | 2.6 | 3.0 | 3.1 |
| | 100 mg/kg | 1.0 | 1.5 | 2.3 | 2.5 | 2.6 | 2.5 | 2.6 | 2.9 |
| siRNA Conjugate 6 | 12 mg/kg | 1.0 | 1.2 | 1.2 | 1.5 | 1.6 | 1.8 | 1.8 | 2.2 |
| | 36 mg/kg | 1.0 | 1.2 | 1.6 | 1.6 | 1.9 | 1.9 | 2.5 | 2.1 |
| | 100 mg/kg | 1.0 | 1.3 | 1.7 | 1.4 | 1.8 | 2.0 | 2.4 | 2.2 |

TABLE 4
hALT levels in humanized liver chimeric mice administered siRNA conjugates
hALT group mean data expressed as fold relative to Day −4 levels

| | | Day | Day | Day | Day | Day | Day | Day | Day |
|---|---|---|---|---|---|---|---|---|---|
| siRNA Conjugate | Dose | −4 | 6 | 13 | 20 | 27 | 34 | 41 | 49 |
| Positive control | 36 mg/kg | 1.0 | 1.3 | 1.9 | 2.1 | 2.2 | 2.4 | 2.7 | 3.4 |
| siRNA conjugate | 100 mg/kg | 1.0 | 1.7 | 2.7 | 3.3 | 3.6 | 3.7 | 3.2 | 4.5 |
| siRNA Conjugate 1 | 12 mg/kg | 1.0 | 0.9 | 1.6 | 2.1 | 2.0 | 2.6 | 2.3 | 3.2 |
| | 36 mg/kg | 1.0 | 1.2 | 1.7 | 2.3 | 2.7 | 2.8 | 2.9 | 4.0 |
| | 100 mg/kg | 1.0 | 1.2 | 2.2 | 2.9 | 3.2 | 3.2 | 3.1 | 4.6 |
| SIRNA Conjugate 6 | 12 mg/kg | 1.0 | 1.0 | 1.3 | 1.7 | 2.3 | 2.1 | 2.1 | 2.7 |
| | 36 mg/kg | 1.0 | 1.0 | 1.8 | 2.0 | 2.0 | 2.4 | 2.3 | 2.6 |
| | 100 mg/kg | 1.0 | 0.9 | 1.5 | 1.9 | 2.4 | 2.2 | 2.3 | 2.4 |

TABLE 5
Liver levels of siRNA conjugates
Total antisense strand levels group mean data

| | | |
|---|---|---|
| siRNA Conjugate | Dose | Day 49 |

| | | | |
|---|---|---|---|
| siRNA Conjugate 1 | 12 | mg/kg | 688 |
| | 36 | mg/kg | 1140 |
| | 100 | mg/kg | 1574 |
| siRNA Conjugate 6 | 12 | mg/kg | 664 |
| | 36 | mg/kg | 1312 |
| | 100 | mg/kg | 2162 |

Results from testing siRNAs 1 and 6 conjugated to GalNAc ligands (siRNA Conjugate 1 and siRNA Conjugate 6, respectively) are presented in Tables 3, 4 and 5. siRNA conjugate 6 containing a single UNA modification at antisense strand position 6 induced lower levels of hALT or total ALT when compared to animals administered siRNA conjugate lacking UNA modification (siRNA 1 and positive control siRNA). Similar levels of siRNA conjugates 1 and 6 were measured in the livers of treated animals, suggesting that the observed differences in the levels of hALT or total ALT were not attributed to differences in siRNA amounts present in the liver. These results demonstrate that UNA-modified siRNA conjugates (e.g., siRNA conjugate 6) are able to mitigate siRNA-associated liver toxicity in a whole-body system.

What is claimed is:

1. A conjugate of Formula (I):
R¹-L-R² (I)
or a salt thereof, wherein:
R¹ is a targeting ligand that comprises one or more saccharide groups;
L is an optional linker; and
R² is an siRNA molecule that comprises at least one unlocked nucleic acid (UNA) of the following formula: 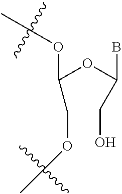
wherein B is a nucleobase.

2. The conjugate or salt of claim 1, wherein R² is an siRNA molecule that comprises at least two UNAs.

3. The conjugate or salt of claim 1, wherein R² is an siRNA molecule that comprises one UNA.

4. The conjugate or salt of any one of claims 1-3, wherein R² is an siRNA molecule that comprises a UNA at position 1, 2, 3, 4, 5, 6, 7, 8, or 9 of the antisense strand.

5. The conjugate or salt of any one of claims 1-3, wherein R² is an siRNA molecule that comprises a UNA at position 6 of the antisense strand.

6. The conjugate or salt of any one of claims 1-5, wherein B is an unnatural nucleobase.

7. The conjugate or salt of any one of claims 1-5, wherein B is a natural nucleobase.

8. The conjugate or salt of any one of claims 1-5, wherein B is a nucleobase that comprises a purine or a pyrimidine.

9. The conjugate or salt of any one of claims 1-5, wherein B is a nucleobase selected from: 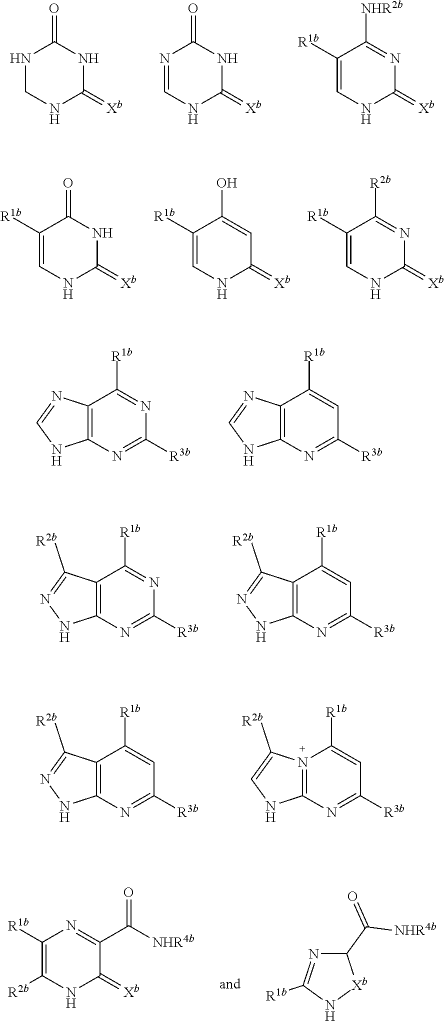
wherein:
R^{1b} is selected from the group consisting of H, Me, F, Cl, Br, I, OH, NH₂, SH, OMe, NO₂, NHOH, NHOMe, NHNH₂, C═ONH₂, C₁-C₈ alkyl, and 5- or 6-membered heteroaryl;
R^{1b} is selected from the group consisting of H, OH, OMe, NH₂, NHMe, C═ONH₂, C₁-C₈ alkyl, and 5- or 6-membered heteroaryl;
R^{3b} is selected from the group consisting of H, F, Cl, Br, I, OH, S, NH₂, SH, OMe, NO₂, NHOH, NHOMe, NHNH₂, C═ONH₂, C₁-C₈ alkyl, and 5- or 6-membered heteroaryl;
R^{4b} is selected from the group consisting of H, NH₂ and C₁-C₈ alkyl; and
Xb is NR^{2b}, O or S.

10. The conjugate or salt of any one of claims 1-5, wherein B is selected from adenine (A), cytosine (C), guanine (G) and uracil (U).

11. The conjugate or salt of any one of claims 1-10, which is a compound of formula (II): 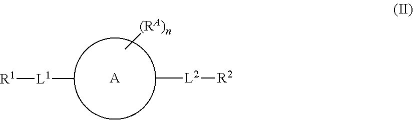
wherein:
R¹ is a targeting ligand that comprises one or more saccharide groups;
L¹ is absent or a linking group;
L² is absent or a linking group;
R² is an siRNA molecule that comprises at least one UNA of the following formula: 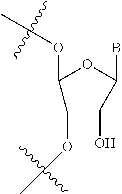
B is a nucleobase the ring A is absent, a 3-20 membered cycloalkyl, a 5-20 membered aryl, a 5-20 membered heteroaryl, or a 3-20 membered heterocycloalkyl;
ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
R^{B} is hydrogen, a protecting group, a covalent bond to a solid support, or a bond to a linking group that is bound to a solid support; and
n is 0, 1, 2, 3, 4, 5, 6, 7, 8, 9, or 10;
or a salt thereof.

12. The conjugate or salt of claim 11, wherein R^{B} is hydrogen.

13. The conjugate or salt of claim 11, wherein R¹ is —C(H)₍₃₋ₚ₎(L³-saccharide)ₚ,
wherein each L³ is independently a linking group;
p is 1, 2, or 3; and
saccharide is a monosaccharide or disaccharide.

14. The conjugate or salt of claim 13, wherein each saccharide is: 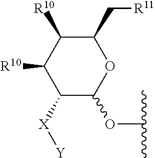
wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁹R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;
or a salt thereof.

15. The conjugate or salt of claim 13 or 14, wherein each saccharide is selected from the group consisting of:

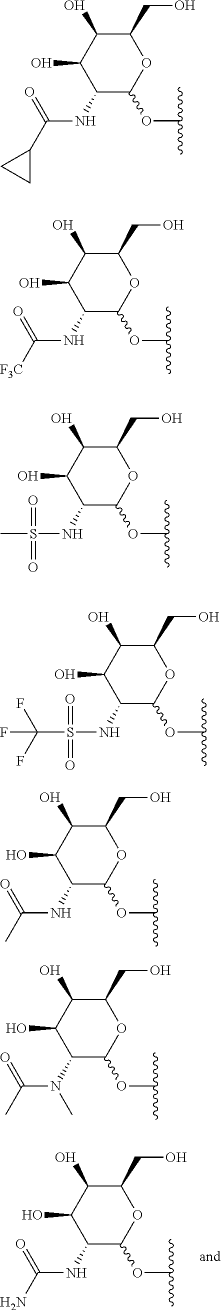

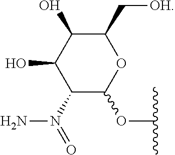

16. The conjugate or salt of any one of claims 13-15, wherein each saccharide is:

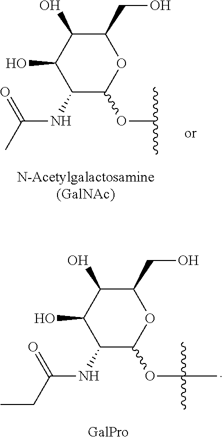 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

17. The conjugate or salt of any one of claims 13-16, wherein each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

18. The conjugate or salt of any one of claims 13-16, wherein each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxy carbonyl, (C₁-C₆)alkylthio, azido, cyano, nitro, halo, hydroxy, oxo (═O), carboxy, aryl, aryloxy, heteroaryl, and heteroaryloxy.

19. The conjugate or salt of any one of claims 13-18, wherein L³ is: 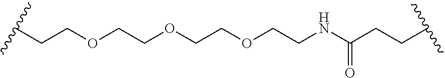 [SMILES: *CCOCCOCCOCCNC(=O)CC*]
or a salt thereof.

20. The conjugate or salt of any one of claims 1-19, wherein R¹ is: 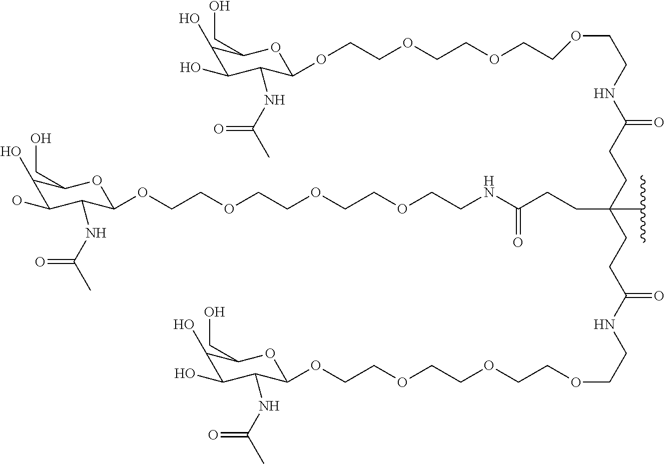 [SMILES: *C(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]
or a salt thereof.

21. The conjugate or salt of any one of claims 11-19, wherein L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

22. The conjugate or salt of any one of claims 11-19, wherein L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

23. The conjugate or salt of any one of claims 11-19, wherein L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

24. The conjugate or salt of any one of claims 11-23, wherein L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

25. The conjugate or salt of any one of claims 11-24, wherein L² is connected to R² through —O—.

26. The conjugate or salt of any one of claims 11-23, wherein L¹ is selected from the group consisting of:

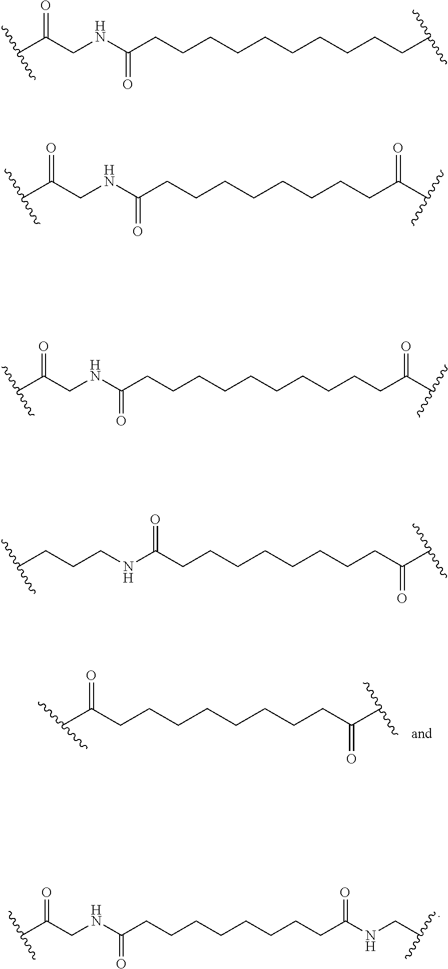 [SMILES: *CCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(*)=O ; *C(=O)CCCCCCCCC(*)=O ; *CNC(=O)CCCCCCCCC(=O)NCC(*)=O]

27. The conjugate or salt of any one of claims 11-24, wherein L² is —CH₂—O— or —CH₂—CH₂—O—.

28. The conjugate or salt of any one of claims 11-27, which is a compound formula (IIa): 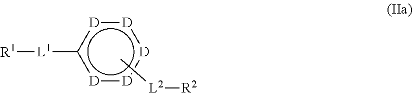
wherein:
each D is independently selected from the group consisting of 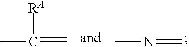
or a salt thereof.

29. The conjugate or salt of claim 28 that is selected from the group consisting of: 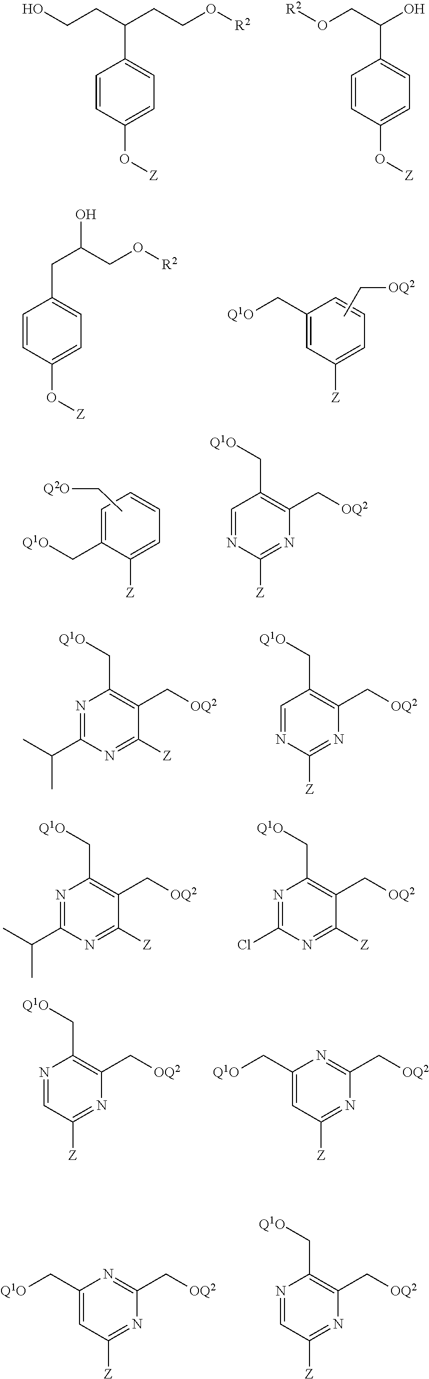 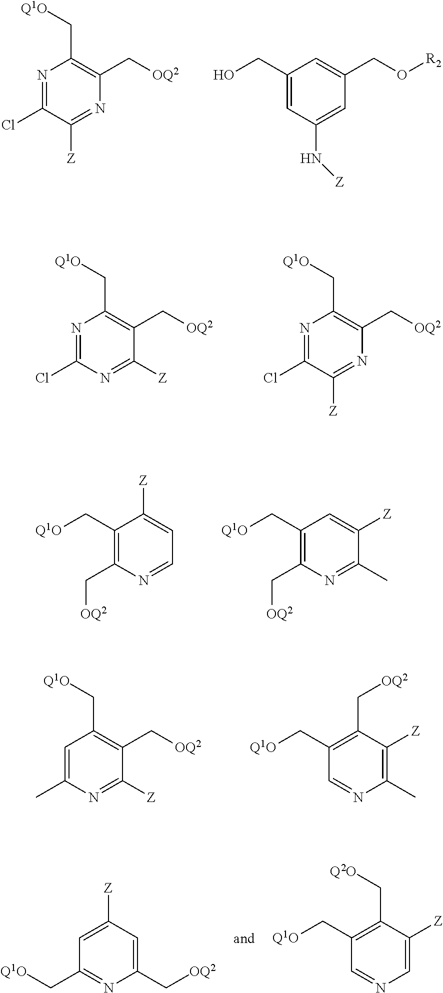
wherein:
Q¹ is hydrogen and Q² is R²; or Q¹ is R² and Q² is hydrogen; and
Z is -L¹-R¹.

30. The conjugate or salt of any one of claims 11-27, which is a compound formula (IIb): 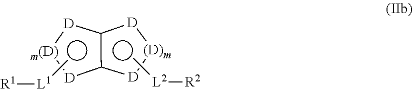
wherein:
each D is independently selected from the group consisting of 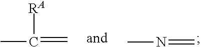
and
each m is independently 1 or 2.

31. The conjugate or salt of any one of claims 11-27, which is selected from the group consisting of: 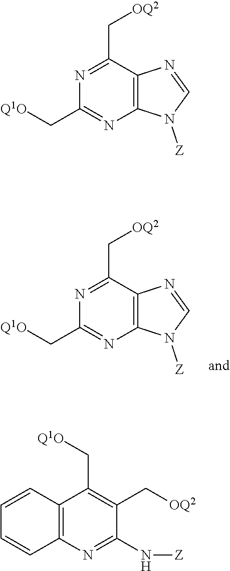
wherein:
Q¹ is hydrogen and Q² is R²; or Q¹ is R² and Q² is hydrogen; and
Z is -L¹-R¹;
and salts thereof.

32. The conjugate or salt of any one of claims 11-27, which a compound formula (Ic): 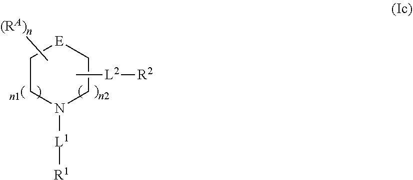
wherein:
E is —O— or —CH₂—;
n is selected from the group consisting of 0, 1, 2, 3, and 4; and
n1 and n2 are each independently selected from the group consisting of 0, 1, 2, and 3;
or a salt thereof.

33. The conjugate or salt of claim 32 that is selected from the group consisting of: 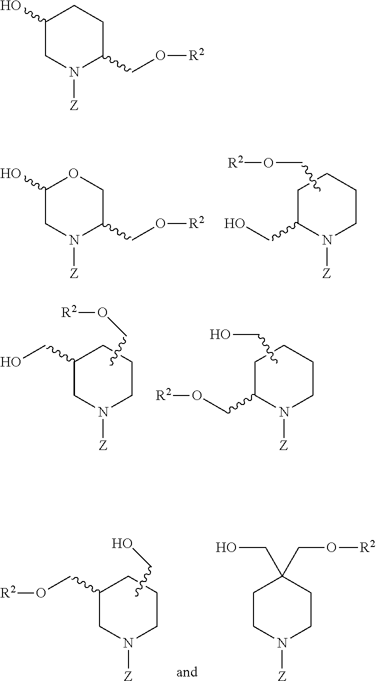
wherein: Z is -L¹-R¹; and salts thereof.

34. The conjugate or salt of any one of claims 11-19, wherein the -A-L²-R² moiety is: 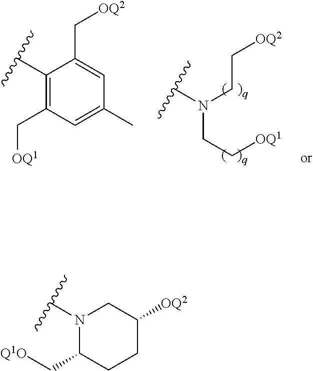
wherein:
Q¹ is hydrogen and Q² is R², or Q¹ is R² and Q² is hydrogen; and
each q is independently 0, 1, 2, 3, 4 or 5.

35. The conjugate or salt of claim 1 that is selected from the group consisting of: 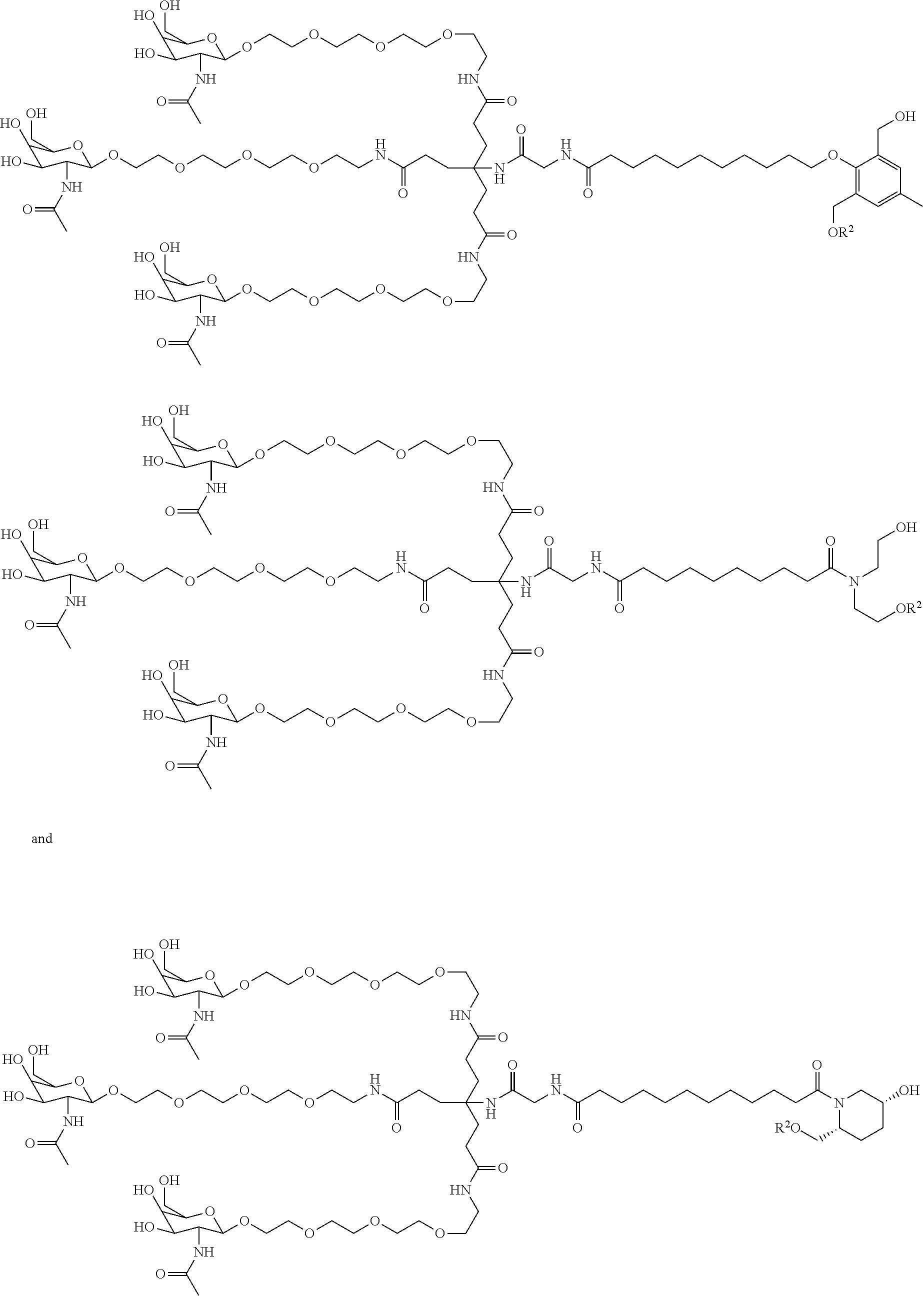
and salts thereof.

36. The conjugate or salt of any one of claims 1-19, wherein R¹ is selected from the group consisting of: 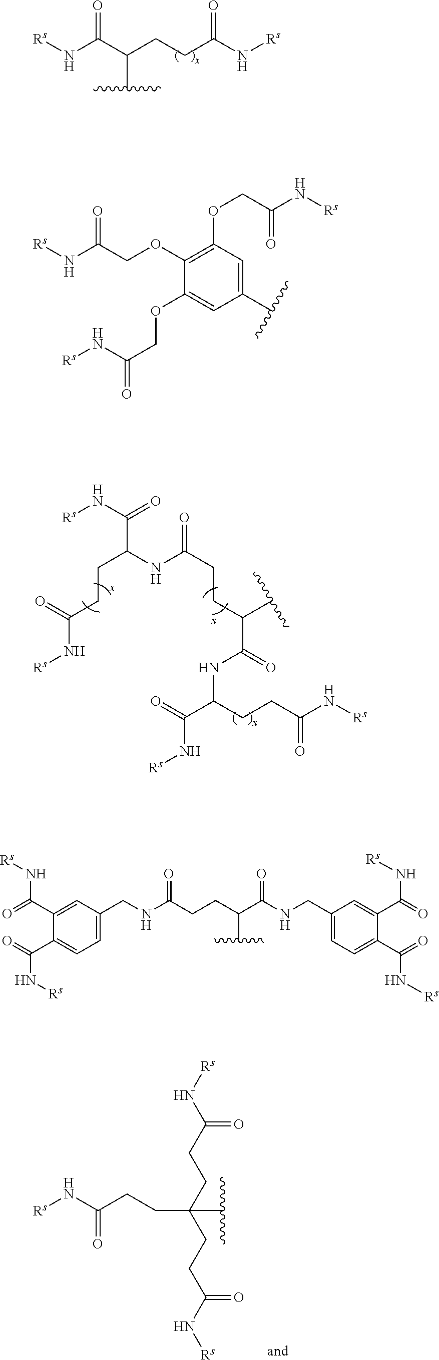 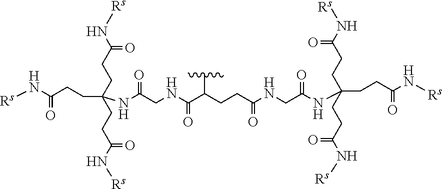
wherein:
R_{S} is 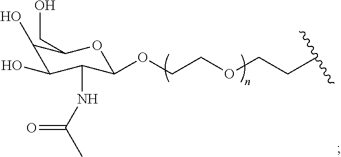
n is 2, 3, or 4; and
x is 1 or 2.

37. The conjugate or salt of any one of claims 1-19, wherein L¹ is selected from the group consisting of:

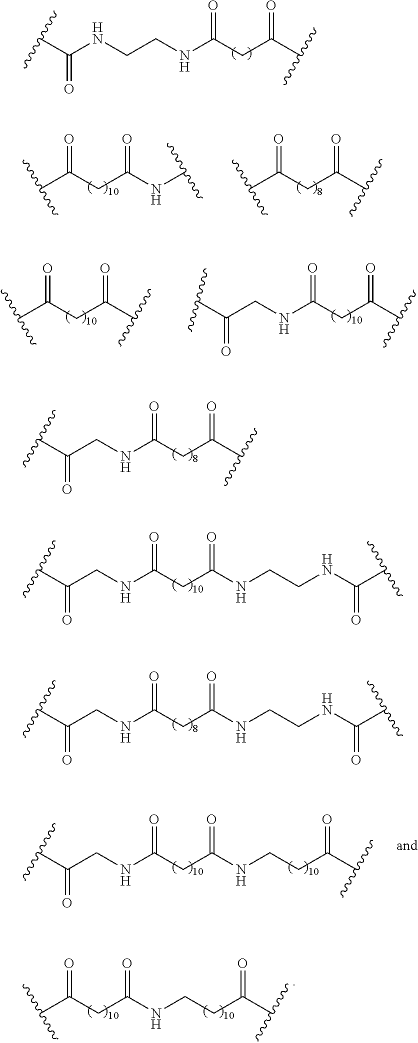

38. The conjugate or salt of any one of claims 1-19, wherein A is absent, phenyl, pyrrolidinyl, or cyclopentyl.

39. The conjugate or salt of any one of claims 1-19, wherein L² is C₁₋₄ allylene-O— that is optionally substituted with hydroxy.

40. The conjugate or salt of any one of claims 1-19, wherein L² is —CH₂O—, —CH₂CH₂O—, or —CH(OH)CH₂O—.

41. The conjugate or salt of any one of claims 11-19, wherein each R^{A} is independently hydroxy or C₁₋₈ alkyl that is optionally substituted with hydroxyl.

42. The conjugate or salt of any one of claims 11-19, wherein each R^{A} is independently selected from the group consisting of hydroxy, methyl and —CH₂OH.

43. The conjugate or salt of any one of claims 11-19, that is a compound formula (IIg): 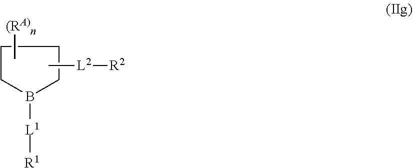
wherein:
B is —N— or —CH—;
L² is C₁₋₄ alkylene-O— that is optionally substituted with hydroxyl or halo; and
n is 0, 1, 2, 3, 4, 5, 6, or 7;
or a salt thereof.

44. The conjugate or salt of claim 43, that is selected from the group consisting of: 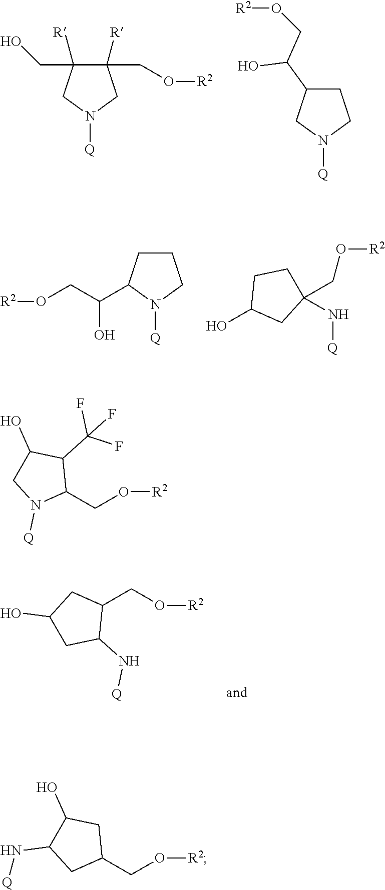
wherein Q is -L¹-R¹; and
R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

45. The conjugate or salt of any one of claims 11-19, that is selected from the group consisting of: 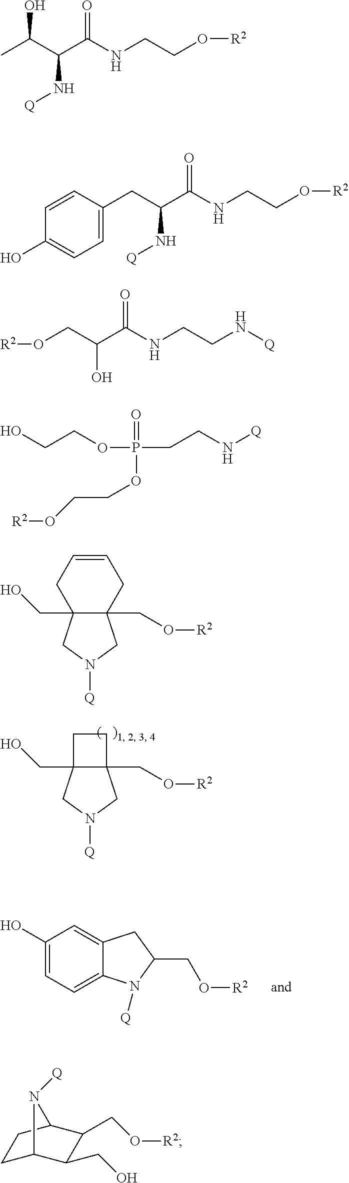
wherein Q is -L¹-R¹.

46. The conjugate or salt of any one of claims 11-19, that is selected from the group consisting of:

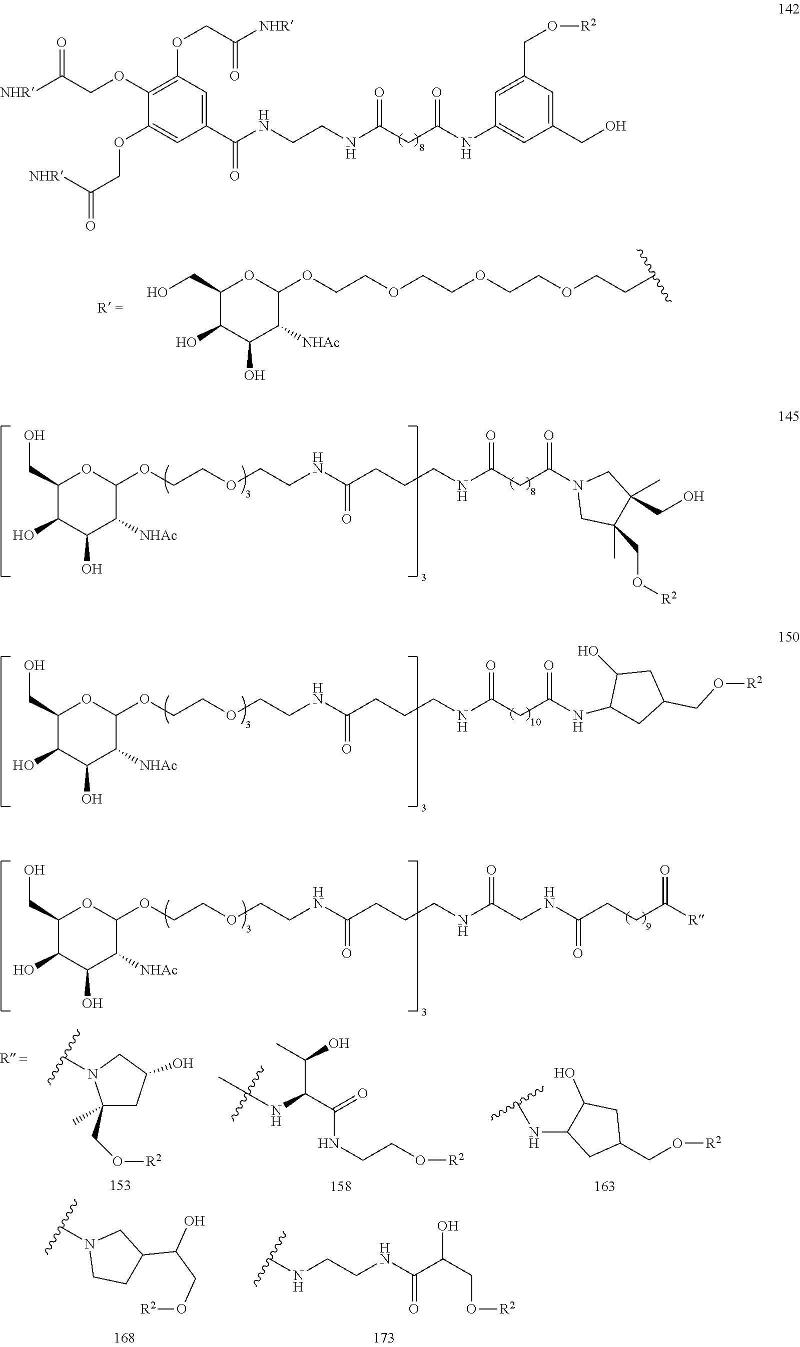

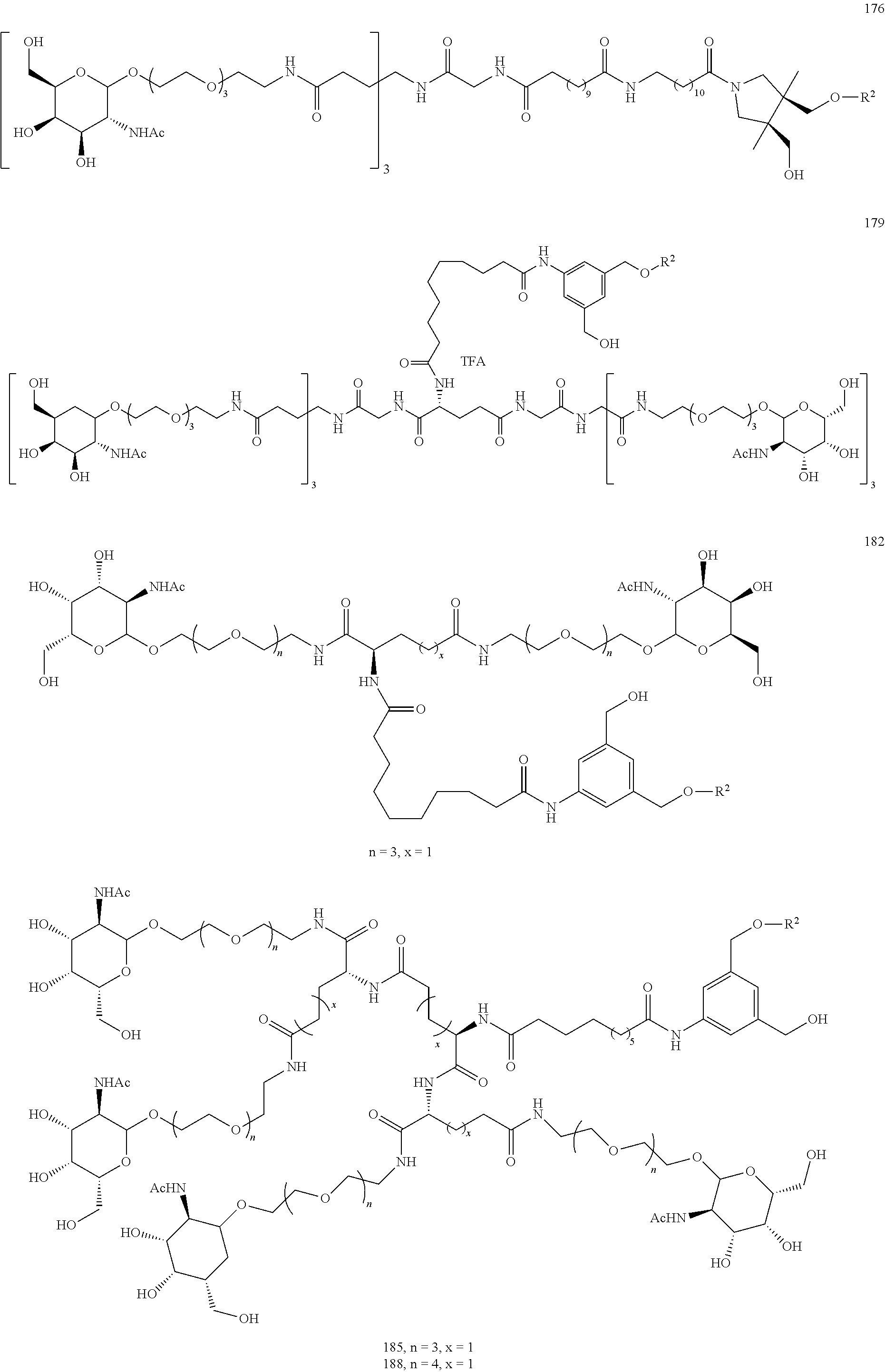

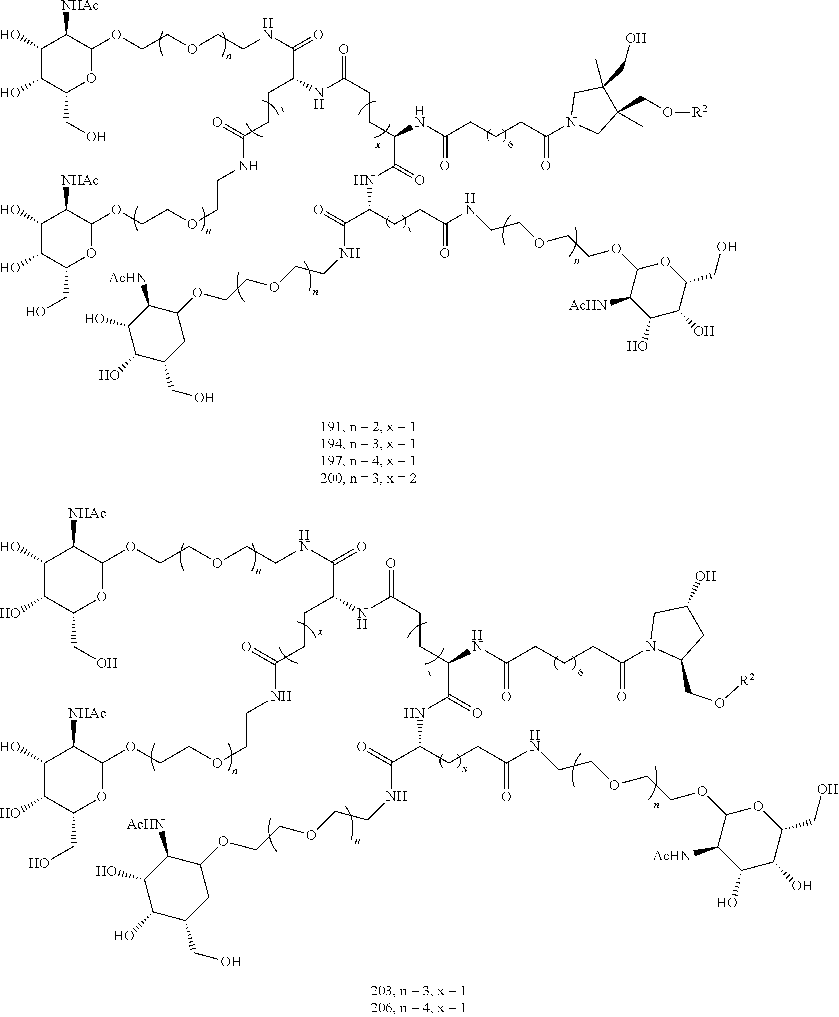

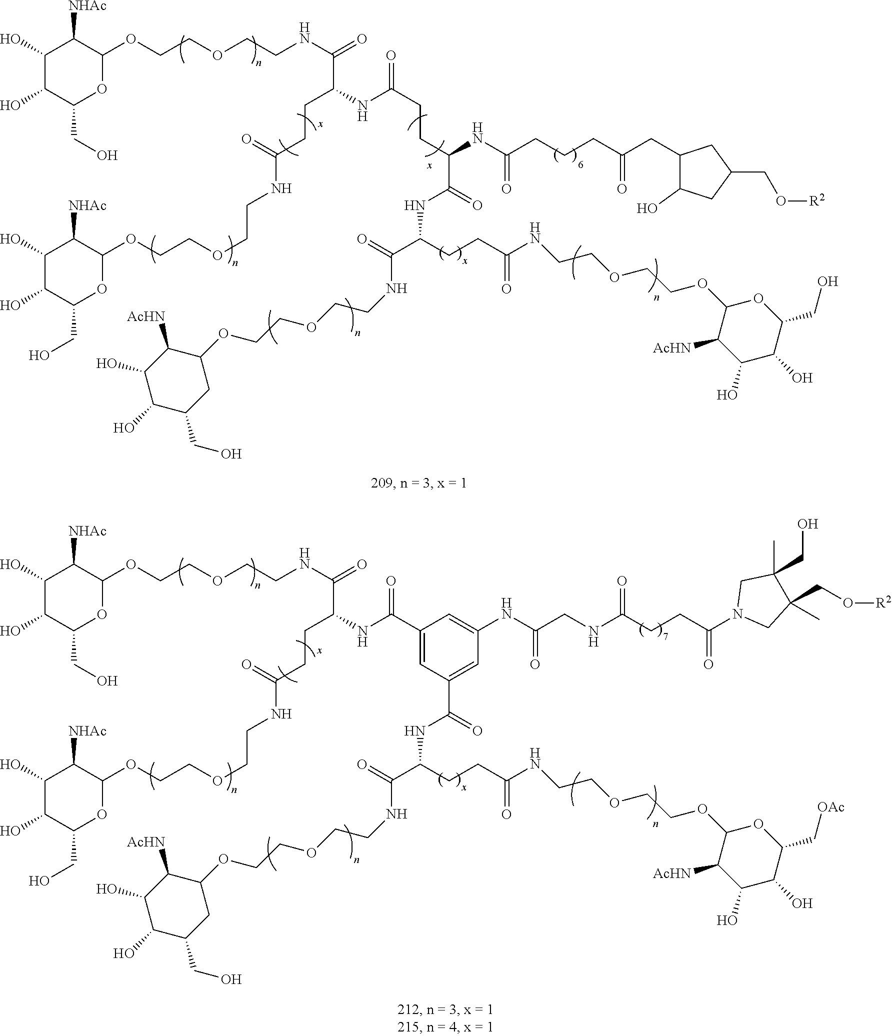

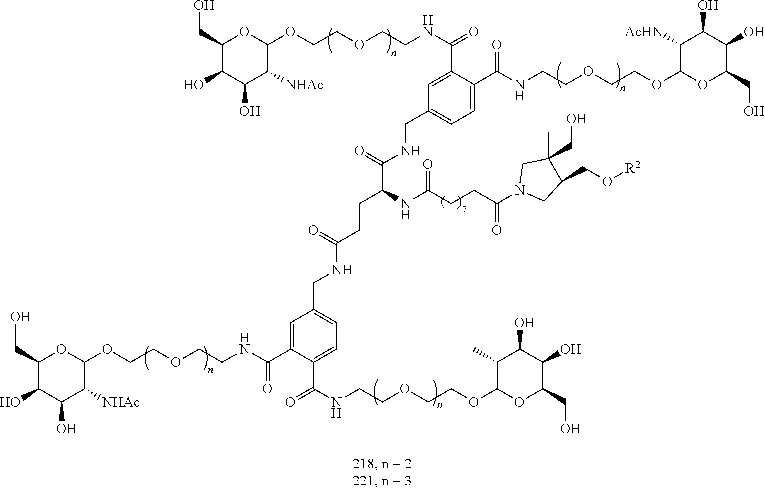

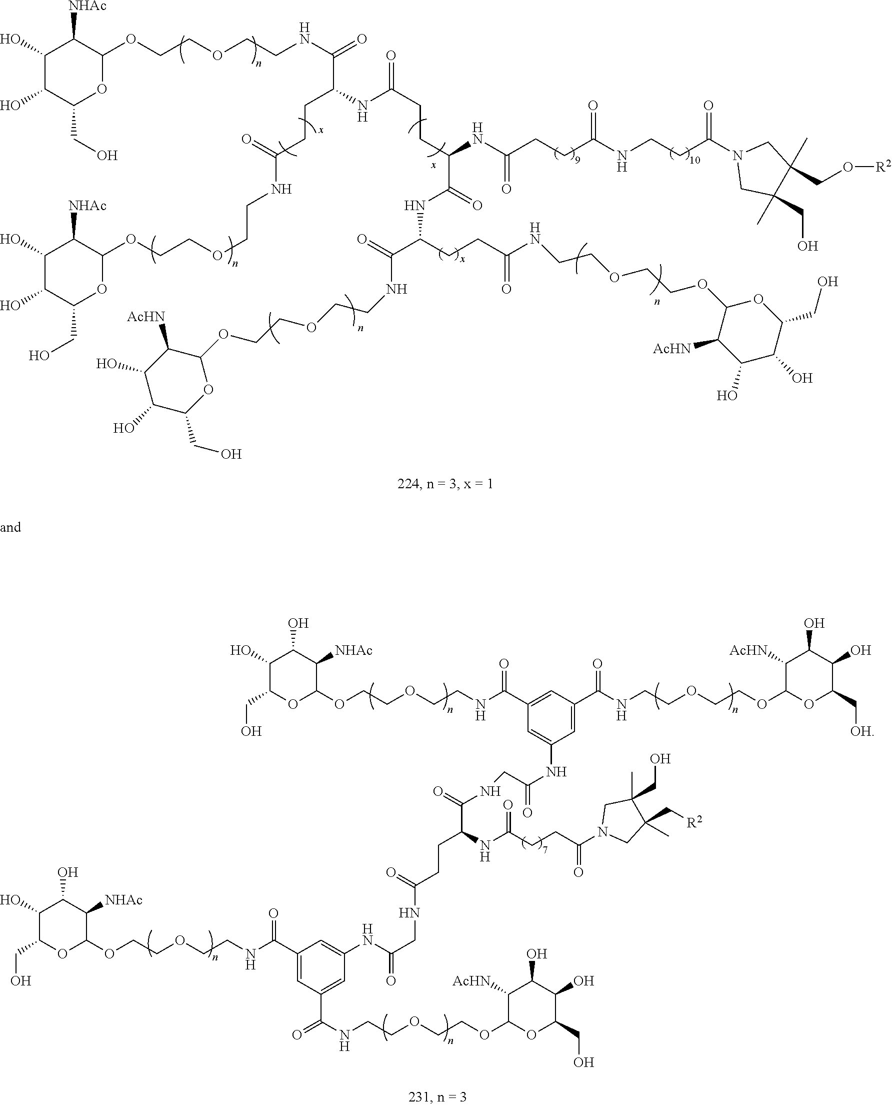

47. The conjugate or salt of any one of claims 1-19, which is a compound formula (IId): 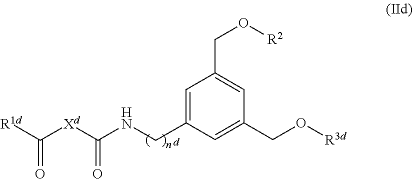
wherein:
R^{1d} is selected from: 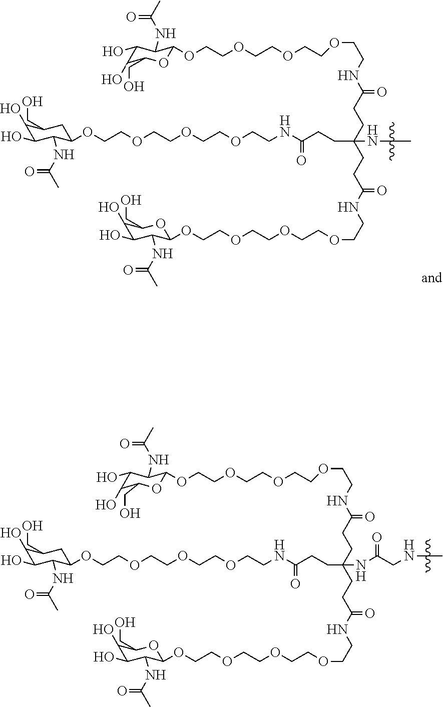 [SMILES: *NC(CCC(=O)NCCOCCOCCOCCOC1CC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1CC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]
X^{d} is C₂₋₁₀ alkylene;
n^{d} is 0 or 1; and
R^{3d} is H, a protecting group, a covalent bond to a solid support, or a bond to a linking group that is bound to a solid support
or a salt thereof.

48. The compound or salt of claim 47, wherein R^{1d} is:

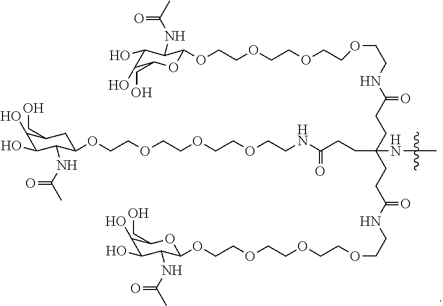 [SMILES: *NC(CCC(=O)NCCOCCOCCOCCOC1CC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

49. The compound or salt of claim 47, wherein R^{1d} is:

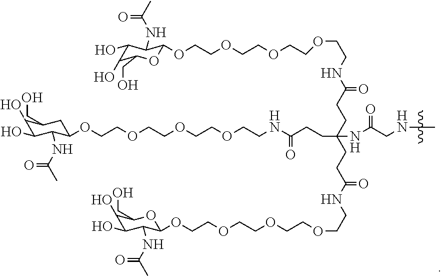 [SMILES: *NCC(=O)NC(CCC(=O)NCCOCCOCCOCCOC1CC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

50. The conjugate or salt of any one of claims 47-49, wherein X^{d} is C₈alkylene.

51. The conjugate or salt of any one of claims 47-49, wherein n^{d} is 0.

52. The conjugate or salt of any one of claims 47-49, wherein R^{3d} is H.

53. The conjugate or salt of any one of claims 47-49, wherein R^{3d} is a covalent bond to a solid support.

54. The conjugate or salt of any one of claims 47-49, wherein R^{3d} is a bond to a linking group that is bound to a solid support, wherein the linking group is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

55. The conjugate or salt of any one of claims 47-49, wherein R^{3d} is a bond to a linking group that is bound to a solid support, wherein the linking group is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo carboxy, aryl, aryloxy, heteroaryl, and heteroaryloxy.

56. The conjugate or salt of any one of claims 47-49, wherein R^{3d} is a bond to a linking group that is bound to a solid support, wherein the linking group
is —C(═O)CH₂CH₂C(═O)N(H)—.

57. The compound or salt of any one of claims 1-10, which is a compound of formula (III): 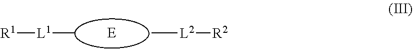
wherein:
R¹ is a targeting ligand that comprises one or more saccharide groups:
L¹ is absent or a linking group:
L² is absent or a linking group;
ring E is divalent and is selected from the group consisting of: 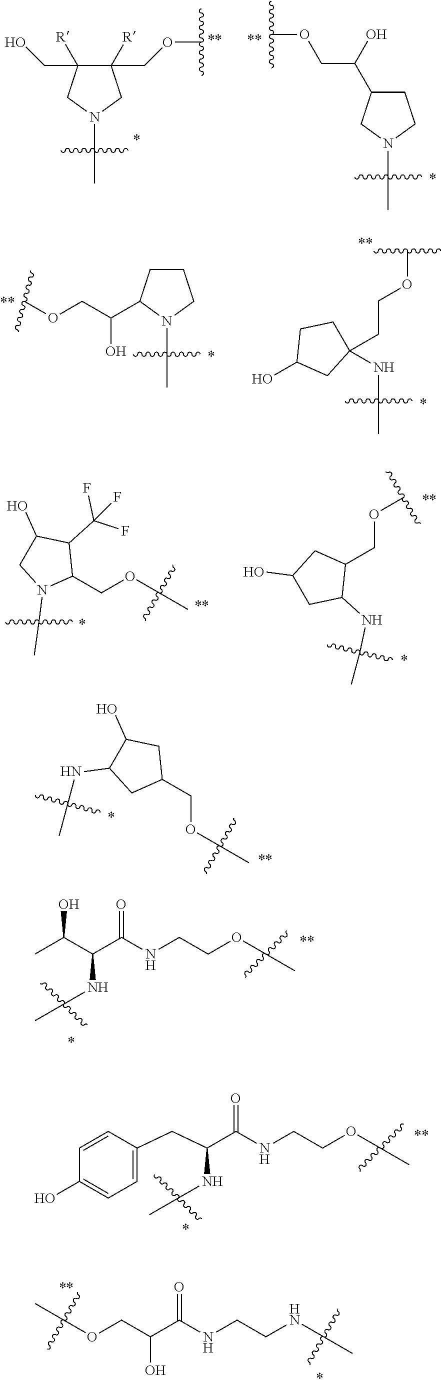 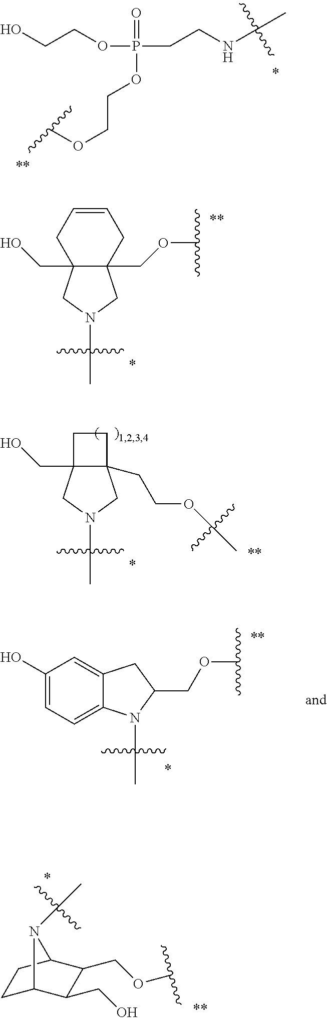
wherein:
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
the valence marked with * is attached to L¹ or is attached to R¹ if L¹ is absent; and
the valence marked with ** is attached to L² or is attached to R² if L² is absent;
or a salt thereof.

58. The compound or salt of claim 57, wherein the targeting ligand R¹ comprises 2-8 saccharides.

59. The compound or salt of claim 57, wherein the targeting ligand R¹ comprises 2-4 saccharides.

60. The compound or salt of claim 57, wherein the targeting ligand R¹ comprises 3-8 saccharides.

61. The compound or salt of claim 57, wherein the targeting ligand R¹ comprises 3-6 saccharides.

62. The compound or salt of claim 57, wherein the targeting ligand R¹ comprises 3 or 4 saccharides.

63. The compound or salt of claim 57, wherein the targeting ligand R¹ comprises 3 saccharides.

64. The compound or salt of claim 57, wherein the targeting ligand R¹ comprises 4 saccharides.

65. The compound or salt of any one of claims 57-64, wherein R¹ has the following formula: 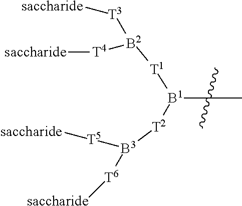
wherein:
B¹ is a trivalent group comprising about 1 to about 20 atoms and is covalently bonded to L¹, T¹, and T².
B² is a trivalent group comprising about 1 to about 20 atoms and is covalently bonded to T¹, T³, and T⁴;
B³ is a trivalent group comprising about 1 to about 20 atoms and is covalently bonded to T², T⁵, and T⁶;
T¹ is absent or a linking group;
T² is absent or a linking group;
T³ is absent or a linking group;
T⁴ is absent or a linking group;
T⁵ is absent or a linking group; and
T⁶ is absent or a linking group.

66. The compound or salt of claim 65, wherein each saccharide is independently selected from: 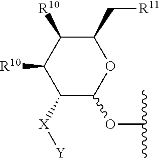
wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy:
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

67. The compound or salt of claim 65, wherein each the saccharide is independently selected from the group consisting of:

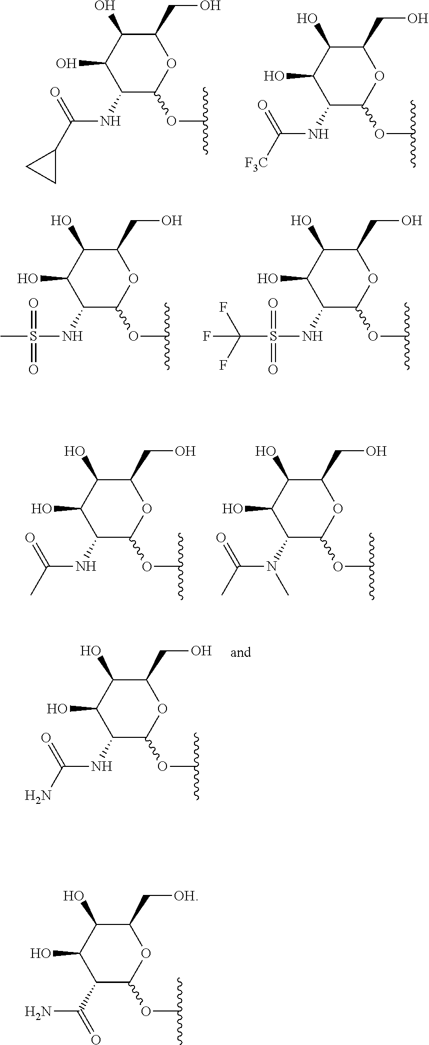

68. The compound or salt of claim 65, wherein each saccharide is independently:

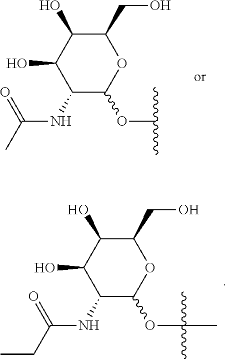 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

69. The compound or salt of any one of claims 65-58, wherein one of T¹ and T² is absent.

70. The compound or salt of any one of claims 65-58, wherein both T¹ and T² are absent.

71. The compound or salt of any one of claims 65-58, wherein each of T¹, T², T³, T⁴, T⁵, and T⁶ is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

72. The compound or salt of any one of claims 65-68, wherein each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

73. The compound or salt of any one of claims 65-68, wherein each of T¹, T², T³, T⁴, T⁵, and T⁶ is independently absent or a branched or unbranched, saturated or unsaturated, hydrocarbon chain, having from 1 to 50 carbon atoms, or a salt thereof, wherein one or more (e.g. 1, 2, 3, or 4) of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

74. The compound or salt of any one of claims 65-68, wherein each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

75. The compound or salt of any one of claims 65-68, wherein each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

76. The compound or salt of any one of claims 65-68, wherein at least one of T³, T⁴, T⁵, and T⁶ is: 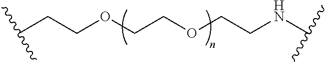
wherein:
n=1, 2, 3.

77. The compound or salt of any one of claims 65-68, wherein each of T³, T⁴, T⁵, and T⁶ is independently selected from the group consisting of: 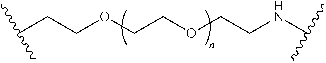
wherein:
n=1, 2, 3.

78. The compound or salt of any one of claims 65-68, wherein at least one of T¹ and T² is glycine.

79. The compound or salt of any one of claims 65-68, wherein each of T¹ and T² is glycine.

80. The compound or salt of any one of claims 65-79, wherein B¹ is a trivalent group comprising 1 to 15 atoms and is covalently bonded to L¹, T¹, and T².

81. The compound or salt of any one of claims 65-79, wherein B¹ is a trivalent group comprising 1 to 10 atoms and is covalently bonded to L¹, T¹, and T².

82. The compound or salt of any one of claims 65-79, wherein B¹ comprises a (C₁-C₆)alkyl.

83. The compound or salt of any one of claims 65-79, wherein B¹ comprises a C₃₋₈ cycloalkyl.

84. The compound or salt of any one of claims 65-79, wherein B¹ comprises a silyl group.

85. The compound or salt of any one of claims 65-79, wherein B¹ comprises a D- or L-amino acid.

86. The compound or salt of any one of claims 65-79, wherein B¹ comprises a saccharide.

87. The compound or salt of any one of claims 65-79, wherein B¹ comprises a phosphate group.

88. The compound or salt of any one of claims 65-79, wherein B¹ comprises a phosphonate group.

89. The compound or salt of any one of claims 65-79, wherein B¹ comprises an aryl.

90. The compound or salt of any one of claims 65-79, wherein B¹ comprises a phenyl ring.

91. The compound or salt of any one of claims 65-79, wherein B¹ is a phenyl ring.

92. The compound or salt of any one of claims 65-79, wherein B¹ is CH.

93. The compound or salt of any one of claims 65-79, wherein B¹ comprises a heteroaryl.

94. The compound or salt of any one of claims 65-79, wherein B¹ is:

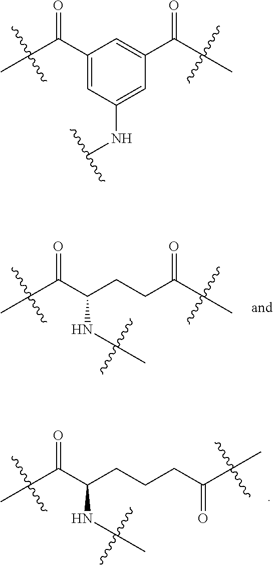 [SMILES: *Nc1cc(C(*)=O)cc(C(*)=O)c1 ; *N[C@@H](CCC(*)=O)C(*)=O ; *N[C@H](CCCC(*)=O)C(*)=O]

95. The compound or salt of any one of claims 65-94, wherein B² is a trivalent group comprising 1 to 15 atoms and is covalently bonded to L¹, T¹, and T².

96. The compound or salt of any one of claims 65-94, wherein B² is a trivalent group comprising 1 to 10 atoms and is covalently bonded to L¹, T¹, and T².

97. The compound or salt of any one of claims 65-94, wherein B² comprises a (C₁-C₆)alkyl.

98. The compound or salt of any one of claims 65-94, wherein B² comprises a C₃₋₈ cycloalkyl.

99. The compound or salt of any one of claims 65-94, wherein B² comprises a silyl group.

100. The compound or salt of any one of claims 65-94, wherein B² comprises a D- or L-amino acid.

101. The compound or salt of any one of claims 65-94, wherein B² comprises a saccharide.

102. The compound or salt of any one of claims 65-94, wherein B² comprises a phosphate group.

103. The compound or salt of any one of claims 65-94, wherein B² comprises a phosphonate group.

104. The compound or salt of any one of claims 65-94, wherein B² comprises an aryl.

105. The compound or salt of any one of claims 65-94, wherein B² comprises a phenyl ring.

106. The compound or salt of any one of claims 65-94, wherein B² is a phenyl ring.

107. The compound or salt of any one of claims 65-94, wherein B² is CH.

108. The compound or salt of any one of claims 65-94, wherein B² comprises a heteroaryl.

109. The compound or salt of any one of claims 65-94, wherein B² is selected from the group consisting of:

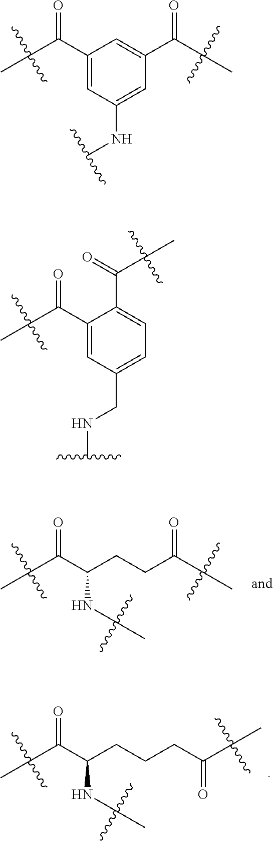 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

110. The compound or salt of any one of claims 65-109, wherein B³ is a trivalent group comprising 1 to 15 atoms and is covalently bonded to L¹, T¹, and T².

111. The compound or salt of any one of claims 65-109, wherein B³ is a trivalent group comprising 1 to 10 atoms and is covalently bonded to L¹, T¹, and T².

112. The compound or salt of any one of claims 65-109, wherein B³ comprises a (C₁-C₆)alkyl.

113. The compound or salt of any one of claims 65-109, wherein B³ comprises a C₃₋₈ cycloalkyl.

114. The compound or salt of any one of claims 65-109, wherein B³ comprises a silyl group.

115. The compound or salt of any one of claims 65-109, wherein B³ comprises a D- or L-amino acid.

116. The compound or salt of any one of claims 65-109, wherein B³ comprises a saccharide.

117. The compound or salt of any one of claims 65-109, wherein B³ comprises a phosphate group.

118. The compound or salt of any one of claims 65-109, wherein B³ comprises a phosphonate group.

119. The compound or salt of any one of claims 65-109, wherein B³ comprises an aryl.

120. The compound or salt of any one of claims 65-109, wherein B³ comprises a phenyl ring.

121. The compound or salt of any one of claims 65-109, wherein B³ is a phenyl ring.

122. The compound or salt of any one of claims 65-109, wherein B³ is CH.

123. The compound or salt of any one of claims 65-109, wherein B³ comprises a heteroaryl.

124. The compound or salt of any one of claims 65-109, wherein B³ is selected from the group consisting of:

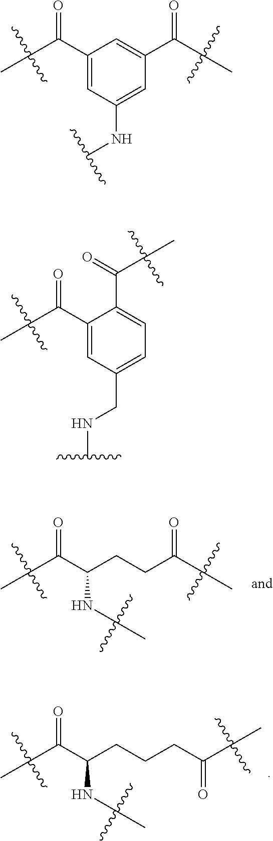 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

125. The compound or salt of any one of claims 65-124, wherein L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

126. The compound or salt of any one of claims 65-124, wherein L¹ is selected from the group consisting of: 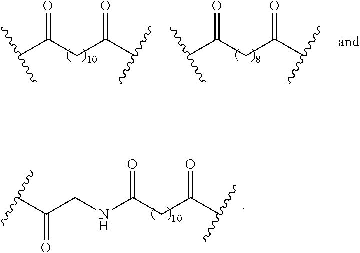
or a salt thereof.

127. The compound or salt of any one of claims 65-124, wherein L¹ is connected to B¹ through a linkage selected from the group consisting of: —O—, —S—, —(C═O)—, —(C═O)—NH—, —NH—(C═O), —NH—(C═O)—NH—, or —NH—(SO₂)—.

128. The compound or salt of any one of claims 65-124, wherein L¹ is selected from the group consisting of:

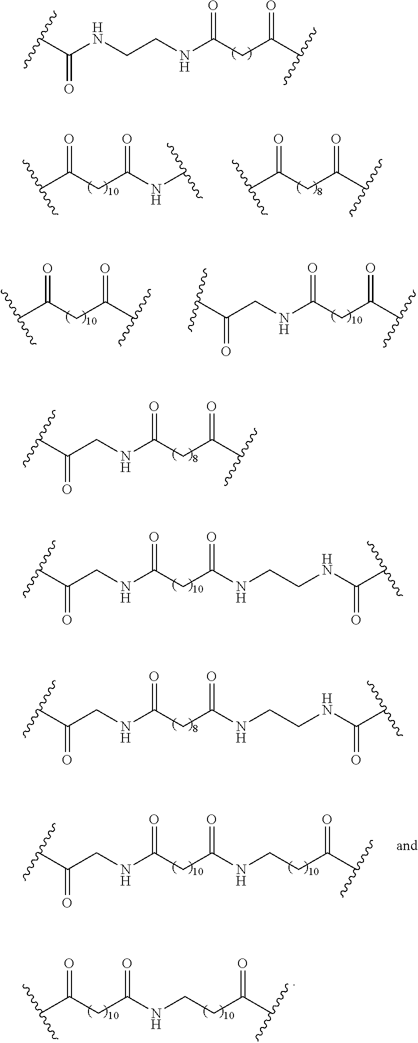

129. The compound or salt of any one of claims 65-128, wherein L² is connected to R² through —O—.

130. The compound or salt of any one of claims 65-128, wherein L² is C₁₋₄ alkylene-O— that is optionally substituted with hydroxy.

131. The compound or salt of any one of claims 65-128, L² is connected to R² through —O—.

132. The compound or salt of any one of claims 65-128, wherein L² is absent.

133. The compound or salt of claim 1 that is selected from the group consisting of: 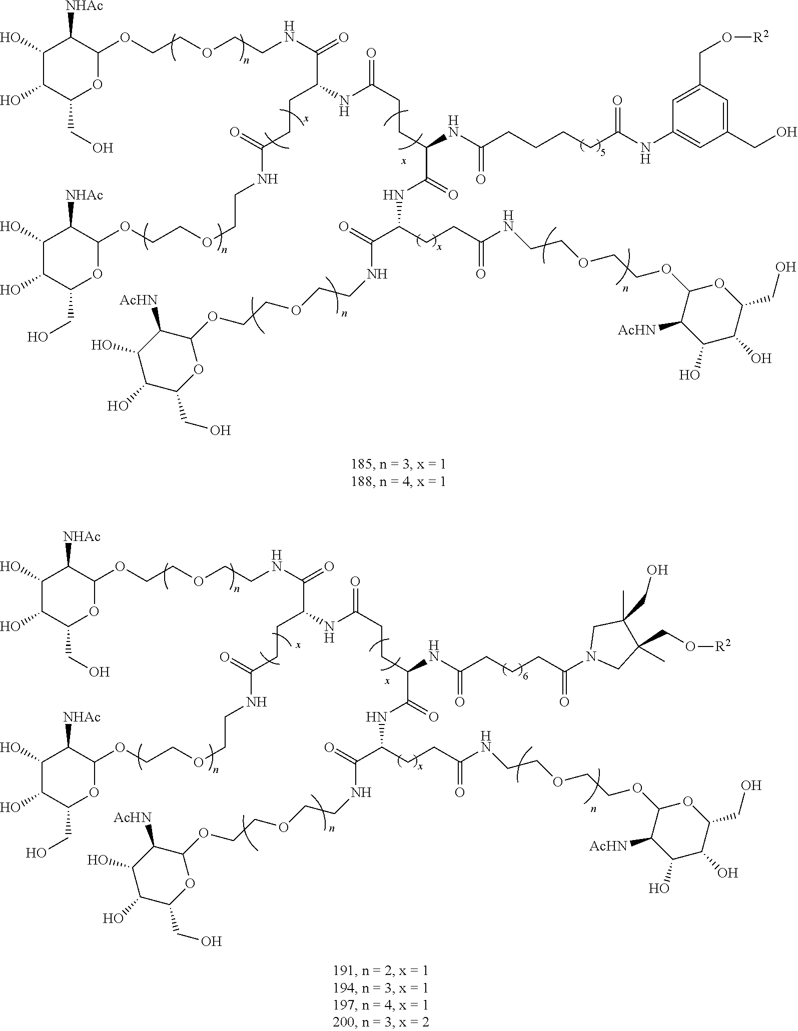 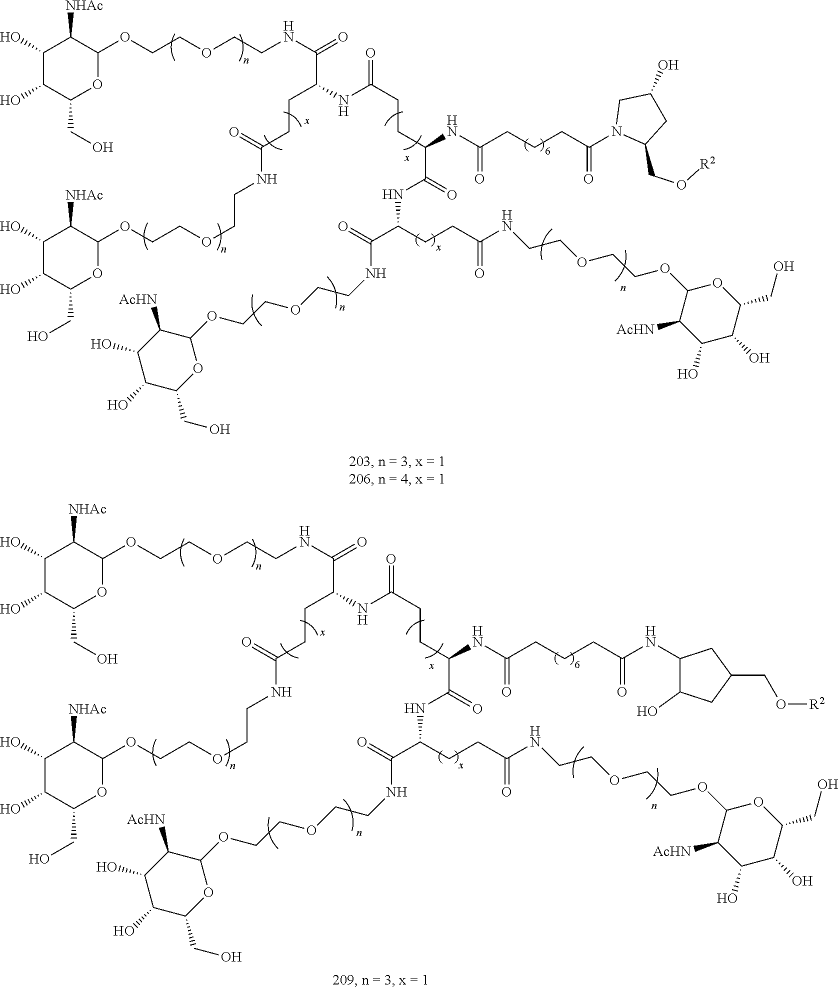 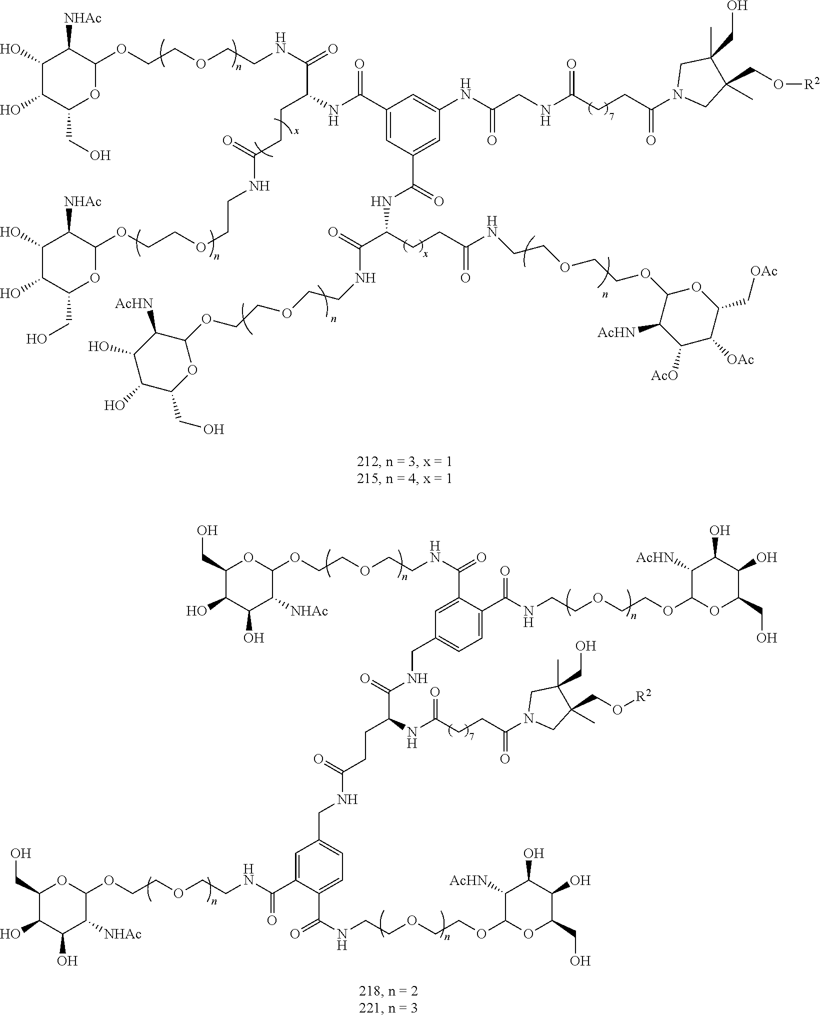 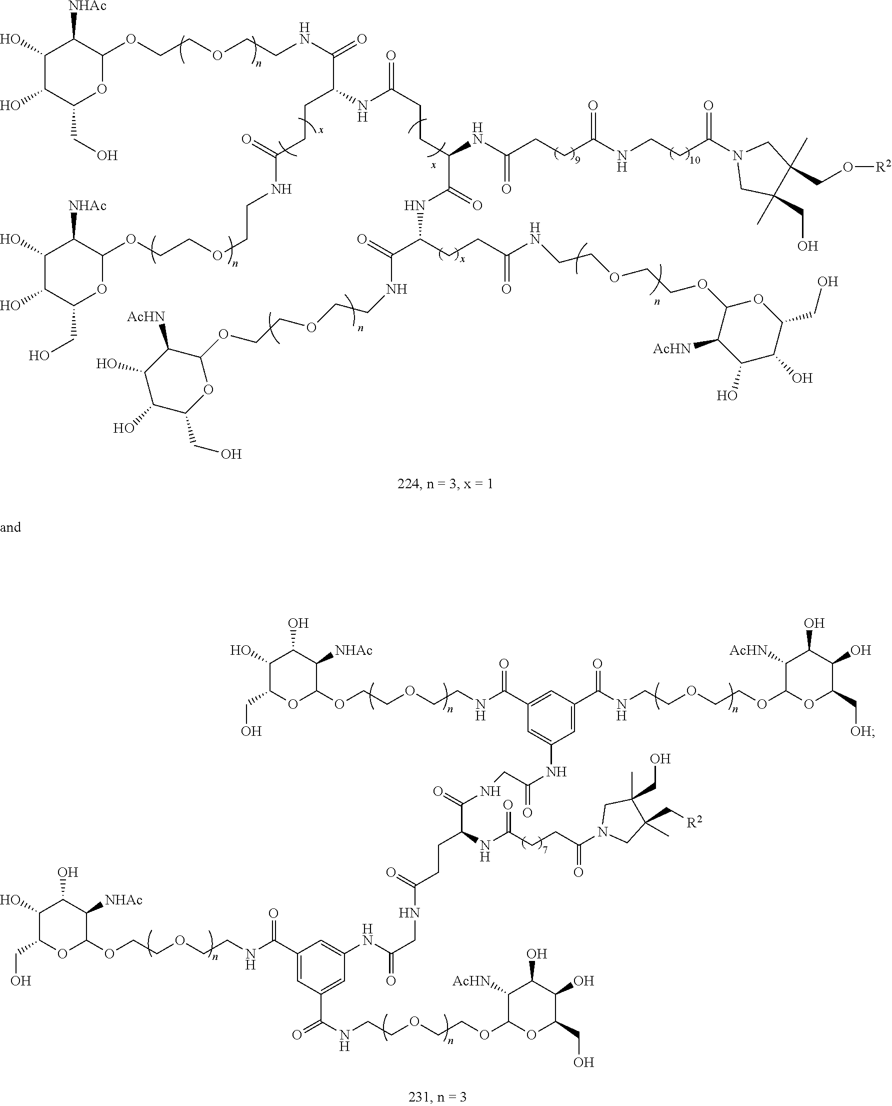
and salts thereof.

134. The compound or salt of claim 1 that is 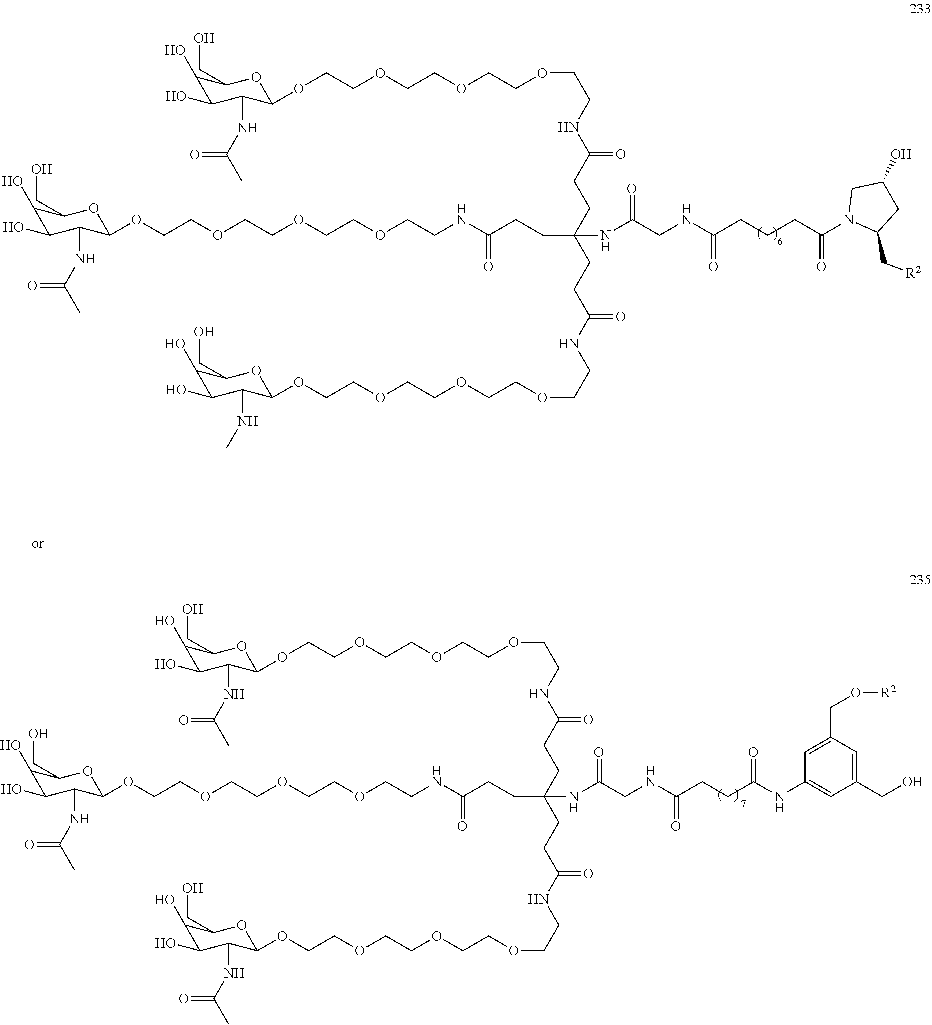
or a salt thereof.

135. The compound or salt of claim 1 that is 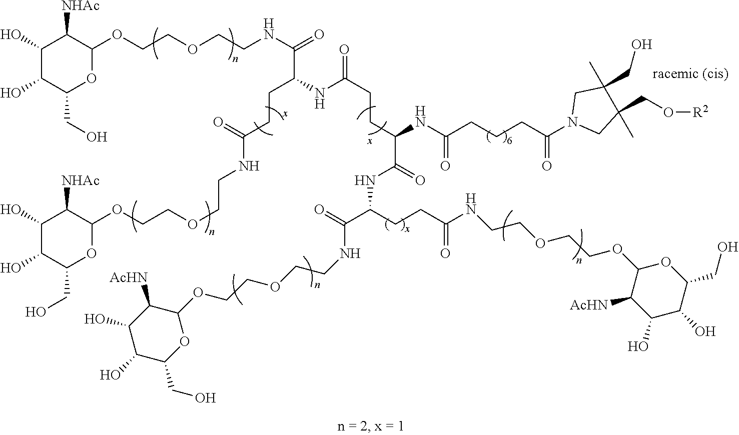
or a salt thereof.

136. The compound or salt of claim 1 that is 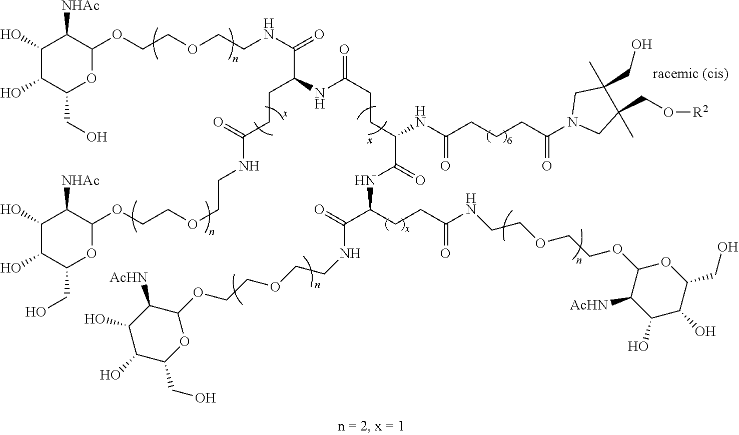
or a salt thereof.

137. The compound or salt of claim 1 that is 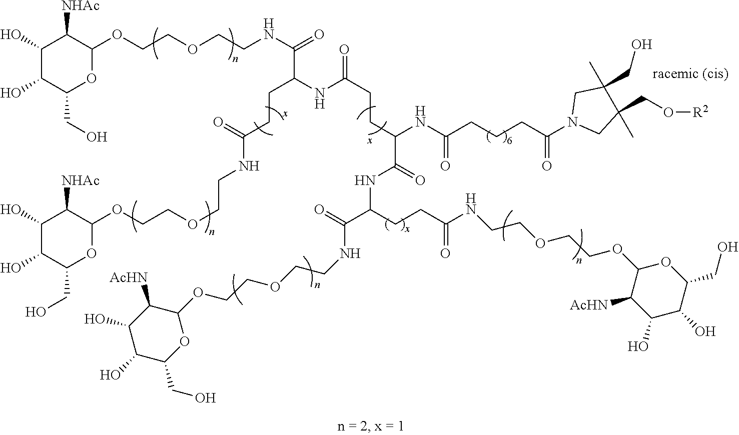
or a salt thereof.

138. The compound or salt of claim 1 that is 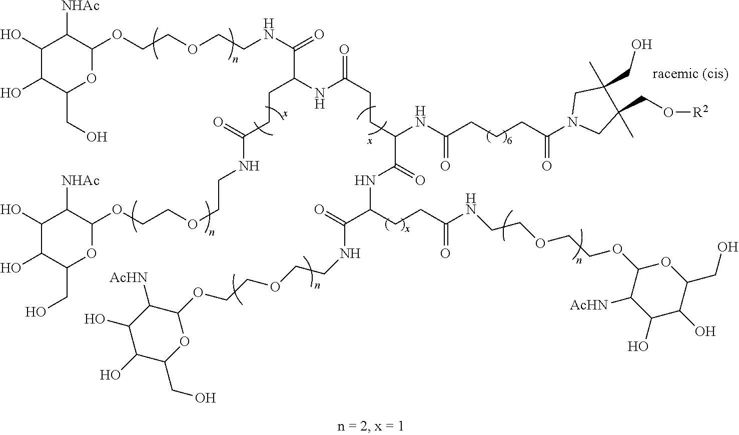
or a salt thereof.

139. The compound or salt of claim 1 that is 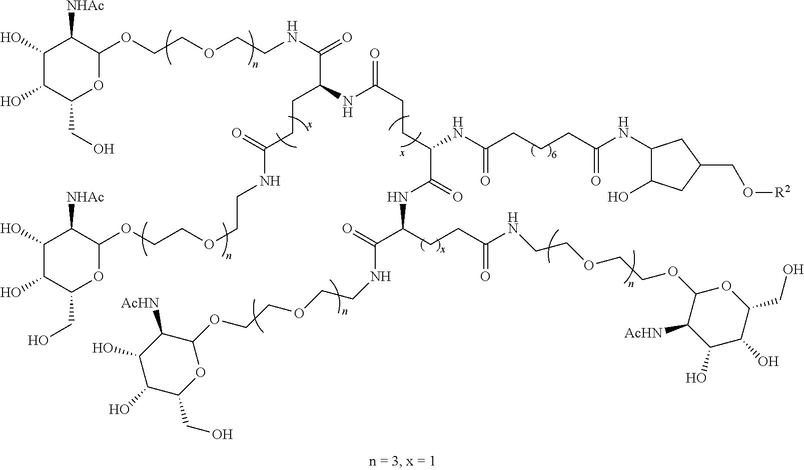
or a salt thereof.

140. The compound or salt of claim 1 that is 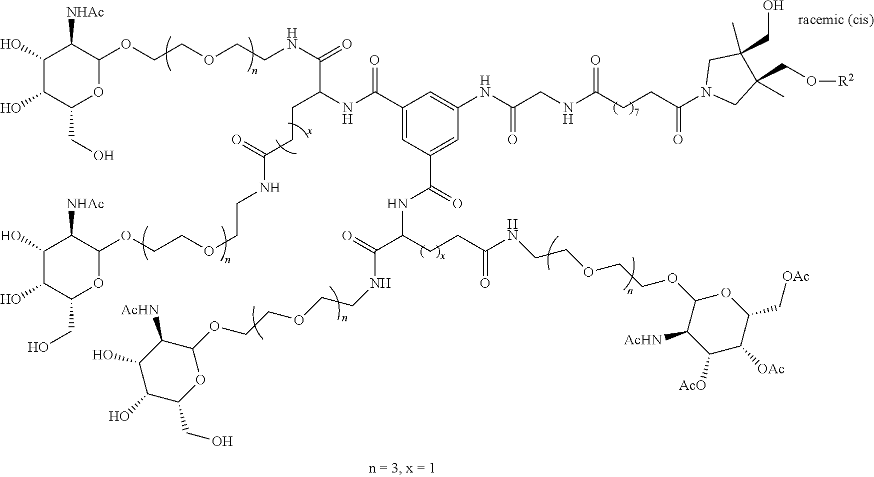
or a salt thereof.

141. The compound or salt of claim 1 that is 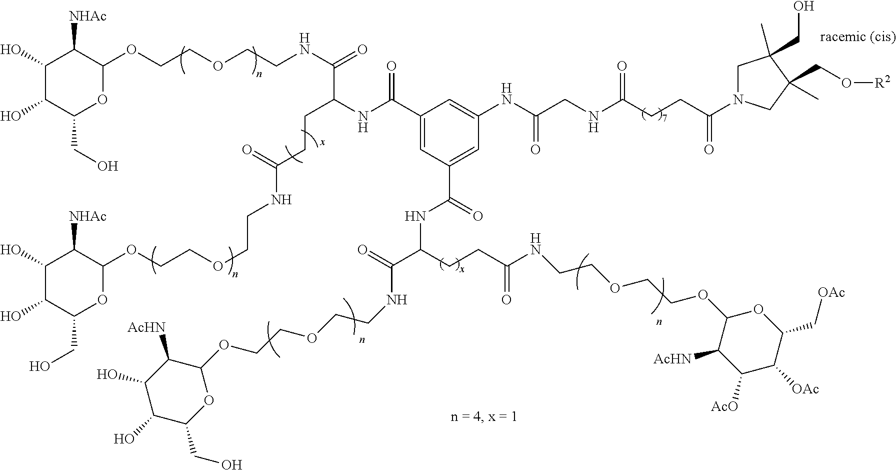
or a salt thereof.

142. The compound or salt of claim 1 that is 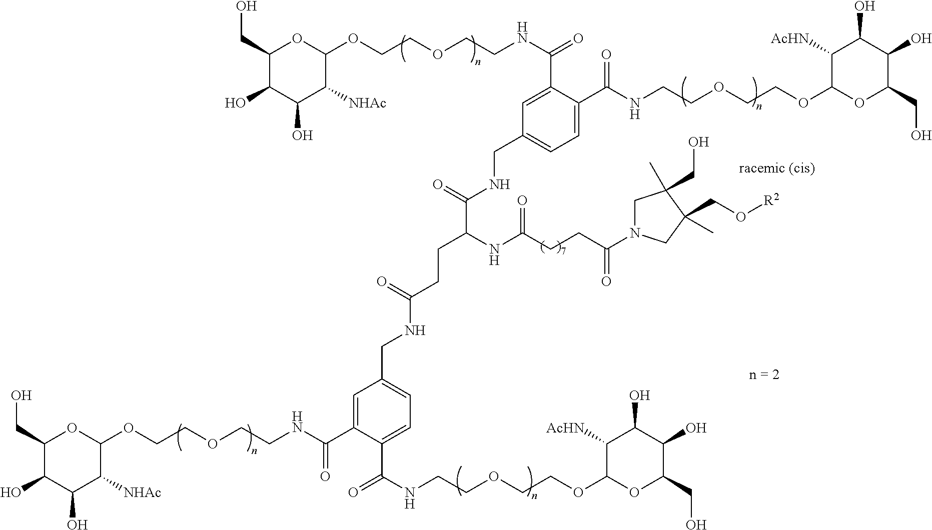
or a salt thereof.

143. The compound or salt of claim 1 that is 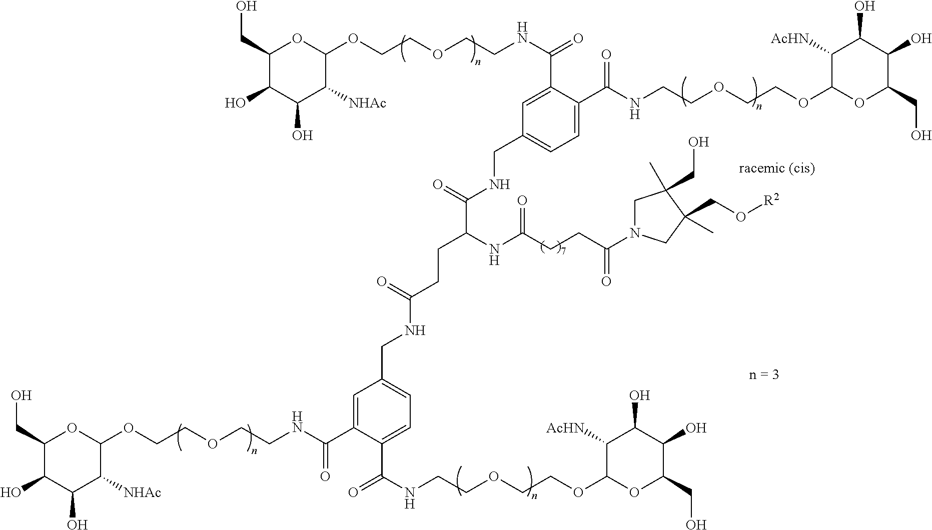
or a salt thereof.

144. The compound or salt of claim 1 that is 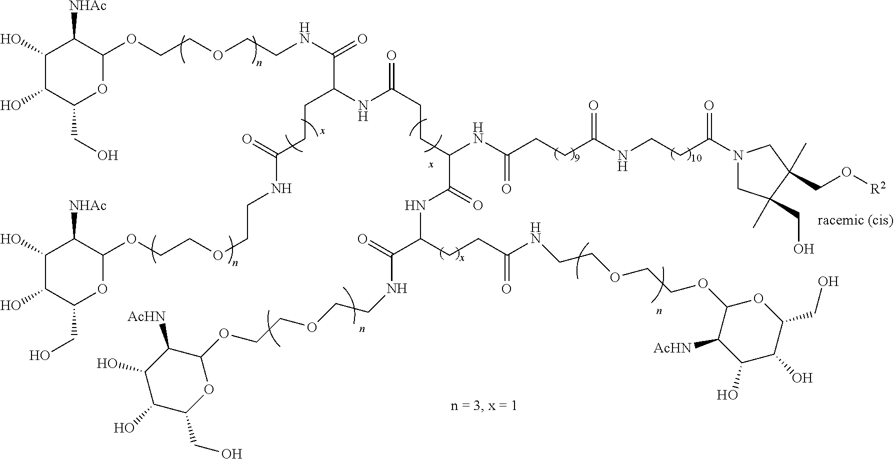
or a salt thereof.

145. The compound or salt of claim 1 that is 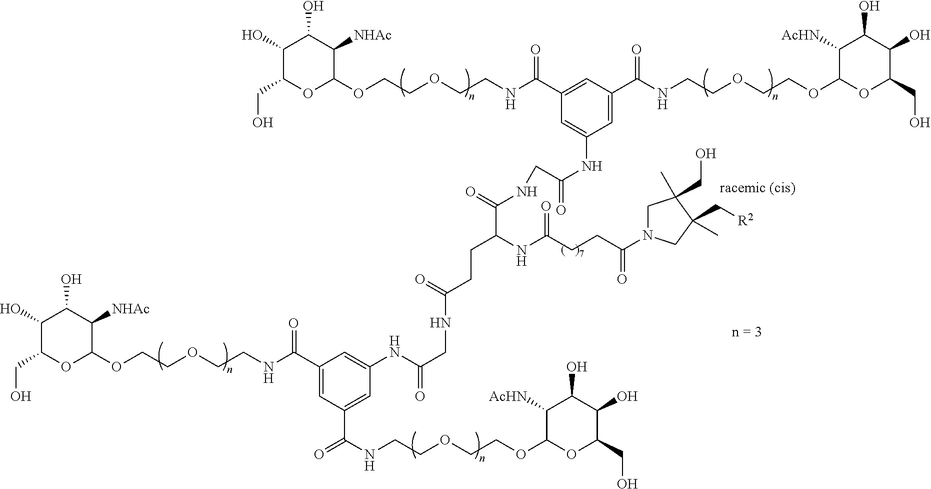
or a salt thereof.

146. The compound or salt of claim 1 that is 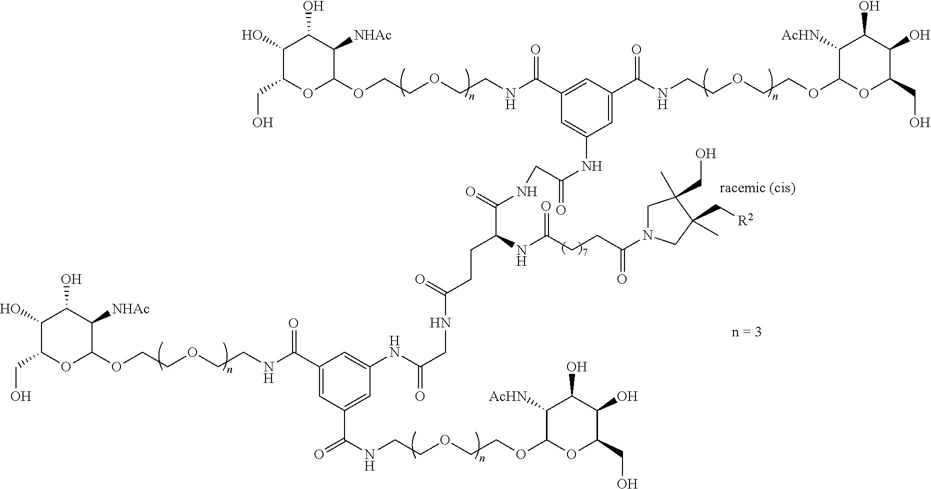
or a salt thereof.

147. The compound or salt of claim 1 that is 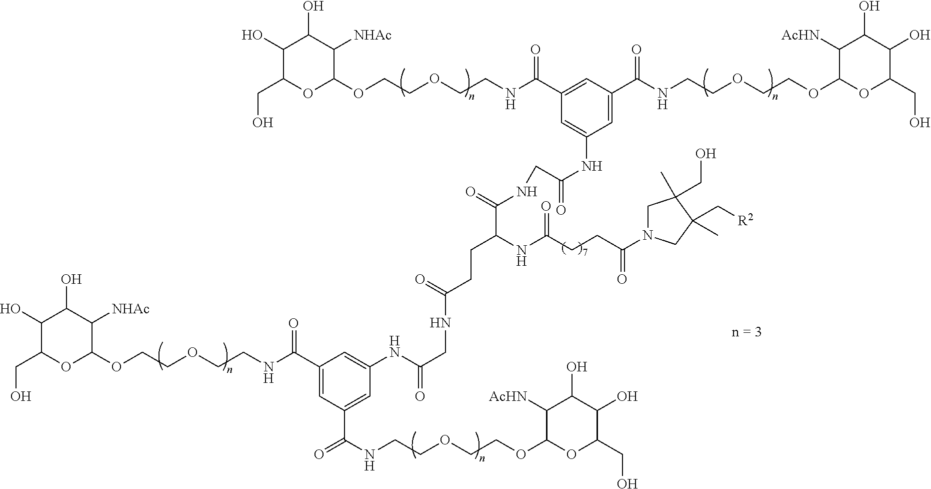
or a salt thereof.

148. The compound or salt of claim 11 that is a compound of formula: 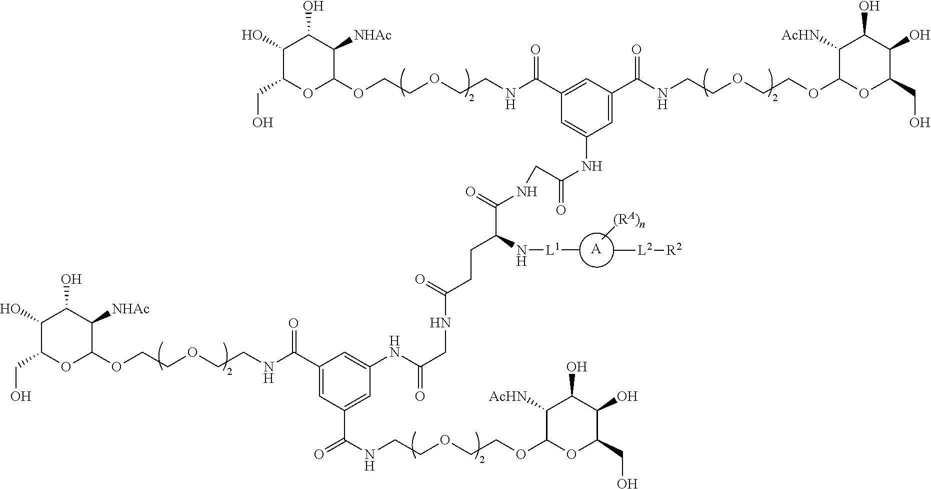
or a salt thereof.

149. The compound or salt of claim 11 that is a compound of formula: 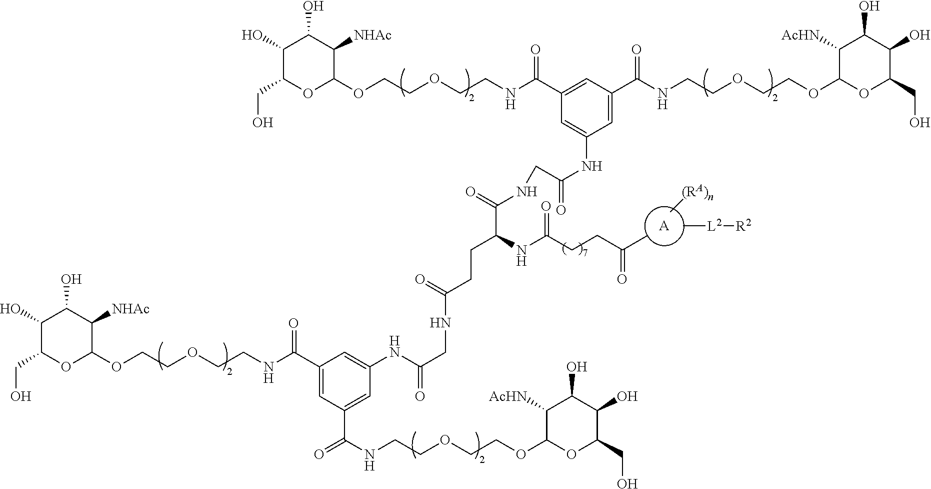
or a salt thereof.

150. A compound or salt of claim 65 that is a compound of formula (IIIa): 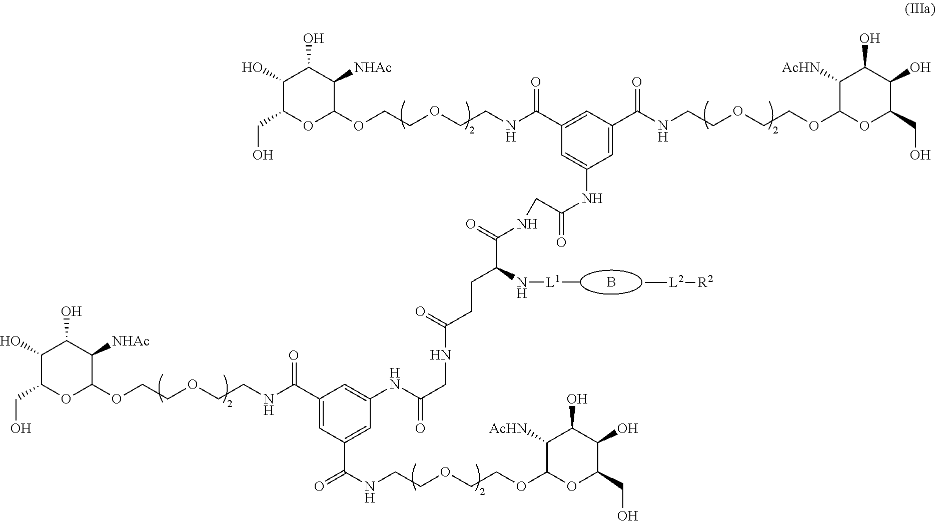
or a salt thereof.

151. The compound or salt of claim 50, wherein L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O), —C(═O)—NR^{X}— or —S—, and wherein R^{X} is hydrogen or (C1-C6)alkyl, and wherein the hydrocarbon chain, is optionally substituted with one or more substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo carboxy, aryl, aryloxy, heteroaryl, and heteroaryloxy.

152. The compound or salt of claim 50, wherein L¹ is selected from the group consisting of: 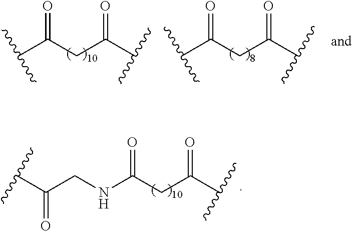
or a salt thereof.

153. The compound or salt of claim 50, wherein L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

154. The compound or salt of claim 50, wherein L¹ is selected from the group consisting of:

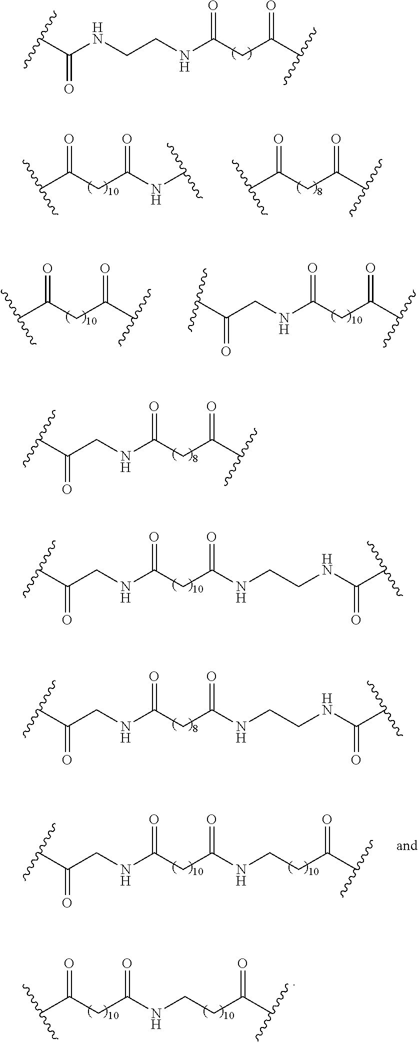

155. The compound or salt of claim 50, wherein L² is connected to R² through —O—.

156. The compound or salt of claim 50, wherein L² is C₁₋₄ alkylene-O— that is optionally substituted with hydroxy.

157. The compound or salt of claim 50, wherein L² is absent.

158. The compound or salt of claim 1 that is 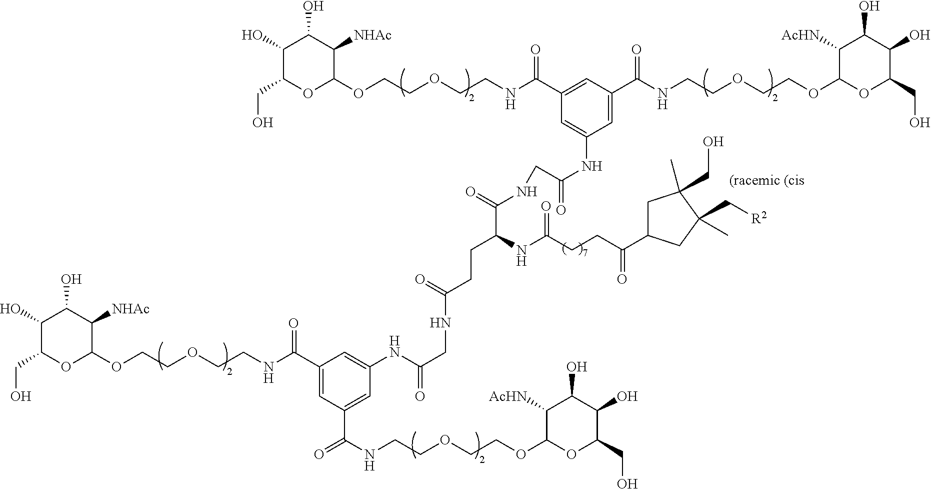
or a salt thereof.

159. The compound or salt of claim 1 that is 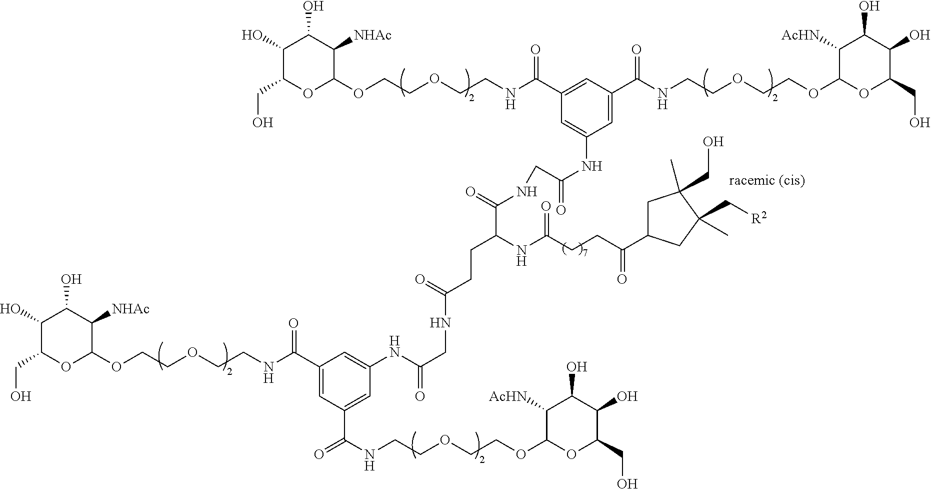
or a salt thereof.

160. The compound or salt of claim 1 that is 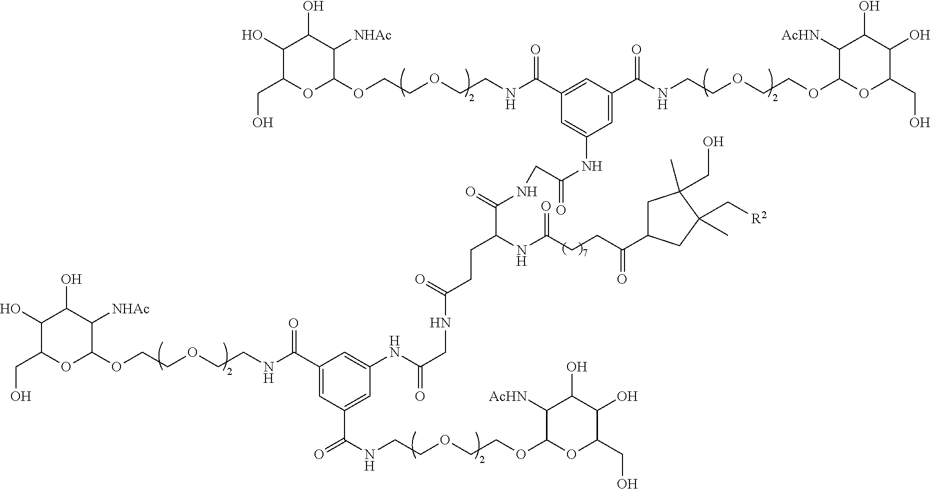
or a salt thereof.

161. The compound or salt of any one of claims 1 and 11-160, wherein R² is an siRNA that comprises SEQ ID NO: 2, 3, 4, 5, 6, 7, 8, 10, or 11 as the antisense strand.

162. The compound or salt of any one of claims 1 and 11-160, wherein R² is an siRNA that comprises SEQ ID NO: 6 or 7 as the antisense strand.

163. The compound or salt of any one of claims 1 and 11-160, wherein R² is an siRNA selected from any one of siRNA 2-9.

164. The compound: 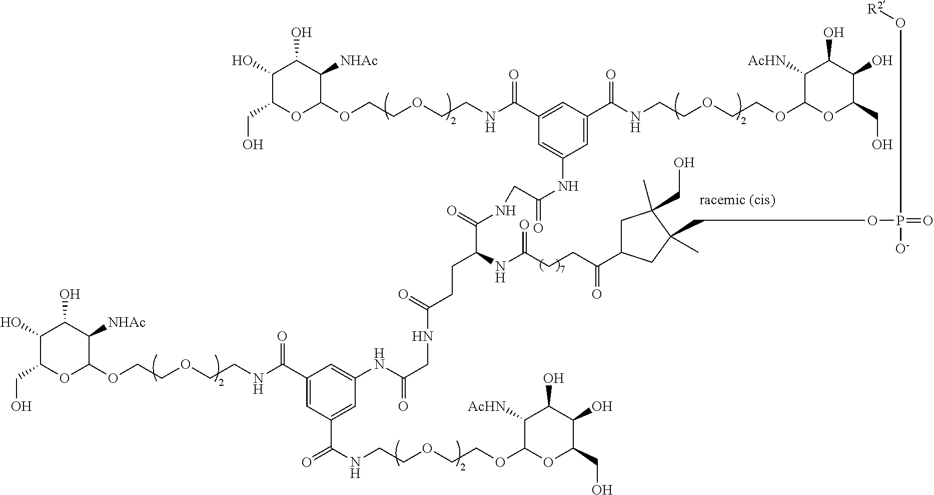
or a salt thereof, wherein R^{2′} is an siRNA that comprises SEQ ID NO: 2, 3, 4, 5, 6, 7, 8, 10, or 11 as the antisense strand.

165. The compound: 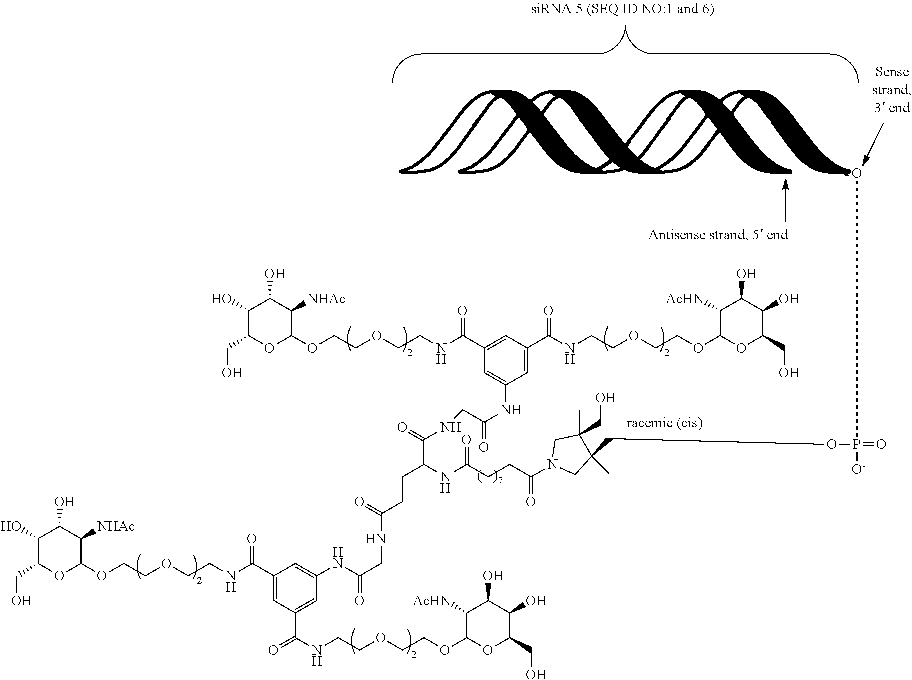
or a salt thereof.

166. The compound: 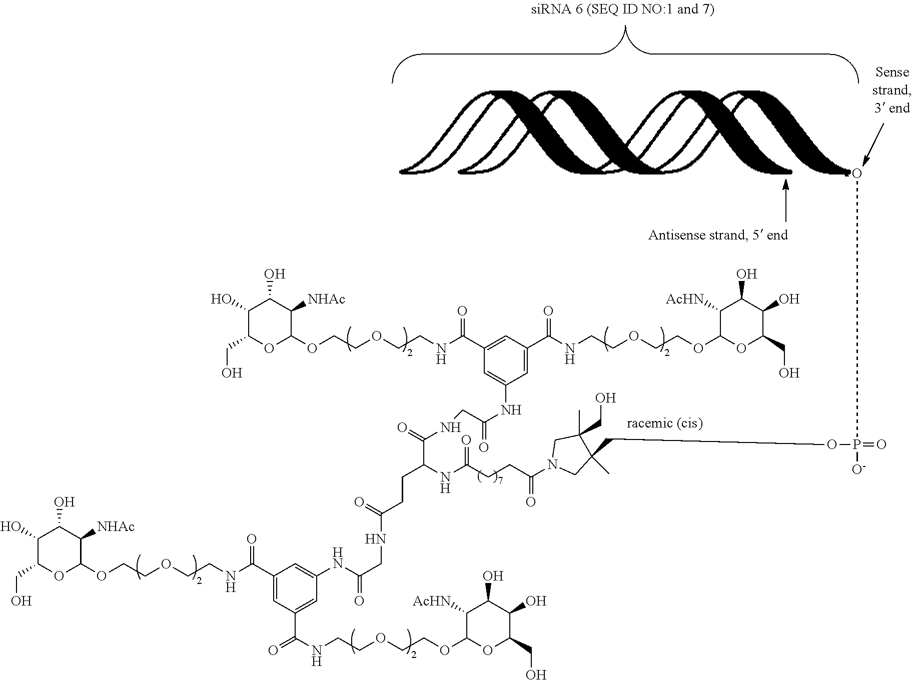
or a salt thereof.

167. A pharmaceutical composition comprising a compound as described in any one of claims 1-166 or a pharmaceutically acceptable salt thereof, and a pharmaceutically acceptable carrier.

168. A method to deliver an siRNA to the liver of an animal comprising administering a compound as described in any one of claims 1-166 or a pharmaceutically acceptable salt thereof to the animal.

169. A method to treat a hepatitis B viral infection in a human comprising administering an effective amount of a compound as described in any one of claims 1-166 or a pharmaceutically acceptable salt thereof to the human.

170. The method of claim 169, wherein the compound or salt thereof is administered subcutaneously.

171. The method of any one of claims 168-170, wherein the compound or a salt thereof is administered in combination with another therapeutic agent.

172. A siRNA molecule that comprises at least one unlocked nucleic acid (UNA) that is any one of the siRNA from Table A that comprises a replacement of a nucleotide with a UNA, e.g., in the antisense strand, e.g., at position(s) 5 and or 6 of the antisense strand.

173. A siRNA molecule selected from any one of siRNA 2-9.
